SEQUENCE LISTING (42 sequences)

SEQ ID NO: 1 - DNA, 2643 bp, Human poliovirus 1
  source 1..2643: mol_type = unassigned DNA
atgggtgctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacagccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgagggacatgggactctttgggcaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacaacaaccagacatcacctgcccgcaggttctgcccggtggattacctccttggaaatggcacgttgttggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgaagtaaagaacatgatggaattggcagaaatcgacaccatgattccctttgacttaagtgccacaaaaaagaacaccatggaaatgtatagggttcggttaagtgacaaaccacatacagacgatcccatactctgcctgtcactctctccagcttcagatcctaggttgtcacatactatgcttggagaaatcctaaattactacacacactgggcaggatccctgaagttcacgtttctgttctgtggattcatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctatttgcagtgtggaagatcacttataaagatactgtccagttacggaggaaattggagttcttcacctattctagatttgatatggaacttacctttgtggttactgcaaatttcactgagactaacaatgggcatgccttaaatcaagtgtaccaaattatgtacgtaccaccaggcgctccagtgcccgagaaatgggacgactacacatggcaaacctcatcaaatccatcaatcttttacacctacggaacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatat

SEQ ID NO: 2 - DNA, 7521 bp, unidentified
  misc_feature 1..7521: note = synthetic
  source 1..7521: mol_type = other DNA
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgctcaggtttcgtcgcaaaaagtaggtgcgcatgaaaactcgaatcgtgcgtatggttcgtcgacgataaattatacgacgataaattattatcgtgattcggcgtctaatgcggcttcgaaacaagatttttcgcaagatccgtcgaaatttacggaaccgataaaagatgtattaataaaaacggcgccgatgttaaattcgccgaatatagaagcgtgtggttattcggatcgtgtattacaattaacgttaggtaattcgacgataacgacgcaagaagcggctaattcggtagtagcgtatggtcgttggccggagtatttacgtgattcggaagcgaatccggtagatcaaccgacggaaccggacgtcgcggcgtgtcgtttttatacgttagatacggtatcgtggacgaaagaatcgcgaggttggtggtggaaattaccggatgcgttacgtgatatgggtttatttggtcaaaatatgtattatcattatttaggtcgttcgggttatacggtacatgtacaatgtaatgcgtcgaaatttcatcaaggtgcgttaggtgtatttgcggtaccggaaatgtgtttagcgggtgactcgaatacgacgacgatgcatacgtcgtatcaaaatgcgaatccgggtgaaaaaggtggtacgtttacgggtacgtttacgccggataataatcaaacgtcgccggcgcgtcgtttttgtccggtagattatttattaggtaatggtacgttattagggaatgcctttgtatttccgcatcaaataataaatttgcgtacgaataattgtgcgacgttagtattaccgtatgtaaattcgttatcgatagattcgatggtaaaacataataattggggtatagcaatattaccgttagcgccgttaaattttgcgtcggaatcgtcgccggaaataccgataacgttaacgatagcgccgatgtgttgtgaatttaatggtttgcgtaatataacgttaccgcgtttacaaggtttaccggtaatgaatacgccgggctcgaatcagtatttaacggcggataattttcaatcgccgtgtgcgttaccggaatttgatgtaacgccgccgatagatataccgggtgaagtaaaaaatatgatggaattagcggaaatagatacgatgataccgtttgatttatcggcgacgaaaaaaaatacgatggaaatgtatcgtgtacgtttatcggataaaccgcatacggatgatccgatattatgtttatcgttatcgccggcgtcggatcctaggttatcgcatacgatgttaggtgagatattaaattattatacgcattgggcgggttcgttaaaatttacgtttttattttgtggttcgatgatggcgacgggtaaattattagtatcgtatgcgccgccgggtgcggatccgccgaaaaaacgtaaagaagcgatgttaggtacgcatgtaatatgggatataggtttacaatcgtcgtgtacgatggtagtaccgtggatatcgaatacgacgtatcgtcaaacgatagatgattcgtttacggaaggtggttatatatcggtattttatcaaacgcgtatagtagtaccgttatcgacgccgcgtgaaatggatatattaggttttgtatcggcgtgtaatgatttttcggtacgtttattacgtgatacgacgcatatagaacaaaaagcgctagcacaaggtttaggtcaaatgttagaatcgatgatagataatacggtacgtgaaacggtaggtgcggctacgtcgcgtgatgcgttaccgaatacggaagcgtcgggtccgacgcactcgaaagaaataccggcgttaacggcggtagaaacgggtgcgacgaatccactagtcccgtcggatacggtacaaacgcgtcatgtagtacaacatcgttcgcgttcggaatcgtcgatagaatcgttttttgcgcgtggtgcgtgtgtaacgataatgacggtagataatccggcgtcgacgacgaataaagataaattatttgcggtatggaaaataacgtataaagatacggtacaattacgtcgtaaattagaatttttcacgtattcgcgttttgatatggaattaacgtttgtagtaacggcgaattttacggaaacgaataatggtcatgcgttaaatcaagtatatcaaataatgtacgtaccgccgggtgcgccggtacccgaaaaatgggatgattatacgtggcaaacgtcgtcgaatccgtcgatattttacacgtatggtacggcgccggcgcgtatatcggtaccgtatgtaggtatatctaatgcgtattcgcatttttatgatggtttctcgaaagtaccgttaaaagatcaatcggcggcgttaggtgattcgttatatggtgcggcgtcgttaaatgattttggtatattagcggtacgtgtagtaaatgatcataatccgacgaaggtcacctcgaaaatacgtgtatatttaaaaccgaaacatatacgtgtatggtgtccgcgtccgccgcgtgcggtagcgtattatggtccgggtgtagattataaagatggtacgttaacgccgttatcgacgaaagatttaacgacgtatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcattttcagacattagagacttgtatgcctacgaagaagaagccatggaacaaggcctcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactcgagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgacgcgttagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagttaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacatagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggacaaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactggtgtaggggtaaatttttctttaattcggaggaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 7521 bp, unidentified
  misc_feature 1..7521: note = synthetic
  source 1..7521: mol_type = other DNA
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgctcaggtttcgtctcagaaggtaggtgctcacgaaaatagcaacagggcatacggatcatccacaatcaactatacaacaatcaactactaccgggactccgcctccaatgcagcttcgaaacaagattttagccaggatccatctaaatttactgaaccaattaaagacgtgctcatcaagactgcacctatgttgaatagccctaacattgaagcctgtggctactcagaccgggtgcttcagctgaccctcggtaattcaaccatcacaacgcaagaagctgcaaacagcgtggtggcatacggaaggtggccagagtacttaagagattctgaggcaaaccctgtcgatcaaccaaccgagccggacgtcgcggcgtgtcgattctacaccctcgatactgtaagctggaccaaggaatcgcgaggttggtggtggaaacttccagacgccctacgcgatatgggtttgttcggtcagaacatgtattatcattatttgggacgctctggatatacagtccacgtgcaatgcaatgcttcaaagtttcatcagggagctttgggtgtgttcgcggtgccggaaatgtgcttagcaggagactcaaatacgaccacaatgcatacttcttaccagaacgcaaaccccggagaaaagggcggtacgtttaccgggacctttaccccagataataatcaaacctccccagcaagacgtttttgtccagttgactatttgttgggcaacggtacactactagggaatgccttcgtatttcctcatcagattatcaatcttagaactaataattgcgcaactctcgtgttgccatatgtaaattctctgtccatcgactcgatggtgaaacataacaactgggggatagcaatattacctctcgcgcctctgaacttcgcgtcagaatcaagtccggaaatcccgataacacttactatcgctcccatgtgttgcgagttcaacgggctcaggaatataacactacccaggctgcaagggctaccagttatgaatacaccaggatctaaccagtacttaaccgccgataacttccaatccccatgcgccttaccagagttcgatgtcacgccgcccatcgatatccctggggaggtgaaaaatatgatggagcttgccgagattgatacaatgataccattcgatctgtccgcaactaagaaaaatacaatggagatgtaccgtgtgagactttccgataagcctcacaccgacgacccaattctgtgtctcagtctatctcctgcaagtgaccctaggcttagccacaccatgctgggcgagatattgaactattatacccactgggcgggcagtcttaaattcaccttcttattttgtgggtcaatgatggctacgggtaagttgctagtaagctacgctccaccaggcgcagatcctccgaagaaaaggaaagaagctatgcttgggacccacgtcatttgggatattggtttacaatcttcgtgcaccatggtggttccttggatatcaaatacgacctacagacagacaatcgacgactcctttacggagggtggttatatatcagtgttttatcagacccgtattgtggtgccactgtctaccccacgggaaatggatattctgggattcgtctccgcctgcaacgacttctcagtcaggctcctaagggacacgacccacattgaacagaaggcgctagcacaaggactgggtcaaatgttggagtcaatgatagataataccgtaagggagaccgtaggagctgccacttcacgggatgcactgccgaatacagaggcatcagggcccacccattcaaaagagatcccagctctgacagctgtagagaccggtgcaaccaacccactagtcccatcggacactgttcagacacggcacgtggtgcagcacagaagtagatccgaatcctcgattgaaagctttttcgccagaggcgcctgtgtaacaataatgacggtagacaatcccgcaagtactaccaacaaagacaaactgttcgctgtctggaaaataacatacaaggacaccgtacaactgagaagaaagcttgaattttttacttacagcaggttcgacatggagttaacattcgtagtgaccgctaacttcaccgaaaccaataacgggcacgccctgaaccaggtttatcagataatgtacgtaccgccgggtgcccccgtacccgaaaagtgggatgattatacttggcagactagctccaacccttccatattctatacttatggcaccgcgccggcgagaatttcagttccatacgtgggaatatcaaatgcatactctcatttctatgatggcttctcaaaggtgcctttaaaagaccaatcagcggccctgggagattcgttgtacggggccgcctccttgaacgattttgggatcctagcagtgagggtggtgaacgaccataatccaacgaaggtcaccagtaagatacgggtctacttgaagcctaagcatattcgtgtgtggtgtccaagacctccacgcgccgtcgcatattatggacccggtgtcgactataaagacgggaccttgaccccactgagtacaaaagacctcacaacttacggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcattttcagacattagagacttgtatgcctacgaagaagaagccatggaacaaggcctcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactcgagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgacgcgttagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagttaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacatagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggacaaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactggtgtaggggtaaatttttctttaattcggaggaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 2643 bp, unidentified
  misc_feature 1..2643: note = synthetic
  source 1..2643: mol_type = other DNA
atgggagctcaggtgtcatcccaaaaagtaggcgcacacgaaaactcgaatcgggcatacggatctagtacgattaactatactacaatcaattactatagggactccgcctctaacgccgcttcgaaacaggacttttcgcaagaccctagcaagtttaccgaaccgattaaggacgtgttgatcaaaaccgcacctatgcttaactcacctaacatagaggcatgcggatactccgatagggtgttgcaactgacactcggcaatagtacgattacgacacaggaagccgctaatagcgtagtcgcatacggaaggtggcccgaataccttagagactccgaagcgaatccagtcgatcagcctaccgaacctgacgtcgccgcatgtcggttttacacactcgataccgtgtcatggactaaggaatcgcgagggtggtggtggaaactgccagacgcattgcgcgatatggggttgttcggacagaatatgtactatcactatctcggaagatccgggtataccgtacacgtgcaatgtaacgcctctaagtttcaccagggagcgttaggcgtattcgcagtgccagagatgtgtctagccggagactccaatacgactactatgcatacctcataccaaaacgctaacccaggcgaaaagggggggacatttaccggaacattcacacccgataacaatcagacatcccccgctagacggttttgcccagtcgactatctactcggaaacggtacactgttagggaatgccttcgtattcccacaccagataattaacttacggactaacaattgcgcaaccctagtgttgccatacgttaactcactgtcaatcgatagtatggtgaaacataacaattgggggatcgcaatattaccgttagcgccactgaatttcgccagcgaatcgtcacctgagataccgattacccttacaatcgcacctatgtgttgcgagttcaacggattgcgtaatataaccctaccacggttgcaggggttgcccgttatgaataccccagggtctaaccaataccttaccgccgataatttccaatccccttgcgcactgccagagttcgacgtaacccctccaatcgacatacccggcgaggttaagaatatgatggagttagccgaaatcgatactatgataccgttcgatctatccgctacgaaaaagaatactatggagatgtatcgcgtgagattgtccgataagccacataccgacgatccgatactgtgtctgtcactgtcacccgccagcgatcctaggttgtcccatacaatgttaggcgagatactgaattactatacccattgggccggtagtctgaaattcacatttctgttttgcggatctatgatggcgaccggaaagctgttagtgtcatacgctccacccggagccgatccacctaaaaaacgcaaggaagcgatgctcggtacacacgtgatatgggatatcggactgcaatcgtcatgtactatggtcgtgccatggatatcgaatacgacttatagacagacaatcgacgatagctttaccgagggggggtatattagcgtattctatcagacacgtatcgtagtgccactgtcaacccctagagagatggacatactcggattcgtatccgcatgtaacgactttagcgtgagactgttacgcgatactacccatatcgaacagaaagcgctagcacaggggttagggcaaatgctagagtcaatgatcgacaatactgtacgcgaaaccgtaggcgcagcgaccagtagggacgcactaccgaataccgaagcgagcggacctacccactctaaagagataccggcactaaccgccgtcgaaaccggagcgactaacccactagtcccatccgataccgtgcaaaccagacacgtagtgcaacatcggtctagatccgagtcatcaatcgaatcctttttcgctagaggcgcatgcgttacgattatgaccgtcgataacccagcgtcaacgactaacaaagacaaattgttcgccgtatggaaaattacctataaggataccgtgcaattgcgacgtaaactagagttctttacatactctagattcgatatggagcttacattcgtagtgaccgctaactttaccgagactaataacggtcacgcccttaatcaggtgtatcagattatgtacgtaccgccaggcgcaccagtgcccgaaaaatgggacgactatacatggcagactagctctaatccgtcaattttctatacatacggtaccgcaccagctaggattagcgtgccatacgtcggtatatcgaacgcttactcacacttttacgacggattctctaaagtgccacttaaggaccaatccgccgcactaggcgatagcctatacggagccgctagtcttaacgatttcggtatactcgccgttagggtcgtgaacgaccataaccctactaaggtcacctctaagattagggtgtatcttaagcctaagcatattagggtgtggtgtcctagaccgcctagagccgttgcgtattacggaccaggcgtcgactataaggacggtacattgaccccactgtcaacgaaagaccttacgacgtac

SEQ ID NO: 5 - DNA, 2643 bp, unidentified
  misc_feature 1..2643: note = synthetic
  source 1..2643: mol_type = other DNA
atgggagctcaagtatcttcacaaaaagttggtgctcatgaaaattcaaacagagcatatggttcttcaacaataaactacaccacaataaattattacagggacagtgcttcaaatgcagcttcgaaacaagatttttctcaagatccttccaagttcacagagcccatcaaagatgttttaataaaaacagcacccatgctgaacagccccaacattgaagcatgtggttatagtgacagagttctacagctcacgctgggaaattcaacaataacaactcaagaagcagcaaattctgtggtggcctatggtcgttggccagagtatctgcgggactcggaggcaaatcctgtggaccagccaacagagccggacgtcgctgcctgccgcttctacacgttagacacggtctcctggaccaaggagtcgcgaggctggtggtggaaacttccagatgctctgcgggacatgggcctctttgggcagaacatgtactatcattatttgggaagaagtggctacacggtccatgtgcagtgtaatgcatccaagtttcatcaaggtgccctgggcgtgttcgcggttcctgagatgtgtttggctggggacagcaacaccaccaccatgcacacgtcctaccaaaatgcaaatccaggagaaaaaggtggcaccttcacgggcaccttcactccagacaacaaccagacctcgccggcgcgccgcttctgcccagtagattatcttcttggaaatggcacgctgctggggaatgcctttgtatttcctcatcaaataataaatttgaggaccaacaactgtgccaccttggttcttccttatgtcaacagcctctccattgactccatggtgaagcacaacaactggggaatagcaatattacctttggcacctttaaattttgcctcggagagttctccagagatccccatcaccctcaccatcgcgccgatgtgctgcgagttcaatgggctgaggaacatcaccctgcccaggctacaaggtcttcctgtcatgaacacaccaggttcaaatcagtatttaacagcagacaacttccagtctccatgtgctttgccagaatttgatgtcactccaccaatagatattccaggagaagtaaagaacatgatggaactagcagaaatagacaccatgattccatttgatctttcagccaccaagaagaacaccatggagatgtaccgtgtcaggctttcagataaaccacacacagatgatccaattctctgcctcagtctttctccagcctcggaccctaggctcagccacaccatgctaggagagatattaaattattacacgcactgggcaggctccctgaagttcaccttcctcttctgtggctccatgatggcaactggaaaattattagtatcatatgcgccgccgggggcagatcctccaaagaagaggaaggaggccatgctaggaactcatgtaatatgggacattgggttgcagagcagctgcaccatggttgttccctggatctccaacaccacctacaggcagaccattgatgacagcttcacagaaggtggttatatttctgtcttctaccagacaagaattgtggtgccgctgtccactccaagagaaatggatattcttggatttgtatcagcctgcaatgacttctctgtgaggctgctgcgggacaccactcatattgagcagaaggcgctagcacaaggacttggacaaatgctggagtccatgattgacaacactgtgcgggagactgtgggggcggccacctcaagagatgctttgccaaatacagaggcttcggggccaacacacagcaaagaaataccagctttgacagcagtagaaactggagcaacaaatccactagtcccttcagatactgtacaaacaagacatgtggtgcagcacagatcaagaagtgaaagttcaatagaatccttctttgctcgtggggcctgtgtcaccatcatgactgtggacaacccagcctccaccaccaacaaagataaattatttgctgtgtggaagatcacctacaaagatactgtgcagctaagaagaaaactagagttcttcacctactcaagatttgacatggaactaacttttgtggtcacagccaacttcacagagaccaacaatggacatgctttgaatcaagtctaccagattatgtacgtaccaccaggggctcctgtgcccgaaaaatgggatgactacacgtggcagacttcatcaaatccttccatcttctacacatatggaacagcgccggccaggatatcagttccttatgtgggcatctcaaatgcttacagccacttctatgatggcttctccaaagtacctttgaaggaccagtcggcggcgctgggggacagtttatatggtgcagcatctctaaatgattttggaattttggctgtccgtgtagtaaatgaccacaaccccaccaaggtcaccagcaagatccgagtatatttaaaaccaaagcacatccgagtgtggtgtccgagaccgccaagagctgtagcgtattacggtcctggtgtggactataaagatgggactttgactcctttgtccaccaaagacctcaccacgtac

SEQ ID NO: 6 - DNA, 7441 bp, unidentified
  misc_feature 1..7441: note = synthetic
  source 1..7441: mol_type = other DNA
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggcgcgcaagtgtcgtctcaaaaggttggagcccacgagaatagtaacagagcatacggatcaagcacaataaattatacgacgatcaattactaccgtgactcggcatccaacgccgctagcaaacaggacttttctcaggatccatctaaattcacggaacccataaaagacgtgcttattaagaccgcacctatgctgaattcaccgaatatcgaagcctgtggttactctgaccgggtgctacagctgacacttggcaactcaacgatcacaacccaagaggccgcaaattccgtagtagcgtacggtaggtggccagagtacctacgtgattcagaggctaaccctgtagatcaacctaccgaaccggacgtggcagcttgtcgtttctacactcttgataccgtgtcctggaccaaggaaagtaggggttggtggtggaaactccccgacgcccttagggatatggggctgttcggtcagaacatgtactatcactatctgggacgctcaggctatacggtacacgtgcaatgcaacgcaagtaagtttcatcagggcgcgctgggagtgttcgcagttccggaaatgtgcctagctggcgactcaaatacaacgactatgcatacaagttatcagaacgcgaacccaggagaaaaggggggaacatttacaggcacatttaccccagataataaccaaacctccccagcacggagattctgtcctgtagactacttgttggggaacggaaccctacttggtaacgcattcgtttttccacatcaaattatcaatcttagaactaataattgtgcaactctcgtgttgccatatgtcaattccttgtcgatagactccatggtcaaacataacaactggggcatagccattctaccgctcgcaccactgaacttcgcatccgaatcctcacccgaaatccccataacactcacaatcgcaccaatgtgttgcgaatttaacggactgcggaatataaccctaccgagactccagggtttacccgtaatgaatacgccaggatcaaatcagtacttaaccgccgataatttccaatccccatgcgccttaccagaattcgacgtaacgcctccaattgatattccaggagaggtgaaaaatatgatggagctcgcagagattgatactatgataccgttcgatctttccgctaccaagaaaaatacgatggagatgtaccgcgtgagactgtccgataagccacacaccgatgacccaatactgtgtttaagtttgtctccagcttccgacccacgtctctctcataccatgttaggggagatacttaactattatactcattgggccgggtcattaaagtttaccttcctgttttgcgggtctatgatggccaccgggaagctgctagttagctatgccccacctggtgcggatcctcctaaaaagagaaaagaagcaatgctcggtacccacgtaatttgggatattggactgcaatcgagctgcaccatggtggtgccttggatatctaatacaacctacagacagacaatcgacgactcattcacagagggtgggtatatatcggtgttttatcagacccgaatcgttgtgccattgtccacccctcgggaaatggatatccttggcttcgtgagtgcttgcaacgacttttcagtgagactactaagggacactacgcatattgaacagaaggcactggctcaaggcctgggccaaatgctcgagtcaatgatcgataataccgtgagagagaccgtaggtgcagcgacctcaagggacgcgttgcctaataccgaggcgtccgggccgactcacagtaaagagataccagcgttaacagcagtcgaaaccggtgcgactaatcccttggtgccaagcgatacagtgcaaacaaggcacgtagtccagcaccgatccaggtcggaaagctctatcgagtccttttttgctaggggggcttgtgtcactataatgacagtcgacaatccggcatcaacaaccaacaaagacaagttattcgctgtatggaaaattacctacaaggacaccgtccaactgagacggaagttagaattctttacatactcacggttcgacatggaattgactttcgtagtgaccgctaactttaccgaaaccaataacggtcacgccctgaatcaggtataccagatcatgtacgtccctcctggggcaccagttccggagaaatgggatgactatacttggcagacatctagcaaccctagcattttctacacttatggtaccgcccccgctagaattagtgtgccttacgtaggaatttcaaacgcatatagccatttctacgacggattctcaaaggtgccattaaaggatcagagcgcggccttgggggattcgttgtacggagccgcttcattgaacgatttcgggatactagcagtcagggtcgtgaacgaccataaccctactaaagtgacatcgaagatacgcgtctaccttaagccaaaacacatcagggtgtggtgtcctaggccgccaagagccgtcgcctattatggtcccggtgtcgattataaagacggaaccctgaccccattgtcaactaaagacctaaccacttatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcattttcagacattagagacttgtatgcctacgaagaagaagccatggaacaaggcctcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactcgagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgacgcgttagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagttaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacatagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggacaaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactggtgtaggggtaaatttttctttaattcggagg

SEQ ID NO: 7 - DNA, 32 bp, unidentified
  misc_feature 1..32: note = synthetic
  source 1..32: mol_type = other DNA
gcactgataatgaactcctctggatctactgg

SEQ ID NO: 8 - DNA, 32 bp, unidentified
  misc_feature 1..32: note = synthetic
  source 1..32: mol_type = other DNA
ccagtagatccagaggagttcattatcagtgc

SEQ ID NO: 9 - DNA, 50 bp, unidentified
  misc_feature 1..50: note = synthetic
  source 1..50: mol_type = other DNA
caagaattcctgaccacatacggtgctcaggtttcatcacagaaagtggg

SEQ ID NO: 10 - DNA, 50 bp, unidentified
  misc_feature 1..50: note = synthetic
  source 1..50: mol_type = other DNA
caagaattcctgaccacatacggtgcgcaagtatcgtcgcaaaaagtagg

SEQ ID NO: 11 - DNA, 40 bp, unidentified
  misc_feature 1..40: note = synthetic
  source 1..40: mol_type = other DNA
ttcgaattctccatatgtggtcagatccttggtggagagg

SEQ ID NO: 12 - DNA, 41 bp, unidentified
  misc_feature 1..41: note = synthetic
  source 1..41: mol_type = other DNA
ttcgaattctccatacgtcgttaaatctttcgtcgataacg

SEQ ID NO: 13 - DNA, 2341 bp, Influenza virus
  source 1..2341: mol_type = unassigned DNA
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgacaagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 14 - DNA, 2341 bp, unidentified
  misc_feature 1..2341: note = synthetic
  source 1..2341: mol_type = other DNA
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggacgttatggagtctatgaaaaaagaggaaatggggattacgacacattttcaacgaaaaagacgggttagggataatatgacaaaaaaaatgattacgcaacgaacaatcggaaagaaaaaacagagactgaataagcgatcatacttgattagggcacttacacttaacactatgactaaggacgccgaaaggggaaagctaaagcgtagagcaattgcaacacccggaatgcaaattagggggttcgtatacttcgtcgagacactcgctagatccatatgcgaaaagttagagcaatccggactgccagtcggggggaacgaaaaaaaagcgaaactcgctaacgtcgttagaaaaatgatgactaatagtcaggataccgaactgtcatttacgattaccggcgataatactaagtggaacgagaatcagaatcctagaatgtttctcgcaatgatcacatatatgacacgtaaccaacccgaatggtttagaaacgtactgtcaatcgcaccaattatgtttagcaataagatggctagattgggcaaggggtatatgtttgaatctaagagtatgaaattgcgaacacagatacctgccgaaatgctagcatcaatcgatctaaagtactttaacgatagtacacgaaaaaaaatcgaaaagattagaccgttactgatagagggaaccgccagcctatcccccggaatgatgatggggatgtttaatatgcttagtaccgtgttaggcgttagcatacttaacttagggcaaaaacgttatactaagactacatattggtgggacggactgcaatctagcgacgatttcgcactaatcgttaacgcacctaaccatgaggggatacaagccggagtcgatagattctatagaacatgcaaactgttagggattaatatgtctaaaaaaaagtcatacataaatagaaccggaacatttgaattcactagctttttttacagatacggattcgttgctaattttagtatggagttacctagtttcggagttagcggaattaacgaatccgccgatatgtcaatcggcgtaaccgttattaagaataatatgattaataacgatctagggccagcaaccgcacaaatggcattgcagttgttcataaaggattatcgttatacatatagatgtcatagaggcgatacacagatacagactagacgatcatttgaaatcaaaaaattgtgggagcaaactaggtctaaagccggactgttagtgtccgacggagggcctaatctatacaatattaggaatctgcatatacccgaagtgtgtctaaagtgggagcttatggacgaagactatcaggggagattgtgcaatccgcttaacccattcgttagccataaagagatagagtcaatgaataacgccgttatgatgccagcacacggacccgctaagaatatggaatacgacgcagtcgcaactacacatagttggataccgaaacggaatcgatccatactgaatacatcccaaagaggcgtactcgaagacgaacaaatgtaccaacggtgttgcaatctatttgaaaaattttttcctagtagtagctatagacgaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 15 - DNA, 2341 bp, unidentified
  misc_feature 1..2341: note = synthetic
  source 1..2341: mol_type = other DNA
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggacgttatggagtctatgaaaaaagaggaaatggggattacgacacattttcaacgaaaaagacgggttagggataatatgacaaaaaaaatgattacgcaacgaacaatcggaaagaaaaaacagagactgaataagcgatcatacttgattagggcacttacacttaacactatgactaaggacgccgaaaggggaaagctaaagcgtagagcaattgcaacacccggaatgcaaattagggggttcgtatacttcgtcgagacactcgctagatccatatgcgaaaagttagagcaatccggactgccagtcggggggaacgaaaaaaaagcgaaactcgctaacgtcgttagaaaaatgatgactaatagtcaggataccgaactgtcatttacgattaccggcgataatactaagtggaacgagaatcagaatcctagaatgtttctcgcaatgatcacatatatgacacgtaaccaacccgaatggtttagaaacgtactgtcaatcgcaccaattatgtttagcaataagatggctagattgggcaaggggtatatgtttgaatctaagagtatgaaattgcgaacacagatacctgccgaaatgctagcatcaatcgatctaaagtactttaacgatagtacacgaaaaaaaatcgaaaagattagaccgttactgatagagggaaccgccagcctatcccccggaatgatgatggggatgtttaatatgcttagtaccgtgttaggcgttagcatacttaacttagggcaaaaacgttatactaagactacatattggtgggacggactgcaatctagcgacgatttcgcactaatcgttaacgcacctaaccatgaggggatacaagccggagtcgatagattctatagaacatgcaaactgttagggattaatatgtctaaaaaaaagtcatacataaatagaaccggaacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 16 - DNA, 2341 bp, Influenza virus
  source 1..2341: mol_type = unassigned DNA
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgcaggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcagcgattgaatcctatgcatcaacttttaagacattttcagaaggatgcgaaagtgctttttcaaaattggggagttgaacctatcgacaatgtgatgggaatgattgggatattgccagacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 17 - DNA, 2341 bp, unidentified
  misc_feature 1..2341: note = synthetic
  source 1..2341: mol_type = other DNA
agcgaaagcaggtcaattatattcaatatggagagaatcaaagagcttaggaatcttatgtcacaatctagaactagagagatactgactaagactacagtcgatcatatggctataatcaaaaaatatactagcggaagacaggaaaaaaatcccgcacttagaatgaaatggatgatggctatgaaataccctattacagccgataagcgaattaccgaaatgataccagagagaaacgaacagggacagacattgtggtctaaaatgaacgacgccggatccgatagagtgatggtttcgccactagccgtaacatggtggaatagaaacggacctattacgaatacagtgcattaccctaagatatacaaaacatatttcgaaagagtcgagagactgaaacacggaacattcggaccagtgcattttcggaatcaggttaagattagacgtagagtcgatattaatccagggcatgcagatctctccgctaaagaggcacaagacgttattatggaggtcgtgtttcctaacgaggtcggcgctaggatactgactagcgaatcgcaattgacaattacgaaagagaaaaaagaggaactccaggattgcaaaattagcccacttatggtcgcatatatgctcgaacgcgaattggttagaaagactagattcctaccagtcgcaggcggaacgtctagcgtgtatatcgaagtgttgcatctaacacagggaacatgttgggagcaaatgtatactccaggaggcgaagtgagaaacgacgacgttgatcaatcgctaatcatagccgctaggaatatagtgagaagggcagccgttagcgcagacccacttgcgtcactactcgaaatgtgccatagtacgcaaatcggagggattagaatggtcgatatccttaggcagaatcctacagaggaacaggccgtagacatatgcaaagccgcaatgggattgcgaattagctcatcattctcattcggagggtttacgtttaaacggactagcggatctagcgtaaaacgcgaagaggaagtgcttactggcaatctgcaaacactaaagattagggtgcatgagggatacgaagagtttacaatggtcggacgtagagcaaccgctatacttagaaaagcgactaggagactgatacaattgatcgttagcggaagggacgaacagtcaatcgccgaagcgataatagtcgcaatggtgttttcgcaagaggattgcatgattaaggccgttaggggggatctgaatttcgttaatagggctaatcagagactgaatcctatgcatcaattgcttagacattttcagaaagacgctaaagtgttgtttcagaattggggagtcgaacctatcgataacgttatgggtatgatagggatactgccagatatgacaccatcaatcgaaatgtcaatgagaggcgttaggattagtaagatgggcgtagacgaatactccagcactgagagagtggtagtgtcaatcgatagatttcttaggattagggatcagagaggcaacgtactgctatcacccgaagaagttagcgaaacacagggaaccgaaaaattgacaattacgtatagtagtagtatgatgtgggagattaacggaccagagtcagtgttagtgaatacatatcaatggataatacggaattgggagacagtgaaaatacaatggtcacagaatcctacaatgctatacaataagatggagttcgaaccttttcaatcgttagtgcctaaggccataagaggccaatatagtgggttcgttagaacattgtttcagcaaatgagagacgtactcggaacattcgataccgcacagataattaagctattgccattcgcagccgcaccacctaagcaatctagaatgcaattttctagctttaccgttaacgttaggggatccggaatgcgaatactcgttagggggaatagtccagtgtttaattacaataaggcaactaagagattgacagtgttaggcaaggacgcaggaacattgaccgaagacccagacgagggaaccgctggagtggaatccgcagtgcttagggggtttctgatactcggaaaggaggataagagatacggacctgcactatcgattaacgaactatctaatctcgctaaaggcgaaaaagcgaatgtgttaatcggacagggagacgtagtgttagtgatgaaacggaaacgcgatagctcaatactgacagactcacaaaccgctactaagagaattcggatggcaattaattagtgtcgaatagtttaaaaacgaccttgtttctact

SEQ ID NO: 18 - DNA, 2233 bp, Influenza virus
  source 1..2233: mol_type = unassigned DNA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgctagcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 19 - DNA, 2233 bp, unidentified
  misc_feature 1..2233: note = synthetic
  source 1..2233: mol_type = other DNA
agcgaaagcaggtactgatccaaaatggaggatttcgttaggcaatgctttaatccaatgatagtcgagttagccgaaaagactatgaaagagtatggcgaagacctaaagattgagactaataaattcgccgcaatttgcacacaccttgaggtttgctttatgtattccgattttcactttattaacgaacagggagagtcaattatagtcgagttaggcgatccgaacgcattgctaaagcatagatttgaaattatagagggacgcgataggacaatggcatggaccgtagttaattcgatttgcaatacaaccggagccgaaaaaccgaaattcttacccgatctatacgattataaagagaataggtttatcgaaatcggagtgactagacgcgaagtgcatatttattatctcgaaaaagcgaataagattaagtccgaaaagacacacatacacatttttagctttaccggagaggaaatggcaacaaaagccgattatacacttgacgaagagtctagggctaggattaagactagactgtttacaattagacaggaaatggctagtagggggttgtgggatagctttagacaatccgaaagaggcgaagagacaatcgaagagagatttgaaattaccggaacaatgcgaaagcttgccgatcaatccctaccccccaatttctctagccttgagaattttagggcatacgttgacggattcgaacctaacggatatatagagggaaagctatcgcaaatgtctaaagaggttaacgctagaatcgaaccattcctaaagacaacacctagaccacttagactgccaaacggaccaccatgctcacagcgatctaagtttctgcttatggacgcactaaagttgtcaatcgaagacccatcacacgagggagaggggataccattgtacgacgcaattaagtgtatgcgaacatttttcggatggaaagagcctaacgtagtgaaaccacacgaaaaagggattaatccgaattatctgcttagttggaaacaggtgttagccgaattgcaggatatcgaaaacgaagagaaaattccgaaaactaagaatatgaaaaaaactagccaactgaaatgggcacttggcgagaatatggcacccgaaaaagtcgatttcgacgattgcaaagacgtcggcgatctaaagcaatacgatagcgacgaacccgaacttagatcactcgctagttggatacagaacgagttcaataaggcatgcgaattgaccgatagctcatggatagagcttgacgagataggcgaagacgtagcaccaatcgaacacatagcctctatgagacggaattattttacatccgaagtgtcacattgtagggcaacagagtatattatgaaaggggtgtatattaataccgcattgcttaacgctagttgcgccgcaatggacgatttccaactgataccgatgatctcgaagtgtagaacaaaagagggacgtagaaagactaatctgtatgggttcattattaagggaaggtctcatttaaggaacgatacagacgtagtgaatttcgttagtatggagtttagccttaccgatccgagactcgaaccacacaaatgggaaaagtattgcgtactagagataggggatatgttgattagatccgcaatcggacaggtttcgagaccaatgtttttgtacgttaggactaacggaacctcgaagattaaaatgaaatggggaatggagatgcgtagatgcctattgcaatcccttcagcaaatcgaatctatgatagaggccgaatctagcgttaaagagaaagatatgacaaaagagttttttgaaaataagtccgaaacatggccaatcggagagtcaccaaaaggggttgaggaatcctcaatcggaaaagtttgtagaacattgctcgcaaaatccgtattcaatagtctatacgccagcccacaactagagggattctctgctgagtcacgaaaactgttactgatagtgcaagcccttagggataatctcgaacccggaacattcgatctaggggggttgtacgaagcaatcgaagagtgtctgattaacgatccatgggtactgcttaacgctagttggtttaattcgttccttacacacgcactatcttagttgtggcagtgctactatttgctatccatactgtccaaaaaagtaccttgtttctact

SEQ ID NO: 20 - DNA, 1775 bp, Influenza virus
  source 1..1775: mol_type = unassigned DNA
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 21 - DNA, 1775 bp, unidentified
  misc_feature 1..1775: note = synthetic
  source 1..1775: mol_type = other DNA
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgttagaggactcacataacggaaagctatgtaggcttaagggaatcgcaccactgcaattgggcaagtgtaatatagccggatggttgttggggaatcccgaatgcgatccactgttacccgttaggtcatggtcatatatagtcgagacacctaatagcgaaaacggaatttgttatcccggcgattttatcgattacgaagagcttagagagcaattgtctagcgttagttcattcgaaagattcgaaatttttccgaaagagtctagttggccaaatcataatactaacggagtgactgccgcatgctcacacgaaggcaagtctagcttttataggaatctgttgtggttgactgagaaagagggatcatatccgaaactgaaaaactcatacgtgaacaaaaagggaaaggaagtgttagtgttgtgggggatacaccatccaccaaatagtaaagagcaacagaatatatatcagaacgaaaacgcatacgttagcgtcgtaactagtaattataatagaaggtttacacccgaaatcgcagagagaccgaaagttagagaccaagccggaagaatgaattattattggacactactgaaacccggcgatacaattatattcgaagcgaacggaaatctgatcgcaccgatgtatgcattcgcactatctagggggttcggatccggaattattactagtaacgctagtatgcacgaatgtaacacgaagtgtcagactccactaggcgcaattaactctagtctgccatatcagaatatacatcccgtaacaatcggcgaatgcccaaaatacgttagatccgctaagcttagaatggttaccggactgagaaatacaccatcaatccaatctagggggttgttcggagcgatagccggatttatcgaaggggggtggacagggatgatagacggttggtacggatatcatcaccaaaacgaacagggatccggatacgcagccgatcagaaatcgacgcaaaacgctattaacggaattactaataaagtgaataccgtaatcgaaaaaatgaatatccaatttaccgcagtcggaaaggaattcaataagcttgagaaaagaatggagaatctgaataaaaaagtcgacgacggatttctagacatatggacttataacgccgaactgttagtgttgctcgaaaacgaaagaacactagactttcacgactcaaacgttaagaatctatacgaaaaagtgaaatcccaattgaaaaataacgctaaagagatagggaacggatgtttcgagttctatcataaatgcgataacgaatgtatggaatccgttaggaacggaacatacgattatcctaagtatagcgaagagtcaaaactgaatagggagaaagtcgacggagtgaaactcgaatcaatggggatatatcagatactggcaatctatagtacagtcgccagctcactggttcttttggtctccctgggggcaatcagtttctggatgtgttctaatggatctttgcagtgcagaatatgcatctgagattagaatttcagaaatatgaggaaaaacacccttgtttctact

SEQ ID NO: 22 - DNA, 1565 bp, Influenza virus
  source 1..1565: mol_type = unassigned DNA
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 23 - DNA, 1565 bp, unidentified
  misc_feature 1..1565: note = synthetic
  source 1..1565: mol_type = other DNA
agcaaaagcagggtagataatcactcactgagtgacatcaaaatcatggcgtcccaaggcaccaaacggtcttacgaacagatggagactgatggagaacgccagaatgccactgaaatcagagctagcgtcggaaaaatgatagggggaatcggaaggttttacatacaaatgtgtaccgaactcaaattgtccgattacgaagggagattgatccaaaatagtctgacaatcgaaagaatggtgttaagcgcattcgacgaaagacggaataagtatctcgaagagcatcctagcgcaggcaaggatccaaaaaaaaccggagggccaatctataggagagtgaacggaaagtggatgcgcgaactgatactgtacgataaagaggagattagacggatatggcgacaagcgaataacggagacgacgctactgccggactgacacatatgatgatatggcactctaatcttaacgacgctacataccaacggactagggcactcgttagaaccggaatggatcctagaatgtgctcacttatgcagggatctacactccctagacgatccggagccgcaggagcagccgttaagggagtcggaactatggttatggaactcgttagaatgataaaaagggggattaacgataggaatttttggagaggcgaaaacggacgtaaaactagaatcgcatacgaaagaatgtgcaatatactcaaagggaaattccaaaccgcagcgcaaaaagctatgatggatcaagttagggagtctaggaatccaggaaatgccgaattcgaagaccttacatttctcgctcggtccgcactaatccttcgcggatcagtcgcacacaaatcttgcttacccgcatgcgtatacggacctgcagtcgctagcggatacgatttcgaacgcgaagggtatagtctagtaggaattgatccatttagattgctccaaaattcgcaagtgtatagtctgattagacctaacgagaatcctgcacacaaatctcaactcgtatggatggcatgccatagtgccgcattcgaagaccttagagtgctatctttcataaagggaacgaaagtgttgcctaggggaaagctatctactaggggagtgcaaatcgctagtaacgagaatatggagactatggagtctagtacactcgaactgagatctagatattgggctattaggactagatccggagggaatacgaatcagcaacgagctagcgccgggcaaatctcaatccaacctacattttccgtgcaacggaatctgccattcgatcggacaacgattatggccgcattcaatgggaataccgagggacggactagcgatatgagaaccgaaattatcagaatgatggaatccgctagaccagaggacgtttcgtttcaaggacggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 24 - DNA, 1413 bp, Influenza virus
  source 1..1413: mol_type = unassigned DNA
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 25 - DNA, 1413 bp, unidentified
  misc_feature 1..1413: note = synthetic
  source 1..1413: mol_type = other DNA
agcgaaagcaggggtttaaaatgaatccaaatcagaaaataacaaccattggatcaatctgtctggtagtcggactaattagcctaatattgcaaatagggaatataatctcaatatggatttcgcattcaatccaaaccggatcacaaaatcatacaggcatatgcaatcagaatataattacttataaaaatagtacatgggtgaaagatactactagcgtgatactaaccggcaattctagtctatgtccgattagggggtgggctatatactctaaagacaatagtatacggatagggtctaagggagacgttttcgtaattagggaaccgtttataagttgttcacatctagagtgtaggaccttttttctgacacaaggcgcactattaaacgataagcattctaacggtacagttaaggataggtcaccttatagggcacttatgtcatgtcccgtaggcgaagcccctagtccatacaatagtagatttgaatccgttgcatggtccgctagcgcatgtcacgacggaatggggtggttgactatagggattagcggacccgataacggagccgttgccgtactgaaatataacggtataattaccgaaactattaagagttggcgtaaaaaaatattgcgtacacaagagtccgaatgcgcatgcgttaacggatcatgttttacaattatgactgacggacctagcgacgggttagcgtcatacaaaatttttaaaatcgaaaaaggcaaggttactaagtcaatcgagttaaacgcacctaattcgcattacgaagagtgttcatgttatcccgataccggaaaggttatgtgcgtttgtagggataattggcacggttcgaacagaccttgggtgtcattcgatcaaaatctagactatcaaatcggatatatatgtagcggagtgttcggcgataatcctagaccagaggacggtacaggcagctgtggaccggtttacgttgacggcgctaacggcgttaaggggtttagttatagatacggcaatggcgtatggatcggtaggactaagtcacatagttctagacacggatttgaaatgatatgggatcctaacggatggaccgaaaccgactcgaagtttagcgttaggcaagacgtagtcgctatgaccgattggtccgggtatagcggatcattcgtgcaacatccagagttaaccggattggattgtatgcgaccatgtttttgggttgagttgattagggggagaccgaaagagaaaactatatggactagcgcgagcagcatttctttttgtggcgtgaatagtgatactgtagattggtcttggccagacggtgctgagttgccattcagcattgacaagtagtctgttcaaaaaactccttgtttctact

SEQ ID NO: 26 - DNA, 1026 bp, Influenza virus
  source 1..1026: mol_type = unassigned DNA
agcgaaagcaggtagatattgaaagatgagtcttctaaccgaggtcgaaacgtacgtactctctatcatcccgtcaggccccctcaaagccgagatcgcacagagacttgaagatgtctttgcagggaagaacactgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctac

SEQ ID NO: 27 - DNA, 890 bp, Influenza virus
  source 1..890: mol_type = unassigned DNA
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttaataataaaaaacacccttgtttctact

SEQ ID NO: 28 - DNA, 890 bp, unidentified
  misc_feature 1..890: note = synthetic
  source 1..890: mol_type = other DNA
agcaaaagcagggtgacaaagacataatggatccaaacactgtgtcaagctttcaggtagattgctttctttggcatgtccgcaaacgagttgcagaccaagaactaggtgatgccccattccttgaccgactgagacgggatcagaaatcccttaggggcaggggatcgaccctaggcctagacatcgaaaccgcaactagggccggaaagcagatcgtggagcgtatactgaaagaggagtccgacgaagcgcttaagatgactatggccagcgtacccgctagtcggtaccttaccgatatgacactcgaagagatgtcacgcgattggtctatgctaatccctaagcagaaagtggccggacctctatgtatacggatggaccaggcgattatggacaaaaacattatccttaaagcgaacttttccgtgatattcgatcgcctagagactctgatactgttgcgtgcattcacagaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttaataataaaaaacacccttgtttctact

SEQ ID NO: 29 - DNA, 7152 bp, Human rhinovirus 89
  source 1..7152: mol_type = unassigned DNA
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatcatgggtgcacaagtatctagacaaaatgttgggacacactccacacaaaattcagtgagcaatggatctagcttaaattatttcaacatcaattattttaaagacgcagcttcaagtggtgcttctagattggatttttctcaagaccctagtaaatttactgatcctgttaaagatgtgttagaaaagggtattccaacacttcaatcaccaacagttgaagcttgtggttattcagacagactaatacagataacccgaggagattccactataacatcccaagatactgcaaatgcagttgttgcttatggtgtgtggccatcatacttgactccagatgatgcgactgctattgacaaacccacacaacctgatacatcatccaacagattctacaccttggacagtcgttcttggacatctgcctcatctggatggtggtggaaattgcctgatgcccttaaaaacatgggtatatttggtgaaaatatgttttaccattttctagggagatctggatacacaatacatgtacaatgtaattctagcaagtttcatcagggtttattaatagttgccgccatcccagaacatcaattggcatctgcaacaagtggaaatgtatcagtcgggtacaatcacacccacccaggtgagcaaggtagagaagtagtaccatcacggacatctagtgataataaaagacctagtgatgacagttggttaaattttgatggaacattacttggtaacttacctatttatccccaccaatacattaatctaaggactaacaattcagctacccttattttaccttatgtcaatgctgtaccaatggactctatgcttagacataataattggagcttggttataatcccaatatgccctcttcaggtccaacctggggggacacaatccatacctataacagtatcaattagccctatgttttcagaattttcagggccaagaagtaaggttgtgtttagtaccactcagggtttaccagttatgttaacacctggatctgggcaattcttaacaactgatgatactcaatccccatcagcgtttccatacttccacccgaccaaggaaatatttatacctggacaagttaggaatttaattgaaatgtgccaagttgacacactcattcctgttaacaatacacaggaaaatgtaagatctgtgaatatgtacactgttgatttacgcacacaagttgatttagctaaagaagtcttttctataccagtagatattgcctcacaacctttagccactactctcataggagaacttgcaagctattacacacactggactggtagtctgcgctttagctttatgttttgtggttctgctagctctactttgaaactattaattgcatacactcctcctggtgttggaaaacctaaatccaggagagaagccatgcttggtacacatttagtgtgggatgtggggttgcagtccaccgcctcactagttgtaccatgggttagtgctagccattttagattcactacacctgacacatattcctcagctggttatattacatgctggtaccagaccaactttgtagtacctgatagtactccagataacgccaaaatggtgtgcatggtttctgcatgcaaagatttttgcttaagattagccagagatactaacctacacacacaagaaggagtactcacacaaaacccagttgaaaattatatagatagtgtattaaatgaagttcttgtggtgccaaatatccaacctagcacatctgtgtcaagtcatgcagcgcctgcattggatgctgcggaaaccggacacaccagctctgttcaacctgaagatatgattgaaactagatatgttataactgatcaaacaagggatgaaacaagtattgagagtttcttaggtaggtcagggtgtatcgctatgatagaatttaatacaagtagtgataaaactgaacatgataaaattggtaaaggattcaaaacatggaaggttagtcttcaagaaatggcacaaatcagaagaaaatatgaattattcacatatacaagatttgattcagagataacaatagtcactgcagccgcagctcaaggaaatgatagtggacatatagtattgcaatttatgtatgtacccccaggagcacctgtccccgaaaaacgtgatgattacacatggcaatcaggaacaaatgcatctgtgttctggcaagaaggacaaccataccccagattcacaatcccttttatgagcattgcatcagcctattacatgttttatgatggttatgatggtgatagtgcagcatcaaaatacggttctgtagtcactaatgatatgggaaccatatgtgttagaatagtgacatccaaccaaaaacacgattcaaatattgtgtgccgcatttaccacaaggccaaacatataaaagcatggtgtcctcgcccaccaagggctgttgcctatcaacacacacactcaaccaattacataccatccaatggtgaggccacaactcagattaaaaccagacctgatgtttttaccgttacaaacgtcggaccatctagtatgtttgtacatgtgggtaacttaatctatagaaatcttcatctctttaattctgatcttgatgattctattcttgtatcatactccagtgatctaatcatatatcgaacaaacactgaaggtaatgatgtgatccctaattgtgattgcactgaatgtacatattactgccaccacaaagataggtattttcctatcagagttactgcacatgattggtatgagattcaagaatcagaatattacccaaaacatatccaatataatctcctgattggagagggtccttgtgaaccaggagattgtggaggaaaactattgtgtaaacatggtgttataggtatgattacagctggaggtgaaggtcacgttgcttttattgacctgagaaaattccagtgtgctgaggagcaagggttatctgattatgtggaacatcttggtcaagtctttggtgtaggcttcgtagacagcatcaaacaacaggtaaactttatcaaccccactagtaaaattggttcaaaagtgattaaatggttgttgaggatagtttcagctatgataataatggtaaggaatagttctgatccacaaactgtaattgccactctcacccttctaggttgttcaggctcaccatggaggtttcttaaagagaaactctgtgcgtggctccagcttagctatgtacataagcagtctgattcatggctcaagaaatttactgaagcgtgtaacgcagcacgtgggctagagtggattggacaaaagatatctaaatttatagattggataaagagtatgttaccacaggctcaattgaaaattgattacctaaccaaattaaaacaacttaatctcttagagaaacaaatagaaacaattagacttgcacctgctagtgttcaggagaaaattttcattgaaataaacacccttcatgatttatccttaaaattcttaccactgtatgcatctgaagcacgtagaattaagaatttatatatcaaatgcagtaatgttattaaagggggaaagaggaatgaaccagttgcagttctaatacatggttctcctggtactggaaaatctcttgccacttctgttcttgctcgaatgctaactgttgagactgatatatattctttgcccccagatcctaaatattttgatgggtatgatcaacagagtgttgttatcatggatgatatcatgcaaaatcctagtggtgaagacatgactttgttttgccaaatggtatcgagtgtccctttcatacctcctatggcagatcttccagataaaggaaaaccatttacatccaagtttgtacttgcaagcactaatcacactctactaacaccaccaacagtatcttcattaccagcaatggcaagaaggttttactttgatctagacattcaagttaagaaagagtatcttttagatggcaaactagatatagcaaaaagctttcgaccatgtgatgttaatattaaaataggcaatgctaagtgctgtccatttatctgtggaaaagctgtagagtttaaagatagaaattcatgtacaaccttgtctttatctcaattgtatagtcatataaaggaagaagataggagaagaagcagtgcagcacaagcaatggaggctatatttcaaggtatagacctccaatctcctccacctccagccatagctgacctccttaggtctgtgaaaacaccagagatcattaagtattgccaagataataattggattgttccagcagagtgttctattgaaagagatttagggatagcaaatatgactataggtataatagctaatgtggtctctatagtaggtgttatctatataatttataaattgttctgtacacttcagggtccatactcaggggaacctaaacccaaaagcagagctccagagagaagagtagttactcagggcccagaggaagagtttggtcgctcactactcaaacataattgctgtgttgtgacaaccgacaaaggcaaattcacaggtcttggcatatatgaccaagtcatggtacttccaacacattctgacccaggctctgagatcttggtagatggagtaaaagttaaggtctctgattcctatgatttgcataaccatgagggtgttaagctagagatcacagttgtgaaattaattagaaatgagaagtttaaagacatcagaaaatatttaccctcacgtgaagatgactatcctgcttgtaaccttgccttactagctaatcaagatgagccaacaataataagtgttggtgatgcagtatcttatggtaacatcttattgagtggtaccaatactgcacgaatgatcaagtaccattacccgacaaaagctggatattgtgggggtgttttgtacaaggttggctctattcttggtatacatgttggtggcaatggtagagatggattttctgcaatgcttctcaaatcttattttggtgaaacccagggtttaatcactaaagaacttcctgtatctgtaaagaacttaccatccgtacatgtttcatctaaaacccgactacaacctagtgtttttcatgatgttttccctggaacaaaagagcctgcagttcttagtagtaatgatccaagactagaaactgactttgactcagcacttttctccaaatataaaggtaatcctgcttgtcaagtgaccccacacatgaaaattgctgtagcacattatgcagcacagttatctacactagacataaatcctcaacccctttcattggaagagagtgtgtttggtattgagggattagaagctttggatttaaatactagtgcaggatttccttatgtttcactgggaataaagaagaaagatcttatagataaaaagaccaaagacatcacaaaacttaggaaagcaattgatgaatatggtattgatttgcctatggttacttttctgaaagatgaacttagaaagaaggaaaaaataaaagatggaaagactagagtcatagaagctaatagtgtgaatgatactgtgttattcagaagtgtatttggaaatcttttctctgctttccacaaaaacccaggtatagtcactggttcagcagtagggtgtgaccctgaagtattctggtcaactatacctctcatgctagatggagaatgtttaatggcttttgattattcaaactatgatggtagcctacatcccgtttggtttaaatgtcttagtatgctcttagaagacataggtttctcctctcaacttattaaccagatctgtaactctaaacatatatacaaatctaagtattatgaagtggaaggaggtatgccatctggatgtgctggtactagtatttttaatacaataatcaacaatattatcattagaactttggtactagatgcttataagaacatagatctagataaactgaaaatcttagcatatggggatgatgtcatcttttcttataattttaaacttgacatggcagttcttgccaaagaaggagaaaaatatggactaacaatcacccctgctgataagtctgatgttttccaagaattgacctataaaaatgtaacttttcttaaaagaggattcagagctgatgagcgccactctttccttatacaccctacctttcctgtggctgagattcatgactccatcagatggaccaaaaacccttcatgtatgcaggaacacgtgctatctttgtgtcatttaatgtggcataatggtagacatgcataccaggaattcattaaaggtatacgcagtgtatctgccggtcgggcactgtatataccagcttatgaagttcttgaacatgaatggtatgaaaaattttagatataaaactgttaaatatagctagtttattagttttat

SEQ ID NO: 30 - DNA, 7152 bp, unidentified
  misc_feature 1..7152: note = synthetic
  source 1..7152: mol_type = other DNA
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatcatgggagcacaagtgtctagacaaaacgtaggtacacatagtacacaaaactcagtgtctaacggatctagtcttaattattttaatattaattattttaaagacgctgcatctagcggagctagtagacttgattttagtcaggatccatctaaatttaccgatccagttaaagacgtactcgaaaaaggtatacctacattgcaatcacctacagtcgaagcatgcggatactctgatagattgatacaaattactaggggggatagtactattactagtcaggataccgctaacgccgtagtcgcatacggagtttggccatcttatcttacacctgatgacgcaaccgcaatcgataaacctacacaacctgatactagttctaatagattctatacacttgattctagatcatggactagtgctagttcaggttggtggtggaaattgcctgacgcacttaaaaatatgggtatattcggtgagaatatgttttatcatttcttaggtaggtcagggtatactatacacgttcaatgtaatagttctaaatttcaccaaggtctattaatcgttgccgctatacccgaacaccaattagctagcgctacatcaggtaacgttagcgtagggtataatcatacacatccaggcgaacagggtagggaagtcgtaccatctagaacatctagcgataataaacgaccatctgacgatagttggcttaatttcgatggtacactattaggtaatctacctatatatccacatcaatatattaatcttagaactaataattccgctacacttatactgccatacgttaacgcagtgcctatggatagtatgcttagacataataattggtcattagtgattataccgatatgtccattgcaagtgcaacctggaggtacacaatctatacctattaccgttagtattagtcctatgtttagcgaatttagcggacctagatctaaggtagtgtttagtactacacaggggttacccgttatgcttacacctggatcaggtcaatttttgactactgacgatacacaatcacctagtgcattcccatattttcaccctactaaagagatttttatacccggtcaggttaggaatctaatcgaaatgtgtcaagtcgatacacttatacccgttaataatacacaggaaaacgttagatccgttaatatgtataccgttgatttgcgtacacaagtcgatctagctaaagaggttttttcgataccagtcgatatcgctagtcaaccattagctactacacttataggcgaactcgcatcatattatacacattggaccggatcacttagattttcttttatgttttgcggatccgctagtagtacacttaaactgttaatcgcatatacacctccaggagtgggtaagcctaaatctagacgcgaagctatgttaggtacacatctagtttgggacgtagggttacaatctaccgctagtttagtcgtaccatgggttagcgcatcacattttagatttactacacctgatacttattctagtgccggatatataacatgttggtatcaaactaatttcgtagtgccagatagtacacctgataacgctaaaatggtttgtatggttagcgcatgtaaagatttttgtcttagattagctagggatactaatttgcatacacaagagggagtgcttacacaaaatccagtcgaaaattatatcgatagcgtacttaacgaagtgttagtcgtacctaatatacaacctagtactagtgtgtcatctcacgctgcaccagcattagacgcagccgaaaccggacatacatctagtgtgcaacctgaggatatgatagagactagatacgttattaccgatcagactagagacgaaacatcaatcgaatcattcttaggtaggtcagggtgtatcgctatgatagagtttaatactagttccgataaaaccgaacacgataagataggtaaggggtttaaaacttggaaagtgtcattgcaggaaatggcacaaattagacgtaaatacgaattgtttacatatactagattcgatagtgagattactatcgttaccgcagccgcagcacaaggtaacgattcaggtcatatagtgttacaatttatgtacgtaccaccaggtgcaccagtacccgaaaaacgcgatgattatacatggcaatccggtactaacgctagcgttttttggcaagagggacaaccatatcctagatttactataccttttatgtcaatcgctagtgcatattatatgttttacgacggatacgatggcgattctgccgcatctaaatacggatctgtagtgactaacgatatgggtactatatgcgttagaatcgttacatctaatcaaaaacacgattctaatatcgtttgtagaatatatcataaagctaaacatattaaggcatggtgtcctagaccacctagagccgttgcatatcaacatacacatagtactaattatataccatctaacggtgaggctactacacaaattaaaactagacctgacgtttttaccgttactaacgtaggtccatctagtatgttcgtacacgtaggtaatctgatatatagaaatttgcatctattcaattccgatttagacgattctatactcgttagttattctagcgatctgattatatatagaactaataccgaaggtaacgatgtgatacctaattgcgattgtaccgaatgtacttattattgtcatcataaagataggtattttccgatacgcgttaccgcacacgattggtacgaaatacaggaatctgagtattatcctaaacatatacaatataatctgttaataggcgaaggtccatgcgaaccaggcgattgcggaggtaagttattgtgtaaacacggagtgataggtatgattaccgcagggggagagggacacgttgcgtttatcgatttgcgaaaatttcaatgcgcagaggaacaggggttatccgattacgttgagcatttagggcaagtgttcggagtcggattcgttgattcaattaaacaacaggttaattttattaatcctacatctaaaatcggatctaaagtgataaaatggttacttagaatcgttagcgctatgataattatggttaggaattctagcgatccacaaaccgtaatcgctacacttacactattagggtgttcaggttcaccttggcgatttcttaaagagaaattatgcgcatggttgcaattgtcatacgttcataaacaatccgatagttggcttaaaaaatttaccgaagcatgtaacgccgctaggggactcgaatggatagggcaaaaaatatctaaatttatcgattggattaaatctatgttaccacaagcgcaattgaaaatcgattatcttactaagcttaaacaattgaacttactcgaaaaacaaatcgaaactattagactcgcaccagctagtgtgcaagagaaaatttttatagagattaatacattacacgatctatcacttaaattcttaccattatacgctagcgaagctagacggattaaaaatctatatattaagtgttctaacgtaattaagggaggtaagcgtaacgaacccgttgccgtacttatacacggatcaccaggtaccggtaagtcattagcgactagcgtactcgctagaatgcttacagtcgaaaccgatatatactctttaccacctgatcctaaatatttcgacggatacgatcaacaatccgtagtgattatggacgatattatgcaaaatcctagtggtgaggatatgacattgttttgtcaaatggtatctagcgtaccatttataccacctatggccgatttacccgataagggtaagccttttacatctaaattcgtactcgcatcaactaatcatacattgcttacaccacctaccgttagttcactaccagctatggctagacggttttatttcgatctagatatacaggttaaaaaagagtatctgttagacggtaagttagatatcgctaaatcttttagaccatgcgatgttaatattaaaatcggtaacgctaaatgttgtccatttatatgcggtaaggcagtcgaatttaaagataggaattcatgtactacactatcactatcacaattgtattcacatattaaggaagaggatagacgtagatctagtgccgcacaagctatggaagctatttttcagggtatcgatctgcaatcaccaccaccaccagctatagccgatctacttagatccgttaaaacacctgagataattaagtattgtcaggataataattggatcgtaccagccgaatgttcaatcgaacgcgatttagggatagcgaatatgacaatcggtataatcgctaacgtagtgtcaatcgtaggcgttatatatattatatataaattgttttgtacattgcagggaccatactctggcgaacctaaacctaaatctagagcacctgaaagacgcgtagtgacacagggacctgaagaggaattcggtaggtcattgcttaaacataattgttgcgtagtgactaccgataagggtaagtttaccggattaggtatatacgatcaggttatggtgttacctacacattccgatccaggatctgagatactcgtagacggagttaaggttaaggttagcgattcatacgatctacataatcacgaaggcgttaagttagagattaccgtagttaagcttatacgtaacgaaaaatttaaagatatacgaaaatatctaccatcacgcgaagacgattatcctgcatgtaatctcgcactattagcgaatcaagacgaacctacaattattagcgtaggcgatgccgtatcatacggtaatatactgttatccggtactaataccgctagaatgattaagtatcattatcctactaaagccggatattgcggaggagtgttatataaagtcggatctatactcggtatacacgtaggcggtaacggtagggacggattttccgctatgttacttaaatcttatttcggtgagacacagggattgattactaaagagttacccgtatccgttaaaaatctacctagcgtacacgttagttctaaaactagattgcaacctagtgtgtttcacgacgtttttccaggtactaaggaacctgccgtactatcttctaacgatcctagactcgaaaccgatttcgatagtgcattattctctaaatataagggtaatcctgcatgtcaggttacaccacatatgaaaatcgcagtcgcacattacgctgcacaattgtctacacttgatattaatccacaaccactatcacttgaggaatccgtattcggtatcgaagggttagaggcacttgatcttaatactagtgccggattcccatacgttagtctaggtattaaaaaaaaagatctaatcgataaaaaaactaaggatattactaaattgcgaaaagcgatagacgaatacggtatcgatctacctatggttacattccttaaagacgaacttagaaaaaaagagaaaattaaagacggtaagactagggttatcgaagctaactctgttaacgataccgtactttttagatccgtattcggtaatctattttccgcattccataaaaatccaggtatcgttaccggatccgcagtcggatgcgatccagaagtgttttggtctactataccacttatgttagacggtgagtgtcttatggcattcgattattctaattacgatgggtcattgcatcccgtatggtttaaatgtctatctatgttactcgaagatatagggtttagttcacaattgattaatcagatttgtaactctaaacatatatataaatctaaatattacgaagtcgaagggggtatgccatcaggttgcgcaggtacatctatttttaatactataattaataatattataattagaacattagtgttagacgcatataaaaatatcgatctagataagcttaagatactcgcatacggagatgacgttatattctcatataattttaaacttgatatggccgtactcgctaaagagggagaaaaatacggacttacaattacacctgccgataagtctgacgtttttcaggaattgacttataaaaacgttacattccttaaaagggggtttcgcgcagacgaacgacattcttttcttatacatcctacatttcccgttgccgaaatacacgattcaattagatggactaaaaatcctagttgtatgcaagaacacgtacttagtctatgtcatcttatgtggcataacggtagacacgcttatcaggaatttattaagggtattagatccgttagtgccggtagggcactatatatacccgcatacgaagtactcgaacacgaatggtacgaaaaattttagatataaaactgttaaatatagctagtttattagttttat

SEQ ID NO: 31 - DNA, 7212 bp, Human rhinovirus 14
  source 1..7212: mol_type = unassigned DNA
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaggatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacatactagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctcttcgatgtatactggtcctgccttgtccagtgctaaactcactctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactggaactcttcacttatgttaggtttgattctgagtataccatactggccactgcatctcaacctgattcagcaaactattcaagcaatttggtggtccaagccatgtatgttccacatggtgccccgaaatccaaaagagtgggcgattacacatggcaaagtgcttcaaaccccagtgtattcttcaaggtgggggatacatcaaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacacaaaactcttgtcaagatcagagtttatcacagggcaaagctcgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaaccgtacatcaggtgtttactattccacatattacagaaagtattaccccataatttgcgaaaagcccaccaacatctggattgaaggaagcccttattacccaagtagatttcaagcaggagtgatgaaaggggttgggccggcagagctaggagactgcggtgggattttgagatgcatacatggtcccattggattgttaacagctgaaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccttggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaataacgtattgtggttggaacaaatgtcgcaaaagtttgccccattttatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctagatatatgtttgcatactacctacacaaagaatggaaaactcaatgcaggcatgtcaaccaagacatgcaaagattgccatcaaccatctaatttcaagaaatgttgccccctagtctgtggaaaagctattagcttggtagacagaactaccaacgttaggtatagtgtggatcaactggtcacggctattataagtgatttcaagagcaaaatgcaaattacagattccctagaaacactgtttcaaggaccagtgtataaagatttagagattgatgtttgcaacacaccaccttcagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacggcaactaagtcaaaattacatcccagtgtattttatgatgtttttccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaataaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactggatcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttat

SEQ ID NO: 32 - DNA, 7212 bp, unidentified
  misc_feature 1..7212: note = synthetic
  source 1..7212: mol_type = other DNA
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatcatgggcgcacaagtgtcaacgcaaaaatccggatcacacgaaaaccaaaacatactgactaacggatctaatcagacttttacagtgattaattattataaagacgccgctagtactagttcagccggtcaatcactatctatggatccatctaaatttaccgaacccgttaaggatcttatgcttaaaggcgcaccagcacttaactcacctaacgttgaggcatgcggatactctgatagagtgcaacaaattacattgggtaatagtacaattactacacaggaagccgctaacgcagtcgtttgttacgccgaatggcccgaatacttacccgatgtcgacgcatcagacgttaataagacatctaaacccgatactagcgtatgtagattctatacactcgatagtaagacttggactaccggtagtaagggatggtgttggaaattgcctgacgcacttaaggatatgggcgtattcggtcagaatatgttttttcactcattgggtaggtcagggtatacagtacacgttcaatgtaacgctactaaatttcactcagggtgtctgttagtcgttgtgatacccgaacaccaattggcatcacacgaagggggtaacgttagcgttaagtatacatttacacatccaggcgaaagaggtatcgatctatcatccgctaacgaagtggggggacccgttaaagacgttatatacaatatgaacggtacactgttaggtaatctgttaatttttccacatcaatttattaatctgagaactaataataccgctacaatcgttataccatatattaactcagtgccaatcgatagtatgactagacataataacgttagtcttatggtgataccaatcgcaccattgacagtgccaaccggtgcaacacctagtctaccaattacggttacaatcgcacctatgtgtactgagttttccggtattaggtctaaatcaatcgtaccacaagggttacctactactacattgcccggatccggtcaatttttgactactgacgataggcaatcacctagcgcattgcctaattacgaacctacaccacgtatacatatactcggtaaggtgcataatctgttagagattatacaggtcgatacactgatacctatgaataatacacatacaaaagacgaagtgaattcgtatctgataccgttaaacgctaataggcaaaacgaacaggtattcggtacgaatctgtttataggcgatggcgtattcaaaactacactgttaggcgaaatcgttcaatactatacacattggtcagggtcacttagattctcatctatgtatacaggtccagcactatctagcgctaaattgacactcgcatatacacctcccggtgctaggggaccacaggataggagagaggctatgttaggtacacacgtagtgtgggatatcggattgcaatcgacaatcgttatgactataccatggacatcaggcgtacaatttagatataccgatcccgatacatatacatcagccggattcctatcatgttggtatcagactagtctgatactgccacccgaaactaccggtcaagtgtatctattgtcattcatatccgcatgtcccgattttaaacttagattgatgaaagatacacaaacaatttcgcaaacagtcgcactaaccgaagggttaggcgacgaattagaggaagtgatagtcgaaaaaactaagcaaacagtcgcatcaattagttccggacctaaacatacacaaaaagtgccaatattgaccgctaacgaaacaggcgcaactatgcccgtactgccatccgattcaatcgaaacacgaactacttatatgcattttaacggatccgaaaccgatgtcgaatgctttctaggtagggccgcatgcgtacacgttacagagatacagaataaagacgcaaccggtatcgataatcatagagaggctaaattgtttaacgattggaaaattaatctatctagtctagtgcaattgcgtaaaaaactcgaattgtttacatacgttagattcgatagcgaatatactatactcgcaaccgcatcacaacccgattccgctaattatagttctaacttagtcgttcaggctatgtacgtaccacacggtgcacctaaatctaaaagagtcggcgattatacatggcaatccgcatctaatccatcagtgttttttaaggtaggcgatactagtagattctcagtgccatacgtagggttagctagcgcatataattgtttttacgacggatactcacacgacgatgccgaaacacaatacggtattacggtactgaatcatatggggtcaatggcatttagaatcgttaacgaacacgacgaacataaaacactagttaagattagagtgtatcatagggctaagttagtcgaagcatggatacctagagcacctagagcactaccatatacatcaatcggtaggactaattatcctaaaaataccgaacccgttattaaaaaacgtaaaggcgatattaagtcatacggattagggcctagatacggaggtatatatacatctaacgttaagattatgaattatcatcttatgacaccagaggatcatcataacttaatcgcaccataccctaatagggatctagcaatcgttagtacagggggacacggagccgaaactataccgcattgtaatagaacatcaggcgtatactatagtacatattatagaaagtattatcctattatatgcgaaaaacctactaatatttggatcgaagggtcaccatattatcctagtagattccaagccggagtgatgaaaggcgtaggtccagccgaattaggcgattgcggaggtatactgagatgtatacacggtccaatcggactgttaactgccgaagggtcaggatacgtttgtttcgccgatattaggcaattggagtgtatagccgaagaacagggactatccgattatattaccggattgggtagggcattcggagtggggtttacggatcagattagtactaaggttaccgaattgcaggaagtcgctaaggattttttgactactaaagtgctatctaaagtcgttaaaatggttagcgcattagtgataatttgtaggaatcacgacgatctagttacggttaccgctacactcgcactattagggtgcgatgggtcaccatggagattccttaaaatgtatattagtaagcattttcaagtgccatatatcgaaagacaggctaacgacggatggtttagaaaatttaacgatgcatgtaacgccgctaagggactcgaatggatcgctaataagattagtaagttaatcgaatggattaaaaataaagtgttaccacaagctaaagagaaactcgaattttgtagtaagcttaagcaattggatatactcgaaagacagattacgactatgcatatatctaaccctacacaagagaaacgcgaacaattgtttaataacgtattgtggttagagcaaatgtcacaaaaattcgcaccattttacgcagtcgaatctaaacgtatacgcgaactgaaaaataaaatggttaactatatgcaatttaaatctaaacaacgaaccgaacccgtatgcgtactgatacacggtacacccggtagcggtaagtcattgactacatcaatcgtaggtagggctatagccgaacactttaactcagccgtatatagtctaccacccgatccaaaacatttcgatgggtatcaacaacaggaagtcgttattatggacgatctgaatcagaatcccgatggtcaggatattagtatgttttgtcaaatggtgtcatcagtcgattttctaccacctatggcatcactcgataataagggtatgttgtttacatctaatttcgtactcgcatcaactaactctaatacactatcaccacctactatactgaatcccgaagcattagtgcgtagattcggattcgatctagatatatgcttgcatacaacttatactaaaaacggtaagcttaacgccggtatgtcaactaagacatgtaaggattgtcaccaacctagtaattttaaaaaatgttgtccactagtttgcggtaaggcaattagtctagtcgatagaactactaacgttaggtattcagtcgatcaattggttaccgcaattatatccgattttaaatctaaaatgcaaattaccgattcactcgaaacattgtttcagggaccagtgtataaggatcttgagatagacgtatgcaatacaccacctagtgagtgtattaacgatctgttaaaatcagtcgatagcgaagagatacgcgaatattgtaaaaaaaaaaaatggattatacccgaaataccgactaatatcgaaagagctatgaatcaggctagtatgattattaatactatacttatgttcgttagtacattgggtatcgtttacgttatatacaaattgttcgcacaaacacagggaccatactcaggtaatccaccacataataagcttaaggcacctacacttagacccgtagtcgttcagggacctaataccgaattcgcactatcactattgcgtaaaaatattatgacaattactacatctaaaggcgaatttacagggttagggatacacgatagggtatgcgttataccgacacacgctcaacccggtgatgacgtactggttaacggtcaaaaaattagggttaaggataagtataagttagtcgatccagagaatattaacttagagttaaccgtactgacactcgatagaaacgaaaaatttagggatattagggggtttatatccgaagatcttgagggagtcgacgctacattagtcgtacactctaataattttacgaatactatactcgaagtcggacccgttactatggccggattgattaatctatctagtacacctactaatagaatgattaggtacgattacgctactaagacagggcaatgcggaggggtgttatgcgcaaccggtaagatattcggtatacacgtagggggtaacggtagacaggggtttagcgcacaattgaaaaaacagtatttcgtcgaaaaacagggtcaggtaatcgctagacataaggttagagagtttaatattaatcccgttaataccgctactaagtctaaattgcatccatcagtgttttacgacgtattcccaggcgataaggaaccagccgtactatccgataacgatcctagacttgaggttaagttaaccgaatcactattctctaagtataagggtaacgttaataccgaacctaccgaaaatatgttagtcgcagtcgatcattacgccggtcaattgctatcacttgatataccgactagcgaattgacacttaaagaggcactatacggagtcgacggactcgaaccaatcgatattactacatcagccggattcccatacgttagtctaggtattaaaaaaagggatatacttaacaaagagacacaggataccgaaaaaatgaaattttatctagataaatacggtatcgatctaccattagtgacttatattaaagacgaattgcgatcagtcgataaggttaggttaggtaagtctagattgatcgaagctagttcacttaacgatagcgttaatatgcgtatgaaattgggtaatctgtataaggcatttcaccaaaatcccggagtgttaaccggatccgcagtcggatgcgatccagacgttttttggtcagtgataccatgtcttatggacggacatcttatggcattcgattactctaatttcgatgcatcactatcacccgtttggttcgtatgtcttgagaaagtgttaactaagttagggttcgcaggatctagtctgatacaatcaatttgtaatacacatcatatttttagagacgaaatatacgtagtcgaagggggtatgcctagcggatgttccggtacatcaatttttaactctatgattaacaatattattattagaacattgatacttgacgcatataagggtatcgatctagataagcttaagatactcgcatacggagacgatctaatcgttagttatccatacgaactcgatccacaagtgttagcgacattgggtaagaattacggattgactattacacctcccgataaatccgaaacttttacgaaaatgacatgggagaatctgacatttctgaaacgatattttaaacccgatcagcaatttccattcttagtgcatcccgttatgcctatgaaagatatacacgaatcaattagatggactaaggatccaaaaaatacacaggatcacgttaggtcactatgtatgttagcatggcattcaggcgaaaaagagtataacgaattcatacaaaaaattagaactaccgatatcggtaagtgtctgatattgcccgaatactcagtgcttagacgtagatggttagatctattctaggttaacaatatagacacttaatttgagtagaagtaggagtttat

SEQ ID NO: 33 - DNA, 10735 bp, Dengue virus type 1
  source 1..10735: mol_type = unassigned DNA
agttgttagtctacgtggaccgacaagaacagtttcgaatcggaagcttgcttaacgtagttctaacagttttttattagagagcagatctctgatgaataatcaacgtaaaaaaacgggtcgtccgtcgtttaatatgttaaaacgtgcgcgtaatcgtgtatcgacggtatcgcaattagcgaaacgtttttcgaaaggtttattatcgggtcaaggtccgatgaaattagtaatggcgtttatagcgtttttacgttttttagcgataccgccgacggcgggtatattagcgcgttggggttcgtttaaaaaaaatggtgcgataaaagtattacgtggttttaaaaaagaaatatcgaatatgttaaatataatgaatcgtcgtaaacgttcggtaacgatgttattaatgttattaccgacggcgttagcgtttcatttaacgacgcgtggtggtgaaccgcatatgatagtatcgaaacaagaacgtggtaaatcgttattatttaaaacgtcggcgggtgtaaatatgtgtacgttaatagcgatggatttaggtgaattatgtgaagatacgatgacgtataaatgtccgcgtataacggaaacggaaccggatgatgtagattgttggtgtaatgcgacggaaacgtgggtaacgtatggtacgtgttcgcaaacgggtgaacatcgtcgtgataaacgttcggtagcgttagcgccgcatgtaggtttaggtttagaaacgcgtacggaaacgtggatgtcgtcggaaggtgcgtggaaacaaatacaaaaagtagaaacgtgggcgttacgtcatccgggttttacggtaatagcgttatttttagcgcatgcgataggtacgtcgataacgcaaaaaggtataatatttatattattaatgttagtaacgccgtcgatggcgatgcgttgtgtaggtataggtaatcgtgattttgtagaaggtttatcgggtgcgacgtgggtagatgtagtattagaacatggttcgtgtgtaacgacgatggcgaaagataaaccgacgttagatatagaattattaaaaacggaagtaacgaatccggcggtattacgtaaattatgtatagaagcgaaaatatcgaatacgacgacggattcgcgttgtccgacgcaaggtgaagcgacgttagtagaagaacaagatacgaattttgtatgtcgtcgtacgtttgtagatcgtggttggggtaatggttgtggtttatttggtaaaggttcgttaataacgtgtgcgaaatttaaatgtgtaacgaaattagaaggtaaaatagtacaatatgaaaatttaaaatattcggtaatagtaacggtacatacgggtgatcaacatcaagtaggtaatgaaacgacggaacatggtacgacggcgacgataacgccgcaagcgccgacgtcggaaatacaattaacggattatggtgcgttaacgttagattgttcgccgcgtacgggtttagattttaatgaaatggtattattaacgatgaaaaaaaaatcgtggttagtacataaacaatggtttttagatttaccgttaccgtggacgtcgggtgcgtcgacgtcgcaagaaacgtggaatcgtcaagatttattagtaacgtttaaaacggcgcatgcgaaaaaacaagaagtagtagtattaggttcgcaagaaggtgcgatgcatacggcgttaacgggtgcgacggaaatacaaacgtcgggtacgacgacgatatttgcgggtcatttaaaatgtcgtttaaaaatggataaattaatattaaaaggtatgtcgtatgtaatgtgtacgggttcgtttaaattagaaaaagaagtagcggaaacgcaacatggtacggtattagtacaagtaaaatatgaaggtacggatgcgccgtgtaaaataccgttttcgtcgcaagatgaaaaaggtgtaacgcaaaatggtcgtttaataacggcgaatccgatagtaacggataaagaaaaaccggtaaatatagaagcggaaccgccgtttggtgaatcgtatatagtagtaggtgcgggtgaaaaagcgttaaaattatcgtggtttaaaaaaggttcgtcgataggtaaaatgtttgaagcgacggcgcgtggtgcgcgtcgtatggcgatattaggtgatacggcgtgggattttggttcgataggtggtgtatttacgtcggtaggtaaattaatacatcaaatatttggtacggcgtatggtgtattattttcgggtgtatcgtggacgatgaaaataggtataggtatattattaacgtggttaggtttaaattcgcgttcgacgtcgttatcgatgacgtgtatagcggtaggtatggtaacgttatatttaggtgtaatggtacaagcggattcgggttgtgtaataaattggaaaggtcgtgaattaaaatgtggttcgggtatatttgtaacgaatgaagtacatacgtggacggaacaatataaatttcaagcggattcgccgaaacgtttatcggcggcgataggtaaagcgtgggaagaaggtgtatgtggtatacgttcggcgacgcgtttagaaaatataatgtggaaacaaatatcgaatgaattaaatcatatattattagaaaatgatatgaaatttacggtagtagtaggtgatgtatcgggtatattagcgcaaggtaaaaaaatgatacgtccgcaaccgatggaacataaatattcgtggaaatcgtggggtaaagcgaaaataataggtgcggatgtacaaaatacgacgtttataatagatggtccgaatacgccggaatgtccggataatcaacgtgcgtggaatatatgggaagtagaagattatggttttggtatatttacgacgaatatatggttaaaattacgtgattcgtatacgcaagtatgtgatcatcgtttaatgtcggcggcgataaaagattcgaaagcggtacatgcggatatgggttattggatagaatcggaaaaaaatgaaacgtggaaattagcgcgtgcgtcgtttatagaagtaaaaacgtgtatatggccgaaatcgcatacgttatggtcgaatggtgtattagaatcggaaatgataataccgaaaatatatggtggtccgatatcgcaacataattatcgtccgggttattttacgcaaacggcgggtccgtggcatttaggtaaattagaattagattttgatttatgtgaaggtacgacggtagtagtagatgaacattgtggtaatcgtggtccgtcgttacgtacgacgacggtaacgggtaaaacgatacatgaatggtgttgtcgttcgtgtacgttaccgccgttacgttttaaaggtgaagatggttgttggtatggtatggaaatacgtccggtaaaagaaaaagaagaaaatttagtaaaatcgatggtatcggcgggttcgggtgaagtagattcgttttcgttaggtttattatgtatatcgataatgatagaagaagtaatgcgttcgcgttggtcgcgtaaaatgttaatgacgggtacgttagcggtatttttattattaacgatgggtcaattaacgtggaatgatttaatacgtttatgtataatggtaggtgcgaatgcgtcggataaaatgggtatgggtacgacgtatttagcgttaatggcgacgtttcgtatgcgtccgatgtttgcggtaggtttattatttcgtcgtttaacgtcgcgtgaagtattattattaacggtaggtttatcgttagtagcgtcggtagaattaccgaattcgttagaagaattaggtgatggtttagcgatgggtataatgatgttaaaattattaacggattttcaatcgcatcaattatgggcgacgttattatcgttaacgtttgtaaaaacgacgttttcgttacattatgcgtggaaaacgatggcgatgatattatcgatagtatcgttatttccgttatgtttatcgacgacgtcgcaaaaaacgacgtggttaccggtattattaggttcgttaggttgtaaaccgttaacgatgtttttaataacggaaaataaaatatggggtcgtaaatcgtggccgttaaatgaaggtataatggcggtaggtatagtatcgatattattatcgtcgttattaaaaaatgatgtaccgttagcgggtccgttaatagcgggtggtatgttaatagcgtgttatgtaatatcgggttcgtcggcggatttatcgttagaaaaagcggcggaagtatcgtgggaagaagaagcggaacattcgggtgcgtcgcataatatattagtagaagtacaagatgatggtacgatgaaaataaaagatgaagaacgtgatgatacgttaacgatattattaaaagcgacgttattagcgatatcgggtgtatatccgatgtcgataccggcgacgttatttgtatggtatttttggcaaaaaaaaaaacaacgttcgggtgtattatgggatacgccgtcgccgccggaagtagaacgtgcggtattagatgatggtatatatcgtatattacaacgtggtttattaggtcgttcgcaagtaggtgtaggtgtatttcaagaaggtgtatttcatacgatgtggcatgtaacgcgtggtgcggtattaatgtatcaaggtaaacgtttagaaccgtcgtgggcgtcggtaaaaaaagatttaatatcgtatggtggtggttggcgttttcaaggttcgtggaatgcgggtgaagaagtacaagtaatagcggtagaaccgggtaaaaatccgaaaaatgtacaaacggcgccgggtacgtttaaaacgccggaaggtgaagtaggtgcgatagcgttagattttaaaccgggtacgtcgggttcgccgatagtaaatcgtgaaggtaaaatagtaggtttatatggtaatggtgtagtaacgacgtcgggtacgtatgtatcggcgatagcgcaagcgaaagcgtcgcaagaaggtccgttaccggaaatagaagatgaagtatttcgtaaacgtaatttaacgataatggatttacatccgggttcgggtaaaacgcgtcgttatttaccggcgatagtacgtgaagcgataaaacgtaaattacgtacgttagtattagcgccgacgcgtgtagtagcgtcggaaatggcggaagcgttaaaaggtatgccgatacgttatcaaacgacggcggtaaaatcggaacatacgggtaaagaaatagtagatttaatgtgtcatgcgacgtttacgatgcgtttattatcgccggtacgtgtaccgaattataatatgataataatggatgaagcgcattttacggatccggcgtcgatagcggcgcgtggttatatatcgacgcgtgtaggtatgggtgaagcggcggcgatatttatgacggcgacgccgccgggttcggtagaagcgtttccgcaatcgaatgcggtaatacaagatgaagaacgtgatataccggaacgttcgtggaattcgggttatgattggataacggattttccgggtaaaacggtatggtttgtaccgtcgataaaatcgggtaatgatatagcgaattgtttacgtaaaaatggtaaacgtgtagtacaattatcgcgtaaaacgtttgatacggaatatcaaaaaacgaaaaataatgattgggattatgtagtaacgacggatatatcggaaatgggtgcgaattttcgtgcggatcgtgtaatagatccgcgtcgttgtttaaaaccggtaatattaaaagatggtccggaacgtgtaatattagcgggtccgatgccggtaacggtagcgtcggcggcgcaacgtcgtggtcgtataggtcgtaatcaaaataaagaaggtgatcaatatatatatatgggtcaaccgttaaataatgatgaagatcatgcgcattggacggaagcgaaaatgttattagataatataaatacgccggaaggtataataccggcgttatttgaaccggaacgtgaaaaatcggcggcgatagatggtgaatatcgtttacgtggtgaagcgcgtaaaacgtttgtagaattaatgcgtcgtggtgatttaccggtatggttatcgtataaagtagcgtcggaaggttttcaatattcggatcgtcgttggtgttttgatggtgaacgtaataatcaagtattagaagaaaatatggatgtagaaatatggacgaaagaaggtgaacgtaaaaaattacgtccgcgttggttagatgcgcgtacgtattcggatccgttagcgttacgtgaatttaaagaatttgcggcgggtcgtcgttcggtatcgggtgatttaatattagaaataggtaaattaccgcaacatttaacgcaacgtgcgcaaaatgcgttagataatttagtaatgttacataattcggaacaaggtggtaaagcgtatcgtcatgcgatggaagaattaccggatacgatagaaacgttaatgttattagcgttaatagcggtattaacgggtggtgtaacgttattttttttatcgggtcgtggtttaggtaaaacgtcgataggtttattatgtgtaatagcgtcgtcggcgttattatggatggcgtcggtagaaccgcattggatagcggcgtcgataatattagaattttttttaatggtattattaataccggaaccggatcgtcaacgtacgccgcaagataatcaattagcgtatgtagtaataggtttattatttatgatattaacggtagcggcgaatgaaatgggtttattagaaacgacgaaaaaagatttaggtataggtcatgcggcggcggaaaatcatcatcatgcggcgatgttagatgtagatttacatccggcgtcggcgtggacgttatatgcggtagcgacgacgataataacgccgatgatgcgtcatacgatagaaaatacgacggcgaatatatcgttaacggcgatagcgaatcaagcggcgatattaatgggtttagataaaggttggccgatatcgaaaatggatataggtgtaccgttattagcgttaggttgttattcgcaagtaaatccgttaacgttaacggcggcggtattaatgttagtagcgcattatgcgataataggtccgggtttacaagcgaaagcgacgcgtgaagcgcaaaaacgtacggcggcgggtataatgaaaaatccgacggtagatggtatagtagcgatagatttagatccggtagtatatgatgcgaaatttgaaaaacaattaggtcaaataatgttattaatattatgtacgtcgcaaatattattaatgcgtacgacgtgggcgttatgtgaatcgataacgttagcgacgggtccgttaacgacgttatgggaaggttcgccgggtaaattttggaatacgacgatagcggtatcgatggcgaatatatttcgtggttcgtatttagcgggtgcgggtttagcgttttcgttaatgaaatcgttaggtggtggtcgtcgtggtacgggtgcgcaaggtgaaacgttaggtgaaaaatggaaacgtcaattaaatcaattatcgaaatcggaatttaatacgtataaacgttcgggtataatagaagtagatcgttcggaagcgaaagaaggtttaaaacgtggtgaaacgacgaaacatgcggtatcgcgtggtacggcgaaattacgttggtttgtagaacgtaatttagtaaaaccggaaggtaaagtaatagatttaggttgtggtcgtggtggttggtcgtattattgtgcgggtttaaaaaaagtaacggaagtaaaaggttatacgaaaggtggtccgggtcatgaagaaccgataccgatggcgacgtatggttggaatttagtaaaattatattcgggtaaagatgtattttttacgccgccggaaaaatgtgatacgttattatgtgatataggtgaatcgtcgccgaatccgacgatagaagaaggtcgtacgttacgtgtattaaaaatggtagaaccgtggttacgtggtaatcaattttgtataaaaatattaaatccgtatatgccgtcggtagtagaaacgttagaacaaatgcaacgtaaacatggtggtatgttagtacgtaatccgttatcgcgtaattcgacgcatgaaatgtattgggtatcgtgtggtacgggtaatatagtatcggcggtaaatatgacgtcgcgtatgttattaaatcgttttacgatggcgcatcgtaaaccgacgtatgaacgtgatgtagatttaggtgcgggtacgcgtcatgtagcggtagaaccggaagtagcgaatttagatataataggtcaacgtatagaaaatataaaaaatgaacataaatcgacgtggcattatgatgaagataatccgtataaaacgtgggcgtatcatggttcgtatgaagtaaaaccgtcgggttcggcgtcgtcgatggtaaatggtgtagtacgtttattaacgaaaccgtgggatgtaataccgatggtaacgcaaatagcgatgacggatacgacgccgtttggtcaacaacgtgtatttaaagaaaaagtagatacgcgtacgccgaaagcgaaacgtggtacggcgcaaataatggaagtaacggcgcgttggttatggggttttttatcgcgtaataaaaaaccgcgtatatgtacgcgtgaagaatttacgcgtaaagtacgttcgaatgcggcgataggtgcggtatttgtagatgaaaatcaatggaattcggcgaaagaagcggtagaagatgaacgtttttgggatttagtacatcgtgaacgtgaattacataaacaaggtaaatgtgcgacgtgtgtatataatatgatgggtaaacgtgaaaaaaaattaggtgaatttggtaaagcgaaaggttcgcgtgcgatatggtatatgtggttaggtgcgcgttttttagaatttgaagcgttaggttttatgaatgaagatcattggttttcgcgtgaaaattcgttatcgggtgtagaaggtgaaggtttacataaattaggttatatattacgtgatatatcgaaaataccgggtggtaatatgtatgcggatgatacggcgggttgggatacgcgtataacggaagatgatttacaaaatgaagcgaaaataacggatataatggaaccggaacatgcgttattagcgacgtcgatatttaaattaacgtatcaaaataaagtagtacgtgtacaacgtccggcgaaaaatggtacggtaatggatgtaatatcgcgtcgtgatcaacgtggttcgggtcaagtaggtacgtatggtttaaatacgtttacgaatatggaagcgcaattaatacgtcaaatggaatcggaaggtatattttcgccgtcggaattagaaacgccgaatttagcggaacgtgtattagattggttaaaaaaacatggtacggaacgtttaaaacgtatggcgatatcgggtgatgattgtgtagtaaaaccgatagatgatcgttttgcgacggcgttaacggcgttaaatgatatgggtaaagtacgtaaagatataccgcaatgggaaccgtcgaaaggttggaatgattggcaacaagtaccgttttgttcgcatcattttcatcaattaataatgaaagatggtcgtgaaatagtagtaccgtgtcgtaatcaagatgaattagtaggtcgtgcgcgtgtatcgcaaggtgcgggttggtcgttacgtgaaacggcgtgtttaggtaaatcgtatgcgcaaatgtggcaattaatgtattttcatcgtcgtgatttacgtttagcggcgaatgcgatatgttcggcggtaccggtagattgggtaccgacgtcgcgtacgacgtggtcgatacatgcgcatcatcaatggatgacgacggaagatatgttatcggtatggaatcgtgtatggatagaagaaaatccgtggatggaagataaaacgcatgtatcgtcgtgggaagatgtaccgtatttaggtaaacgtgaagatcaatggtgtggttcgttaataggtttaacggcgcgtgcgacgtgggcgacgaatatacaagtagcgataaatcaagtacgtcgtttaataggtaatgaaaattatttagattttatgacgtcgatgaaacgttttaaaaatgaatcggatccggaaggtgcgttatggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 34 - DNA, 10735 bp, unidentified
  misc_feature 1..10735: note = synthetic
  source 1..10735: mol_type = other DNA
agttgttagtctacgtggaccgacaagaacagtttcgaatcggaagcttgcttaacgtagttctaacagttttttattagagagcagatctctgatgaataaccaacgaaaaaaaaccggacgaccatcattcaatatgcttaaacgcgctaggaatagggtgtcaaccgtatcgcaattggctaagagattctctaagggactgttaagcggacagggaccaatgaaactggttatggcattcatagcgtttctgagatttctcgcaataccgccaaccgccggaatactcgctagatgggggtcattcaaaaaaaacggcgcaattaaagtgcttagggggttcaaaaaagagatatcgaatatgcttaacattatgaatagacgtaagagatccgttacaatgctattgatgctattgccaaccgcactagcctttcacctaacgactagggggggggaaccacatatgatagtgagtaagcaagagagaggtaagtcactattgttcaaaacatccgccggagtgaatatgtgtacactaatcgctatggacttaggcgaattgtgcgaagacactatgacatataagtgtcctaggattaccgaaaccgaaccagacgacgtcgattgttggtgtaacgcaaccgaaacatgggtgacatacggaacatgttcgcaaaccggagagcatagacgcgataagagatcagtcgcactcgcaccacacgtcggattggggttagagactagaaccgaaacatggatgtctagcgaaggcgcatggaaacagatacagaaagtcgagacatgggcccttagacacccagggtttaccgtaatcgcactatttctcgcacacgcaatcggaacgtcaattacgcaaaaagggattatattcatactgcttatgttagtgacaccatctatggctatgagatgcgtcggaatcggaaatagggatttcgttgagggactatccggagcgacatgggtcgacgtagtgctcgaacacggatcatgcgttacgactatggccaaagacaaaccgacacttgacatagagttactgaaaaccgaagtgactaacccagccgtactgagaaagttgtgtatcgaagctaagatatcgaatacgactaccgatagtaggtgtccaacgcaaggcgaagcgacactagtcgaagagcaggatacgaatttcgtatgtagacgtacattcgtcgatagggggtggggaaacggatgcggattgttcggtaagggatcactgattacatgcgctaaattcaaatgcgttacgaaactcgagggtaagatagtgcaatacgagaatctgaaatatagcgtaatcgttacagtgcataccggagaccaacaccaagtgggaaacgagactaccgaacacggaactaccgcaacgattacgccacaagcccctactagcgaaatccaattgaccgattacggcgcactaacactcgattgttcacctagaaccggattggactttaacgaaatggtgctattgactatgaaaaaaaaatcatggttagtgcataagcaatggttcttagacctaccactaccatggactagcggagctagtacgtcacaggagacatggaatagacaggatctgttagtgacattcaaaaccgcacacgctaaaaaacaggaggtcgtagtgttagggtcacaagagggagctatgcataccgcactaacaggcgcaaccgagatacagactagcggaacgactacgatattcgccggacaccttaagtgtagactgaaaatggacaaactgatactgaaaggtatgtcatacgttatgtgtaccggatcattcaaactcgaaaaagaggtcgccgaaacgcaacacggaacagtgttagtgcaagtgaaatacgagggaactgacgcaccatgtaagataccgttttcgtcacaggacgaaaaaggcgttacgcaaaacggtagactgattaccgctaacccaatcgttaccgataaagagaaaccggttaatatcgaagccgaaccaccattcggcgaatcatatatcgtagtcggagccggagagaaagcccttaagctatcatggtttaaaaaagggtcatcaatcggtaagatgttcgaagcaaccgctagaggcgctagacgtatggccatactaggcgataccgcatgggatttcggatcaatcggaggagtgtttacgtcagtcggaaaactgatacaccaaattttcggaaccgcatacggagtgttgtttagcggagtgtcatggactatgaaaatcggaatcggaatactgttgacatggttagggttgaactctagatcgactagtctatctatgacatgtatcgcagtcggaatggtgacactatacttaggcgtaatggtgcaagccgatagcggatgcgttattaattggaaagggagagagcttaaatgcggatccggaatattcgttacgaacgaagtgcatacatggaccgaacagtataaattccaagccgatagtccgaaaagactatctgccgcaatcggtaaggcatgggaggagggagtgtgcggaattaggtcagcgactagactcgaaaacattatgtggaaacagatatctaacgaactgaatcacatactgttagagaacgatatgaaatttacagtcgtagtcggagacgttagcggaatactcgcacagggtaagaaaatgattagaccgcaacctatggagcataagtatagttggaaatcatggggaaaagcgaaaattatcggagccgacgtgcaaaatacgacattcataatcgacggaccgaatacgccagagtgtccagacaatcagagagcatggaacatatgggaggtcgaagactacggattcggaatttttacgactaacatatggcttaagcttagagactcatatacacaggtatgcgatcatagattgatgtctgccgcaatcaaagactctaaagccgtacacgccgatatggggtattggatcgaatccgaaaaaaacgagacatggaaactcgctagagcgtcattcatagaggttaagacatgtatttggcctaagtcacatacattgtggtctaacggagtgcttgagtctgagatgataatccctaagatatacggcggaccgatatcgcaacataactatagaccagggtattttacgcaaacagccggaccatggcatctcggtaagcttgagttagacttcgatctatgcgaagggactaccgtagtggtcgacgaacattgcggtaataggggacctagtctgagaacgactaccgttaccggtaagactatacacgaatggtgttgtaggtcatgtacactaccaccacttagattcaaaggcgaagacggatgttggtacggaatggagattagaccagtgaaagagaaagaggaaaacctagtgaaatctatggtgtcagccggatcaggcgaagtcgactcattctcactcggactgttatgcatatcgattatgatcgaagaggttatgcgatctagatggtcacgaaaaatgcttatgaccggaacactagccgtttttctgttactgactatggggcaattgacatggaacgatctgattaggttgtgtattatggtcggagctaacgctagcgataagatgggtatgggaacgacatacttagcccttatggctacatttagaatgcgaccaatgttcgccgtagggttactgtttagacggttgactagtagggaagtgctattgttgacagtcggactgtcactagtcgctagcgttgagttgcctaactcactcgaagagttaggcgacggactcgcaatggggattatgatgcttaagctattgaccgatttccaatctcaccaattgtgggctacactattgtcactgacattcgttaagactacattctcattgcattacgcatggaaaactatggctatgatactgtcaatcgttagtctattcccactatgtctatctacaactagtcagaaaactacatggttgccagtgctattggggtcactagggtgtaaaccattgactatgtttctgattaccgaaaacaaaatttgggggagaaagtcatggccacttaacgagggaattatggccgtagggatagtgtcaatactgctatctagtctgcttaaaaacgacgtgccactagccggaccactgatagccggaggtatgctaatcgcatgttacgtgatatccggatctagcgccgatctgtcactcgaaaaagccgccgaagtgtcatgggaagaggaagccgaacactctggcgcatcacataacatactagtcgaagtgcaagacgacggaactatgaaaattaaagacgaagagagagacgatacacttacgatactgcttaaagcgacactgttagcgatatccggagtgtatcctatgtcaatacccgctacactattcgtttggtatttttggcaaaaaaaaaaacagagatccggagtgttgtgggatacacctagtccacccgaagtcgagagagccgtactcgacgacggaatatataggatactgcaacggggattgctcggtaggtcacaggtcggagtgggagtgtttcaggagggagtgtttcacactatgtggcacgttacgagaggcgccgtactgatgtatcagggtaagagactcgaacctagttgggctagcgttaaaaaagacctaatctcatacggaggggggtggagatttcaggggtcatggaacgccggagaggaagtgcaagtgatagccgtcgaacccggtaagaaccctaaaaacgtgcaaaccgcacccggaacgtttaagacacccgaaggcgaagtgggcgcaatcgcactcgactttaaacccggaactagcggatcaccaatcgttaatagagagggtaagatagtcggattgtacggaaacggagtggttacgactagcggaacatacgttagcgcaatagcgcaagcgaaagcgtcacaagagggaccactaccagagatagaggacgaagtgtttagaaagcgtaatctgacaattatggacctacatcccggatccggtaagactagacggtatctgccagcaatcgttagggaggcaattaagagaaagttgcgtacactagtgttagccccaactagagtggtcgctagcgaaatggccgaagcccttaagggaatgccaattaggtatcagactaccgccgttaagtccgaacataccggtaaggagatagtcgaccttatgtgtcacgctacattcacaatgagactgctatcaccagtgagagtgcctaactataatatgataattatggacgaagcccattttacagacccagcctcaatcgccgctagggggtatattagtactagggtgggaatgggcgaagccgccgcaatttttatgacagcgacaccacccggatccgtcgaagcgtttccgcaatctaacgccgttatacaggacgaagagagagacatacccgaacggtcatggaatagcggatacgattggataaccgattttcccggtaagacagtgtggttcgtaccgtcaattaagtccggaaacgatatcgctaattgtcttagaaaaaacggtaagagagtggtgcaattgtctagaaagacattcgataccgaataccaaaaaactaagaataacgattgggattacgtagtgacaaccgacatatccgaaatgggcgctaactttagagccgatagggtgatagaccctagacggtgtctgaaaccagtgatactgaaagacggaccagagagagtgatactagccggaccaatgccagtgacagtcgcatccgccgcacaacgtaggggacggatagggagaaaccaaaacaaagagggagaccaatacatatatatgggacagccactgaataacgacgaagaccacgcacattggaccgaagcgaaaatgctattagacaatattaacacacccgaagggattatacccgcactattcgaacccgaacgcgaaaaatccgccgcaatcgacggcgaatatagactgagaggcgaagctagaaagacattcgttgagcttatgcgtagaggcgatctacccgtatggttgtcatacaaagtcgctagcgaagggtttcagtattccgataggagatggtgtttcgacggcgaacggaataaccaagtgctcgaagagaatatggacgttgagatatggactaaagagggcgaacgaaaaaaactgagacctagatggttagacgcacgtacatattccgatccgttagcccttagagagtttaaagagttcgcagccggtaggagatccgttagcggagatctgatactcgaaatcggtaagctaccacaacacctaacgcaacgagcccaaaacgcactagacaatctggttatgttgcataactccgaacagggcggtaaggcatatagacacgctatggaagagttacccgatacaatcgaaacacttatgctactcgcactgatagccgtactgacaggcggagtgacactattctttctatccggtagggggttaggtaagacatcaatcggactgttatgcgtaatcgcatctagcgcactattgtggatggctagcgtcgaaccacattggatagccgcatcaattatactcgaattctttcttatggtgttactgatacccgaacccgataggcaacgaacaccacaggataaccaattggcatacgtcgtaatcggactattgtttatgatactgacagtcgccgctaacgaaatgggattgctcgagacaacgaaaaaagacctagggatcggacacgccgcagccgaaaatcaccatcacgccgcaatgttagacgtcgatctgcatcccgctagcgcatggacactatacgcagtcgcaacgacaattattacgcctatgatgcgacatacaatcgagaatactaccgctaacatatcgctaaccgctatagcgaatcaggccgcaatactgatggggttagacaaagggtggccaattagtaagatggatatcggagtgccactgttagccttagggtgttatagtcaggttaacccattgacattgaccgcagccgtacttatgctagtcgcacattacgctataatcggaccaggactgcaagcgaaagcgacacgcgaagcgcaaaaaagaaccgcagccggaattatgaaaaaccctacagtcgacggaatagtcgcaatcgacttagacccagtggtgtacgacgctaaattcgaaaaacagttgggacagattatgctactgatactgtgtacgtcacagatactgcttatgcgaactacatgggcactatgcgaatcaattacactcgcaaccggaccattgactacattgtgggagggatcacccggtaagttttggaatacgactatagccgttagtatggctaacatttttagggggtcatatctagccggagccggactcgcattctcacttatgaaatcactcggagggggacgtagggggacaggcgcacagggagagacactaggcgaaaaatggaaacgacaattgaaccaattgtctaaatccgaatttaacacttataagagatccggaataatcgaagtcgatagatccgaagcgaaagagggactgaaacgcggagagacaacgaaacacgccgttagtaggggaaccgctaagcttagatggttcgttgagcgtaacttagtgaaaccagagggtaaggtaatcgatctcggatgcggtaggggggggtggtcatactattgcgccggactgaaaaaagtgaccgaagtgaaagggtatactaagggcggacccggacacgaagagccaataccgatggcaacatacggatggaacctagtgaaactgtattccggtaaggacgtattctttacaccacccgaaaaatgcgatacactgttatgcgatataggcgaatctagtcctaaccctacaatcgaagagggacgtacacttagagtgcttaaaatggtcgaaccatggttgagggggaatcagttttgtattaagatactgaatccatatatgcctagcgtagtcgagacactcgaacagatgcaacgaaaacacggcggaatgctagtgagaaacccactatctaggaattcgacacacgaaatgtattgggtgtcatgcggaaccggaaacatagtgtcagccgttaatatgactagtagaatgctactgaatcggtttacaatggcccatagaaaaccgacatacgaaagagacgtcgacctaggagccggaactagacacgttgccgtcgaacccgaagtcgctaacttagacataatcggacagagaatcgagaatattaaaaacgaacacaaatcgacatggcattacgacgaagacaatccgtataagacatgggcatatcacggatcatacgaagtgaaacctagcggatccgctagttctatggttaacggagtggtgagactgttgactaagccatgggacgtgataccgatggtgacacaaatcgctatgaccgatacgacaccattcggacagcaaagagtgtttaaagagaaagtcgacactagaacacctaaagcgaaacgcggaaccgcacagattatggaggttaccgctagatggttgtgggggttcctatctagaaacaaaaaacctaggatttgtacacgcgaagagtttacacgaaaagtgagatctaacgccgcaatcggagccgtattcgtagacgaaaaccaatggaattccgctaaagaggccgtcgaagacgaacggttttgggacttagtgcatagggaacgcgaattgcataaacagggtaagtgcgcaacatgcgtatacaatatgatgggtaagcgcgaaaaaaagttaggcgaattcggtaaggctaagggatctagagcgatatggtatatgtggttaggcgctaggtttctcgaattcgaagcactcggatttatgaacgaagaccattggttttctagagagaatagtctatccggagtcgagggagagggattgcataaactcggatatatactgagagacatatctaagatacccggagggaatatgtacgccgacgatacagccggatgggatacacgaattaccgaagacgatctgcaaaacgaagcgaaaattaccgatattatggaacccgaacacgcactgttagcgacatctatttttaagttgacatatcagaataaggtcgttagggtgcaacgacccgctaaaaacggaacggttatggacgtgattagtaggagagaccaacgcggatccggacaggtcggaacatacggactgaatacgtttacgaatatggaagcgcaattgattagacagatggagtctgaggggatattctcacctagcgaactcgagacacctaacctagccgaacgggtactcgattggttgaaaaaacacggaaccgaaagactgaaaagaatggcaattagcggagacgattgcgtagtgaaaccaatcgacgatagattcgcaaccgcactaaccgcacttaacgatatgggtaaggtgagaaaagacataccgcaatgggagccatctaagggatggaacgattggcaacaggtgccattttgctcacatcactttcaccaattgattatgaaagacggtagggagatagtcgtgccatgtaggaatcaggacgaactcgtaggtagggctagagtgtcacagggagccggatggtcacttagggaaaccgcatgcctaggtaagtcatacgctcaaatgtggcaattgatgtatttccatagacgcgatctgagactagccgctaacgctatatgctctgccgtaccagtcgattgggtgccaactagtagaacgacatggtctatacacgcacatcaccaatggatgacaaccgaagacatgctatccgtttggaatagggtgtggatcgaagagaatccatggatggaggataagacacacgtgtctagttgggaggacgtgccatatctcggaaaacgcgaagaccaatggtgcggatcactaatcggattgaccgctagagcgacatgggcaactaacatacaggtcgcaattaaccaagtgagacgattgatcggaaacgagaattatctcgactttatgacatctatgaaaagattcaaaaacgaatccgatcccgaaggcgcactatggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 35 - DNA, 9181 bp, Human immunodeficiency virus type 1
  source 1..9181: mol_type = unassigned DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgcgtgcgtcggtattatcgggtggtgaattagatcgttgggaaaaaatacgtttacgtccgggtggtaaaaaaaaatataaattaaaacatatagtatgggcgtcgcgtgaattagaacgttttgcggtaaatccgggtttattagaaacgtcggaaggttgtcgtcaaatattaggtcaattacaaccgtcgttacaaacgggttcggaagaattacgttcgttatataatacggtagcgacgttatattgtgtacatcaacgtatagaaataaaagatacgaaagaagcgttagataaaatagaagaagaacaaaataaatcgaaaaaaaaagcgcaacaagcggcggcggatacgggtcattcgaatcaagtatcgcaaaattatccgatagtacaaaatatacaaggtcaaatggtacatcaagcgatatcgccgcgtacgttaaatgcgtgggtaaaagtagtagaagaaaaagcgttttcgccggaagtaataccgatgttttcggcgttatcggaaggtgcgacgccgcaagatttaaatacgatgttaaatacggtaggtggtcatcaagcggcgatgcaaatgttaaaagaaacgataaatgaagaagcggcggaatgggatcgtgtacatccggtacatgcgggtccgatagcgccgggtcaaatgcgtgaaccgcgtggttcggatatagcgggtacgacgtcgacgttacaagaacaaataggttggatgacgaataatccgccgataccggtaggtgaaatatataaacgttggataatattaggtttaaataaaatagtacgtatgtattcgccgacgtcgatattagatatacgtcaaggtccgaaagaaccgtttcgtgattatgtagatcgtttttataaaacgttacgtgcggaacaagcgtcgcaagaagtaaaaaattggatgacggaaacgttattagtacaaaatgcgaatccggattgtaaaacgatattaaaagcgttaggtccggcggcgacgttagaagaaatgatgacggcgtgtcaaggtgtaggtggtccgggtcataaagcgcgtgtattagcggaagcgatgtcgcaagtaacgaattcggcgacgataatgatgcaacgtggtaattttcgtaatcaacgtaaaatagtaaaatgttttaattgtggtaaagaaggtcatacggcgcgtaattgtcgtgcgccgcgtaaaaaaggttgttggaaatgtggtaaagaaggtcatcaaatgaaagattgtacggaacgtcaagcgaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggtggtcaattaaaagaagcgttattagatacgggtgcggatgatacggtattagaagaaatgtcgttaccgggtcgttggaaaccgaaaatgataggtggtataggtggttttataaaagtacgtcaatatgatcaaatattaatagaaatatgtggtcataaagcgataggtacggtattagtaggtccgacgccggtaaatataataggtcgtaatttattaacgcaaataggttgtacgttaaattttccgatatcgccgatagaaacggtaccggtaaaattaaaaccgggtatggatggtccgaaagtaaaacaatggccgttaacggaagaaaaaataaaagcgttagtagaaatatgtacggaaatggaaaaagaaggtaaaatatcgaaaataggtccggaaaatccgtataatacgccggtatttgcgataaaaaaaaaagattcgacgaaatggcgtaaattagtagattttcgtgaattaaataaacgtacgcaagatttttgggaagtacaattaggtataccgcatccggcgggtttaaaaaaaaaaaaatcggtaacggtattagatgtaggtgatgcgtatttttcggtaccgttagatgaagattttcgtaaatatacggcgtttacgataccgtcgataaataatgaaacgccgggtatacgttatcaatataatgtattaccgcaaggttggaaaggttcgccggcgatatttcaatcgtcgatgacgaaaatattagaaccgtttcgtaaacaaaatccggatatagtaatatatcaatatatggatgatttatatgtaggttcggatttagaaataggtcaacatcgtacgaaaatagaagaattacgtcaacatttattacgttggggtttaacgacgccggataaaaaacatcaaaaagaaccgccgtttttatggatgggttatgaattacatccggataaatggacggtacaaccgatagtattaccggaaaaagattcgtggacggtaaatgatatacaaaaattagtaggtaaattaaattgggcgtcgcaaatatatccgggtataaaagtacgtcaattatgtaaattattacgtggtacgaaagcgttaacggaagtaataccgttaacggaagaagcggaattagaattagcggaaaatcgtgaaatattaaaagaaccggtacatggtgtatattatgatccgtcgaaagatttaatagcggaaatacaaaaacaaggtcaaggtcaatggacgtatcaaatatatcaagaaccgtttaaaaatttaaaaacgggtaaatatgcgcgtatgcgtggtgcgcatacgaatgatgtaaaacaattaacggaagcggtacaaaaaataacgacggaatcgatagtaatatggggtaaaacgccgaaatttaaattaccgatacaaaaagaaacgtgggaaacgtggtggacggaatattggcaagcgacgtggataccggaatgggaatttgtaaatacgccgccgttagtaaaattatggtatcaattagaaaaagaaccgatagtaggtgcggaaacgttttatgtagatggtgcggcgaatcgtgaaacgaaattaggtaaagcgggttatgtaacgaatcgtggtcgtcaaaaagtagtaacgttaacggatacgacgaatcaaaaaacggaattacaagcgatatatttagcgttacaagattcgggtttagaagtaaatatagtaacggattcgcaatatgcgttaggtataatacaagcgcaaccggatcaatcggaatcggaattagtaaatcaaataatagaacaattaataaaaaaagaaaaagtatatttagcgtgggtaccggcgcataaaggtataggtggtaatgaacaagtagataaattagtatcggcgggtatacgtaaagtattatttttagatggtatagataaagcgcaagatgaacatgaaaaatatcattcgaattggcgtgcgatggcgtcggattttaatttaccgccggtagtagcgaaagaaatagtagcgtcgtgtgataaatgtcaattaaaaggtgaagcgatgcatggtcaagtagattgttcgccgggtatatggcaattagattgtacgcatttagaaggtaaagtaatattagtagcggtacatgtagcgtcgggttatatagaagcggaagtaataccggcggaaacgggtcaagaaacggcgtattttttattaaaattagcgggtcgttggccggtaaaaacgatacatacggataatggttcgaattttacgggtgcgacggtacgtgcggcgtgttggtgggcgggtataaaacaagaatttggtataccgtataatccgcaatcgcaaggtgtagtagaatcgatgaataaagaattaaaaaaaataataggtcaagtacgtgatcaagcggaacatttaaaaacggcggtacaaatggcggtatttatacataattttaaacgtaaaggtggtataggtggttattcggcgggtgaacgtatagtagatataatagcgacggatatacaaacgaaagaattacaaaaacaaataacgaaaatacaaaattttcgtgtatattatcgtgattcgcgtaatccgttatggaaaggtccggcgaaattattatggaaaggtgaaggtgcggtagtaatacaagataattcggatataaaagtagtaccgcgtcgtaaagcgaaaataatacgtgattatggaaaacagatggcaggtgatgattgtgtggcaagtagacaggatgaggattagaacgtggaaatcgttagtaaaacatcatatgtatgtatcgggtaaagcgcgtggttggttttatcgtcatcattatgaatcgccgcatccgcgtatatcgtcggaagtacatataccgttaggtgatgcgcgtttagtaataacgacgtattggggtttacatacgggtgaacgtgattggcatttaggtcaaggtgtatcgatagaatggcgtaaaaaacgttattcgacgcaagtagatccggaattagcggatcaattaatacatttatattattttgattgtttttcggattcggcgatacgtaaagcgttattaggtcatatagtatcgccgcgttgtgaatatcaagcgggtcataataaagtaggttcgttacaatatttagcgttagcggcgttaataacgccgaaaaaaataaaaccgccgttaccgtcggtaacgaaattaacggaagatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgcgacggaaaaattatgggtaacggtatattatggtgtaccggtatggaaagaagcgacgacgacgttattttgtgcgtcggatgcgaaagcgtatgatacggaagtacataatgtatgggcgacgcatgcgtgtgtaccgacggatccgaatccgcaagaagtagtattagtaaatgtaacggaaaattttaatatgtggaaaaatgatatggtagaacaaatgcatgaagatataatatcgttatgggatcaatcgttaaaaccgtgtgtaaaattaacgccgttatgtgtatcgttaaaatgtacggatttaaaaaatgatacgaatacgaattcgtcgtcgggtcgtatgataatggaaaaaggtgaaataaaaaattgttcgtttaatatatcgacgtcgatacgtggtaaagtacaaaaagaatatgcgtttttttataaattagatataataccgatagataatgatacgacgtcgtataaattaacgtcgtgtaatacgtcggtaataacgcaagcgtgtccgaaagtatcgtttgaaccgataccgatacattattgtgcgccggcgggttttgcgatattaaaatgtaataataaaacgtttaatggtacgggtccgtgtacgaatgtatcgacggtacaatgtacgcatggtatacgtccggtagtatcgacgcaattattattaaatggttcgttagcggaagaagaagtagtaatacgttcggtaaattttacggataatgcgaaaacgataatagtacaattaaatacgtcggtagaaataaattgtacgcgtccgaataataatacgcgtaaacgtatacgtatacaacgtggtccgggtcgtgcgtttgtaacgataggtaaaataggtaatatgcgtcaagcgcattgtaatatatcgcgtgcgaaatggaataatacgttaaaacaaatagcgtcgaaattacgtgaacaatttggtaataataaaacgataatatttaaacaatcgtcgggtggtgatccggaaatagtaacgcattcgtttaattgtggtggtgaatttttttattgtaattcgacgcaattatttaattcgacgtggtttaattcgacgtggtcgacggaaggttcgaataatacggaaggttcggatacgataacgttaccgtgtcgtataaaacaaataataaatatgtggcaaaaagtaggtaaagcgatgtatgcgccgccgatatcgggtcaaatacgttgttcgtcgaatataacgggtttattattaacgcgtgatggtggtaattcgaataatgaatcggaaatatttcgtccgggtggtggtgatatgcgtgataattggcgttcggaattatataaatataaagtagtaaaaatagaaccgttaggtgtagcgccgacgaaagcgaaacgtcgtgtagtacaacgtgaaaaacgtgcggtaggtataggtgcgttatttttaggttttttaggtgcggcgggttcgacgatgggtgcggcgtcgatgacgttaacggtacaagcgcgtcaattattatcgggtatagtacaacaacaaaataatttattacgtgcgatagaagcgcaacaacatttattacaattaacggtatggggtataaaacaattacaagcgcgtatattagcggtagaacgttatttaaaagatcaacaattattaggtatatggggttgttcgggtaaattaatatgtacgacggcggtaccgtggaatgcgtcgtggtcgaataaatcgttagaacaaatatggaatcatacgacgtggatggaatgggatcgtgaaataaataattatacgtcgttaatacattcgttaatagaagaatcgcaaaatcaacaagaaaaaaatgaacaagaattattagaattagataaatgggcgtcgttatggaattggtttaatataacgaattggttatggtatataaaattatttataatgatagtaggtggtttagtaggtttacgtatagtatttgcggtattatcgatagtaaatcgtgtacgtcaaggttattcgccgttatcgtttcaaacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggtaaatggtcgaaatcgtcggtaataggttggccgacggtacgtgaacgtatgcgtcgtgcggaaccggcggcggatcgtgtaggtgcggcgtcgcgtgatttagaaaaacatggtgcgataacgtcgtcgaatacggcggcgacgaatgcggcgtgtgcgtggttagaagcgcaagaagaagaagaagtaggttttccggtaacgccgcaagtaccgttacgtccgatgacgtataaagcggcggtagatttatcgcattttttaaaagaaaaaggtggtttagaaggtttaatacattcgcaacgtcgtcaagatatattagatttatggatatatcatacgcaaggttattttccggattaacaaaattatacgccgggtccgggtgtacgttatccgttaacgtttggttggtgttataaattagtaccggtagaaccggataaaatagaagaagcgaataaaggtgaaaatacgtcgttattacatccggtatcgttacatggtatggatgatccggaacgtgaagtattagaatggcgttttgattcgcgtttagcgtttcatcatgtagcgcgtgaattacatccggaatattttaaaaattgttaacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 36 - DNA, 9181 bp, unidentified
  misc_feature 1..9181: note = synthetic
  source 1..9181: mol_type = other DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggagctagagctagcgtacttagcggaggcgaactcgatagatgggaaaaaattagactgagaccagggggaaaaaaaaaatataaactgaaacatatagtatgggctagtagagagttagagagattcgcagtaaacccagggttactcgaaactagcgaagggtgtagacagatactgggacaattgcaacctagtctgcaaaccggatccgaagagcttagatcactatataatacagtcgcaacactatattgtgtacaccaacgaatcgaaattaaggatacgaaagaggcattagacaaaatcgaagaggaacagaataagtctaaaaaaaaagcgcaacaggcagcagccgataccggacatagtaatcaggtatcgcaaaattatccaatcgtacagaatatacagggacaaatggtacaccaagcgatatcacctagaacacttaacgcatgggttaaggtagtcgaagagaaagcatttagtccagaggtaatacctatgtttagcgcattaagcgaaggcgcaacaccacaggatctgaatactatgcttaataccgtaggggggcatcaagccgcaatgcaaatgcttaaagagacaattaacgaagaggcagccgaatgggatagagtgcatcccgtacacgcaggaccaatcgcaccaggacaaatgagagaacctaggggatccgatatagccggaactactagtacattgcaggaacagatagggtggatgactaataatccacctatacccgtaggcgaaatatacaaaagatggataatactgggactgaataagatagttagaatgtatagtccaactagtatactcgatattagacagggacctaaagaaccttttagggattacgtagatagattctataaaacacttagagccgaacaggctagtcaagaggttaagaattggatgacagagacactattagtgcaaaacgctaatcccgattgtaaaactatacttaaggcactaggaccagcagcaacactcgaagaaatgatgacagcatgtcagggagtagggggaccaggacataaagctagagtgttagccgaagctatgtcacaggtaacgaatagcgcaacaattatgatgcaaagaggtaattttagaaaccaacgaaaaatcgttaagtgttttaattgcggaaaagagggacataccgctagaaattgtagggcacctagaaaaaaagggtgttggaaatgcggaaaagagggacatcaaatgaaagattgtaccgaaagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagatagggggacaattgaaagaggcactattagacacaggagcagacgatacagtactcgaagaaatgtcactaccaggtagatggaaacctaaaatgataggggggataggggggtttataaaggtaagacaatacgatcagatactaatcgaaatttgcggacataaagcaatcggaacagtgttagtcggacctacacccgtaaacataatcggtaggaatctgttaacacagatagggtgtacattgaattttccaattagtccaatcgaaaccgtacccgtaaaactgaaaccaggtatggacggacctaaggttaagcaatggccattaaccgaagagaaaattaaggcattagtcgaaatttgtaccgaaatggaaaaagagggtaagattagtaagataggaccagagaatccatataatacacccgtattcgcaattaaaaaaaaagatagtacgaaatggagaaagttagtcgattttagagagttaaacaaaagaacacaagacttttgggaggtacagttagggataccacatccagcaggactgaaaaaaaaaaaaagcgtaaccgtattagacgtaggagacgcatactttagcgtaccattagacgaagactttagaaagtataccgcatttacgatacctagtattaataacgaaacaccaggaattaggtatcaatataacgtactgccacagggatggaaagggtcaccagctatattccaatctagtatgactaagatactcgaaccttttagaaaacagaatccagacatagtgatataccaatatatggacgatctatacgtaggtagcgatctagagatagggcaacatagaacgaaaatcgaagagcttagacagcatctgttaagatggggattgactacacctgacaaaaaacaccaaaaagaaccaccattcttatggatgggatacgaattgcatcccgataaatggacagtgcaacctatagtgttacccgaaaaagactcatggacagtaaacgatatacaaaaactggtaggtaagttgaattgggctagtcagatatatcccggaattaaggttaggcaattgtgtaaactgttaaggggaactaaggcactaaccgaagtgataccgttaaccgaagaggcagagttagagttagccgaaaatagagagatacttaaggaacccgtacacggagtgtattacgatcctagtaaggatctgatagccgaaatacaaaaacagggacagggacaatggacatatcagatatatcaggaaccttttaaaaatctgaaaaccggtaagtacgctagaatgaggggagcacatacaaacgacgttaagcaattgaccgaagccgtacaaaaaattacaaccgaatcaatcgtaatttggggtaagacacctaaatttaaattgcctatacaaaaagagacatgggaaacatggtggactgagtattggcaagctacatggatacccgaatgggaattcgtaaatacaccaccattagtgaaattgtggtatcaactcgaaaaagaaccaatcgtaggagccgaaacattttacgtagacggagcagctaatagggaaactaagttaggtaaggcaggatacgttacgaatagggggagacagaaagtcgttacactaaccgatacaactaaccaaaaaaccgaactgcaagcgatatacttagcgttacaggatagcggattagaggtaaacatagtgacagactcacaatacgcattagggattatacaggcacaacccgatcaatccgaaagcgaactagttaatcagataatcgaacaattgattaaaaaagagaaagtgtatctagcatgggtaccagcacataagggaatagggggaaacgaacaggtagataagttagttagcgcaggaattagaaaggtactgtttttagacggaatagacaaagcgcaagacgaacacgaaaaatatcatagtaattggagagctatggctagcgattttaatctaccacccgtagtcgctaaagagatagtcgcatcatgtgataagtgtcaattgaaaggcgaagctatgcacggacaggtcgattgtagtccagggatatggcaattggattgtacacacttagagggtaaggtaatactagtcgcagtacacgtagctagcggatatatcgaagccgaagtgataccagccgaaaccggacaggaaaccgcatactttctgttaaagttagccggtagatggccagttaagactatacataccgataacggatctaattttacaggagcaaccgttagggcagcatgttggtgggcaggaattaaacaggaattcggaataccatataatccacaatcacagggagtagtcgaatctatgaataaagagttaaaaaaaattatcggacaggttagagaccaagccgaacaccttaaaaccgcagtgcaaatggccgtattcatacataattttaaaagaaaaggggggataggggggtatagcgcaggcgaaagaatcgtagacataatcgcaactgacatacagactaaagagttacagaaacagattactaagatacagaattttagagtgtattatagagactctagaaatccattatggaaaggaccagctaaactgttatggaaaggcgaaggcgcagtagtgatacaggataatagcgatattaaggtagtgcctagacgaaaagcgaaaattattagggattatggaaaacagatggcaggtgatgattgtgtggcaagtagacaggatgaggattagaacatggaaatcactagttaagcatcatatgtacgttagcggtaaggctaggggatggttttatagacatcattacgaatcaccacatcctagaatatctagcgaagtgcatataccgttaggcgacgctagattagtgataactacatattggggattgcatacaggcgaaagggattggcatctaggacagggagtgtcaatcgaatggcgaaaaaaaagatatagtacacaggtagacccagagttagccgatcaattgatacacctatattatttcgattgttttagcgatagcgcaattagaaaggcactattagggcatatagtgtcacctagatgcgaatatcaagccggacataataaggtagggtcactgcaatatctagcattagccgcactaattacacctaaaaaaattaaaccaccactacctagcgtaactaagttaaccgaagacagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgcaaccgaaaaattgtgggttacggtatattacggagtacccgtatggaaagaggcaactactacactattttgcgctagcgacgctaaagcatacgataccgaagtgcataacgtatgggctacacacgcatgcgtacctaccgatcctaatccacaagaggtagtgttagttaacgtaaccgaaaattttaatatgtggaaaaacgatatggtcgaacaaatgcacgaagacataattagcttatgggaccaatcacttaaaccatgtgttaagttgacaccactatgcgtaagtcttaagtgtacagaccttaaaaacgatactaatactaactctagtagcggaagaatgattatggaaaagggagagataaaaaattgttcattcaatattagtacatcaattaggggtaaggtacaaaaagaatacgcatttttttataagttagacataataccaatcgataacgatacaacatcatataagttaactagttgtaatactagcgtaattacacaggcatgtcctaaggtatcattcgaacctataccgatacactattgtgcaccagccggattcgcaatactgaaatgtaataataagacatttaacggaaccggaccatgtactaacgtaagtacagtgcaatgtacacacggaattagacccgtagtgagtacgcaattactgttaaacggatcattagccgaagaggaagtggtaattagatccgttaattttaccgataacgctaaaacaattatagtgcaattgaatactagcgtagagattaattgtactagacctaataataatactagaaaacggattaggatacaaaggggaccaggtagggcattcgtaacaatcggtaagatagggaatatgagacaggcacattgtaatattagtagggctaaatggaataatacacttaaacagatagctagtaagcttagagagcaattcggtaataataagacaattatattcaaacaatctagcggaggggatccagaaatcgtaacacatagttttaattgcggaggcgaatttttttattgtaatagtacacaattgttcaatagtacatggtttaatagtacatggtcaaccgaaggatctaataatacagagggatccgatacaattacactaccatgtaggattaaacagataattaatatgtggcaaaaggtaggtaaggcaatgtacgcaccacctattagcggacaaattagatgtagttcgaatataaccggattactgttaactagagacggagggaattcgaataacgaatccgaaatttttagaccaggggggggagatatgagagacaattggagatccgaattgtataagtataaagtcgttaaaatcgaaccattaggcgtagcacctacaaaagcgaaaagacgggtagtgcaacgcgaaaaaagagccgtagggataggcgcactattcttagggtttttaggcgcagccggatcaactatgggagcagctagtatgacattgacagtgcaagctagacaactgttaagcggaatagtgcaacaacagaataatctgttaagggcaatcgaagcacaacagcatctgttacaattgacagtatgggggattaagcaattgcaggctagaatactcgcagtcgaacggtatctgaaagaccaacaactgttagggatatggggatgtagcggtaagttgatatgtacaaccgcagtaccatggaacgctagttggtctaataaatcactcgaacagatatggaatcatacaacatggatggaatgggatagagagattaacaattatactagtctgatacactcactaatcgaagagtcacaaaaccaacaggaaaaaaacgaacaggaactgttagagttagacaaatgggctagcttatggaattggtttaatattactaattggttatggtatataaaactgtttattatgatagtaggggggttagtcggattgagaatcgtattcgcagtactatcaatcgttaatagggttagacagggatatagtccactatcattccaaacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggaggtaagtggtctaaatctagcgtaatcggatggcctacagttagggaaagaatgcgtagagccgaacctgcagccgatagggtaggcgcagctagtagggatctcgaaaaacacggagcaattactagtagtaatacagccgcaactaacgcagcatgcgcatggttagaggcacaggaagaggaagaggtagggtttcccgtaacaccacaggtaccacttagacctatgacatataaagccgcagtcgatctatcacattttctgaaagagaaaggggggttagagggactgatacattcacagagacgacaagacatactagacttatggatatatcatacacagggatattttccagactagcaaaattatacaccaggaccaggcgtaagatatccattgacattcggatggtgttataaactggtaccagtcgaaccagacaaaatcgaagaggctaacaaaggcgaaaatactagtctgttacatcccgtaagcttacacggaatggacgatccagaaagagaggtactcgaatggagattcgatagtagactcgcattccatcacgtagctagggaattgcatccagagtattttaaaaattgttgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 37 - DNA, 3309 bp, Rotavirus sp.
  source 1..3309: mol_type = unassigned DNA
ggctaaaaaaaatggcgcaatcgatagtagtagatggtgattatgatgcgttagcgtcgcgttatttaaaatttgtatatgattttgaaaatgtaacgtatcaaaataattattttgcgacggataaatttaaaaaagatatagaacaatatttaaaatcgatacatgatggtgaaaaaataacgcaatcgaaaatagatgaaaaagaaaaaatattattagatcgtgtaccggcggaagaacgttgtttaatatcgaaattagtatttgcgtatggtaaacatggtaatgtagaaaataaattagtaaaatatggtgtaaaagatgcgttatcgcatgcgccgcaaaaagatgcgaaaccgtatgaaaataatataataacgtcggaaatatttaaagaaaaatcggaatatacggatatatatatggatccgtcgataaatacgtcgtgtcaatcgaattgtcaagcgatgatgtttacgatatcggaaatgaaattaaataatataaaaaatgcggcgcgtttagaaaaattatttacgataatagcggcgacgataaataaatatggtatgccgcgtcataatacgcgttatcgttatgaatgggaaacgatgaaaaataaaccgtatcatttagcggcgtggataaattcgtcgatagaaatgatagcgtgtgtagtagatcatcatacgtatatgatagcgcgtgaattaatagtaaaatcgtttacgaatcgtacgtcgttagcgaaattagtatcgtcgccgatgacggtattaacggcgatgttaccgatacgtggtacgtttataacgacggaaaatttagaattagaatattcgaataaatcgataaattatttaatatcgaaagaaatggcggaagattttatgcaagcgataaaacaattacgtgatgaaggtttagaatatataccggattattatgaaaaatggtttaaatcgccggatccgttaacgtttccgaatatagcgttaatatattcgttttcgtttcatgtaggttatcgtaaacaagcgttatcggatgcggtatatgatcaaataacggtaacgtattcggataatgtaaatatggaaatgtataaagaatattcggaacgtatagaaaatgaaatatttacgatattaaaagataaaataatacatgaagataaacgtttagaagaatatgaattatcggcgttattatcgatgtcgtcggcgtcgaatggtatattacgtgaaataaattttggtggtcaaaaagtacgttcgacgaaaaaaaatatgcatgtaatagatgatatatatcataaaaaatatacgacggatataccgccggtagatgcgcgtaatccgataccgttaggtcgtcgtgatgtaccgggtcgtcgtacgcgtgcgatatttatattaccgtatcaatattttatagcgcaacattcgtttgcggaaataatgttaaattatgcgaaacgtgaacgtgaatattcggaattttattcgcaagcgaatcaagtattatcgtatggtgatgtaacgcgttatttagattcgaattcgatattatgttttacggatgtatcgcaatgggatgcgtcgcaacataatacgaaagtattacgtcgttcgataatacgtgcgatgaaacgtttaaaacaattaacgcataatataaatatacataaagcgataaatatatatatacaatcgcaagaaaatttagaaaattcgtatgtattaatagataaaaaagcgatacaatatggtgcgacggcgtcgggtgaaaaacaaacgaaaataatgaattcgatagcgaataaagcgttaatacaaacggtattaggtaaattaatgacggattatacgtttgatgtaaaaatgatacgtgtagatggtgatgataattatgcgatagtacgttttccgatagcgataacggaaaaattattatcggaatttacgtcgaaattacgttcgtattattcggaaatgaatgtaaaagtaaaagcgttagcgtcgttaacgggttgtgaaatagcgaaacgttatgtagcgggtggtatgttattttttcgtgcgggtgtaaatatattacatcatgaaaaacgtaatcaagattcggcgtatgatatggcggcgacgttatatgcgaattatatagtaaatgcgttacgtggtttaacgatgtcgcgtacgtttatattaacgaaaatatgtcaaatgacgtcgataaaaataacgggtacgttacgtttatttccgatgaaatcgatattagcgttaaattcggcgtttaaagtatttgatgaagtagattatgtaataaattatccgatatcgaatttatttatacaattacaacgtaaattatcgtcgataaaagcgaaatcgaaaatagcggataatatagcgaaatcgccgcaatttaaatcgtatgtagaattattaaataaatcgttaacgacggatgaaaatccgatagtatcggatggtatacgtttaacggaaaaagcgaaattaaattcgtatgcgccgatagcgttagaaaaacgtcgtgatcaattttcgataatggtatcgtttttacaaaatccgacgacgtttaaatcggaaacggtagtaacgataaatgatgtattatattttatatcgggttttataaaaatagattcgtcgacggtattaccgaaagaagaaaataatacgatgccgttattaccggcgataataaaacgtacgttatcgtattttggtttacgtacgcatgattatgatataaaaggttcgtcgtcgacggtatcgaaaataataaaacaatattcggtatatacgccgggtatagaagaattatatgaaatagtaaataaatcggtagatacgatacgtggttattttgcgtcgtttaatgtaccgaaagcggatgtagatacgtatatatcgacgcaaatgtataaacatgatcgttttaaaatattacaagcgtatatatataatttattatcggtaaattatggtatgtatcaattagtagatttaaattcggcgcgtttttttgatcatgtaatacatacgccgatggcgaaaacgccgacggcggtatttatgatagatttagcgttacgtttaaaaataataaatcattgtatagaaaaaggtgaaataataacggtatcggtacatgcgaataaaacggattatttaaaattatggcgtatgttatggaatgtaaaaacgatgaattcgccgtattcgaaaaattcgatgtttgatgaataagagaagtggattgcatattgtggct

SEQ ID NO: 38 - DNA, 3309 bp, unidentified
  misc_feature 1..3309: note = synthetic
  source 1..3309: mol_type = other DNA
ggctaaaaaaaatggcacaatcaatcgtagttgacggagattatgatgcattagctagtagatatctaaaattcgtatacgatttcgaaaacgtaacttatcagaataattatttcgcaactgataaatttaaaaaagatatcgaacaatatcttaaatcaatacacgatggagaaaaaattacgcaatctaagatagacgaaaaagaaaaaatactgttagatagagtaccagctgaagaaagatgtctaatatctaagttagtattcgcatatggtaagcatggtaacgttgagaataagttagttaagtatggagttaaggatgcactatcacatgcaccacaaaaagacgctaaaccatacgaaaataatataattactagcgaaatttttaaagaaaaatccgaatatactgatatatatatggatccatcaattaatacatcatgtcaatctaattgtcaagctatgatgtttacgatatccgaaatgaaattgaataatataaaaaacgctgctagactcgaaaaattgtttacaattatagccgctacaattaataagtatggtatgcctagacataatactagatatagatatgaatgggaaactatgaaaaataaaccatatcatttagccgcatggattaattcgtcaatcgaaatgatagcatgcgtagtcgatcatcatacatatatgatagctagagagttaatcgttaaatcatttactaatagaacatcattagcgaaattagtgtcatcacctatgacagtgttaaccgctatgttaccaattagaggtacatttataactactgaaaatctcgaactcgaatattctaataaatcaattaattatctgatatcgaaagaaatggctgaagattttatgcaagctataaaacaattgagagacgaaggattagagtatataccagattattacgaaaaatggtttaaatcaccagatccattaacttttcctaatatagcgttaatatattcattttcatttcacgtagggtatagaaaacaggcactatctgatgcagtatacgatcagattacggttacatattcagataacgttaatatggaaatgtataaagagtattcagaaagaatcgaaaacgaaatttttacgatactgaaagataaaattatacacgaagataaacggttagaggaatacgaactatcagcattactatctatgtcatcagcatcaaacggaatacttagagaaattaatttcggaggacaaaaagttagatcaactaaaaaaaatatgcatgtaatcgatgatatatatcataaaaaatatactactgatataccaccagtagacgctagaaatccgataccattaggtagaagagacgtaccaggtagacgaactagagctatttttatactaccatatcaatattttatagcgcaacattcattcgcagaaattatgcttaattatgctaaaagagaacgcgaatatagcgaattttattcacaggctaatcaggtactatcatacggagatgtaactagatatctagattctaattcgatactatgttttacggatgtatcacaatgggatgctagtcagcataatactaaggtacttagacgatcaattattagagctatgaaaagattgaaacaattgacacataatattaatatacataaagcgataaatatatatatacaatcacaggaaaatctcgaaaattcatatgtgttaatcgataaaaaagcgatacaatacggagctactgcatcaggcgaaaaacagactaaaattatgaattcaatcgctaataaggcattgatacagacagtgttaggtaagttaatgactgattatacattcgatgttaaaatgattagagtagacggagatgataattatgctatagttagatttccgatagcgataaccgaaaaactgttaagcgaatttacatctaaattgcgatcatattattcagaaatgaacgttaaggttaaggcattagcgtcattaacgggatgtgagatagcgaaaagatatgtagccggaggtatgttattttttagagccggagtgaatatattgcatcatgaaaaacgtaatcaggatagtgcatacgatatggcagctacattatacgctaattatatagttaacgcacttagagggttaactatgtcacgaacttttatattgactaagatatgtcaaatgacatcaattaaaattacgggaacacttagattgtttccaatgaaatcaatactcgcacttaatagtgcatttaaggtattcgatgaggtagattatgtaattaattatcctatatctaatctatttatacaattgcaacgaaaactatcatcaattaaagcgaaatctaagatagccgataatatagcgaaatcaccacaatttaaatcatacgttgagttactgaataaatcattgactactgacgaaaatccgatagtgtcagacggaatacggttaacggaaaaagctaaattgaattcatacgcaccaatcgcattagaaaaacgtagagatcaattttcaattatggtatcatttttacagaatcctactacttttaaatccgaaacagtagttacaattaacgatgtattgtattttatatcaggatttataaaaatcgattctagtacagtgttacctaaagaggaaaataatactatgccactattaccagctataattaaacgtacactatcatatttcggattgcgtacacatgattatgatattaagggatcatctagtacagtatcgaaaattataaaacagtattcagtatatacaccaggaatcgaagagttatacgaaatcgttaataaatcagtagatacaattaggggatattttgctagttttaacgtacctaaagctgatgtagatacatatatatcgacacaaatgtataaacatgatagatttaaaatactgcaagcatatatatataatctgttatcagttaattatggtatgtatcaattagtcgatctgaattcagctagattttttgatcatgtaatacatacacctatggctaaaacacctacagcagtttttatgatagatttagcgttaagattgaaaattataaatcattgtatcgaaaaaggcgaaattataacagttagcgtacacgctaataaaactgattatcttaagttatggcgtatgttatggaacgttaagactatgaatagtccatattctaaaaattcaatgtttgacgaataagagaagtggattgcatattgtggct

SEQ ID NO: 39 - DNA, 2736 bp, Rotavirus sp.
  source 1..2736: mol_type = unassigned DNA
ggcttaaaaagatcagttgaggacaaatcgttcaagatgatatcgcgtaatcgtcgtcgtaataatcaacaaaaaaatatagaaaaagaaaaacaattagaaacgataataaataaagaagtaaaagaaaataaagattcgatgaaagaagataaattagtagtaacggaagaatcgaatggtgatgtaacgacggcgaaagaacaatcgaataatataaatttacaaaaaaatgatttagtaaaagaagtaatgaatatacaaaatcaaacgttaaatacggtagtaacggaaaataaagtagaaatagaagaaatagtaaaaaaatatataccgtcgtataatacggattcgttaatagtaaaaaaattaacggaaatacaagaatcgtcggcgaaaacgtataatacgttatttcgtttatttacgccggtaaaatcgtatttatatgatataaatggtgaaaaaaaattatcgacgcgttggtattggaaattattaaaagatgatttaccggcgggtgattattcggtacgtcaattttttttatcgttatatttaaatgtattagaagaaatgccggattatataatgttacgtgatatggcggtagataatccgtattcggcggaagcgggtaaaatagtagatggtaaatcgaaagaaatattaatagaattatatcaagatcaaatgacggaaggttatatacgtcgttatatgtcggaattacgtcataaaatatcgggtgaaacgaatacggcgaaatatccggcgatattacatccggtagataatgaattaaatcaatattttttagaacatcaattaatacaaccgttaacgacgcgtaatatagcggaattaataccgacgcaattatatcatgatccgaattatgtatttaatatagatgcggcgtttttaacgaattcgcgttttgtaccgccgtatttaacgcaagatcgtataggtttacatgatggttttgaatcgatatgggattcgaaaacgcatgcggattatgtatcggcgcgtcgttttataccggatttaacggaattagtagatgcggaaaaacaaataaaagaaatggcggcgcatttacaattagaagcgataacggtacaagtagaatcgcaatttttagcgggtatatcggcggcggcggcgaatgaagcgtttaaatttataataggttcggtattatcgacgcgtacgatagcggtagaatttataacgtcgaattatatgtcgttagcgtcgtgtatgtatttaatgacgataatgccgtcggaaatatttttacgtgaatcgttagtagcgatgcaattagcgataataaatacgttaatatatccggcgttaggtttagcgcaaatgcattatcaagcgggtgaagtacgtacgccgtttgaattagcggaaatgcaagtagcgaatcgttcgatacgtcaatggttacatcattgtaatacgttacaatttggtcgtcaaataacggaaggtataatacatttacgttttacgaatgatataatgacgggtcgtatagtaaatttattttcgacgatgttagtagcgttatcgtcgcaaccgtttgcgacgtatccgttagattataaacgttcggtacaacgtgcgttacaattattatcgaatcgtacggcgcaaatagcggatttaacgcgtttaatagtatataattatacgacgttatcggcgtgtatagtaatgaatatgcatttagtaggtacgttaacggtagaacgtatacaagcgacgtcgttaacgtcgttaatgatgttaatatcgaataaaacggtaataccggaaccgtcgtcgttattttcgtatttttcgtcgaatataaattttttaacgaattataatgaacaaatagataatgtagtagcggaaataatggcggcgtatcgtttaaatttatatcaacaaaaaatgttaatgttagtaacgcgttttgtatcgaaattatatatatttgatgcgccgaaaataccgccggatcaaatgtatcgtttacgtaatcgtttacgtaatataccggtagaacgtcgtcgtgcggatgtatttcgtataataatgaataatcgtgatttaatagaaaaaacgtcggaacgtatatgtcaaggtgtattattatcgtatacgccgatgccgttaacgtatgtagaagatgtaggtttaacgaatgtaataaatgatacgaattcgtttcaaataataaatatagaagaaatagaaaaaacgggtgattattcggcgataacgaatgcgttattacgtgatacgccgataatattaaaaggtgcgataccgtatgtaacgaattcgtcggtaatagatgtattatcgaaagtagatacgacggtatttgcgtcgatagtaaaagatcgtgatatatcgaaattaaaaccgataaaatttataataaattcggattcgtcggaatattatttagtacataataataaatggacgccgacgacgacgacggcggtatataaagcgcgttcgcaacaatttgatatacaacattcggtatcgatgttagaatcgaatttattttttgtagtatataatgatttatttaaatatataaaaacgacgacggtattaccgataaatgcggtatcgtatgatggtgcgcgtataatgcaagaaacgtaaatgattgtatagtatcatcttgtgacgacctcaaactttgtggct

SEQ ID NO: 40 - DNA, 2736 bp, unidentified
  misc_feature 1..2736: note = synthetic
  source 1..2736: mol_type = other DNA
ggcttaaaaagatcagttgaggacaaatcgttcaagatgatatctagaaatagacgtagaaataatcagcaaaaaaatatcgaaaaagaaaaacagttagagacaattattaataaagaggttaaagagaataaggattcaatgaaagaggataagttagtggtaaccgaagaatctaacggagacgttacaaccgctaaagagcaatctaataatattaatctgcaaaaaaacgatttagttaaagaggttatgaatatacagaatcagacacttaatacagtagttaccgaaaataaggttgagatagaagagatagttaaaaaatatataccgtcatataatactgattcattaatcgttaaaaaattaaccgaaatacaggaatctagtgctaaaacttataatacactatttagattgtttacaccagttaaatcatacttatacgatattaatggcgaaaaaaaactatctactagatggtattggaaactgttaaaagacgatctaccagcaggtgattactcagttagacaattttttctatcattatacttaaacgtattagaggaaatgccagattatattatgcttagagatatggcagttgataatccatactcagctgaagcaggtaagatagttgacggtaagtctaaagagatattaatcgaattgtatcaggatcaaatgacagagggatatattagacggtatatgtcagaacttagacataaaatttcaggcgaaactaataccgctaaatatccagctatactgcatccagtagataacgaattgaatcaatattttctcgaacatcaattgatacaaccattaactactagaaatatagccgaattgataccgacacaattgtatcatgatcctaattacgtttttaatatagacgctgcatttttaactaattctagattcgtaccaccatacttaacgcaagataggatagggttacatgacggattcgaatcaatttgggattctaaaacacatgctgattacgtatcagctagacgatttatacccgatttaaccgaattagttgacgctgaaaaacagattaaggaaatggctgcacatttacaactcgaagctattacggtacaggttgaatcacaatttttagccggtattagcgcagcagcagctaacgaagcatttaaatttattataggatcagtgttatcaactagaactatagcagtcgaatttattacatctaattatatgtcattagctagttgtatgtatcttatgacaattatgcctagcgaaatttttttacgcgaatcattagtcgctatgcaattagctattattaatacacttatatatccagcattagggttagcgcaaatgcattatcaggcaggtgaggttagaacaccattcgaattagccgaaatgcaagttgcgaatagatcaattagacaatggttacatcattgtaatacattacaattcggtaggcaaattaccgaaggtattatacatcttagatttactaacgatattatgacaggtaggatagttaatctattttcgactatgttagttgcgttatcatcacaaccattcgctacatatccattagattataaaagatcagtgcaacgagcattacaattgttatctaatagaaccgctcaaatagctgatttaactagactgatagtgtataattatactacattatccgcatgcatagttatgaatatgcatttagtgggtacacttacagtcgaacgtatacaggctacatcattaacatcattaatgatgttaatttctaataaaacagttatacccgaaccatcatcattattttcatatttttcatctaatattaattttttaactaattataacgaacagatagataacgtagttgcggaaattatggctgcatatagattgaatctatatcaacaaaaaatgttaatgttagtgactagattcgtaagtaagttatatatattcgatgcacctaaaattccaccagatcaaatgtatagacttagaaatagacttagaaatataccagtcgaaagacgtagagcagacgtatttagaattattatgaataatagggatttaatcgaaaaaactagcgaaagaatttgtcagggagtgttactatcatatacacctatgccattaacatacgttgaagacgtaggattaactaacgtaattaacgatactaattcgtttcaaattattaatatcgaagaaatcgaaaaaacaggtgattactcagctattactaatgcactattaagggatacacctattatacttaagggagctataccatacgttacgaattcgtcagtgatagacgtactatctaaggtagatactacagtattcgctagtatagttaaggatagggatatatctaagttaaaaccaattaaatttataattaattctgattctagcgaatattatttagtgcataataataaatggacacctactactactactgcagtgtataaagctagatcacaacaattcgatatacaacatagcgtatctatgctcgaatctaatctatttttcgtagtgtataacgatctatttaaatatattaaaactactacagtgttaccaattaacgctgtatcatacgatggagctagaattatgcaagaaacataaatgattgtatagtatcatcttgtgacgacctcaaactttgtggct

SEQ ID NO: 41 - DNA, 29751 bp, Severe acute respiratory syndrome-related coronavirus
  source 1..29751: mol_type = unassigned DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggaatcgttagtattaggtgtaaatgaaaaaacgcatgtacaattatcgttaccggtattacaagtacgtgatgtattagtacgtggttttggtgattcggtagaagaagcgttatcggaagcgcgtgaacatttaaaaaatggtacgtgtggtttagtagaattagaaaaaggtgtattaccgcaattagaacaaccgtatgtatttataaaacgttcggatgcgttatcgacgaatcatggtcataaagtagtagaattagtagcggaaatggatggtatacaatatggtcgttcgggtataacgttaggtgtattagtaccgcatgtaggtgaaacgccgatagcgtatcgtaatgtattattacgtaaaaatggtaataaaggtgcgggtggtcattcgtatggtatagatttaaaatcgtatgatttaggtgatgaattaggtacggatccgatagaagattatgaacaaaattggaatacgaaacatggttcgggtgcgttacgtgaattaacgcgtgaattaaatggtggtgcggtaacgcgttatgtagataataatttttgtggtccggatggttatccgttagattgtataaaagattttttagcgcgtgcgggtaaatcgatgtgtacgttatcggaacaattagattatatagaatcgaaacgtggtgtatattgttgtcgtgatcatgaacatgaaatagcgtggtttacggaacgttcggataaatcgtatgaacatcaaacgccgtttgaaataaaatcggcgaaaaaatttgatacgtttaaaggtgaatgtccgaaatttgtatttccgttaaattcgaaagtaaaagtaatacaaccgcgtgtagaaaaaaaaaaaacggaaggttttatgggtcgtatacgttcggtatatccggtagcgtcgccgcaagaatgtaataatatgcatttatcgacgttaatgaaatgtaatcattgtgatgaagtatcgtggcaaacgtgtgattttttaaaagcgacgtgtgaacattgtggtacggaaaatttagtaatagaaggtccgacgacgtgtggttatttaccgacgaatgcggtagtaaaaatgccgtgtccggcgtgtcaagatccggaaataggtccggaacattcggtagcggattatcataatcattcgaatatagaaacgcgtttacgtaaaggtggtcgtacgcgttgttttggtggttgtgtatttgcgtatgtaggttgttataataaacgtgcgtattgggtaccgcgtgcgtcggcggatataggttcgggtcatacgggtataacgggtgataatgtagaaacgttaaatgaagatttattagaaatattatcgcgtgaacgtgtaaatataaatatagtaggtgattttcatttaaatgaagaagtagcgataatattagcgtcgttttcggcgtcgacgtcggcgtttatagatacgataaaatcgttagattataaatcgtttaaaacgatagtagaatcgtgtggtaattataaagtaacgaaaggtaaaccggtaaaaggtgcgtggaatataggtcaacaacgttcggtattaacgccgttatgtggttttccgtcgcaagcggcgggtgtaatacgttcgatatttgcgcgtacgttagatgcggcgaatcattcgataccggatttacaacgtgcggcggtaacgatattagatggtatatcggaacaatcgttacgtttagtagatgcgatggtatatacgtcggatttattaacgaattcggtaataataatggcgtatgtaacgggtggtttagtacaacaaacgtcgcaatggttatcgaatttattaggtacgacggtagaaaaattacgtccgatatttgaatggatagaagcgaaattatcggcgggtgtagaatttttaaaagatgcgtgggaaatattaaaatttttaataacgggtgtatttgatatagtaaaaggtcaaatacaagtagcgtcggataatataaaagattgtgtaaaatgttttatagatgtagtaaataaagcgttagaaatgtgtatagatcaagtaacgatagcgggtgcgaaattacgttcgttaaatttaggtgaagtatttatagcgcaatcgaaaggtttatatcgtcaatgtatacgtggtaaagaacaattacaattattaatgccgttaaaagcgccgaaagaagtaacgtttttagaaggtgattcgcatgatacggtattaacgtcggaagaagtagtattaaaaaatggtgaattagaagcgttagaaacgccggtagattcgtttacgaatggtgcgatagtaggtacgccggtatgtgtaaatggtttaatgttattagaaataaaagataaagaacaatattgtgcgttatcgccgggtttattagcgacgaataatgtatttcgtttaaaaggtggtgcgccgataaaaggtgtaacgtttggtgaagatacggtatgggaagtacaaggttataaaaatgtacgtataacgtttgaattagatgaacgtgtagataaagtattaaatgaaaaatgttcggtatatacggtagaatcgggtacggaagtaacggaatttgcgtgtgtagtagcggaagcggtagtaaaaacgttacaaccggtatcggatttattaacgaatatgggtatagatttagatgaatggtcggtagcgacgttttatttatttgatgatgcgggtgaagaaaatttttcgtcgcgtatgtattgttcgttttatccgccggatgaagaagaagaagatgatgcggaatgtgaagaagaagaaatagatgaaacgtgtgaacatgaatatggtacggaagatgattatcaaggtttaccgttagaatttggtgcgtcggcggaaacggtacgtgtagaagaagaagaagaagaagattggttagatgatacgacggaacaatcggaaatagaaccggaaccggaaccgacgccggaagaaccggtaaatcaatttacgggttatttaaaattaacggataatgtagcgataaaatgtgtagatatagtaaaagaagcgcaatcggcgaatccgatggtaatagtaaatgcggcgaatatacatttaaaacatggtggtggtgtagcgggtgcgttaaataaagcgacgaatggtgcgatgcaaaaagaatcggatgattatataaaattaaatggtccgttaacggtaggtggttcgtgtttattatcgggtcataatttagcgaaaaaatgtttacatgtagtaggtccgaatttaaatgcgggtgaagatatacaattattaaaagcggcgtatgaaaattttaattcgcaagatatattattagcgccgttattatcggcgggtatatttggtgcgaaaccgttacaatcgttacaagtatgtgtacaaacggtacgtacgcaagtatatatagcggtaaatgataaagcgttatatgaacaagtagtaatggattatttagataatttaaaaccgcgtgtagaagcgccgaaacaagaagaaccgccgaatacggaagattcgaaaacggaagaaaaatcggtagtacaaaaaccggtagatgtaaaaccgaaaataaaagcgtgtatagatgaagtaacgacgacgttagaagaaacgaaatttttaacgaataaattattattatttgcggatataaatggtaaattatatcatgattcgcaaaatatgttacgtggtgaagatatgtcgtttttagaaaaagatgcgccgtatatggtaggtgatgtaataacgtcgggtgatataacgtgtgtagtaataccgtcgaaaaaagcgggtggtacgacggaaatgttatcgcgtgcgttaaaaaaagtaccggtagatgaatatataacgacgtatccgggtcaaggttgtgcgggttatacgttagaagaagcgaaaacggcgttaaaaaaatgtaaatcggcgttttatgtattaccgtcggaagcgccgaatgcgaaagaagaaatattaggtacggtatcgtggaatttacgtgaaatgttagcgcatgcggaagaaacgcgtaaattaatgccgatatgtatggatgtacgtgcgataatggcgacgatacaacgtaaatataaaggtataaaaatacaagaaggtatagtagattatggtgtacgtttttttttttatacgtcgaaagaaccggtagcgtcgataataacgaaattaaattcgttaaatgaaccgttagtaacgatgccgataggttatgtaacgcatggttttaatttagaagaagcggcgcgttgtatgcgttcgttaaaagcgccggcggtagtatcggtatcgtcgccggatgcggtaacgacgtataatggttatttaacgtcgtcgtcgaaaacgtcggaagaacattttgtagaaacggtatcgttagcgggttcgtatcgtgattggtcgtattcgggtcaacgtacggaattaggtgtagaatttttaaaacgtggtgataaaatagtatatcatacgttagaatcgccggtagaatttcatttagatggtgaagtattatcgttagataaattaaaatcgttattatcgttacgtgaagtaaaaacgataaaagtatttacgacggtagataatacgaatttacatacgcaattagtagatatgtcgatgacgtatggtcaacaatttggtccgacgtatttagatggtgcggatgtaacgaaaataaaaccgcatgtaaatcatgaaggtaaaacgttttttgtattaccgtcggatgatacgttacgttcggaagcgtttgaatattatcatacgttagatgaatcgtttttaggtcgttatatgtcggcgttaaatcatacgaaaaaatggaaatttccgcaagtaggtggtttaacgtcgataaaatgggcggataataattgttatttatcgtcggtattattagcgttacaacaattagaagtaaaatttaatgcgccggcgttacaagaagcgtattatcgtgcgcgtgcgggtgatgcggcgaatttttgtgcgttaatattagcgtattcgaataaaacggtaggtgaattaggtgatgtacgtgaaacgatgacgcatttattacaacatgcgaatttagaatcggcgaaacgtgtattaaatgtagtatgtaaacattgtggtcaaaaaacgacgacgttaacgggtgtagaagcggtaatgtatatgggtacgttatcgtatgataatttaaaaacgggtgtatcgataccgtgtgtatgtggtcgtgatgcgacgcaatatttagtacaacaagaatcgtcgtttgtaatgatgtcggcgccgccggcggaatataaattacaacaaggtacgtttttatgtgcgaatgaatatacgggtaattatcaatgtggtcattatacgcatataacggcgaaagaaacgttatatcgtatagatggtgcgcatttaacgaaaatgtcggaatataaaggtccggtaacggatgtattttataaagaaacgtcgtatacgacgacgataaaaccggtatcgtataaattagatggtgtaacgtatacggaaatagaaccgaaattagatggttattataaaaaagataatgcgtattatacggaacaaccgatagatttagtaccgacgcaaccgttaccgaatgcgtcgtttgataattttaaattaacgtgttcgaatacgaaatttgcggatgatttaaatcaaatgacgggttttacgaaaccggcgtcgcgtgaattatcggtaacgttttttccggatttaaatggtgatgtagtagcgatagattatcgtcattattcggcgtcgtttaaaaaaggtgcgaaattattacataaaccgatagtatggcatataaatcaagcgacgacgaaaacgacgtttaaaccgaatacgtggtgtttacgttgtttatggtcgacgaaaccggtagatacgtcgaattcgtttgaagtattagcggtagaagatacgcaaggtatggataatttagcgtgtgaatcgcaacaaccgacgtcggaagaagtagtagaaaatccgacgatacaaaaagaagtaatagaatgtgatgtaaaaacgacggaagtagtaggtaatgtaatattaaaaccgtcggatgaaggtgtaaaagtaacgcaagaattaggtcatgaagatttaatggcggcgtatgtagaaaatacgtcgataacgataaaaaaaccgaatgaattatcgttagcgttaggtttaaaaacgatagcgacgcatggtatagcggcgataaattcggtaccgtggtcgaaaatattagcgtatgtaaaaccgtttttaggtcaagcggcgataacgacgtcgaattgtgcgaaacgtttagcgcaacgtgtatttaataattatatgccgtatgtatttacgttattatttcaattatgtacgtttacgaaatcgacgaattcgcgtatacgtgcgtcgttaccgacgacgatagcgaaaaattcggtaaaatcggtagcgaaattatgtttagatgcgggtataaattatgtaaaatcgccgaaattttcgaaattatttacgatagcgatgtggttattattattatcgatatgtttaggttcgttaatatgtgtaacggcggcgtttggtgtattattatcgaattttggtgcgccgtcgtattgtaatggtgtacgtgaattatatttaaattcgtcgaatgtaacgacgatggatttttgtgaaggttcgtttccgtgttcgatatgtttatcgggtttagattcgttagattcgtatccggcgttagaaacgatacaagtaacgatatcgtcgtataaattagatttaacgatattaggtttagcggcggaatgggtattagcgtatatgttatttacgaaatttttttatttattaggtttatcggcgataatgcaagtattttttggttattttgcgtcgcattttatatcgaattcgtggttaatgtggtttataatatcgatagtacaaatggcgccggtatcggcgatggtacgtatgtatatattttttgcgtcgttttattatatatggaaatcgtatgtacatataatggatggttgtacgtcgtcgacgtgtatgatgtgttataaacgtaatcgtgcgacgcgtgtagaatgtacgacgatagtaaatggtatgaaacgttcgttttatgtatatgcgaatggtggtcgtggtttttgtaaaacgcataattggaattgtttaaattgtgatacgttttgtacgggttcgacgtttatatcggatgaagtagcgcgtgatttatcgttacaatttaaacgtccgataaatccgacggatcaatcgtcgtatatagtagattcggtagcggtaaaaaatggtgcgttacatttatattttgataaagcgggtcaaaaaacgtatgaacgtcatccgttatcgcattttgtaaatttagataatttacgtgcgaataatacgaaaggttcgttaccgataaatgtaatagtatttgatggtaaatcgaaatgtgatgaatcggcgtcgaaatcggcgtcggtatattattcgcaattaatgtgtcaaccgatattattattagatcaagcgttagtatcggatgtaggtgattcgacggaagtatcggtaaaaatgtttgatgcgtatgtagatacgttttcggcgacgttttcggtaccgatggaaaaattaaaagcgttagtagcgacggcgcattcggaattagcgaaaggtgtagcgttagatggtgtattatcgacgtttgtatcggcggcgcgtcaaggtgtagtagatacggatgtagatacgaaagatgtaatagaatgtttaaaattatcgcatcattcggatttagaagtaacgggtgattcgtgtaataattttatgttaacgtataataaagtagaaaatatgacgccgcgtgatttaggtgcgtgtatagattgtaatgcgcgtcatataaatgcgcaagtagcgaaatcgcataatgtatcgttaatatggaatgtaaaagattatatgtcgttatcggaacaattacgtaaacaaatacgttcggcggcgaaaaaaaataatataccgtttcgtttaacgtgtgcgacgacgcgtcaagtagtaaatgtaataacgacgaaaatatcgttaaaaggtggtaaaatagtatcgacgtgttttaaattaatgttaaaagcgacgttattatgtgtattagcggcgttagtatgttatatagtaatgccggtacatacgttatcgatacatgatggttatacgaatgaaataataggttataaagcgatacaagatggtgtaacgcgtgatataatatcgacggatgattgttttgcgaataaacatgcgggttttgatgcgtggttttcgcaacgtggtggttcgtataaaaatgataaatcgtgtccggtagtagcggcgataataacgcgtgaaataggttttatagtaccgggtttaccgggtacggtattacgtgcgataaatggtgattttttacattttttaccgcgtgtattttcggcggtaggtaatatatgttatacgccgtcgaaattaatagaatattcggattttgcgacgtcggcgtgtgtattagcggcggaatgtacgatatttaaagatgcgatgggtaaaccggtaccgtattgttatgatacgaatttattagaaggttcgatatcgtattcggaattacgtccggatacgcgttatgtattaatggatggttcgataatacaatttccgaatacgtatttagaaggttcggtacgtgtagtaacgacgtttgatgcggaatattgtcgtcatggtacgtgtgaacgttcggaagtaggtatatgtttatcgacgtcgggtcgttgggtattaaataatgaacattatcgtgcgttatcgggtgtattttgtggtgtagatgcgatgaatttaatagcgaatatatttacgccgttagtacaaccggtaggtgcgttagatgtatcggcgtcggtagtagcgggtggtataatagcgatattagtaacgtgtgcggcgtattattttatgaaatttcgtcgtgtatttggtgaatataatcatgtagtagcggcgaatgcgttattatttttaatgtcgtttacgatattatgtttagtaccggcgtattcgtttttaccgggtgtatattcggtattttatttatatttaacgttttattttacgaatgatgtatcgtttttagcgcatttacaatggtttgcgatgttttcgccgatagtaccgttttggataacggcgatatatgtattttgtatatcgttaaaacattgtcattggttttttaataattatttacgtaaacgtgtaatgtttaatggtgtaacgttttcgacgtttgaagaagcggcgttatgtacgtttttattaaataaagaaatgtatttaaaattacgttcggaaacgttattaccgttaacgcaatataatcgttatttagcgttatataataaatataaatatttttcgggtgcgttagatacgacgtcgtatcgtgaagcggcgtgttgtcatttagcgaaagcgttaaatgatttttcgaattcgggtgcggatgtattatatcaaccgccgcaaacgtcgataacgtcggcggtattacaatcgggttttcgtaaaatggcgtttccgtcgggtaaagtagaaggttgtatggtacaagtaacgtgtggtacgacgacgttaaatggtttatggttagatgatacggtatattgtccgcgtcatgtaatatgtacggcggaagatatgttaaatccgaattatgaagatttattaatacgtaaatcgaatcattcgtttttagtacaagcgggtaatgtacaattacgtgtaataggtcattcgatgcaaaattgtttattacgtttaaaagtagatacgtcgaatccgaaaacgccgaaatataaatttgtacgtatacaaccgggtcaaacgttttcggtattagcgtgttataatggttcgccgtcgggtgtatatcaatgtgcgatgcgtccgaatcatacgataaaaggttcgtttttaaatggttcgtgtggttcggtaggttttaatatagattatgattgtgtatcgttttgttatatgcatcatatggaattaccgacgggtgtacatgcgggtacggatttagaaggtaaattttatggtccgtttgtagatcgtcaaacggcgcaagcggcgggtacggatacgacgataacgttaaatgtattagcgtggttatatgcggcggtaataaatggtgatcgttggtttttaaatcgttttacgacgacgttaaatgattttaatttagtagcgatgaaatataattatgaaccgttaacgcaagatcatgtagatatattaggtccgttatcggcgcaaacgggtatagcggtattagatatgtgtgcggcgttaaaagaattattacaaaatggtatgaatggtcgtacgatattaggttcgacgatattagaagatgaatttacgccgtttgatgtagtacgtcaatgttcgggtgtaacgtttcaaggtaaatttaaaaaaatagtaaaaggtacgcatcattggatgttattaacgtttttaacgtcgttattaatattagtacaatcgacgcaatggtcgttatttttttttgtatatgaaaatgcgtttttaccgtttacgttaggtataatggcgatagcggcgtgtgcgatgttattagtaaaacataaacatgcgtttttatgtttatttttattaccgtcgttagcgacggtagcgtattttaatatggtatatatgccggcgtcgtgggtaatgcgtataatgacgtggttagaattagcggatacgtcgttatcgggttatcgtttaaaagattgtgtaatgtatgcgtcggcgttagtattattaatattaatgacggcgcgtacggtatatgatgatgcggcgcgtcgtgtatggacgttaatgaatgtaataacgttagtatataaagtatattatggtaatgcgttagatcaagcgatatcgatgtgggcgttagtaatatcggtaacgtcgaattattcgggtgtagtaacgacgataatgtttttagcgcgtgcgatagtatttgtatgtgtagaatattatccgttattatttataacgggtaatacgttacaatgtataatgttagtatattgttttttaggttattgttgttgttgttattttggtttattttgtttattaaatcgttattttcgtttaacgttaggtgtatatgattatttagtatcgacgcaagaatttcgttatatgaattcgcaaggtttattaccgccgaaatcgtcgatagatgcgtttaaattaaatataaaattattaggtataggtggtaaaccgtgtataaaagtagcgacggtacaatcgaaaatgtcggatgtaaaatgtacgtcggtagtattattatcggtattacaacaattacgtgtagaatcgtcgtcgaaattatgggcgcaatgtgtacaattacataatgatatattattagcgaaagatacgacggaagcgtttgaaaaaatggtatcgttattatcggtattattatcgatgcaaggtgcggtagatataaatcgtttatgtgaagaaatgttagataatcgtgcgacgttacaagcgatagcgtcggaattttcgtcgttaccgtcgtatgcggcgtatgcgacggcgcaagaagcgtatgaacaagcggtagcgaatggtgattcggaagtagtattaaaaaaattaaaaaaatcgttaaatgtagcgaaatcggaatttgatcgtgatgcggcgatgcaacgtaaattagaaaaaatggcggatcaagcgatgacgcaaatgtataaacaagcgcgttcggaagataaacgtgcgaaagtaacgtcggcgatgcaaacgatgttatttacgatgttacgtaaattagataatgatgcgttaaataatataataaataatgcgcgtgatggttgtgtaccgttaaatataataccgttaacgacggcggcgaaattaatggtagtagtaccggattatggtacgtataaaaatacgtgtgatggtaatacgtttacgtatgcgtcggcgttatgggaaatacaacaagtagtagatgcggattcgaaaatagtacaattatcggaaataaatatggataattcgccgaatttagcgtggccgttaatagtaacggcgttacgtgcgaattcggcggtaaaattacaaaataatgaattatcgccggtagcgttacgtcaaatgtcgtgtgcggcgggtacgacgcaaacggcgtgtacggatgataatgcgttagcgtattataataattcgaaaggtggtcgttttgtattagcgttattatcggatcatcaagatttaaaatgggcgcgttttccgaaatcggatggtacgggtacgatatatacggaattagaaccgccgtgtcgttttgtaacggatacgccgaaaggtccgaaagtaaaatatttatattttataaaaggtttaaataatttaaatcgtggtatggtattaggttcgttagcggcgacggtacgtttacaagcgggtaatgcgacggaagtaccggcgaattcgacggtattatcgttttgtgcgtttgcggtagatccggcgaaagcgtataaagattatttagcgtcgggtggtcaaccgataacgaattgtgtaaaaatgttatgtacgcatacgggtacgggtcaagcgataacggtaacgccggaagcgaatatggatcaagaatcgtttggtggtgcgtcgtgttgtttatattgtcgttgtcatatagatcatccgaatccgaaaggtttttgtgatttaaaaggtaaatatgtacaaataccgacgacgtgtgcgaatgatccggtaggttttacgttacgtaatacggtatgtacggtatgtggtatgtggaaaggttatggttgttcgtgtgatcaattacgtgaaccgttaatgcaatcggcggatgcgtcgacgtttttaaatgggtttgcggtgtaagtgcggcgcgtttaacgccgtgtggtacgggtacgtcgacggatgtagtatatcgtgcgtttgatatatataatgaaaaagtagcgggttttgcgaaatttttaaaaacgaattgttgtcgttttcaagaaaaagatgaagaaggtaatttattagattcgtattttgtagtaaaacgtcatacgatgtcgaattatcaacatgaagaaacgatatataatttagtaaaagattgtccggcggtagcggtacatgatttttttaaatttcgtgtagatggtgatatggtaccgcatatatcgcgtcaacgtttaacgaaatatacgatggcggatttagtatatgcgttacgtcattttgatgaaggtaattgtgatacgttaaaagaaatattagtaacgtataattgttgtgatgatgattattttaataaaaaagattggtatgattttgtagaaaatccggatatattacgtgtatatgcgaatttaggtgaacgtgtacgtcaatcgttattaaaaacggtacaattttgtgatgcgatgcgtgatgcgggtatagtaggtgtattaacgttagataatcaagatttaaatggtaattggtatgattttggtgattttgtacaagtagcgccgggttgtggtgtaccgatagtagattcgtattattcgttattaatgccgatattaacgttaacgcgtgcgttagcggcggaatcgcatatggatgcggatttagcgaaaccgttaataaaatgggatttattaaaatatgattttacggaagaacgtttatgtttatttgatcgttattttaaatattgggatcaaacgtatcatccgaattgtataaattgtttagatgatcgttgtatattacattgtgcgaattttaatgtattattttcgacggtatttccgccgacgtcgtttggtccgttagtacgtaaaatatttgtagatggtgtaccgtttgtagtatcgacgggttatcattttcgtgaattaggtgtagtacataatcaagatgtaaatttacattcgtcgcgtttatcgtttaaagaattattagtatatgcggcggatccggcgatgcatgcggcgtcgggtaatttattattagataaacgtacgacgtgtttttcggtagcggcgttaacgaataatgtagcgtttcaaacggtaaaaccgggtaattttaataaagatttttatgattttgcggtatcgaaaggtttttttaaagaaggttcgtcggtagaattaaaacattttttttttgcgcaagatggtaatgcggcgatatcggattatgattattatcgttataatttaccgacgatgtgtgatatacgtcaattattatttgtagtagaagtagtagataaatattttgattgttatgatggtggttgtataaatgcgaatcaagtaatagtaaataatttagataaatcggcgggttttccgtttaataaatggggtaaagcgcgtttatattatgattcgatgtcgtatgaagatcaagatgcgttatttgcgtatacgaaacgtaatgtaataccgacgataacgcaaatgaatttaaaatatgcgatatcggcgaaaaatcgtgcgcgtacggtagcgggtgtatcgatatgttcgacgatgacgaatcgtcaatttcatcaaaaattattaaaatcgatagcggcgacgcgtggtgcgacggtagtaataggtacgtcgaaattttatggtggttggcataatatgttaaaaacggtatattcggatgtagaaacgccgcatttaatgggttgggattatccgaaatgtgatcgtgcgatgccgaatatgttacgtataatggcgtcgttagtattagcgcgtaaacataatacgtgttgtaatttatcgcatcgtttttatcgtttagcgaatgaatgtgcgcaagtattatcggaaatggtaatgtgtggtggttcgttatatgtaaaaccgggtggtacgtcgtcgggtgatgcgacgacggcgtatgcgaattcggtatttaatatatgtcaagcggtaacggcgaatgtaaatgcgttattatcgacggatggtaataaaatagcggataaatatgtacgtaatttacaacatcgtttatatgaatgtttatatcgtaatcgtgatgtagatcatgaatttgtagatgaattttatgcgtatttacgtaaacatttttcgatgatgatattatcggatgatgcggtagtatgttataattcgaattatgcggcgcaaggtttagtagcgtcgataaaaaattttaaagcggtattatattatcaaaataatgtatttatgtcggaagcgaaatgttggacggaaacggatttaacgaaaggtccgcatgaattttgttcgcaacatacgatgttagtaaaacaaggtgatgattatgtatatttaccgtatccggatccgtcgcgtatattaggtgcgggttgttttgtagatgatatagtaaaaacggatggtacgttaatgatagaacgttttgtatcgttagcgatagatgcgtatccgttaacgaaacatccgaatcaagaatatgcggatgtatttcatttatatttacaatatatacgtaaattacatgatgaattaacgggtcatatgttagatatgtattcggtaatgttaacgaatgataatacgtcgcgttattgggaaccggaattttatgaagcgatgtatacgccgcatacggtattacaagcggtaggtgcgtgtgtattatgtaattcgcaaacgtcgttacgttgtggtgcgtgtatacgtcgtccgtttttatgttgtaaatgttgttatgatcatgtaatatcgacgtcgcataaattagtattatcggtaaatccgtatgtatgtaatgcgccgggttgtgatgtaacggatgtaacgcaattatatttaggtggtatgtcgtattattgtaaatcgcataaaccgccgatatcgtttccgttatgtgcgaatggtcaagtatttggtttatataaaaatacgtgtgtaggttcggataatgtaacggattttaatgcgatagcgacgtgtgattggacgaatgcgggtgattatatattagcgaatacgtgtacggaacgtttaaaattatttgcggcggaaacgttaaaagcgacggaagaaacgtttaaattatcgtatggtatagcgacggtacgtgaagtattatcggatcgtgaattacatttatcgtgggaagtaggtaaaccgcgtccgccgttaaatcgtaattatgtatttacgggttatcgtgtaacgaaaaattcgaaagtacaaataggtgaatatacgtttgaaaaaggtgattatggtgatgcggtagtatatcgtggtacgacgacgtataaattaaatgtaggtgattattttgtattaacgtcgcatacggtaatgccgttatcggcgccgacgttagtaccgcaagaacattatgtacgtataacgggtttatatccgacgttaaatatatcggatgaattttcgtcgaatgtagcgaattatcaaaaagtaggtatgcaaaaatattcgacgttacaaggtccgccgggtacgggtaaatcgcattttgcgataggtttagcgttatattatccgtcggcgcgtatagtatatacggcgtgttcgcatgcggcggtagatgcgttatgtgaaaaagcgttaaaatatttaccgatagataaatgttcgcgtataataccggcgcgtgcgcgtgtagaatgttttgataaatttaaagtaaattcgacgttagaacaatatgtattttgtacggtaaatgcgttaccggaaacgacggcggatatagtagtatttgatgaaatatcgatggcgacgaattatgatttatcggtagtaaatgcgcgtttacgtgcgaaacattatgtatatataggtgatccggcgcaattaccggcgccgcgtacgttattaacgaaaggtacgttagaaccggaatattttaattcggtatgtcgtttaatgaaaacgataggtccggatatgtttttaggtacgtgtcgtcgttgtccggcggaaatagtagatacggtatcggcgttagtatatgataataaattaaaagcgcataaagataaatcggcgcaatgttttaaaatgttttataaaggtgtaataacgcatgatgtatcgtcggcgataaatcgtccgcaaataggtgtagtacgtgaatttttaacgcgtaatccggcgtggcgtaaagcggtatttatatcgccgtataattcgcaaaatgcggtagcgtcgaaaatattaggtttaccgacgcaaacggtagattcgtcgcaaggttcggaatatgattatgtaatatttacgcaaacgacggaaacggcgcattcgtgtaatgtaaatcgttttaatgtagcgataacgcgtgcgaaaataggtatattatgtataatgtcggatcgtgatttatatgataaattacaatttacgtcgttagaaataccgcgtcgtaatgtagcgacgttacaagcggaaaatgtaacgggtttatttaaagattgttcgaaaataataacgggtttacatccgacgcaagcgccgacgcatttatcggtagatataaaatttaaaacggaaggtttatgtgtagatataccgggtataccgaaagatatgacgtatcgtcgtttaatatcgatgatgggttttaaaatgaattatcaagtaaatggttatccgaatatgtttataacgcgtgaagaagcgatacgtcatgtacgtgcgtggataggttttgatgtagaaggttgtcatgcgacgcgtgatgcggtaggtacgaatttaccgttacaattaggtttttcgacgggtgtaaatttagtagcggtaccgacgggttatgtagatacggaaaataatacggaatttacgcgtgtaaatgcgaaaccgccgccgggtgatcaatttaaacatttaataccgttaatgtataaaggtttaccgtggaatgtagtacgtataaaaatagtacaaatgttatcggatacgttaaaaggtttatcggatcgtgtagtatttgtattatgggcgcatggttttgaattaacgtcgatgaaatattttgtaaaaataggtccggaacgtacgtgttgtttatgtgataaacgtgcgacgtgtttttcgacgtcgtcggatacgtatgcgtgttggaatcattcggtaggttttgattatgtatataatccgtttatgatagatgtacaacaatggggttttacgggtaatttacaatcgaatcatgatcaacattgtcaagtacatggtaatgcgcatgtagcgtcgtgtgatgcgataatgacgcgttgtttagcggtacatgaatgttttgtaaaacgtgtagattggtcggtagaatatccgataataggtgatgaattacgtgtaaattcggcgtgtcgtaaagtacaacatatggtagtaaaatcggcgttattagcggataaatttccggtattacatgatataggtaatccgaaagcgataaaatgtgtaccgcaagcggaagtagaatggaaattttatgatgcgcaaccgtgttcggataaagcgtataaaatagaagaattattttattcgtatgcgacgcatcatgataaatttacggatggtgtatgtttattttggaattgtaatgtagatcgttatccggcgaatgcgatagtatgtcgttttgatacgcgtgtattatcgaatttaaatttaccgggttgtgatggtggttcgttatatgtaaataaacatgcgtttcatacgccggcgtttgataaatcggcgtttacgaatttaaaacaattaccgtttttttattattcggattcgccgtgtgaatcgcatggtaaacaagtagtatcggatatagattatgtaccgttaaaatcggcgacgtgtataacgcgttgtaatttaggtggtgcggtatgtcgtcatcatgcgaatgaatatcgtcaatatttagatgcgtataatatgatgatatcggcgggtttttcgttatggatatataaacaatttgatacgtataatttatggaatacgtttacgcgtttacaatcgttagaaaatgtagcgtataatgtagtaaataaaggtcattttgatggtcatgcgggtgaagcgccggtatcgataataaataatgcggtatatacgaaagtagatggtatagatgtagaaatatttgaaaataaaacgacgttaccggtaaatgtagcgtttgaattatgggcgaaacgtaatataaaaccggtaccggaaataaaaatattaaataatttaggtgtagatatagcggcgaatacggtaatatgggattataaacgtgaagcgccggcgcatgtatcgacgataggtgtatgtacgatgacggatatagcgaaaaaaccgacggaatcggcgtgttcgtcgttaacggtattatttgatggtcgtgtagaaggtcaagtagatttatttcgtaatgcgcgtaatggtgtattaataacggaaggttcggtaaaaggtttaacgccgtcgaaaggtccggcgcaagcgtcggtaaatggtgtaacgttaataggtgaatcggtaaaaacgcaatttaattattttaaaaaagtagatggtataatacaacaattaccggaaacgtattttacgcaatcgcgtgatttagaagattttaaaccgcgttcgcaaatggaaacggattttttagaattagcgatggatgaatttatacaacgttataaattagaaggttatgcgtttgaacatatagtatatggtgatttttcgcatggtcaattaggtggtttacatttaatgataggtttagcgaaacgttcgcaagattcgccgttaaaattagaagattttataccgatggattcgacggtaaaaaattattttataacggatgcgcaaacgggttcgtcgaaatgtgtatgttcggtaatagatttattattagatgattttgtagaaataataaaatcgcaagatttatcggtaatatcgaaagtagtaaaagtaacgatagattatgcggaaatatcgtttatgttatggtgtaaagatggtcatgtagaaacgttttatccgaaattacaagcgtcgcaagcgtggcaaccgggtgtagcgatgccgaatttatataaaatgcaacgtatgttattagaaaaatgtgatttacaaaattatggtgaaaatgcggtaataccgaaaggtataatgatgaatgtagcgaaatatacgcaattatgtcaatatttaaatacgttaacgttagcggtaccgtataatatgcgtgtaatacattttggtgcgggttcggataaaggtgtagcgccgggtacggcggtattacgtcaatggttaccgacgggtacgttattagtagattcggatttaaatgattttgtatcggatgcggattcgacgttaataggtgattgtgcgacggtacatacggcgaataaatgggatttaataatatcggatatgtatgatccgcgtacgaaacatgtaacgaaagaaaatgattcgaaagaaggtttttttacgtatttatgtggttttataaaacaaaaattagcgttaggtggttcgatagcggtaaaaataacggaacattcgtggaatgcggatttatataaattaatgggtcatttttcgtggtggacggcgtttgtaacgaatgtaaatgcgtcgtcgtcggaagcgtttttaataggtgcgaattatttaggtaaaccgaaagaacaaatagatggttatacgatgcatgcgaattatatattttggcgtaatacgaatccgatacaattatcgtcgtattcgttatttgatatgtcgaaatttccgttaaaattacgtggtacggcggtaatgtcgttaaaagaaaatcaaataaatgatatgatatattcgttattagaaaaaggtcgtttaataatacgtgaaaataatcgtgtagtagtatcgtcggatatattagtaaataattaaacgaacatgtttatatttttattatttttaacgttaacgtcgggttcggatttagatcgttgtacgacgtttgatgatgtacaagcgccgaattatacgcaacatacgtcgtcgatgcgtggtgtatattatccggatgaaatatttcgttcggatacgttatatttaacgcaagatttatttttaccgttttattcgaatgtaacgggttttcatacgataaatcatacgtttggtaatccggtaataccgtttaaagatggtatatattttgcggcgacggaaaaatcgaatgtagtacgtggttgggtatttggttcgacgatgaataataaatcgcaatcggtaataataataaataattcgacgaatgtagtaatacgtgcgtgtaattttgaattatgtgataatccgttttttgcggtatcgaaaccgatgggtacgcaaacgcatacgatgatatttgataatgcgtttaattgtacgtttgaatatatatcggatgcgttttcgttagatgtatcggaaaaatcgggtaattttaaacatttacgtgaatttgtatttaaaaataaagatggttttttatatgtatataaaggttatcaaccgatagatgtagtacgtgatttaccgtcgggttttaatacgttaaaaccgatatttaaattaccgttaggtataaatataacgaattttcgtgcgatattaacggcgttttcgccggcgcaagatatatggggtacgtcggcggcggcgtattttgtaggttatttaaaaccgacgacgtttatgttaaaatatgatgaaaatggtacgataacggatgcggtagattgttcgcaaaatccgttagcggaattaaaatgttcggtaaaatcgtttgaaatagataaaggtatatatcaaacgtcgaattttcgtgtagtaccgtcgggtgatgtagtacgttttccgaatataacgaatttatgtccgtttggtgaagtatttaatgcgacgaaatttccgtcggtatatgcgtgggaacgtaaaaaaatatcgaattgtgtagcggattattcggtattatataattcgacgtttttttcgacgtttaaatgttatggtgtatcggcgacgaaattaaatgatttatgtttttcgaatgtatatgcggattcgtttgtagtaaaaggtgatgatgtacgtcaaatagcgccgggtcaaacgggtgtaatagcggattataattataaattaccggatgattttatgggttgtgtattagcgtggaatacgcgtaatatagatgcgacgtcgacgggtaattataattataaatatcgttatttacgtcatggtaaattacgtccgtttgaacgtgatatatcgaatgtaccgttttcgccggatggtaaaccgtgtacgccgccggcgttaaattgttattggccgttaaatgattatggtttttatacgacgacgggtataggttatcaaccgtatcgtgtagtagtattatcgtttgaattattaaatgcgccggcgacggtatgtggtccgaaattatcgacggatttaataaaaaatcaatgtgtaaattttaattttaatggtttaacgggtacgggtgtattaacgccgtcgtcgaaacgttttcaaccgtttcaacaatttggtcgtgatgtatcggattttacggattcggtacgtgatccgaaaacgtcggaaatattagatatatcgccgtgtgcgtttggtggtgtatcggtaataacgccgggtacgaatgcgtcgtcggaagtagcggtattatatcaagatgtaaattgtacggatgtatcgacggcgatacatgcggatcaattaacgccggcgtggcgtatatattcgacgggtaataatgtatttcaaacgcaagcgggttgtttaataggtgcggaacatgtagatacgtcgtatgaatgtgatataccgataggtgcgggtatatgtgcgtcgtatcatacggtatcgttattacgttcgacgtcgcaaaaatcgatagtagcgtatacgatgtcgttaggtgcggattcgtcgatagcgtattcgaataatacgatagcgataccgacgaatttttcgatatcgataacgacggaagtaatgccggtatcgatggcgaaaacgtcggtagattgtaatatgtatatatgtggtgattcgacggaatgtgcgaatttattattacaatatggttcgttttgtacgcaattaaatcgtgcgttatcgggtatagcggcggaacaagatcgtaatacgcgtgaagtatttgcgcaagtaaaacaaatgtataaaacgccgacgttaaaatattttggtggttttaatttttcgcaaatattaccggatccgttaaaaccgacgaaacgttcgtttatagaagatttattatttaataaagtaacgttagcggatgcgggttttatgaaacaatatggtgaatgtttaggtgatataaatgcgcgtgatttaatatgtgcgcaaaaatttaatggtttaacggtattaccgccgttattaacggatgatatgatagcggcgtatacggcggcgttagtatcgggtacggcgacggcgggttggacgtttggtgcgggtgcggcgttacaaataccgtttgcgatgcaaatggcgtatcgttttaatggtataggtgtaacgcaaaatgtattatatgaaaatcaaaaacaaatagcgaatcaatttaataaagcgatatcgcaaatacaagaatcgttaacgacgacgtcgacggcgttaggtaaattacaagatgtagtaaatcaaaatgcgcaagcgttaaatacgttagtaaaacaattatcgtcgaattttggtgcgatatcgtcggtattaaatgatatattatcgcgtttagataaagtagaagcggaagtacaaatagatcgtttaataacgggtcgtttacaatcgttacaaacgtatgtaacgcaacaattaatacgtgcggcggaaatacgtgcgtcggcgaatttagcggcgacgaaaatgtcggaatgtgtattaggtcaatcgaaacgtgtagatttttgtggtaaaggttatcatttaatgtcgtttccgcaagcggcgccgcatggtgtagtatttttacatgtaacgtatgtaccgtcgcaagaacgtaattttacgacggcgccggcgatatgtcatgaaggtaaagcgtattttccgcgtgaaggtgtatttgtatttaatggtacgtcgtggtttataacgcaacgtaattttttttcgccgcaaataataacgacggataatacgtttgtatcgggtaattgtgatgtagtaataggtataataaataatacggtatatgatccgttacaaccggaattagattcgtttaaagaagaattagataaatattttaaaaatcatacgtcgccggatgtagatttaggtgatatatcgggtataaatgcgtcggtagtaaatatacaaaaagaaatagatcgtttaaatgaagtagcgaaaaatttaaatgaatcgttaatagatttacaagaattaggtaaatatgaacaatatataaaatggccgtggtatgtatggttaggttttatagcgggtttaatagcgatagtaatggtaacgatattattatgttgtatgacgtcgtgttgttcgtgtttaaaaggtgcgtgttcgtgtggttcgtgttgtaaatttgatgaagatgattcggaaccggtattaaaaggtgtaaaattacattatacgtaaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcggataatggtacgataacggtagaagaattaaaacaattattagaacaatggaatttagtaataggttttttatttttagcgtggataatgttattacaatttgcgtattcgaatcgtaatcgttttttatatataataaaattagtatttttatggttattatggccggtaacgttagcgtgttttgtattagcggcggtatatcgtataaattgggtaacgggtggtatagcgatagcgatggcgtgtatagtaggtttaatgtggttatcgtattttgtagcgtcgtttcgtttatttgcgcgtacgcgttcgatgtggtcgtttaatccggaaacgaatatattattaaatgtaccgttacgtggtacgatagtaacgcgtccgttaatggaatcggaattagtaataggtgcggtaataatacgtggtcatttacgtatggcgggtcattcgttaggtcgttgtgatataaaagatttaccgaaagaaataacggtagcgacgtcgcgtacgttatcgtattataaattaggtgcgtcgcaacgtgtaggtacggattcgggttttgcggcgtataatcgttatcgtataggtaattataaattaaatacggatcatgcgggttcgaatgataatatagcgttattagtacaataagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 42 - DNA, 29751 bp, unidentified
  misc_feature 1..29751: note = synthetic
  source 1..29751: mol_type = other DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagtcattagtgttaggcgttaacgaaaaaactcacgttcaattgtcactaccagtgttacaggttagagacgtactcgttagggggttcggtgattcagtcgaagaggcactatccgaagctagagagcatcttaaaaacggtacatgcggattagtcgaactcgaaaaaggcgtactaccacaattggagcaaccatacgtttttattaaacggtctgacgcacttagtactaatcacggtcataaggtagtcgagttagtcgccgaaatggacggtattcaatacggtaggtcaggtattacactcggagtgttagtgccacacgtaggcgaaacacctatcgcttatcgtaacgttctattgcgtaaaaacggtaataagggcgcaggcggacattcatacggtatcgatcttaagtcatacgatttaggcgacgaactcggtacggatccaatcgaagattacgaacagaattggaatactaaacacggatctggcgcattacgcgaacttacacgcgaacttaacggaggcgcagtgactagatacgtcgataataatttttgcggtccagacggatatccactcgattgtattaaggattttctcgctagggcaggtaagtctatgtgtacacttagcgaacaactcgattatatcgaatctaaaagaggcgtatattgttgtcgcgatcacgaacacgaaatcgcttggtttactgagcgatctgataagtcatacgaacatcagactccattcgaaattaagtctgctaaaaaattcgatacttttaaaggcgaatgtcctaaattcgtttttccacttaactctaaggttaaggttattcaacctagagtcgaaaaaaaaaaaactgagggttttatgggtaggattaggtcagtgtatccagtcgctagtccacaagagtgtaataatatgcatctatctacacttatgaaatgtaatcattgtgacgaagtgtcatggcaaacatgcgatttccttaaggctacatgcgaacattgcggtaccgaaaatctcgtaatcgagggacctactacatgcggatacttacctactaacgcagtcgttaaaatgccatgtccagcttgtcaggatccagagataggtccagagcatagcgttgccgattatcataatcattctaatatcgaaactagattgcgtaaaggcggtaggacacgttgtttcggcggatgcgtattcgcatacgtaggttgttataataagagagcgtattgggtgcctagagctagtgccgatatcggtagcggacataccggtattacaggcgataacgttgagacacttaacgaggatctgttagagattctatcacgcgaacgcgttaatattaacatagtcggcgattttcatcttaacgaagaggtcgctattatactcgctagtttttccgctagtacatccgcttttatcgatactattaagtctcttgactataagtcttttaaaactatcgttgagtcatgcggtaattataaggttactaagggtaagccagtgaaaggcgcatggaatatcggtcagcaacgttcagtgcttacaccactatgcggttttcctagtcaagccgcaggcgtaattagatctattttcgcacgtacacttgacgctgctaatcattctattcccgatctgcaacgtgctgccgttacgatactcgacggtattagcgaacagtcacttagactcgttgacgctatggtgtatacatccgatctgttaacgaatagtgtgattattatggcttacgttacaggcggattagtgcaacagactagtcaatggttgtctaatctgttaggtactacagtcgaaaaattgcgacctattttcgaatggattgaggctaaattgtctgccggagtcgaattccttaaagacgcttgggagatacttaaatttctgattaccggcgtattcgatatcgttaagggtcagattcaggtcgctagcgataatattaaggattgcgttaagtgttttatcgacgtagtgaataaggcactcgaaatgtgtatcgatcaggttacaattgccggagctaagcttagatcacttaacttaggcgaagtgtttatcgctcaatctaagggattgtatcgtcaatgtatacgcggtaaggagcaattgcaattgcttatgccacttaaggctcctaaagaggttacattccttgagggcgattcacacgatacagtgcttactagcgaagaggttgtgcttaaaaacggcgaactcgaagcacttgagacaccagtcgattcttttactaacggcgcaatcgtaggtacacccgtatgcgttaacggtcttatgttgcttgagattaaggataaagagcaatattgcgcacttagtccaggtctgttagcgactaataacgtttttagacttaagggaggcgcacctattaaaggcgttacattcggtgaggatacagtttgggaagtgcaagggtataaaaacgttaggattacattcgaactcgacgaacgtgtcgataaggtacttaacgaaaagtgtagcgtatatacagtcgaatccggtactgaggttactgagttcgcatgcgtagtcgctgaggcagtcgttaagacattgcaaccggttagcgatctgttaactaatatgggtatcgatcttgacgaatggtcagtcgctactttttatctattcgatgacgctggcgaagagaatttttcgtcacgtatgtattgttctttttaccctcctgacgaagaggaagaggacgacgctgaatgcgaagaggaagagatagacgaaacatgcgaacacgaatacggtacggaagacgattatcagggattgccactcgaattcggcgctagcgctgagactgttagagtcgaagaggaagaggaagaggattggttagacgatactactgagcaatccgaaatcgaacccgaacccgaacctacacctgaggaacccgttaatcaatttacagggtatcttaagcttaccgataacgttgcgattaaatgcgttgatatcgttaaagaggctcaatccgctaatcctatggtaatcgttaacgctgctaatatacatcttaagcatggcggaggcgttgcaggcgcacttaataaggctactaacggcgctatgcaaaaagagtctgacgattatattaagcttaacggtccacttaccgtaggcggatcatgtctattgtcaggtcataatctcgctaaaaaatgtcttcacgtagtcggacctaatcttaacgctggcgaagacatacaattgcttaaagccgcatacgagaattttaattcgcaagacatattgctcgcaccactgttatccgcaggtattttcggagctaaaccattgcaatcattgcaggtatgcgttcagacagtgcgtacacaggtatatatcgcagttaacgataaggcactatacgaacaggtcgttatggattatctcgataatcttaagcctagagtcgaagctcctaaacaggaagagccacctaatactgaggattctaagactgaggaaaaatccgttgtgcaaaaaccagtcgacgttaagcctaaaattaaggcatgtattgacgaagtgactactacacttgaggagactaagtttcttactaataagttactgttattcgctgatattaacggtaagttgtatcacgattcacagaatatgcttagaggcgaagatatgtcttttctcgaaaaagacgctccatatatggtaggcgacgttattacatctggcgatattacatgcgtagtgatacctagtaaaaaagccggaggtactaccgaaatgctatcacgtgcacttaaaaaagtgccagtcgacgaatacattactacttatcccggtcagggatgcgcaggttatacactcgaagaggctaaaaccgcacttaaaaaatgtaaatccgctttttacgttctgccatctgaggcacctaacgctaaagaggagatactcggtacagtgtcatggaatttacgcgaaatgcttgcgcatgccgaagagacacgtaagcttatgcctatttgtatggacgttagggctattatggctactattcaacgtaagtataagggtattaagattcaagagggtatagtcgattacggagtgagattttttttttatacatctaaagagccagtcgctagtattattactaagcttaactcacttaacgaaccattagtgactatgcctatcggatacgttacacacggttttaatctcgaagaggccgctagatgtatgcgatcacttaaggcaccagccgtagtgtcagtgtcatcacctgacgccgttactacttataacggataccttacatctagttctaagactagtgaggaacatttcgttgagacagtgtcacttgccggttcatatcgcgattggtcttattcaggtcaacgtactgagttaggcgttgagtttcttaaacgcggagataagatagtgtatcatacacttgagtcaccagtcgagtttcacttagacggcgaagtgcttagtctcgataagcttaagtctctattgtcacttagagaggttaagactattaaagtgtttactacagtcgataatactaatctgcatacacagttagtcgatatgtctatgacatacggtcagcaattcggacctacttatcttgacggtgcagacgttactaagattaagcctcacgttaatcacgaaggtaagacttttttcgtattgccatctgacgatacacttagatccgaagcattcgaatactatcatacacttgacgaatcttttctcggtaggtatatgtcagcacttaaccatacaaaaaaatggaaattcccacaggtaggcggacttacatctattaaatgggccgataataattgttatctgtcatcagtgttactcgcattgcagcaactcgaagtgaaatttaacgctcctgcattgcaagaggcatactatagggctagagccggtgacgctgctaatttttgcgcacttatactcgcatactctaataaaaccgtaggcgaactcggtgacgttagagagactatgacacatctattgcaacacgctaatctcgaatccgctaaaagagtgcttaacgtagtgtgtaaacattgcggtcagaaaactactacacttaccggagtcgaagccgttatgtatatgggtacactatcatacgataatcttaaaaccggagtgtcaattccatgcgtatgcggtagggacgctacacaatacttagtgcaacaagagtctagtttcgttatgatgtcagctccacctgccgaatacaaattgcaacagggtacattcttatgcgctaacgagtataccggtaattatcaatgcggtcattatacacatattaccgctaaagagacattgtatcgtatagacggtgcacatcttactaaaatgtctgagtataagggtccagttactgacgttttttacaaagagactagttatactactactattaaaccggttagttataagttagacggagtgacttataccgaaatcgaacctaagttagacggatactataaaaaagataacgcttattataccgaacagccaatcgatttagtgcctacacaaccattgcctaacgctagtttcgataattttaagcttacatgttctaatactaaattcgcagacgatcttaatcagatgaccggttttactaagcctgctagtagagagttatccgttactttttttcccgatcttaacggtgacgtagtcgcaatcgattatcgtcattactctgctagttttaaaaaaggcgctaagctattgcataaacctatcgtttggcatattaatcaggctactactaagactacttttaaacctaatacttggtgtcttagatgtctttggtctactaagccagtcgatacatctaattcattcgaagtgcttgcagtcgaggatacacagggtatggataatctcgcatgcgaatcgcaacaacctacatccgaagaggtagtcgagaatcctacaattcagaaagaggtaatcgaatgcgacgttaagactaccgaagtggtaggtaacgttatacttaaaccgtctgacgaaggcgttaaggttacgcaagagttaggccatgaggatcttatggccgcatacgttgagaatacatctattactattaaaaaacctaacgaactatcactcgcattaggtcttaagactatcgctacacacggtatagccgctattaattcggttccatggtctaagatactcgcatacgttaagccttttctcggtcaagccgctattactacatctaattgcgctaaacgacttgcgcaacgcgtttttaataattatatgccatacgtttttacattgctttttcaattgtgtacttttactaagtctactaactcacgtatacgcgctagtctacctactactatcgctaaaaattccgttaaatccgttgcgaaattgtgtcttgacgcaggtattaattacgttaagtcacctaaattttctaaattgtttacaatcgctatgtggttactgttattgtctatttgtctcggttcattgatttgcgttaccgctgcattcggagtgttactatctaatttcggcgcacctagttattgtaacggagtgagagagttgtatctgaatagttctaacgttactactatggatttttgcgaaggatcttttccatgttcaatttgtctatctggtctcgattcactcgattcatatcccgctctcgaaactattcaggttacgattagttcttataaactcgatcttactatactcggtctagccgctgaatgggtgttagcgtatatgctttttactaagtttttttacttactcggtctatccgctattatgcaagtgtttttcggatatttcgctagtcattttatttctaatagttggcttatgtggtttattatttcgattgtgcaaatggcaccagttagcgctatggtacgtatgtatatttttttcgctagtttttactatatttggaaatcatacgttcatattatggacggatgtacatctagtacatgtatgatgtgttataaacgtaatcgcgctactagagtcgagtgtactactatcgttaacggtatgaaacgatctttttacgtttacgctaacggaggtagggggttttgtaagactcataattggaattgtcttaattgcgatactttttgtaccggtagtacttttatttctgacgaagtcgcacgcgatctatcattgcaatttaaacgtccaattaaccctaccgatcaatctagttatatagtcgatagcgttgcggttaaaaacggcgcattgcatctatatttcgataaagccggtcagaaaacatacgaacgtcatccactatcacatttcgttaacttagacaatcttagggctaataatactaagggtagtctgccaattaacgtaatcgtattcgacggtaagtctaaatgcgacgaatccgctagtaagtctgctagtgtgtattactctcagcttatgtgtcaacctatactgttactcgatcaggcattagtgtcagacgtaggcgatagtactgaggttagcgttaaaatgttcgacgcttacgtcgatacttttagcgctacattctcagtgcctatggagaaacttaaggcattagtcgctaccgctcattctgagttagcgaaaggcgttgcgttagacggagtgctatctacattcgtatctgccgctagacagggcgtagtcgatactgacgtcgatactaaagacgtaatcgaatgtcttaagctatcacatcattccgatcttgaggttacaggcgattcatgtaataattttatgcttacatataataaggtcgagaatatgacacctagagacttaggcgcatgtatcgattgtaacgctagacatattaacgctcaggttgcgaaatcacataacgtatcattgatttggaacgttaaagactatatgtcactatctgagcaattgcgtaaacagatacgatccgctgctaaaaaaaataatataccgtttagacttacatgcgctactactagacaggtcgttaacgttattactactaagattagtcttaagggaggtaagatcgttagtacatgttttaagcttatgcttaaggctacactgttatgcgtactcgctgcactcgtatgttatatcgttatgccagtgcatacactatctattcacgacggatatactaacgaaattatcggatataaggctattcaagacggagtgacacgcgatattattagtactgacgattgtttcgctaataaacacgctggattcgacgcttggttttcgcaacgcggagggtcatataaaaacgataagtcatgtccagtcgttgccgctattattacacgcgaaatcggattcatagtgccagggttacccggtacagtgttgcgtgctattaacggcgattttttgcattttctgccacgcgtttttagcgcagtcggtaatatttgttatacacctagtaagttaatcgaatactctgatttcgctactagcgcatgcgtacttgccgctgagtgtactatttttaaagacgctatgggtaagccagtgccatattgttacgatactaatctgttagagggatctatttcatactctgagttgcgacctgatactaggtacgtacttatggacggatctattattcaattccctaatacttatcttgagggatccgttagagtcgttactacattcgacgctgagtattgtagacacggtacatgcgaacgatctgaggtcggtatttgtctatctacatctggtagatgggtacttaataacgaacattatcgcgcactatctggcgttttttgcggcgtagacgctatgaatcttatcgctaatatttttacaccattagtgcaaccagtcggcgcacttgacgtatccgctagcgtagtcgcaggcggtattatcgctatactcgttacatgcgctgcatactattttatgaaatttcgtagggtattcggtgagtataatcacgtagtcgctgctaacgctctattattccttatgtcttttacgatactgtgtctcgtacctgcatactcttttctgccaggcgtatactctgtgttttatctgtatcttacattttactttactaacgacgtttcgtttctcgcacatctgcaatggttcgctatgttttcacctatagtgccattttggattaccgctatttacgttttttgtatttcgcttaagcattgtcattggttttttaataattatttgcgtaaacgcgttatgtttaacggagtgactttttcgacattcgaagaggccgcactttgtacttttctgcttaataaagagatgtatcttaagttacggtctgagacactgttaccacttacacaatacaataggtatctcgcactatataataagtataagtatttttcaggcgcactcgatactacatcttatcgcgaagccgcatgttgtcatctcgctaaagcgcttaacgatttttctaactctggcgcagacgtactgtatcaacctccacaaacatctattacgtctgccgtactgcaatccggttttcgtaaaatggcttttccatcaggtaaggtcgagggatgtatggtgcaagttacatgcggtactactacacttaacggtctatggttagacgatacggtttattgtcctagacacgttatttgtactgccgaagatatgcttaaccctaattacgaagacttactgatacgtaaatctaatcactcttttttagtgcaagccggtaacgttcaattgcgcgttatcggtcattctatgcaaaattgtctgttacgtcttaaagtcgatacatctaatcctaaaactcctaagtataaattcgttaggattcaacccggtcagacattctcagtactcgcatgttataacggttcacctagcggagtgtatcaatgcgctatgcgacctaatcatacaattaagggatcttttcttaacggttcatgcggatcagtcggttttaatatcgattacgattgcgttagtttttgttatatgcatcatatggagttacctacaggcgtacacgctggtactgatcttgagggtaagttttacggtccattcgttgatcgtcaaaccgctcaagccgcaggtaccgatactactattacacttaacgtactcgcatggttatacgctgccgttattaacggcgatagatggtttcttaatagatttactactacacttaacgattttaacttagtcgctatgaaatataattacgaaccacttacacaggatcacgtcgatatactcggtccacttagtgcgcaaaccggtatagccgtactcgatatgtgcgctgcacttaaagagttactgcaaaacggtatgaacggtaggactatactcggtagtactatactcgaagacgaatttacaccattcgacgtcgttagacagtgttcaggcgttacatttcagggtaagtttaaaaaaatcgttaagggtacacatcattggatgttgcttacattccttactagtctattgatactcgtacaatctacacaatggtcattgttttttttcgtatacgaaaacgcttttttgccttttacactcggtattatggctatagccgcatgcgctatgttgttagtgaaacataaacacgcattcttatgtctattcctattgcctagtctcgctacagtcgcatactttaatatggtgtatatgcctgctagttgggttatgcgtattatgacttggcttgagttagccgatacatctctatctggttatagacttaaggattgcgttatgtacgctagtgcattagtgttactgatacttatgactgcacgtacagtttacgacgacgctgctagacgcgtttggacacttatgaacgttattacacttgtgtataaagtgtattacggtaacgctctcgatcaggctattagtatgtgggcattagtgatatccgttacgtctaattactctggagtcgttacgactattatgtttctcgctagggctatcgtattcgtatgcgttgagtattatccactattgtttattaccggtaatacattgcaatgtattatgttagtgtattgttttttagggtattgttgttgttgttatttcggattgttttgtctacttaataggtattttagacttacattaggcgtatacgattatctcgttagtacgcaagagtttagatatatgaattcacagggactgttaccacctaaatcttcaatcgacgcttttaagcttaacattaagttactcggtataggcggtaagccttgtattaaggttgcgacagtgcaatctaaaatgtctgacgttaagtgtactagtgtcgtactgttatcagtgttacagcaattgcgagtcgaatctagttctaaattgtgggctcaatgcgttcaattgcataacgatatcttactcgctaaggatactactgaggcattcgaaaaaatggttagtctgttaagcgtactgttatctatgcaaggcgcagtcgatattaatcgattatgcgaagagatgttagacaatagggctacattgcaggctattgcgtcagagttttctagtctgccatcatacgctgcatacgctaccgctcaagaggcatacgaacaggcagtcgctaacggcgattctgaggtagtgcttaaaaaacttaaaaaatcacttaacgttgcgaaatctgagttcgatagagacgctgctatgcaacgtaaactcgaaaaaatggccgatcaggctatgacacaaatgtataaacaggctagatctgaggataagcgtgctaaggttacatccgctatgcaaactatgttgtttactatgttgcgtaaactcgataacgacgcacttaacaatattattaataacgctagagacggatgcgtaccacttaatattataccgcttactactgccgctaagcttatggtagtcgttcccgattacggtacttataagaatacatgcgacggtaatacttttacatacgctagtgcattgtgggagattcaacaggtcgttgacgctgattctaaaatcgttcaattgtctgagattaatatggataactcacctaatctcgcatggccattaatcgttacagcgttacgcgctaactctgccgttaagttacagaataacgaattgtcaccagtcgcattgcgtcaaatgtcatgcgctgccggtactacacaaaccgcttgtactgacgataacgctctcgcatactataataactctaaaggcggtagattcgtactcgcactattatccgatcatcaggatcttaaatgggctagattccctaagtctgacggtacaggtacaatttataccgaactcgaaccaccatgtagattcgttaccgatacacctaagggacctaaggttaagtatctatactttattaagggactgaataatctgaataggggtatggtgttagggtcactcgcagctaccgttagattgcaagccggtaacgctactgaggttcccgctaactcaaccgtacttagtttttgcgcattcgcagtcgatccagctaaggcttataaggattacttagctagcggaggtcaacctattactaattgcgttaaaatgctatgtacacataccggtacaggtcaggctattacggttacacctgaggctaatatggatcaggagtcattcggaggcgcatcatgttgtctatattgtagatgtcatatcgatcatcctaatcctaagggtttttgcgatcttaagggtaagtacgttcagattcctactacatgcgctaacgatccagtcggttttacattgcgtaataccgtttgtacagtatgcggtatgtggaaaggttacggatgttcatgcgatcagttacgcgaaccacttatgcaatccgctgacgctagtacattccttaatgggtttgcggtgtaagtgccgctagattgactccatgcggtacaggtactagtactgacgtagtgtatcgcgcattcgatatttataacgaaaaggttgccggattcgctaaattccttaagactaattgttgtagatttcaggaaaaagacgaagagggtaatctgttagactcatatttcgtagtgaaacgtcatactatgtctaattatcaacacgaagagactatttataatctcgttaaggattgtccagccgttgcggtacacgatttttttaaatttagagtcgacggtgatatggtgccacatatttctagacagagattgactaagtatactatggccgatctagtttacgctcttagacatttcgacgaaggtaattgcgatacacttaaagagatactcgttacatataattgttgcgacgacgattactttaataaaaaagattggtacgatttcgttgagaatcccgatatacttagggtttacgctaacttaggcgaacgcgttagacagtcattgcttaagacagtgcaattttgcgacgctatgcgtgacgcaggtatagtcggcgtacttacactcgataatcaggatcttaacggtaattggtacgatttcggcgatttcgttcaggtcgcaccaggttgcggagtgcctatagtcgattcatactattcattgcttatgcctatacttacacttacacgtgcacttgccgctgagtcacatatggacgctgatctcgctaaaccattgattaaatgggatctacttaaatacgattttactgaggaacgattgtgtctattcgataggtattttaagtattgggatcagacttatcatcctaattgtattaattgtcttgacgatagatgtatactgcattgcgctaattttaacgtattgttctcaaccgtattcccacctacatcattcggtccactcgtacgtaagattttcgttgacggcgtaccattcgtagtgtcaaccggttatcattttagagagttaggcgtagtgcataatcaggacgttaatctgcattctagtagactatcttttaaagagttgttagtgtacgctgccgatccagctatgcatgccgctagcggtaatctgttactcgataagcgtactacatgttttagcgttgccgcacttactaataacgttgcgtttcagactgttaaacccggtaattttaataaggatttttacgatttcgcagtgagtaagggtttttttaaagagggatctagcgttgagcttaagcatttttttttcgctcaagacggtaacgctgctattagcgattacgattactatagatataatctgccaactatgtgcgatatacgtcaattgttgttcgtcgttgaggtagtcgataagtatttcgattgttacgacggaggttgtattaacgctaatcaggtaatcgttaataatctcgataagtctgccggattcccattcaataaatggggtaaggctagattgtattacgattctatgtcatacgaggatcaggacgctctattcgcatatactaagcgtaacgttataccgacaattacgcaaatgaatcttaaatacgctattagcgctaaaaatcgtgcacgtacagtcgcaggcgtatcaatttgtagtactatgactaatcgtcaatttcaccaaaaattgcttaagtctattgccgctactagaggcgctacagtcgttatcggtacatctaaattttacggaggttggcataatatgcttaagacagtgtattctgacgttgagactccacatcttatgggttgggattaccctaaatgcgatagggctatgcctaatatgttgcgtattatggctagtctcgtactcgcacgtaaacataatacatgttgtaatctatcacatagattctatagactcgctaacgaatgcgctcaagtgcttagcgaaatggttatgtgtggcggatcactttacgttaagcctggcggtacatctagcggagacgctactaccgcttacgctaattccgtttttaatatttgtcaagccgttaccgctaacgttaacgctctattgtctactgacggtaataagattgccgataaatacgtacgtaatctgcaacatagattgtacgaatgtctgtatcgtaatcgcgatgtcgatcacgaattcgttgacgaattttacgcttacttacgtaaacatttttctatgatgattctatctgacgacgcagtcgtttgttataactctaattacgctgcacaggggttagtcgctagtattaagaattttaaagccgtactgtattatcagaataacgtttttatgtctgaggctaagtgttggaccgaaaccgatctgactaagggtccacacgaattttgttcacagcatactatgctcgttaaacagggtgacgattacgtttatctaccatatcccgatccgtcacgtatattaggcgcaggttgtttcgttgacgatatcgttaagactgacggtacacttatgattgagagattcgttagtctcgcaatcgacgcttatccattgactaagcatcctaatcaggaatacgctgacgtttttcacttatacttacagtatatacgtaaattgcacgacgaacttacagggcatatgcttgatatgtattcggttatgcttactaacgataatactagtaggtattgggaacctgagttttacgaagctatgtatacaccacataccgtactgcaagccgtaggcgcatgcgtactatgtaattcacagactagtcttagatgtggcgcatgtatacgtagaccattcttatgttgtaagtgttgttacgatcacgttattagtacatcacataagttagtgttatccgttaacccatacgtttgtaacgctccaggttgcgacgttactgacgttacgcaattgtacttaggcggtatgtcatactattgtaaatcacataaacctcctattagttttccgttatgcgctaacggtcaggtattcggtctatataagaatacatgcgtagggtctgataacgttaccgattttaacgctatcgctacatgcgattggactaacgctggcgattatatactcgctaatacatgtactgagcgacttaagttattcgcagccgaaacacttaaggctaccgaagagacttttaaattgtcatacggtatcgctaccgtacgcgaagtgcttagcgatagagagttgcatctatcatgggaggtcggtaagcctagaccaccacttaatcgtaattacgtttttacagggtatcgggttactaagaattcgaaagtgcaaattggcgaatatacattcgaaaaaggcgattacggtgacgccgtagtgtatagaggtactactacttataagcttaacgtaggcgattatttcgtacttacatcacataccgttatgccattatccgctcctacattagtgccacaagagcattacgttaggattaccggattgtatcctacacttaatatttctgacgaattttcatctaacgttgcgaattatcagaaagtgggtatgcaaaaatactctacattgcagggacctcccggtaccggtaagtctcatttcgcaatcggactcgcattgtattatcctagtgctaggattgtgtataccgcttgttcacacgctgcagtcgacgctctatgcgaaaaagctcttaagtatctaccaatcgataagtgttcacgtattatacccgctagggctagagtcgaatgtttcgataagtttaaagtgaattcgacactcgaacaatacgttttttgtacggttaacgctctaccagagactactgccgatatcgtagtgttcgacgaaatttctatggctactaattacgatctatcagtcgttaacgctagacttagggctaagcattacgtttatataggcgatccagcgcaattgcctgcaccacgtacattgttgactaagggtacactcgaacctgagtattttaattcagtgtgtagacttatgaaaactatcggacctgatatgtttctcggtacatgtcgtagatgtcctgccgaaatagtcgatacagttagcgcactcgtatacgataataagcttaaggcacataaggataagtctgcgcaatgttttaaaatgttttacaaaggcgtaattacacacgacgttagttccgctattaatagaccacagataggcgtagtgagagagtttcttacacgtaatcccgcttggcgtaaagccgtttttattagtccatataactctcaaaacgcagtcgctagtaagatactcggattgcctacacagacagtcgattctagtcagggtagcgaatacgattacgttatttttacgcaaactaccgaaaccgctcattcatgtaacgttaatagatttaacgttgcgattactagggctaaaatcggtattctatgtattatgtctgatcgcgatctatacgataagttgcaatttacatcacttgagatacctagacgtaacgttgcgacattgcaagccgaaaacgttaccggattgtttaaggattgttctaagattattaccggattgcatcctacacaggcacctacacatctatcagtcgatattaagtttaagactgagggtctatgcgttgacatacccggtatacctaaggatatgacttatcgtagattgattagtatgatgggttttaaaatgaattatcaggttaacggttatcctaatatgtttattacacgcgaagaggctattagacacgttagggcttggatagggttcgacgttgagggatgtcacgctacacgtgacgcagtcggtactaatctgccattgcaattagggttttcgacaggcgttaatctggttgccgtacctaccggatacgtcgatactgagaataatactgagtttactagggttaacgctaaacctccacctggcgatcaatttaaacatctgattccacttatgtataagggattgccttggaacgtagtgcgtattaagattgtgcaaatgcttagcgatacacttaagggattgtctgatagggttgtgttcgtactttgggctcacggattcgaattgacatctatgaaatatttcgttaaaatcggacctgagcgtacatgttgtctatgcgataagcgtgctacatgttttagtacatctagcgatacatacgcttgttggaatcattcagtcggattcgattacgtttataatccttttatgattgacgttcagcaatgggggtttaccggtaatctgcaatctaatcacgatcagcattgtcaggtacacggtaacgctcacgttgcgtcatgcgacgctattatgactagatgtcttgccgtacacgaatgtttcgttaagagagtcgattggtcagtcgaataccctattataggcgacgaacttagggttaattccgcttgtcgtaaagtgcaacatatggtcgttaagtctgctctattagccgataagtttcccgtattgcacgatatcggtaatcctaaagcgattaaatgcgtaccacaagccgaagtcgaatggaaattttacgacgctcaaccatgttctgataaggcatacaaaatcgaagagttgttttactcatacgctacacatcacgataagtttactgacggcgtatgtctgttttggaattgtaacgttgataggtatcccgctaacgctatcgtttgtagattcgatactagggttctatctaatctgaatctgccaggttgcgatggcggatcactatacgttaataaacacgcttttcatacacctgcattcgataaatccgcttttacgaatcttaagcaattgccttttttttattactctgatagtccatgcgaatctcacggtaagcaagtcgttagcgatatcgattacgttccacttaaatccgctacatgtattacacgttgtaatctcggaggcgcagtttgtagacatcacgctaacgaatatcgtcaatacttagacgcttataatatgatgattagtgccggtttttcattgtggatttataagcaattcgatacttataatctatggaatacttttactagattgcaatctctcgaaaacgttgcgtataacgtcgttaataagggtcatttcgacggtcacgctggcgaagctcccgtatcaattattaataacgcagtgtatactaaggtcgacggtattgacgtcgagattttcgaaaataagactacattacccgttaacgttgcattcgaattgtgggctaaacgtaatattaaaccagtgccagagattaagatacttaataacttaggcgttgatatcgctgctaataccgttatttgggattataaacgcgaagctcctgcacatgtgtcaactataggcgtatgtactatgactgatatcgctaaaaaacctaccgaatccgcttgttcatcacttacagtgttattcgacggtagggttgagggtcaggtcgatctgtttcgtaacgcacgtaacggcgtactgattactgagggatcggttaagggattgacacctagtaagggacctgctcaagctagcgttaacggagtgacacttataggcgaatcagttaagactcaatttaattactttaaaaaagtcgacggtattattcaacagttaccagagacttattttacacaatcacgcgatcttgaggattttaaacctagatctcaaatggagactgattttctcgaactcgctatggacgaatttattcaacgatataagcttgagggatacgcattcgaacatatcgtttacggcgatttttcacacggtcagttaggcggattgcatcttatgatcggtctagctaaacgatcacaggatagtccacttaagcttgaggattttatacctatggattcaaccgttaagaattactttattaccgatgcgcaaaccggttcatctaaatgcgtttgtagcgtaatcgatttactgttagacgatttcgttgagattattaagtctcaggatctatcagtgattagtaaggtagtgaaagtgacaatcgattacgctgagatttcttttatgctttggtgtaaagacggtcacgttgagactttttaccctaaattgcaggctagtcaggcatggcaacctggagtcgctatgcctaatttgtataaaatgcaacgtatgttactcgaaaaatgcgatctgcaaaattacggtgagaacgcagtgatacctaagggtattatgatgaacgtcgctaagtatacacaattgtgtcaataccttaatacacttacacttgccgtaccatataatatgcgagtgatacatttcggagccggatctgataagggcgttgcgccaggtactgccgtattgcgtcaatggttgcctaccggtacactgttagtcgattccgatcttaacgatttcgtatctgacgctgatagtacacttataggcgattgcgctacagtgcataccgctaataaatgggatctgattattagcgatatgtacgatccacgtactaaacacgttacgaaagagaacgattctaaagagggtttttttacatatctatgcggttttattaaacagaaactcgcattaggcggatctattgccgttaagattactgagcatagttggaacgctgatctgtataagcttatgggtcattttagttggtggaccgcattcgttactaacgttaacgctagttctagcgaagcattcttaatcggcgctaattatctcggtaagcctaaagagcaaatcgacggatatactatgcacgctaattatattttttggcgtaatactaatcctattcaattgtctagttattcattgttcgatatgtctaaatttccacttaagttacgcggtactgccgttatgtcacttaaagagaatcagattaacgatatgatttactctttactcgaaaagggtaggttgattatacgcgaaaataatagggtagtcgttagttctgacatactcgttaataattaaacgaacatgtttatttttctgttattccttacacttacatccggttcagatctcgatagatgtactacattcgacgacgttcaggcacctaattatacacagcatacatctagtatgagaggcgtatactatcctgacgaaatttttagatccgatacattgtatcttacacaggatctattcttacctttttactctaacgttacagggtttcatacaattaatcatacattcggtaatcccgttataccgtttaaagacggtatttatttcgcagctaccgaaaaatctaacgtagtgagaggttgggtattcggtagtactatgaataataagtctcaatcagtgattattattaataattcgactaacgtagtgatacgcgcatgtaatttcgaattgtgcgataatccttttttcgcagtgtctaaacctatgggtacacagactcatactatgattttcgataacgcttttaattgtacattcgaatatatttctgacgctttttcacttgacgtatccgaaaaatctggtaattttaaacacttacgcgaattcgtttttaaaaataaagacggttttttgtacgtttataagggataccaacctatcgacgtcgttagggatctgccatcaggttttaatacacttaagcctatttttaagttaccgttaggcattaatattactaattttagagctatacttaccgcttttagtccagctcaggatatttggggtactagcgctgccgcatatttcgtcggatatcttaaacctactacttttatgcttaaatacgacgaaaacggtacaattactgacgcagtcgattgttcacagaatccattagccgaacttaagtgttcagttaagtcattcgaaatcgataagggtatttatcagactagtaattttagagtcgtacctagcggtgacgtagtgagattccctaatattacgaatctatgtccattcggcgaagtgtttaacgctactaaattccctagcgtatacgcttgggagcgtaaaaaaattagtaattgcgttgccgattactctgtgttgtataattcgactttttttagtacttttaagtgttacggagtgtcagctactaagcttaacgatctatgtttttctaacgtatacgctgattcattcgtagtgaaaggcgatgacgttaggcaaatcgctcccggtcagactggcgtaatcgctgattataattataagttacctgacgattttatgggttgcgtactcgcatggaatacacgtaatatcgacgctactagtaccggtaattataattataagtatagatatcttagacacggtaagcttagaccattcgaacgcgatatttctaacgtaccattctcacctgacggtaagccttgtacacctcctgcacttaattgttattggccacttaacgattacggtttttacactactaccggtatagggtatcaaccttatagggtagtcgttctatcattcgaattgcttaacgctccagctaccgtatgcggacctaaattgtcaaccgatctgattaagaatcaatgcgttaattttaattttaacggtcttaccggtacaggcgtacttacacctagttctaaacggtttcaaccttttcagcaattcggtagggacgttagcgattttaccgatagcgttagggatcctaaaactagtgagatactcgatattagtccatgcgcattcggaggcgtaagtgtgattactcccggtactaacgctagttctgaggttgccgtactgtatcaggacgttaattgtactgacgtatcaaccgctatacacgctgatcaattgacacctgcatggcgtatatactctaccggtaataacgtttttcagacacaagccggttgtctgataggcgcagagcatgtcgatacatcatacgaatgcgatataccgataggcgcaggtatttgcgctagttatcatacagtgtcattgcttagatctactagtcagaaatcaatcgttgcgtatactatgtcattaggcgctgatagttcaatcgcatactctaataatactatcgctatacctactaatttttcaatttcgattactactgaggttatgccagtgtctatggctaagactagtgtcgattgtaatatgtatatttgtggcgattcaaccgaatgcgctaatctgttattgcaatacggatctttttgtacgcaattgaatcgtgcactatcaggtatagccgctgaacaggatcgtaatactagagaggtattcgcacaggttaaacagatgtataagactcctacacttaagtatttcggagggtttaatttttcacagattttacccgatccacttaaacctactaaacgatcttttattgaggatctgttattcaataaggttacactcgcagacgcaggttttatgaaacaatacggcgaatgtttaggcgatattaacgctagggatctgatttgcgctcaaaaatttaacggtcttacagtgttaccaccactattgactgacgatatgatagccgcatatactgccgcattagtgtcaggtaccgctaccgcaggttggacattcggtgccggtgccgcattgcagattccattcgctatgcaaatggcttatagatttaacggtataggcgttacgcaaaacgtactttacgagaatcagaaacaaatcgctaaccaatttaataaggctattagtcagattcaagagtcacttactactactagtaccgcactcggtaagttgcaagacgtcgttaatcagaacgctcaggcacttaatacactcgttaagcaattgtctagtaatttcggagctattagttcagtgcttaacgatattctatctagactcgataaggtcgaagccgaagtgcaaatcgatagattgattaccggtaggttgcaatctctgcaaacatacgttacgcaacaattgatacgcgctgctgagattagggctagcgctaatctcgcagctactaaaatgtctgagtgcgtactcggtcaatctaaaagagtcgatttttgcggtaaggggtatcatcttatgtcttttccacaagccgcaccacacggagtggtttttttacacgttacatacgtacctagtcaggaacgtaattttactaccgctccagctatttgtcacgaaggtaaggcatactttccacgcgaaggcgtattcgtttttaacggtacatcatggtttattacgcaacgtaatttttttagtccacaaattattactactgataatacattcgttagcggtaattgcgatgtcgttatcggtattattaataataccgtttacgatccattgcaacctgagttagactcttttaaagaggaactcgataagtattttaaaaatcatacatcacctgacgttgacttaggcgatatttcaggtattaacgcttcagtcgttaatattcagaaagagattgatagacttaacgaagtcgctaaaaatcttaacgaatcacttatcgatctgcaagagttaggtaagtacgaacagtatattaaatggccttggtacgtttggttagggtttatagccggtctaatcgctatcgttatggttacgatactgttatgttgtatgacatcatgttgttcatgtcttaaaggcgcatgttcatgcggatcatgttgtaaattcgacgaagacgattctgagccagtgcttaagggagtgaaattgcattatacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggccgataacggtacaattaccgttgaggaacttaaacagttactcgaacaatggaatctcgtaatcggttttctgtttctcgcatggattatgctattgcaattcgcatactctaatcgtaatcggtttttgtatattattaaactcgtattcttatggttattgtggccagttacactcgcatgtttcgtactcgcagccgtttatcgtattaattgggttacaggcggtatcgctatcgctatggcttgtatagtcggacttatgtggttgtcttatttcgttgcgtcatttagattgttcgcacgtactagatctatgtggtcttttaatcccgaaactaatatactgcttaacgtaccacttagaggtacaatcgttactagaccacttatggagtctgagttagtgataggcgcagtgattattagggggcatttgcgtatggccggtcatagtctaggtagatgcgatattaaggatctacctaaagagattaccgttgcgactagtcgtacactatcttattataagttaggcgctagtcaacgtgtcggtactgatagcggattcgcagcttataataggtatcgtatcggtaattataagcttaataccgatcacgctggatctaacgataatatcgcattactcgtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

(19) United States
(12) Patent Application Publication
(10) Pub. No.: US 2024/0228981 A1
(43) Pub. Date: Jul. 11, 2024

(54) ATTENUATED VIRUSES USEFUL FOR VACCINES

(71) Applicant: The Research Foundation for The State of University New York, Albany, NY (US)

(72) Inventors: Eckard Wimmer, East Setauket, NY (US); Steve Skiena, Setauket, NY (US); Steffen Mueller, Kings Point, NY (US); Bruce Futcher, Setauket, NY (US); Dimitris Papamichail, Newtown, PA (US); John Robert Coleman, Blauvelt, NY (US); Jeronimo Cello, Port Jefferson, NY (US)

(21) Appl. No.: 18/512,196

(22) Filed: Nov. 17, 2023

Related U.S. Application Data
(63) Continuation of application No. 17/513,032, filed on Oct. 28, 2021, now abandoned.
Application No. 17/513,032 is a division of application No. 16/036,348, filed on Jul. 16, 2018, now Pat. No. 11,162,080.
Application No. 16/036,348 is a continuation of application No. 15/258,584, filed on Sep. 7, 2016, now Pat. No. 10,023,845.
Application No. 15/258,584 is a division of application No. 12/594,173, filed on Mar. 29, 2010, now Pat. No. 9,476,032.
(60) Provisional application No. 60/909,389, filed on Mar. 30, 2007.
(60) Provisional application No. 61/068,666, filed on Mar. 7, 2008.

(51) Int. Cl. C12N 7/00; A61K 39/00; A61K 39/12; A61K 39/13; A61K 39/145; C07K 14/005

(52) U.S. Cl. CPC C12N 7/00; A61K 39/12; A61K 39/13; A61K 39/145; C07K 14/005; A61K 2039/5254; C12N 2720/12361; C12N 2740/15061; C12N 2760/16061; C12N 2760/16121; C12N 2760/16134; C12N 2760/16162; C12N 2770/20061; C12N 2770/24161; C12N 2770/32061; C12N 2770/32621; C12N 2770/32634; C12N 2770/32661; C12N 2770/32662; C12N 2770/32671; C12N 2770/32721; C12N 2770/32761; C12N 2770/32762; Y02A 50/30

(57) ABSTRACT

This invention provides an attenuated virus which comprises a modified viral genome containing nucleotide substitutions engineered in multiple locations in the genome, wherein the substitutions introduce synonymous deoptimized codons into the genome. The instant attenuated virus may be used in a vaccine composition for inducing a protective immune response in a subject. The invention also provides a method of synthesizing the instant attenuated virus. Further, this invention further provides a method for preventing a subject from becoming afflicted with a virus-associated disease comprising administering to the subject a prophylactically effective dose of a vaccine composition comprising the instant attenuated virus.

### CROSS REFERENCE TO RELATED APPLICATIONS

This application is continuation of U.S. application Ser. No. 17/513,032, filed Oct. 28, 2021, which is a divisional of U.S. application Ser. No. 16/036,348, filed Jul. 16, 2018, now U.S. Pat. No. 11,162,080, issued Nov. 2, 2021, which is a continuation of U.S. application Ser. No. 15/258,584, filed Sep. 7, 2016, now U.S. patent Ser. No. 10/023,845, issued Jul. 17, 2018, which is a divisional of U.S. application Ser. No. 12/594,173, filed Mar. 29, 2010, now U.S. Pat. No. 9,476,032, issued Oct. 25, 2016, which is the national phase application of International application number PCT/US2008/058952, filed Mar. 31, 2008, which claims the benefit of priority to U.S. Application No. 60/909,389, filed Mar. 30, 2007, and U.S. Application No. 61/068,666, filed Mar. 7, 2008, which are incorporated herein by reference in their entireties.

### FEDERAL FUNDING

This invention was made with government support under Grant Nos. AI15122 and T32-CA009176 awarded by the National Institutes of Health, and EIA0325123 awarded by the National Science Foundation. The government has certain rights in the invention.

### REFERENCE TO A SEQUENCE LISTING

This application contains a Sequence Listing which has been submitted electronically in xml format and is hereby incorporated by reference in its entirety. Said xml copy, created on Nov. 14, 2023, is named SeqList-162152-46207.xml and is 246,232 bytes in size.

### COPYRIGHT NOTICE

A portion of the disclosure of this patent document contains material which is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### FIELD OF THE INVENTION

The present invention relates to the creation of an attenuated virus comprising a modified viral genome containing a plurality of nucleotide substitutions. The nucleotide substitutions result in the exchange of codons for other synonymous codons and/or codon rearrangement and variation of codon pair bias.

### BACKGROUND OF THE INVENTION

Rapid improvements in DNA synthesis technology promise to revolutionize traditional methods employed in virology. One of the approaches traditionally used to eliminate the functions of different regions of the viral genome makes extensive but laborious use of site-directed mutagenesis to explore the impact of small sequence variations in the genomes of virus strains. However, viral genomes, especially of RNA viruses, are relatively short, often less than 10,000 bases long, making them amenable to whole genome synthesis using currently available technology. Recently developed microfluidic chip-based technologies can perform de novo synthesis of new genomes designed to specification for only a few hundred dollars each. This permits the generation of entirely novel coding sequences or the modulation of existing sequences to a degree practically impossible with traditional cloning methods.

Such freedom of design provides tremendous power to perform large-scale redesign of DNA/RNA coding sequences to: (1) study the impact of changes in parameters such as codon bias, codon-pair bias, and RNA secondary structure on viral translation and replication efficiency; (2) perform efficient full genome scans for unknown regulatory elements and other signals necessary for successful viral reproduction; and (3) develop new biotechnologies for genetic engineering of viral strains and design of anti-viral vaccines.

As a result of the degeneracy of the genetic code, all but two amino acids in the protein coding sequence can be encoded by more than one codon. The frequencies with which such synonymous codons are used are unequal and have coevolved with the cell's translation machinery to avoid excessive use of suboptimal codons that often correspond to rare or otherwise disadvantaged tRNAs (Gustafsson et al., 2004). This results in a phenomenon termed “synonymous codon bias,” which varies greatly between evolutionarily distant species and possibly even between different tissues in the same species (Plotkin et al., 2004).

Codon optimization by recombinant methods (that is, to bring a gene's synonymous codon use into correspondence with the host cell's codon bias) has been widely used to improve cross-species expression (see, e.g., Gustafsson et al., 2004). Though the opposite objective of reducing expression by intentional introduction of suboptimal synonymous codons has not been extensively investigated, isolated reports indicate that replacement of natural codons by rare codons can reduce the level of gene expression in different organisms. See, e.g., Robinson et al., 1984; Hoekema et al., 1987; Carlini and Stephan, 2003; Zhou et al., 1999. Accordingly, the introduction of deoptimized synonymous codons into a viral genome may adversely affect protein translation and thereby provide a method for producing attenuated viruses that would be useful for making vaccines against viral diseases.

### Viral Disease and Vaccines

Viruses have always been one of the main causes of death and disease in man. Unlike bacterial diseases, viral diseases are not susceptible to antibiotics and are thus difficult to treat. Accordingly, vaccination has been humankind's main and most robust defense against viruses. Today, some of the oldest and most serious viral diseases such as smallpox and poliomyelitis (polio) have been eradicated (or nearly so) by world-wide programs of immunization. However, many other old viruses such as rhinovirus and influenza virus are poorly controlled, and still create substantial problems, though these problems vary from year to year and country to country. In addition, new viruses, such as Human Immunodeficiency Virus (HIV) and Severe Acute Respiratory Syndrome (SARS) virus, regularly appear in human populations and often cause deadly pandemics. There is also potential for lethal man-made or man-altered viruses for intentional introduction as a means of warfare or terrorism.

Effective manufacture of vaccines remains an unpredictable undertaking. There are three major kinds of vaccines: subunit vaccines, inactivated (killed) vaccines, and attenuated live vaccines. For a subunit vaccine, one or several proteins from the virus (e.g., a capsid protein made using recombinant DNA technology) are used as the vaccine. Subunit vaccines produced in Escherichia coli or yeast are very safe and pose no threat of viral disease. Their efficacy, however, can be low because not all of the immunogenic viral proteins are present, and those that are present may not exist in their native conformations.

Inactivated (killed) vaccines are made by growing more-or-less wild type (wt) virus and then inactivating it, for instance, with formaldehyde (as in the Salk polio vaccine). A great deal of experimentation is required to find an inactivation treatment that kills all of the virus and yet does not damage the immunogenicity of the particle. In addition, residual safety issues remain in that the facility for growing the virus may allow virulent virus to escape or the inactivation may fail.

An attenuated live vaccine comprises a virus that has been subjected to mutations rendering it less virulent and usable for immunization. Live, attenuated viruses have many advantages as vaccines: they are often easy, fast, and cheap to manufacture; they are often easy to administer (the Sabin polio vaccine, for instance, was administered orally on sugar cubes); and sometimes the residual growth of the attenuated virus allows “herd” immunization (immunization of people in close contact with the primary patient). These advantages are particularly important in an emergency, when a vaccine is rapidly needed. The major drawback of an attenuated vaccine is that it has some significant frequency of reversion to wt virulence. For this reason, the Sabin vaccine is no longer used in the United States.

Accordingly, there remains a need for a systematic approach to generating attenuated live viruses that have practically no possibility of reversion and thus provide a fast, efficient, and safe method of manufacturing a vaccine. The present invention fulfills this need by providing a systematic approach, Synthetic Attenuated Virus Engineering (SAVE), for generating attenuated live viruses that have essentially no possibility of reversion because they contain hundreds or thousands of small defects. This method is broadly applicable to a wide range of viruses and provides an effective approach for producing a wide variety of anti-viral vaccines.

### SUMMARY OF THE INVENTION

The present invention provides an attenuated virus which comprises a modified viral genome containing nucleotide substitutions engineered in multiple locations in the genome, wherein the substitutions introduce a plurality of synonymous codons into the genome. This substitution of synonymous codons alters various parameters, including codon bias, codon pair bias, density of deoptimized codons and deoptimized codon pairs, RNA secondary structure, CpG dinucleotide content, C+G content, translation frameshift sites, translation pause sites, the presence or absence of tissue specific microRNA recognition sequences, or any combination thereof, in the genome. Because of the large number of defects involved, the attenuated virus of the invention provides a means of producing stably attenuated, live vaccines against a wide variety of viral diseases.

In one embodiment, an attenuated virus is provided which comprises a nucleic acid sequence encoding a viral protein or a portion thereof that is identical to the corresponding sequence of a parent virus, wherein the nucleotide sequence of the attenuated virus contains the codons of a parent sequence from which it is derived, and wherein the nucleotide sequence is less than 90% identical to the nucleotide sequence of the parent virus. In another embodiment, the nucleotide sequence is less that 80% identical to the sequence of the parent virus. The substituted nucleotide sequence which provides for attenuation is at least 100 nucleotides in length, or at least 250 nucleotides in length, or at least 500 nucleotides in length, or at least 1000 nucleotides in length. The codon pair bias of the attenuated sequence is less than the codon pair bias of the parent virus, and is reduced by at least about 0.05, or at least about 0.1, or at least about 0.2.

The virus to be attenuated can be an animal or plant virus. In certain embodiments, the virus is a human virus. In another embodiment, the virus infects multiple species. Particular embodiments include, but are not limited to, poliovirus, influenza virus, Dengue virus, HIV, rotavirus, and SARS.

This invention also provides a vaccine composition for inducing a protective immune response in a subject comprising the instant attenuated virus and a pharmaceutically acceptable carrier. The invention further provides a modified host cell line specially engineered to be permissive for an attenuated virus that is inviable in a wild type host cell.

In addition, the subject invention provides a method of synthesizing the instant attenuated virus comprising (a) identifying codons in multiple locations within at least one non-regulatory portion of the viral genome, which codons can be replaced by synonymous codons; (b) selecting a synonymous codon to be substituted for each of the identified codons; and (c) substituting a synonymous codon for each of the identified codons.

Moreover, the subject invention provides a method of synthesizing the instant attenuated virus comprising changing the order, within the coding region, of existing codons encoding the same amino acid in order to modulate codon pair bias.

Even further, the subject invention provides a method of synthesizing the instant attenuated virus that combines the previous two methods.

According to the invention, attenuated virus particles are made by transfecting viral genomes into host cells, whereby attenuated virus particles are produced. The invention further provides pharmaceutical compositions comprising attenuated virus which are suitable for immunization.

This invention further provides methods for eliciting a protective immune response in a subject, for preventing a subject from becoming afflicted with a virus-associated disease, and for delaying the onset, or slowing the rate of progression, of a virus-associated disease in a virus-infected subject, comprising administering to the subject a prophylactically or therapeutically effective dose of the instant vaccine composition.

The present invention further provides an attenuated virus which comprises a modified viral genome containing nucleotide substitutions engineered in multiple locations in the genome, wherein the substitutions introduce a plurality of synonymous codons into the genome, wherein the nucleotide substitutions are selected by a process comprising the steps of initially creating a coding sequence by randomly assigning synonymous codons in respective amino acid allowed positions, calculating a codon pair score of the coding sequence randomly selecting and exchanging either (a) pairs of codons encoding the same amino acids or (b) substituting synonymous codons in accordance with a simulated annealing optimization function and repeating the previous step until no further improvement (no change in pair score or bias) is observed for a specific or sufficient number of iterations, until the solution converges on an optima or near optimal value

### BRIEF DESCRIPTION OF THE FIGURES

FIG. 1. Codon use statistics in synthetic P1 capsid designs. PV-SD maintains nearly identical codon frequencies compared to wt, while maximizing codon positional changes within the sequence. In PV-AB capsids, the use of nonpreferred codons was maximized. The lengths of the bars and the numbers behind each bar indicate the occurrence of each codon in the sequence. As a reference, the normal human synonymous codon frequencies (“Freq.” expressed as a percentage) for each amino acid are given in the third column.

FIGS. 2A-2B. Sequence alignment of PV(M), PV-AB and PV-SD capsid coding regions. The nucleotide sequences of PV(M) (SEQ ID NO:1), PV-AB (SEQ ID NO:2) and PV-SD (SEQ ID NO:3) were aligned using the MultAlin online software tool (Corpet, 1988). Numbers above the sequence refer to the position within the capsid sequence. (FIG. 2A) Nucleotide 1 to nucleotide 1300; (FIG. 2B) nucleotide 1301 to nucleotide 2643. Nucleotide 1 corresponds to nucleotide 743 in the PV(M) virus genome. In the consensus sequence, the occurrence of the same nucleotide in all three sequences is indicated by an upper case letter; the occurrence of the same nucleotide in two of the three sequences is indicated by a lower case letter; and the occurrence of three different nucleotides in the three sequences is indicated by a period.

FIGS. 3A-J. Codon-deoptimized virus phenotypes. (FIG. 3A) Overview of virus constructs used in this study. (FIG. 3B) One-step growth kinetics in HeLa cell monolayers. (FIGS. 3C to H) Plaque phenotypes of codon-deoptimized viruses after 48 h (FIGS. 3C to F) or 72 h (FIGS. 3G and H) of incubation; stained with anti-3D^{pol} antibody to visualize infected cells. (FIG. 3C) PV(M), (FIG. 3D) PV-SD, (FIG. 3E) PV-AB, (FIG. 3F) PV-AB⁷⁵⁵⁻¹⁵¹³ (FIGS. 3G and H) PV-AB²⁴⁷⁰⁻²⁹⁵⁴. Cleared plaque areas are outlined by a rim of infected cells (FIGS. 3C and D). (FIG. 3H) No plaques are apparent with PV-AB²⁴⁷⁰⁻²⁹⁵⁴ after subsequent crystal violet staining of the well shown in panel FIG. 3G. (FIGS. 31 and J) Microphotographs of the edge of an immunostained plaque produced by PV(M) (FIG. 3I) or an infected focus produced by PV-AB²⁴⁷⁰⁻²⁹⁵⁴ (FIG. 3J) after 48 h of infection.

FIGS. 4A-E. Codon deoptimization leads to a reduction of specific infectivity. (FIG. 4A) Agarose gel electrophoresis of virion genomic RNA isolated from purified virus particles of PV(M) (lane 1), PV-AB⁷⁵⁵⁻¹⁵¹³ (lane 2), and PV-AB²⁴⁷⁰⁻²⁹⁵⁴ (lane 3). (FIG. 4B) Silver-stained SDS-PAGE protein gel of purified PV(M) (lane 1), PV-AB⁷⁵⁵⁻¹⁵¹³ (lane 2), and PV-AB²⁴⁷⁰⁻²⁹⁵⁴ (lane 3) virus particles. The three larger of the four capsid proteins (VP1, VP2, and VP3) are shown, demonstrating the purity and relative amounts of virus preparations. (FIG. 4C) Development of a virus capture ELISA using a poliovirus receptor-alkaline phosphatase (CD155-AP) fusion protein probe. Virus-specific antibodies were used to coat ELISA plates, and samples containing an unknown virus concentration were applied followed by detection with CD155-AP. Virus concentrations were calculated using a standard curve prepared in parallel with known amounts of purified wt virus (FIG. 4E). (FIG. 4D) The amounts of purified virus and extracted virion RNA were spectrophotometrically quantified, and the number of particles or genome equivalents (1 genome=1 virion) was calculated. In addition, virion concentrations were determined by ELISA. The infectious titer of each virus was determined by plaque/infected-focus assay, and the specific infectivity was calculated as PFU/particle or FFU/particle.

FIGS. 5A-B. In vitro translation of codon-deoptimized and wild type viruses. The PV-AB phenotype is determined at the level of genome translation. (FIG. 5A) A standard in vitro translation in HeLa S10 extract, in the presence of exogenously added amino acids and tRNAs reveals no differences in translation capacities of codon-deoptimized genomes compared to the PV(M) wt. Shown is an autoradiograph of [³⁵S]methionine-labeled translation products resolved on a 12.5% SDS-PAGE gel. The identity of an aberrant band (*) is not known. (FIG. 5B) In vitro translation in nondialyzed HeLa S10 extract without the addition of exogenous amino acids and tRNA and in the presence of competing cellular mRNAs uncovers a defect in translation capacities of codon-deoptimized PV genomes. Shown is a Western blot of poliovirus 2C reactive translation products (2C^{ATPase}, 2BC, and P2) resolved on a 10% SDS-PAGE gel. The relative amounts of the 2BC translation products are expressed below each lane as percentages of the wt band.

FIGS. 6A-B. Analysis of in vivo translation using dicistronic reporter replicons confirms the detrimental effect of codon deoptimization on PV translation. (FIG. 6A) Schematic of dicistronic replicons. Various P1 capsid coding sequences were inserted upstream of the firefly luciferase gene (F-Luc). Determination of changing levels of F-Luc expression relative to an internal control (R-Luc) allows for the quantification of ribosome transit through the P1 capsid region. (FIG. 6B) Replicon RNAs were transfected into HeLa cells and incubated for 7 h in the presence of 2 mM guanidine-hydrochloride to block RNA replication. The relative rate of translation through the P1 region was inversely proportional to the extent of codon deoptimization. While the capsid coding sequences of two viable virus constructs, PV-AB²⁴⁷⁰⁻²⁹⁵⁴ and PV-AB²⁹⁵⁴⁻³³⁸⁶, allow between 60 and 80% of wt translation, translation efficiency below 20% is associated with the lethal phenotypes observed with the PV-AB, PV-AB²⁴⁷⁰⁻³³⁸⁶, and PV-AB¹⁵¹³⁻²⁴⁷⁰ genomes. Values represents the average of 6 assays from 3 independent experiments.

FIG. 7. Determining codon pair bias of human and viral ORFs. Dots represent the average codon-pair score per codon pair for one ORF plotted against its length. Codon pair bias (CPB) was calculated for 14,795 annotated human genes. Under-represented codon pairs yield negative scores. CPB is plotted for various poliovirus P1 constructs, represented by symbols with arrows. The figure illustrates that the bulk of human genes clusters around 0.1. CPB is shown for PV(M)-wt (labeled “WT”) (−0.02), customized synthetic poliovirus capsids PV-Max (+0.25), PV-Min (−0.48), and PV(M)-wt:PV-Min chimera capsids PV-Min⁷⁵⁵⁻²⁴⁷⁰ (=“PV-MinXY”) (−0.31) and PV-Min²⁴⁷⁰⁻³³⁸⁶ (=“PV-MinZ”) (−0.20). Viruses PV-SD and PV-AB are the result of altered codon bias, but not altered codon pair bias.

FIGS. 8A-B. Characteristics of codon-pair deoptimized polio. (FIG. 8A) One-step growth kinetics reveals PFU production for PV-Min⁷⁵⁵⁻²⁴⁷⁰ and PV-Min²⁴⁷⁰⁻³³⁸⁵ that is reduced on the order of 2.5 orders of magnitude by comparison to PV(M)-wt. However, all viruses produce a similar number of viral particles (not shown in this Figure). (FIG. 8B) As a result the PFU/particle ratio is reduced, similar to codon deoptimized viruses PV-AB⁷⁵⁵⁻¹⁵¹³ and PV-AB²⁴⁷⁰⁻²⁹⁵⁴ (see FIG. 3B) (PFU is “Plaque Forming Unit”).

FIG. 9. Assembly of chimeric viral genomes. To “scan” through a target genome (red) small segments are amplified or synthesized and introduced into the wt genome (black) by overlapping PCR.

FIG. 10. The eight-plasmid pol I-pol II system for the generation of influenza A virus. Eight expression plasmids containing the eight viral cDNAs inserted between the human pol I promoter and the pol II promoter are transfected into eukaryotic cells. Because each plasmid contains two different promoters, both cellular pol I and pol II will transcribe the plasmid template, presumably in different nuclear compartments, which will result in the synthesis of viral mRNAs and vRNAs. After synthesis of the viral polymerase complex proteins (PB1, PB2, PA, nucleoproteins), the viral replication cycle is initiated. Ultimately, the assembly of all viral molecules directly (pol II transcription) or indirectly (pol I transcription and viral replication) derived from the cellular transcription and translation machinery results in the interaction of all synthesized molecules (vRNPs and the structural proteins HA, NA, M1, M2, NS2/NEP) to generate infectious influenza A virus. (Reproduced from Neumann et al., 2000.) (Note: there are other ways of synthesizing influenza de novo).

FIGS. 11A-B. Poliovirus Genome and Synthetic Viral Constructs. The poliovirus genome and open reading frames of chimeric virus constructs. (FIG. 11A) Top, a schematic of the full-length PV(M)-wt genomic RNA. (FIG. 11B) Below, the open reading frames of PV(M)-wt, the CPB customized synthetic viruses PV-Max, PV-Min, and the PV(M)-wt:PV-Min chimera viruses. Black corresponds to PV(M)-wt sequence, Gray to PV-Min synthetic sequence, and Thatched to PV-Max. The viral constructs highlighted, PV-Min⁷⁵⁵⁻²⁴⁷⁰ (PV-MinXY) and PV-Min²⁴⁷⁰⁻³³⁸⁵ (PV-MinZ), were further characterized due to a markedly attenuated phenotype.

FIGS. 12A-B. On-Step growth curves display similar kinetics yielding a similar quantity of particles with decreased infectivity. (FIG. 12A) An MOI of 2 was used to infect a monolayer of HeLa R19 cells, the PFU at the given time points (0, 2, 4, 7, 10, 24, 48 hrs) was measured by plaque assay. Corresponding symbols: (□) PV(M)-wt, (●) PV-Max, (⋄) PV-Min755-1513, (x) PV-Min1513-2470, (♦) PV-MinXY, (Δ) PV-MinZ. (FIG. 12B) Displays the conversion of the calculated PFU/ml at each time point to particles/ml. This achieved by multiplying the PFU/ml by the respective viruses specific infectivity. Corresponding symbols as in (FIG. 12A)

FIGS. 13A-B. In vivo modulation of translation by alteration of CPB. (FIG. 13A) The dicistronic RNA construct used to quantify the in vivo effect CPB has on translation. The first cistron utilizes a hepatitis C virus (HCV) Internal Ribosome Entry Site (IRES) inducing the translation of Renilla Luciferase (R-Luc). This first cistron is the internal control used to normalize the amount of input RNA. The second cistron controlled by the PV(M)-wt IRES induces the translation of Firefly Luciferase (F-Luc). The region labeled “P1” in the construct was replaced by the cDNA of each respective viruses P1. (FIG. 13B) Each respective RNA construct was transfected, in the presence of 2 mM guanidine hydrochloride, into HeLa R19 cells and after 6 hours the R-Luc and F-Luc were measured. The F-Luc/R-Luc values were normalized relative to PV(M)-wt translation (100%).

FIG. 14. The heat inactivation profile of the synthetic viruses is unchanged. To rule out that large scale codon-pair bias modification alters the gross morphology of virions, as one might expect if capsid proteins were misfolded, the thermal stability of PVMinXY and PV-MinZ was tested. An equal number of particles were incubated at 50° C. and the remaining infectivity quantified after given periods of time via plaque assay. If the capsids of the synthetic viruses were destabilized we would expect increased loss of viability at 50° C. in comparison to wt PV(M). This was not the case. The thermal inactivation kinetics of both synthetic viruses was identical to the wt. In contrast, the Sabin-1 virus carries numerous mutations in the genome region encoding the capsid, which, fittingly, rendered this virus less heat stabile as compared to wt PV1(M).

FIG. 15. Neutralizing antibody titer following vaccination. A group of eight CD155tg mice, seven of which completed the regimen, were each inoculated by intraperitoneal injection three times at weekly intervals with 10⁸ particles of PV-MinZ (●) and PV-MinXY (♦) and the serum conversion was measured 10 days after the final vaccination. A horizontal lines across each data set marks the average neutralizing antibody titer for each virus construct. The anti-poliovirus antibody titer was measured via micro-neutralization assay. (*) No virus neutralization for mock-vaccinated animals was detected at the lowest tested 1:8.

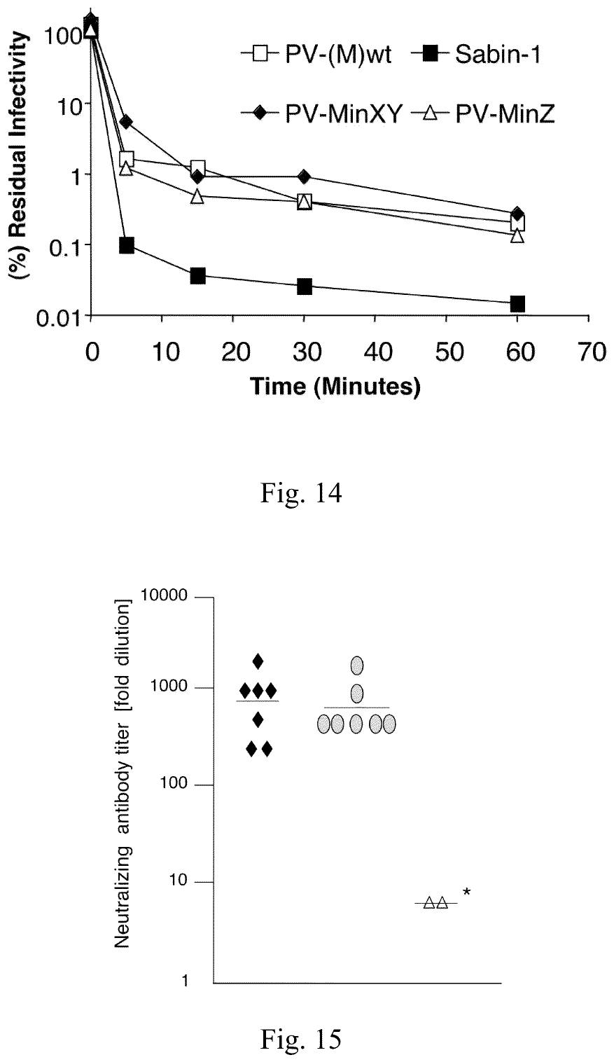

FIGS. 16A-B. Influenza virus carrying codon pair-deoptimized NP segment. (FIG. 16A) A/PR8-NP^{Min} virus are viable and produce smaller plaques on MDCK cells compared to the A/PR8 wt. (FIG. 16B) A/PR8-NP^{Min} virus display delayed growth kinetics and final titers 3-5 fold below wild type A/PR8.

FIGS. 17A-B. Influenza virus carrying codon pair-deoptimized PB1 or HA and NP segments. (FIG. 17A) A/PR8-PB1^{Min-RR} and A/PR8-HA^{Min}/NP^{Min} virus are viable and produce smaller plaques on MDCK cells as compared to the A/PR8 wild type. (FIG. 17B) A/PR8-PB1^{Min-RR} and A/PR8-HA^{Min}/NP^{Min} virus display delayed growth kinetics and final titers about 10 fold below wild type A/PR8.

FIGS. 18A-C. Attenuation of A/PR8-NP^{Min} in BALB/c mouse model. (FIG. 18A) A/PR8-NP^{Min} virus has reduced pathogenicity compared to wild type A/PR8 virus as determined by weight loss upon vaccination. (FIG. 18B) All mice (eight of eight) vaccinated with A/PR8-NP^{Min} virus survived, where as only 25% (two of eight) mice infected with A/PR8 were alive 13 days post vaccination. (FIG. 18C) Mice vaccinated with A/PR8-NP^{Min} virus are protected from challenge with 100×LD₅₀ of A/PR8 wild type virus.

### DETAILED DESCRIPTION OF THE INVENTION

The present invention relates to the production of attenuated viruses that may be used as vaccines to protect against viral infection and disease. Accordingly, the invention provides an attenuated virus, which comprises a modified viral genome containing nucleotide substitutions engineered in multiple locations in the genome, wherein the substitutions introduce a plurality of synonymous codons into the genome and/or a change of the order of existing codons for the same amino acid (change of codon pair utilization). In both cases, the original, wild-type amino acid sequences of the viral gene products are retained.

Most amino acids are encoded by more than one codon. See the genetic code in Table 1. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy. Thus, to replace a given codon in a nucleic acid by a synonymous but less frequently used codon is to substitute a “deoptimized” codon into the nucleic acid.

TABLE 1
Genetic Code

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a} The first nucleotide in each codon encoding a particular amino acid is shown in the left-most column; the second nucleotide is shown in the top row; and the third nucleotide is shown in the right-most column.

In addition, a given organism has a preference for the nearest codon neighbor of a given codon A, referred to a bias in codon pair utilization. A change of codon pair bias, without changing the existing codons, can influence the rate of protein synthesis and production of a protein.

In various embodiments of the present invention, the virus is a DNA, RNA, double-stranded, or single-stranded virus. In further embodiments, the virus infects an animal or a plant. In preferred embodiments, the animal is a human. A large number of animal viruses are well known to cause diseases (see below). Certain medically important viruses, such as those causing rabies, severe acute respiratory syndrome (SARS), and avian flu, can also spread to humans from their normal non-human hosts.

Viruses also constitute a major group of plant pathogens, and research is ongoing to develop viral vectors for producing transgenic plants. The advantages of such vectors include the ease of transforming plants, the ability to transform mature plants which obviates the need for regeneration of a transgenic plant from a single transformed cell, and high levels of expression of foreign genes from the multiple copies of virus per cell. However, one of the main disadvantages of these vectors is that it has not been possible to separate essential viral replicative functions from pathogenic determinants of the virus. The SAVE strategy disclosed herein may afford a means of engineering non-pathogenic viral vectors for plant transformation.

### Major Viral Pathogens in Humans

Viral pathogens are the causative agents of many diseases in humans and other animals. Well known examples of viral diseases in humans include the common cold (caused by human rhinoviruses, HRV), influenza (influenza virus), chickenpox (varicella-zoster virus), measles (a paramyxovirus), mumps (a paramyxovirus), poliomyelitis (poliovirus, PV), rabies (Lyssavirus), cold sores (Herpes Simplex Virus [HSV] Type 1), and genital herpes (HSV Type 2). Prior to the introduction of vaccination programs for children, many of these were common childhood diseases worldwide, and are still a significant threat to health in some developing countries. Viral diseases also include more serious diseases such as acquired immunodeficiency syndrome (AIDS) caused by Human Immunodeficiency Virus (HIV), severe acute respiratory syndrome (SARS) caused by SARS coronavirus, avian flu (H5N1 subtype of influenza A virus), Ebola (ebolavirus), Marburg haemorrhagic fever (Marburg virus), dengue fever (Flavivirus serotypes), West Nile encephalitis (a flavivirus), infectious mononucleosis (Epstein-Barr virus, EBV), hepatitis (Hepatitis C Virus, HCV; hepatitis B virus, HBV), and yellow fever (flavivirus). Certain types of cancer can also be caused by viruses. For example, although most infections by human papillomavirus (HPV) are benign, HPV has been found to be associated with cervical cancer, and Kaposi's sarcoma (KS), a tumor prevalent in AIDS patients, is caused by Kaposi's sarcoma-associated herpesvirus (KSHV).

Because viruses reside within cells and use the machinery of the host cell to reproduce, they are difficult to eliminate without killing the host cell. The most effective approach to counter viral diseases has been the vaccination of subjects at risk of infection in order to provide resistance to infection. For some diseases (e.g., chickenpox, measles, mumps, yellow fever), effective vaccines are available. However, there is a pressing need to develop vaccines for many other viral diseases. The SAVE (Synthetic Attenuated Virus Engineering) approach to making vaccines described herein is in principle applicable to all viruses for which a reverse genetics system (see below) is available. This approach is exemplified herein by focusing on the application of SAVE to develop attenuated virus vaccines for poliomyelitis, the common cold, and influenza.

Any virus can be attenuated by the methods disclosed herein. The virus can be a dsDNA viruss (e.g. Adenoviruses, Herpesviruses, Poxviruses), a single stranded “plus” sense DNA virus (e.g., Parvoviruses) a double stranded RNA virus (e.g., Reoviruses), a single stranded + sense RNA virus (e.g. Picornaviruses, Togaviruses), a single stranded “minus” sense RNA virus (e.g. Orthomyxoviruses, Rhabdoviruses), a single stranded + sense RNA virus with a DNA intermediate (e.g. Retroviruses), or a double stranded reverse transcribing virus (e.g. Hepadnaviruses). In certain non-limiting embodiments of the present invention, the virus is poliovirus (PV), rhinovirus, influenza virus including avian flu (e.g. H5N1 subtype of influenza A virus), severe acute respiratory syndrome (SARS) coronavirus, Human Immunodeficiency Virus (HIV), Hepatitis B Virus (HBV), Hepatitis C Virus (HCV), infectious bronchitis virus, ebolavirus, Marburg virus, dengue fever virus (Flavivirus serotypes), West Nile disease virus, Epstein-Barr virus (EBV), yellow fever virus, Ebola (ebolavirus), chickenpox (varicella-zoster virus), measles (a paramyxovirus), mumps (a paramyxovirus), rabies (Lyssavirus), human papillomavirus, Kaposi's sarcoma-associated herpesvirus, Herpes Simplex Virus (HSV Type 1), or genital herpes (HSV Type 2).

The term “parent” virus or “parent” protein encoding sequence is used herein to refer to viral genomes and protein encoding sequences from which new sequences, which may be more or less attenuated, are derived. Parent viruses and sequences are usually “wild type” or “naturally occurring” prototypes or isolates of variants for which it is desired to obtain a more highly attenuated virus. However, parent viruses also include mutants specifically created or selected in the laboratory on the basis of real or perceived desirable properties. Accordingly, parent viruses that are candidates for attenuation include mutants of wild type or naturally occurring viruses that have deletions, insertions, amion acid substitutions and the like, and also include mutants which have codon substitutions. In one embodiment, such a parent sequence differs from a natural isolate by about 30 amino acids or fewer. In another embodiment, the parent sequence differes from a natural isolate by about 20 amino acids or fewer. In yet another embodiment, the parent sequence differs from a natural isolate by about 10 amino acids or fewer.

The attenuated PV may be derived from poliovirus type 1 (Mahoney; “PV(M)”), poliovirus type 2 (Lansing), poliovirus type 3 (Leon), monovalent oral poliovirus vaccine (OPV) virus, or trivalent OPV virus. In certain embodiments, the poliovirus is PV-AB having the genomic sequence set forth in SEQ ID NO:2, or PV-AB⁷⁵⁵⁻¹⁵¹³, PV-AB⁷⁵⁵⁻²⁴⁷⁰ PV-AB¹⁵¹³⁻³³⁸⁶, PV-AB²⁴⁷⁰⁻³³⁸⁶, PV-AB¹⁵¹³⁻²⁴⁷⁰, PV-AB²⁴⁷⁰⁻²⁹⁵⁴, or PV-AB²⁹⁵⁴⁻³³⁸⁶. The nomenclature reflects a PV(M) genome in which portions of the genome, are substituted with nucleotides of PV-AB. The superscript provides the nucleotide numbers of PV-AB that are substituted.

In various embodiments, the attenuated rhinovirus is a human rhinovirus (HRV) derived from HRV2, HRV14, Human rhinovirus 10 Human rhinovirus 100; Human rhinovirus 11; Human rhinovirus 12; Human rhinovirus 13; Human rhinovirus 15; Human rhinovirus 16; Human rhinovirus 18; Human rhinovirus 19; Human rhinovirus 1A; Human rhinovirus 1B; Human rhinovirus 2; Human rhinovirus 20; Human rhinovirus 21; Human rhinovirus 22; Human rhinovirus 23; Human rhinovirus 24; Human rhinovirus 25; Human rhinovirus 28; Human rhinovirus 29; Human rhinovirus 30; Human rhinovirus 31 Human rhinovirus 32; Human rhinovirus 33; Human rhinovirus 34; Human rhinovirus 36; Human rhinovirus 38; Human rhinovirus 39; Human rhinovirus 40; Human rhinovirus 41; Human rhinovirus 43; Human rhinovirus 44; Human rhinovirus 45; Human rhinovirus 46; Human rhinovirus 47; Human rhinovirus 49; Human rhinovirus 50; Human rhinovirus 51; Human rhinovirus 53; Human rhinovirus 54; Human rhinovirus 55; Human rhinovirus 56; Human rhinovirus 57; Human rhinovirus 58; Human rhinovirus 59; Human rhinovirus 60; Human rhinovirus 61; Human rhinovirus 62; Human rhinovirus 63; Human rhinovirus 64; Human rhinovirus 65; Human rhinovirus 66; Human rhinovirus 67; Human rhinovirus 68; Human rhinovirus 7; Human rhinovirus 71; Human rhinovirus 73; Human rhinovirus 74; Human rhinovirus 75; Human rhinovirus 76; Human rhinovirus 77; Human rhinovirus 78; Human rhinovirus 8; Human rhinovirus 80; Human rhinovirus 81; Human rhinovirus 82; Human rhinovirus 85; Human rhinovirus 88; Human rhinovirus 89; Human rhinovirus 9; Human rhinovirus 90; Human rhinovirus 94; Human rhinovirus 95; Human rhinovirus 96 Human rhinovirus 98; Human rhinovirus 14; Human rhinovirus 17; Human rhinovirus 26; Human rhinovirus 27; Human rhinovirus 3; Human rhinovirus 8001 Finland Nov1995; Human rhinovirus 35; Human rhinovirus 37; +Human rhinovirus 6253 Finland Sep1994; Human rhinovirus 9166 Finland Sep1995; Human rhinovirus 4; Human rhinovirus 42; Human rhinovirus 48; Human rhinovirus 9864 Finland Sep1996; Human rhinovirus 5; Human rhinovirus 52; Human rhinovirus 6; Human rhinovirus 7425 Finland Dec1995; Human rhinovirus 69; Human rhinovirus 5928 Finland May1995; Human rhinovirus 70; Human rhinovirus 72; Human rhinovirus 79; Human rhinovirus 83; Human rhinovirus 84; Human rhinovirus 8317 Finland Aug1996; Human rhinovirus 86; Human rhinovirus 91; Human rhinovirus 7851 Finland Sep1996; Human rhinovirus 92; Human rhinovirus 93; Human rhinovirus 97; Human rhinovirus 99; Antwerp rhinovirus 98/99; Human rhinovirus 263 Berlin 2004; Human rhinovirus 3083/rhino/Hyogo/2005; Human rhinovirus NY-003; Human rhinovirus NY-028; Human rhinovirus NY-041; Human rhinovirus NY-042; Human rhinovirus NY-060; Human rhinovirus NY-063; Human rhinovirus NY-074; Human rhinovirus NY-1085; Human rhinovirus strain Hanks; Untyped human rhinovirus OK88-8162; Human enterovirus sp. ex Amblyomma americanum; Human rhinovirus sp. or Human rhinovirus UC.

In other embodiments, the attenuated influenza virus is derived from influenza virus A, influenza virus B, or influenza virus C. In further embodiments, the influenza virus A belongs to but is not limited to subtype H10N7, H10N1, H10N2, H10N3, H10N4, H10N5, H10N6, H10N7, H10N8, H10N9, H11N1, H11N2, H11N3, H11N4, H11N6, H11N8, H11N9, H12N1, H12N2, H12N4, H12N5, H12N6, H12N8, H12N9, H13N2, H13N3, H13N6, H13N9, H14N5, H14N6, H15N2, H15N8, H15N9, H16N3, H1N1, H1N2, H1N3, H1N5, H1N6, H1N8, H1N9, H2N1, H2N2, H2N3, H2N4, H2N5, H2N6, H2N7, H2N8, H2N9, H3N1, H3N2, H3N3, H3N4, H3N5, H3N6, H3N8, H3N9, H4N1, H4N2, H4N3, H4N4, H4N5, H4N6, H4N7, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N2, H8N4, H8N5, H9N1, H9N2, H9N3, H9N4, H9N5, H9N6, H9N7, H9N8, H9N9 and unidentified subtypes.

In further embodiments, the influenza virus B belongs to but is not limited to subtype Influenza B virus (B/Aichi/186/2005), Influenza B virus (B/Aichi/5/88), Influenza B virus (B/Akita/27/2001), Influenza B virus (B/Akita/5/2001), Influenza B virus (B/Alabama/1/2006), Influenza B virus (B/Alabama/2/2005), Influenza B virus (B/Alaska/03/1992), Influenza B virus (B/Alaska/12/1996), Influenza B virus (B/Alaska/16/2000), Influenza B virus (B/Alaska/16/2003), Influenza B virus (B/Alaska/1777/2005), Influenza B virus (B/Alaska/2/2004), Influenza B virus (B/Alaska/6/2005), Influenza B virus (B/Ann Arbor/1/1986), Influenza B virus (B/Ann Arbor/1994), Influenza B virus (B/Argentina/132/2001), Influenza B virus (B/Argentina/3640/1999), Influenza B virus (B/Argentina/69/2001), Influenza B virus (B/Arizona/1/2005), Influenza B virus (B/Arizona/12/2003), Influenza B virus (B/Arizona/13/2003), Influenza B virus (B/Arizona/135/2005), Influenza B virus (B/Arizona/14/2001), Influenza B virus (B/Arizona/14/2005), Influenza B virus (B/Arizona/140/2005), Influenza B virus (B/Arizona/146/2005), Influenza B virus (B/Arizona/148/2005), Influenza B virus (B/Arizona/15/2005), Influenza B virus (B/Arizona/16/2005), Influenza B virus (B/Arizona/162/2005), Influenza B virus (B/Arizona/163/2005), Influenza B virus (B/Arizona/164/2005), Influenza B virus (B/Arizona/2/2000), Influenza B virus (B/Arizona/2/2005), Influenza B virus (B/Arizona/2e/2006), Influenza B virus (B/Arizona/3/2006), Influenza B virus (B/Arizona/4/2002), Influenza B virus (B/Arizona/4/2006), Influenza B virus (B/Arizona/48/2005), Influenza B virus (B/Arizona/5/2000), Influenza B virus (B/Arizona/59/2005), Influenza B virus (B/Arizona/7/2000), Influenza B virus (B/Auckland/01/2000), Influenza B virus (B/Bangkok/141/1994), Influenza B virus (B/Bangkok/143/1994), Influenza B virus (B/Bangkok/153/1990), Influenza B virus (B/Bangkok/163/1990), Influenza B virus (B/Bangkok/163/90), Influenza B virus (B/Bangkok/34/99), Influenza B virus (B/Bangkok/460/03), Influenza B virus (B/Bangkok/54/99), Influenza B virus (B/Barcelona/215/03), Influenza B virus (B/Beijing/15/84), Influenza B virus (B/Beijing/184/93), Influenza B virus (B/Beijing/243/97), Influenza B virus (B/Beijing/43/75), Influenza B virus (B/Beijing/5/76), Influenza B virus (B/Beijing/76/98), Influenza B virus (B/Belgium/WV106/2002), Influenza B virus (B/Belgium/WV107/2002), Influenza B virus (B/Belgium/WV109/2002), Influenza B virus (B/Belgium/WV114/2002), Influenza B virus (B/Belgium/WV122/2002), Influenza B virus (B/Bonn/43), Influenza B virus (B/Brazil/017/00), Influenza B virus (B/Brazil/053/00), Influenza B virus (B/Brazil/055/00), Influenza B virus (B/Brazil/064/00), Influenza B virus (B/Brazil/074/00), Influenza B virus (B/Brazil/079/00), Influenza B virus (B/Brazil/110/01), Influenza B virus (B/Brazil/952/2001), Influenza B virus (B/Brazil/975/2000), Influenza B virus (B/Brisbane/32/2002), Influenza B virus (B/Bucharest/311/1998), Influenza B virus (B/Bucharest/795/03), Influenza B virus (B/Buenos Aires/161/00), Influenza B virus (B/Buenos Aires/9/95), Influenza B virus (B/Buenos Aires/SW16/97), Influenza B virus (B/Buenos Aires/VL518/99), Influenza B virus (B/California/01/1995), Influenza B virus (B/California/02/1994), Influenza B virus (B/California/02/1995), Influenza B virus (B/California/1/2000), Influenza B virus (B/California/10/2000), Influenza B virus (B/California/11/2001), Influenza B virus (B/California/14/2005), Influenza B virus (B/California/2/2002), Influenza B virus (B/California/2/2003), Influenza B virus (B/California/3/2000), Influenza B virus (B/California/3/2004), Influenza B virus (B/California/6/2000), Influenza B virus (B/California/7/2005), Influenza B virus (B/Canada/16188/2000), Influenza B virus (B/Canada/464/2001), Influenza B virus (B/Canada/464/2002), Influenza B virus (B/Chaco/366/00), Influenza B virus (B/Chaco/R113/00), Influenza B virus (B/Chantaburi/218/2003), Influenza B virus (B/Cheju/303/03), Influenza B virus (B/Chiba/447/98), Influenza B virus (B/Chile/3162/2002), Influenza B virus (B/Chongqing/3/2000), Influenza B virus (B/clinical isolate SA1 Thailand/2002), Influenza B virus (B/clinical isolate SA10 Thailand/2002), Influenza B virus (B/clinical isolate SA100 Philippines/2002), Influenza B virus (B/clinical isolate SA101 Philippines/2002), Influenza B virus (B/clinical isolate SA102 Philippines/2002), Influenza B virus (B/clinical isolate SA103 Philippines/2002), Influenza B virus (B/clinical isolate SA104 Philippines/2002), Influenza B virus (B/clinical isolate SA105 Philippines/2002), Influenza B virus (B/clinical isolate SA106 Philippines/2002), Influenza B virus (B/clinical isolate SA107 Philippines/2002), Influenza B virus (B/clinical isolate SA108 Philippines/2002), Influenza B virus (B/clinical isolate SA109 Philippines/2002), Influenza B virus (B/clinical isolate SA11 Thailand/2002), Influenza B virus (B/clinical isolate SA110 Philippines/2002), Influenza B virus (B/clinical isolate SA112 Philippines/2002), Influenza B virus (B/clinical isolate SA113 Philippines/2002), Influenza B virus (B/clinical isolate SA114 Philippines/2002), Influenza B virus (B/clinical isolate SA115 Philippines/2002), Influenza B virus (B/clinical isolate SA116 Philippines/2002), Influenza B virus (B/clinical isolate SA12 Thailand/2002), Influenza B virus (B/clinical isolate SA13 Thailand/2002), Influenza B virus (B/clinical isolate SA14 Thailand/2002), Influenza B virus (B/clinical isolate SA15 Thailand/2002), Influenza B virus (B/clinical isolate SA16 Thailand/2002), Influenza B virus (B/clinical isolate SA17 Thailand/2002), Influenza B virus (B/clinical isolate SA18 Thailand/2002), Influenza B virus (B/clinical isolate SA19 Thailand/2002), Influenza B virus (B/clinical isolate SA2 Thailand/2002), Influenza B virus (B/clinical isolate SA20 Thailand/2002), Influenza B virus (B/clinical isolate SA21 Thailand/2002), Influenza B virus (B/clinical isolate SA22 Thailand/2002), Influenza B virus (B/clinical isolate SA23 Thailand/2002), Influenza B virus (B/clinical isolate SA24 Thailand/2002), Influenza B virus (B/clinical isolate SA25 Thailand/2002), Influenza B virus (B/clinical isolate SA26 Thailand/2002), Influenza B virus (B/clinical isolate SA27 Thailand/2002), Influenza B virus (B/clinical isolate SA28 Thailand/2002), Influenza B virus (B/clinical isolate SA29 Thailand/2002), Influenza B virus (B/clinical isolate SA3 Thailand/2002), Influenza B virus (B/clinical isolate SA30 Thailand/2002), Influenza B virus (B/clinical isolate SA31 Thailand/2002), Influenza B virus (B/clinical isolate SA32 Thailand/2002), Influenza B virus (B/clinical isolate SA33 Thailand/2002), Influenza B virus (B/clinical isolate SA34 Thailand/2002), Influenza B virus (B/clinical isolate SA37 Thailand/2002), Influenza B virus (B/clinical isolate SA38 Philippines/2002), Influenza B virus (B/clinical isolate SA39 Thailand/2002), Influenza B virus (B/clinical isolate SA40 Thailand/2002), Influenza B virus (B/clinical isolate SA41 Philippines/2002), Influenza B virus (B/clinical isolate SA42 Philippines/2002), Influenza B virus (B/clinical isolate SA43 Thailand/2002), Influenza B virus (B/clinical isolate SA44 Thailand/2002), Influenza B virus (B/clinical isolate SA45 Philippines/2002), Influenza B virus (B/clinical isolate SA46 Philippines/2002), Influenza B virus (B/clinical isolate SA47 Philippines/2002), Influenza B virus (B/clinical isolate SA5 Thailand/2002), Influenza B virus (B/clinical isolate SA50 Philippines/2002), Influenza B virus (B/clinical isolate SA51 Philippines/2002), Influenza B virus (B/clinical isolate SA52 Philippines/2002), Influenza B virus (B/clinical isolate SA53 Philippines/2002), Influenza B virus (B/clinical isolate SA57 Philippines/2002), Influenza B virus (B/clinical isolate SA58 Philippines/2002), Influenza B virus (B/clinical isolate SA59 Philippines/2002), Influenza B virus (B/clinical isolate SA6 Thailand/2002), Influenza B virus (B/clinical isolate SA60 Philippines/2002), Influenza B virus (B/clinical isolate SA61 Philippines/2002), Influenza B virus (B/clinical isolate SA62 Philippines/2002), Influenza B virus (B/clinical isolate SA63 Philippines/2002), Influenza B virus (B/clinical isolate SA64 Philippines/2002), Influenza B virus (B/clinical isolate SA65 Philippines/2002), Influenza B virus (B/clinical isolate SA66 Philippines/2002), Influenza B virus (B/clinical isolate SA67 Philippines/2002), Influenza B virus (B/clinical isolate SA68 Philippines/2002), Influenza B virus (B/clinical isolate SA69 Philippines/2002), Influenza B virus (B/clinical isolate SA7 Thailand/2002), Influenza B virus (B/clinical isolate SA70 Philippines/2002), Influenza B virus (B/clinical isolate SA71 Philippines/2002), Influenza B virus (B/clinical isolate SA73 Philippines/2002), Influenza B virus (B/clinical isolate SA74 Philippines/2002), Influenza B virus (B/clinical isolate SA76 Philippines/2002), Influenza B virus (B/clinical isolate SA77 Philippines/2002), Influenza B virus (B/clinical isolate SA78 Philippines/2002), Influenza B virus (B/clinical isolate SA79 Philippines/2002), Influenza B virus (B/clinical isolate SA8 Thailand/2002), Influenza B virus (B/clinical isolate SA80 Philippines/2002), Influenza B virus (B/clinical isolate SA81 Philippines/2002), Influenza B virus (B/clinical isolate SA82 Philippines/2002), Influenza B virus (B/clinical isolate SA83 Philippines/2002), Influenza B virus (B/clinical isolate SA84 Philippines/2002), Influenza B virus (B/clinical isolate SA85 Thailand/2002), Influenza B virus (B/clinical isolate SA86 Thailand/2002), Influenza B virus (B/clinical isolate SA87 Thailand/2002), Influenza B virus (B/clinical isolate SA88 Thailand/2002), Influenza B virus (B/clinical isolate SA89 Thailand/2002), Influenza B virus (B/clinical isolate SA9 Thailand/2002), Influenza B virus (B/clinical isolate SA90 Thailand/2002), Influenza B virus (B/clinical isolate SA91 Thailand/2002), Influenza B virus (B/clinical isolate SA92 Thailand/2002), Influenza B virus (B/clinical isolate SA93 Thailand/2002), Influenza B virus (B/clinical isolate SA94 Thailand/2002), Influenza B virus (B/clinical isolate SA95 Philippines/2002), Influenza B virus (B/clinical isolate SA96 Thailand/2002), Influenza B virus (B/clinical isolate SA97 Philippines/2002), Influenza B virus (B/clinical isolate SA98 Philippines/2002), Influenza B virus (B/clinical isolate SA99 Philippines/2002), Influenza B virus (B/CNIC/27/2001), Influenza B virus (B/Colorado/04/2004), Influenza B virus (B/Colorado/11e/2004), Influenza B virus (B/Colorado/12e/2005), Influenza B virus (B/Colorado/13/2004), Influenza B virus (B/Colorado/13e/2004), Influenza B virus (B/Colorado/15/2004), Influenza B virus (B/Colorado/16e/2004), Influenza B virus (B/Colorado/17e/2004), Influenza B virus (B/Colorado/2/2004), Influenza B virus (B/Colorado/2597/2004), Influenza B virus (B/Colorado/4e/2004), Influenza B virus (B/Colorado/5/2004), Influenza B virus (B/Connecticut/02/1995), Influenza B virus (B/Connecticut/07/1993), Influenza B virus (B/Cordoba/2979/1991), Influenza B virus (B/Cordoba/VA418/99), Influenza B virus (B/Czechoslovakia/16/89), Influenza B virus (B/Czechoslovakia/69/1990), Influenza B virus (B/Czechoslovakia/69/90), Influenza B virus (B/Daeku/10/97), Influenza B virus (B/Daeku/45/97), Influenza B virus (B/Daeku/47/97), Influenza B virus (B/Daeku/9/97), Influenza B virus (B/Delaware/1/2006), Influenza B virus (B/Du/4/78), Influenza B virus (B/Durban/39/98), Influenza B virus (B/Durban/43/98), Influenza B virus (B/Durban/44/98), Influenza B virus (B/Durban/52/98), Influenza B virus (B/Durban/55/98), Influenza B virus (B/Durban/56/98), Influenza B virus (B/Egypt/2040/2004), Influenza B virus (B/England/1716/2005), Influenza B virus (B/England/2054/2005), Influenza B virus (B/England/23/04), Influenza B virus (B/EspiritoSanto/55/01), Influenza B virus (B/EspiritoSanto/79/99), Influenza B virus (B/Finland/154/2002), Influenza B virus (B/Finland/159/2002), Influenza B virus (B/Finland/160/2002), Influenza B virus (B/Finland/161/2002), Influenza B virus (B/Finland/162/03), Influenza B virus (B/Finland/162/2002), Influenza B virus (B/Finland/162/91), Influenza B virus (B/Finland/164/2003), Influenza B virus (B/Finland/172/91), Influenza B virus (B/Finland/173/2003), Influenza B virus (B/Finland/176/2003), Influenza B virus (B/Finland/184/91), Influenza B virus (B/Finland/188/2003), Influenza B virus (B/Finland/190/2003), Influenza B virus (B/Finland/191/2003), Influenza B virus (B/Finland/192/2003), Influenza B virus (B/Finland/193/2003), Influenza B virus (B/Finland/199/2003), Influenza B virus (B/Finland/202/2003), Influenza B virus (B/Finland/203/2003), Influenza B virus (B/Finland/204/2003), Influenza B virus (B/Finland/205/2003), Influenza B virus (B/Finland/206/2003), Influenza B virus (B/Finland/220/2003), Influenza B virus (B/Finland/223/2003), Influenza B virus (B/Finland/225/2003), Influenza B virus (B/Finland/227/2003), Influenza B virus (B/Finland/231/2003), Influenza B virus (B/Finland/235/2003), Influenza B virus (B/Finland/239/2003), Influenza B virus (B/Finland/244/2003), Influenza B virus (B/Finland/245/2003), Influenza B virus (B/Finland/254/2003), Influenza B virus (B/Finland/254/93), Influenza B virus (B/Finland/255/2003), Influenza B virus (B/Finland/260/93), Influenza B virus (B/Finland/268/93), Influenza B virus (B/Finland/270/2003), Influenza B virus (B/Finland/275/2003), Influenza B virus (B/Finland/767/2000), Influenza B virus (B/Finland/84/2002), Influenza B virus (B/Finland/886/2001), Influenza B virus (B/Finland/WV4/2002), Influenza B virus (B/Finland/WV5/2002), Influenza B virus (B/Florida/02/1998), Influenza B virus (B/Florida/02/2006), Influenza B virus (B/Florida/1/2000), Influenza B virus (B/Florida/1/2004), Influenza B virus (B/Florida/2/2004), Influenza B virus (B/Florida/2/2005), Influenza B virus (B/Florida/2/2006), Influenza B virus (B/Florida/7e/2004), Influenza B virus (B/Fujian/36/82), Influenza B virus (B/Geneva/5079/03), Influenza B virus (B/Genoa/11/02), Influenza B virus (B/Genoa/2/02), Influenza B virus (B/Genoa/21/02), Influenza B virus (B/Genoa/33/02), Influenza B virus (B/Genoa/41/02), Influenza B virus (B/Genoa/52/02), Influenza B virus (B/Genoa/55/02), Influenza B virus (B/Genoa/56/02), Influenza B virus (B/Genoa/7/02), Influenza B virus (B/Genoa/8/02), Influenza B virus (B/Genoa12/02), Influenza B virus (B/Genoa3/02), Influenza B virus (B/Genoa48/02), Influenza B virus (B/Genoa49/02), Influenza B virus (B/Genoa5/02), Influenza B virus (B/Genoa53/02), Influenza B virus (B/Genoa6/02), Influenza B virus (B/Genoa65/02), Influenza B virus (B/Genova/1294/03), Influenza B virus (B/Genova/1603/03), Influenza B virus (B/Genova/2/02), Influenza B virus (B/Genova/20/02), Influenza B virus (B/Genova/2059/03), Influenza B virus (B/Genova/26/02), Influenza B virus (B/Genova/30/02), Influenza B virus (B/Genova/54/02), Influenza B virus (B/Genova/55/02), Influenza B virus (B/Georgia/02/1998), Influenza B virus (B/Georgia/04/1998), Influenza B virus (B/Georgia/09/2005), Influenza B virus (B/Georgia/1/2000), Influenza B virus (B/Georgia/1/2005), Influenza B virus (B/Georgia/2/2005), Influenza B virus (B/Georgia/9/2005), Influenza B virus (B/Guangdong/05/94), Influenza B virus (B/Guangdong/08/93), Influenza B virus (B/Guangdong/5/94), Influenza B virus (B/Guangdong/55/89), Influenza B virus (B/Guangdong/8/93), Influenza B virus (B/Guangzhou/7/97), Influenza B virus (B/Guangzhou/86/92), Influenza B virus (B/Guangzhou/87/92), Influenza B virus (B/Gyeonggi/592/2005), Influenza B virus (B/Hannover/2/90), Influenza B virus (B/Harbin/07/94), Influenza B virus (B/Hawaii/1/2003), Influenza B virus (B/Hawaii/10/2001), Influenza B virus (B/Hawaii/10/2004), Influenza B virus (B/Hawaii/11/2004), Influenza B virus (B/Hawaii/11e/2004), Influenza B virus (B/Hawaii/Ile/2005), Influenza B virus (B/Hawaii/12e/2005), Influenza B virus (B/Hawaii/13/2004), Influenza B virus (B/Hawaii/13e/2004), Influenza B virus (B/Hawaii/17/2001), Influenza B virus (B/Hawaii/18e/2004), Influenza B virus (B/Hawaii/1990/2004), Influenza B virus (B/Hawaii/1993/2004), Influenza B virus (B/Hawaii/19e/2004), Influenza B virus (B/Hawaii/2/2000), Influenza B virus (B/Hawaii/2/2003), Influenza B virus (B/Hawaii/20e/2004), Influenza B virus (B/Hawaii/21/2004), Influenza B virus (B/Hawaii/26/2001), Influenza B virus (B/Hawaii/31e/2004), Influenza B virus (B/Hawaii/32e/2004), Influenza B virus (B/Hawaii/33e/2004), Influenza B virus (B/Hawaii/35/2001), Influenza B virus (B/Hawaii/36/2001), Influenza B virus (B/Hawaii/37/2001), Influenza B virus (B/Hawaii/38/2001), Influenza B virus (B/Hawaii/4/2006), Influenza B virus (B/Hawaii/43/2001), Influenza B virus (B/Hawaii/44/2001), Influenza B virus (B/Hawaii/9/2001), Influenza B virus (B/Hebei/19/94), Influenza B virus (B/Hebei/3/94), Influenza B virus (B/Hebei/4/95), Influenza B virus (B/Henan/22/97), Influenza B virus (B/Hiroshima/23/2001), Influenza B virus (B/Hong Kong/02/1993), Influenza B virus (B/Hong Kong/03/1992), Influenza B virus (B/Hong Kong/05/1972), Influenza B virus (B/Hong Kong/06/2001), Influenza B virus (B/Hong Kong/110/99), Influenza B virus (B/Hong Kong/1115/2002), Influenza B virus (B/Hong Kong/112/2001), Influenza B virus (B/Hong Kong/123/2001), Influenza B virus (B/Hong Kong/1351/02), Influenza B virus (B/Hong Kong/1351/2002), Influenza B virus (B/Hong Kong/1434/2002), Influenza B virus (B/Hong Kong/147/99), Influenza B virus (B/Hong Kong/156/99), Influenza B virus (B/Hong Kong/157/99), Influenza B virus (B/Hong Kong/167/2002), Influenza B virus (B/Hong Kong/22/1989), Influenza B virus (B/Hong Kong/22/2001), Influenza B virus (B/Hong Kong/22/89), Influenza B virus (B/Hong Kong/28/2001), Influenza B virus (B/Hong Kong/293/02), Influenza B virus (B/Hong Kong/310/2004), Influenza B virus (B/Hong Kong/329/2001), Influenza B virus (B/Hong Kong/330/2001 egg adapted), Influenza B virus (B/Hong Kong/330/2001), Influenza B virus (B/Hong Kong/330/2002), Influenza B virus (B/Hong Kong/335/2001), Influenza B virus (B/Hong Kong/336/2001), Influenza B virus (B/Hong Kong/497/2001), Influenza B virus (B/Hong Kong/542/2000), Influenza B virus (B/Hong Kong/548/2000), Influenza B virus (B/Hong Kong/553a/2003), Influenza B virus (B/Hong Kong/557/2000), Influenza B virus (B/Hong Kong/6/2001), Influenza B virus (B/Hong Kong/666/2001), Influenza B virus (B/Hong Kong/692/01), Influenza B virus (B/Hong Kong/70/1996), Influenza B virus (B/Hong Kong/8/1973), Influenza B virus (B/Hong Kong/9/89), Influenza B virus (B/Houston/1/91), Influenza B virus (B/Houston/1/92), Influenza B virus (B/Houston/1/96), Influenza B virus (B/Houston/2/93), Influenza B virus (B/Houston/2/96), Influenza B virus (B/Houston/B15/1999), Influenza B virus (B/Houston/B56/1997), Influenza B virus (B/Houston/B57/1997), Influenza B virus (B/Houston/B58/1997), Influenza B virus (B/Houston/B59/1997), Influenza B virus (B/Houston/B60/1997), Influenza B virus (B/Houston/B61/1997), Influenza B virus (B/Houston/B63/1997), Influenza B virus (B/Houston/B65/1998), Influenza B virus (B/Houston/B66/2000), Influenza B virus (B/Houston/B67/2000), Influenza B virus (B/Houston/B68/2000), Influenza B virus (B/Houston/B69/2002), Influenza B virus (B/Houston/B70/2002), Influenza B virus (B/Houston/B71/2002), Influenza B virus (B/Houston/B720/2004), Influenza B virus (B/Houston/B74/2002), Influenza B virus (B/Houston/B745/2005), Influenza B virus (B/Houston/B75/2002), Influenza B virus (B/Houston/B756/2005), Influenza B virus (B/Houston/B77/2002), Influenza B virus (B/Houston/B787/2005), Influenza B virus (B/Houston/B79/2003), Influenza B virus (B/Houston/B81/2003), Influenza B virus (B/Houston/B84/2003), Influenza B virus (B/Houston/B846/2005), Influenza B virus (B/Houston/B850/2005), Influenza B virus (B/Houston/B86/2003), Influenza B virus (B/Houston/B87/2003), Influenza B virus (B/Houston/B88/2003), Influenza B virus (B/Hunan/4/72), Influenza B virus (B/Ibaraki/2/85), Influenza B virus (B/Idaho/1/2005), Influenza B virus (B/Illinois/1/2004), Influenza B virus (B/Illinois/13/2004), Influenza B virus (B/Illinois/13/2005), Influenza B virus (B/Illinois/13e/2005), Influenza B virus (B/Illinois/3/2001), Influenza B virus (B/Illinois/3/2005), Influenza B virus (B/Illinois/33/2005), Influenza B virus (B/Illinois/36/2005), Influenza B virus (B/Illinois/4/2005), Influenza B virus (B/Illinois/47/2005), Influenza B virus (B/Incheon/297/2005), Influenza B virus (B/India/3/89), Influenza B virus (B/India/7526/2001), Influenza B virus (B/India/7569/2001), Influenza B virus (B/India/7600/2001), Influenza B virus (B/India/7605/2001), Influenza B virus (B/India/77276/2001), Influenza B virus (B/Indiana/01/1995), Influenza B virus (B/Indiana/3/2006), Influenza B virus (B/Indiana/5/2006), Influenza B virus (B/Iowa/03/2002), Influenza B virus (B/Iowa/1/2001), Influenza B virus (B/Iowa/1/2005), Influenza B virus (B/Israel/95/03), Influenza B virus (B/Israel/WV124/2002), Influenza B virus (B/Israel/WV126/2002), Influenza B virus (B/Israel/WV133/2002), Influenza B virus (B/Israel/WV135/2002), Influenza B virus (B/Israel/WV137/2002), Influenza B virus (B/Israel/WV142/2002), Influenza B virus (B/Israel/WV143/2002), Influenza B virus (B/Israel/WV145/2002), Influenza B virus (B/Israel/WV146/2002), Influenza B virus (B/Israel/WV150/2002), Influenza B virus (B/Israel/WV153/2002), Influenza B virus (B/Israel/WV158/2002), Influenza B virus (B/Israel/WV161/2002), Influenza B virus (B/Israel/WV166/2002), Influenza B virus (B/Israel/WV169/2002), Influenza B virus (B/Israel/WV170/2002), Influenza B virus (B/Israel/WV174/2002), Influenza B virus (B/Israel/WV183/2002), Influenza B virus (B/Israel/WV187/2002), Influenza B virus (B/Istanbul/CTF-132/05), Influenza B virus (B/Japan/1224/2005), Influenza B virus (B/Japan/1905/2005), Influenza B virus (B/Jiangsu/10/03), Influenza B virus (B/Jiangsu/10/2003 (recomb)), Influenza B virus (B/Jiangsu/10/2003), Influenza B virus (B/Jilin/20/2003), Influenza B virus (B/Johannesburg/05/1999), Influenza B virus (B/Johannesburg/06/1994), Influenza B virus (B/Johannesburg/1/99), Influenza B virus (B/Johannesburg/113/010), Influenza B virus (B/Johannesburg/116/01), Influenza B virus (B/Johannesburg/119/01), Influenza B virus (B/Johannesburg/123/01), Influenza B virus (B/Johannesburg/163/99), Influenza B virus (B/Johannesburg/187/99), Influenza B virus (B/Johannesburg/189/99), Influenza B virus (B/Johannesburg/2/99), Influenza B virus (B/Johannesburg/27/2005), Influenza B virus (B/Johannesburg/33/01), Influenza B virus (B/Johannesburg/34/01), Influenza B virus (B/Johannesburg/35/01), Influenza B virus (B/Johannesburg/36/01), Influenza B virus (B/Johannesburg/41/99), Influenza B virus (B/Johannesburg/5/99), Influenza B virus (B/Johannesburg/69/2001), Influenza B virus (B/Johannesburg/77/01), Influenza B virus (B/Johannesburg/94/99), Influenza B virus (B/Johannesburg/96/01), Influenza B virus (B/Kadoma/1076/99), Influenza B virus (B/Kadoma/122/99), Influenza B virus (B/Kadoma/122/99-V1), Influenza B virus (B/Kadoma/122/99-V10), Influenza B virus (B/Kadoma/122/99-V11), Influenza B virus (B/Kadoma/122/99-V2), Influenza B virus (B/Kadoma/122/99-V3), Influenza B virus (B/Kadoma/122/99-V4), Influenza B virus (B/Kadoma/122/99-V5), Influenza B virus (B/Kadoma/122/99-V6), Influenza B virus (B/Kadoma/122/99-V7), Influenza B virus (B/Kadoma/122/99-V8), Influenza B virus (B/Kadoma/122/99-V9), Influenza B virus (B/Kadoma/136/99), Influenza B virus (B/Kadoma/409/2000), Influenza B virus (B/Kadoma/506/99), Influenza B virus (B/kadoma/642/99), Influenza B virus (B/Kadoma/647/99), Influenza B virus (B/Kagoshima/15/94), Influenza B virus (B/Kanagawa/73), Influenza B virus (B/Kansas/1/2005), Influenza B virus (B/Kansas/22992/99), Influenza B virus (B/Kentucky/4/2005), Influenza B virus (B/Khazkov/224/91), Influenza B virus (B/Kisumu/2036/2006), Influenza B virus (B/Kisumu/2037/2006), Influenza B virus (B/Kisumu/2038/2006), Influenza B virus (B/Kisumu/2039/2006), Influenza B virus (B/Kisumu/2040/2006), Influenza B virus (B/Kisumu/7/2005), Influenza B virus (B/Kobe/1/2002), Influenza B virus (B/Kobe/1/2002-V1), Influenza B virus (B/Kobe/1/2002-V2), Influenza B virus (B/Kobe/1/2003), Influenza B virus (B/Kobe/1/94), Influenza B virus (B/Kobe/2/2002), Influenza B virus (B/Kobe/2/2003), Influenza B virus (B/Kobe/25/2003), Influenza B virus (B/Kobe/26/2003), Influenza B virus (B/Kobe/28/2003), Influenza B virus (B/Kobe/3/2002), Influenza B virus (B/Kobe/3/2003), Influenza B virus (B/Kobe/4/2002), Influenza B virus (B/Kobe/4/2003), Influenza B virus (B/Kobe/5/2002), Influenza B virus (B/Kobe/6/2002), Influenza B virus (B/Kobe/64/2001), Influenza B virus (B/Kobe/65/2001), Influenza B virus (B/Kobe/69/2001), Influenza B virus (B/Kobe/7/2002), Influenza B virus (B/Kobe/79/2001), Influenza B virus (B/Kobe/83/2001), Influenza B virus (B/Kobe/87/2001), Influenza B virus (B/Kouchi/193/1999), Influenza B virus (B/Kouchi/193/99), Influenza B virus (B/Lazio/1/02), Influenza B virus (B/Lee/40), Influenza B virus (B/Leningrad/129/91), Influenza B virus (B/Leningrad/148/91), Influenza B virus (B/Lisbon/02/1994), Influenza B virus (B/Lissabon/2/90), Influenza B virus (B/Los Angeles/1/02), Influenza B virus (B/Lusaka/270/99), Influenza B virus (B/Lusaka/432/99), Influenza B virus (B/Lyon/1271/96), Influenza B virus (B/Malaysia/83077/2001), Influenza B virus (B/Maputo/1/99), Influenza B virus (B/Maputo/2/99), Influenza B virus (B/Mar del Plata/595/99), Influenza B virus (B/Mar del Plata/VL373/99), Influenza B virus (B/Mar del Plata/VL385/99), Influenza B virus (B/Maryland/i/01), Influenza B virus (B/Maryland/1/2002), Influenza B virus (B/Maryland/2/2001), Influenza B virus (B/Maryland/7/2003), Influenza B virus (B/Massachusetts/1/2004), Influenza B virus (B/Massachusetts/2/2004), Influenza B virus (B/Massachusetts/3/2004), Influenza B virus (B/Massachusetts/4/2001), Influenza B virus (B/Massachusetts/5/2003), Influenza B virus (B/Memphis/i/01), Influenza B virus (B/Memphis/10/97), Influenza B virus (B/Memphis/11/2006), Influenza B virus (B/Memphis/12/2006), Influenza B virus (B/Memphis/12/97), Influenza B virus (B/Memphis/12/97-MA), Influenza B virus (B/Memphis/13/03), Influenza B virus (B/Memphis/18/95), Influenza B virus (B/Memphis/19/96), Influenza B virus (B/Memphis/20/96), Influenza B virus (B/Memphis/21/96), Influenza B virus (B/Memphis/28/96), Influenza B virus (B/Memphis/3/01), Influenza B virus (B/Memphis/3/89), Influenza B virus (B/Memphis/3/93), Influenza B virus (B/Memphis/4/93), Influenza B virus (B/Memphis/5/93), Influenza B virus (B/Memphis/7/03), Influenza B virus (B/Memphis/8/99), Influenza B virus (B/Mexico/84/2000), Influenza B virus (B/Michigan/04/2006), Influenza B virus (B/Michigan/i/2005), Influenza B virus (B/Michigan/i/2006), Influenza B virus (B/Michigan/2/2004), Influenza B virus (B/Michigan/20/2005), Influenza B virus (B/Michigan/22572/99), Influenza B virus (B/Michigan/22587/99), Influenza B virus (B/Michigan/22596/99), Influenza B virus (B/Michigan/22631/99), Influenza B virus (B/Michigan/22659/99), Influenza B virus (B/Michigan/22687/99), Influenza B virus (B/Michigan/22691/99), Influenza B virus (B/Michigan/22721/99), Influenza B virus (B/Michigan/22723/99), Influenza B virus (B/Michigan/2e/2006), Influenza B virus (B/Michigan/3/2004), Influenza B virus (B/Michigan/4/2006), Influenza B virus (B/Michigan/e3/2006), Influenza B virus (B/micona/1/1989), Influenza B virus (B/Mie/01/1993), Influenza B virus (B/Mie/i/93), Influenza B virus (B/Milano/1/01), Influenza B virus (B/Milano/1/02), Influenza B virus (B/Milano/5/02), Influenza B virus (B/Milano/6/02), Influenza B virus (B/Milano/66/04), Influenza B virus (B/Milano/7/02), Influenza B virus (B/Minnesota/1/1985), Influenza B virus (B/Minnesota/14/2001), Influenza B virus (B/Minnesota/2/2001), Influenza B virus (B/Minsk/318/90), Influenza B virus (B/Mississippi/1/2001), Influenza B virus (B/Mississippi/2/2005), Influenza B virus (B/Mississippi/3/2001), Influenza B virus (B/Mississippi/3/2005), Influenza B virus (B/Mississippi/4/2003), Influenza B virus (B/Mississippi/4e/2005), Influenza B virus (B/Missouri/1/2006), Influenza B virus (B/Missouri/11/2003), Influenza B virus (B/Missouri/2/2005), Influenza B virus (B/Missouri/20/2003), Influenza B virus (B/Missouri/6/2005), Influenza B virus (B/Montana/1/2003), Influenza B virus (B/Montana/1/2006), Influenza B virus (B/Montana/1e/2004), Influenza B virus (B/Moscow/16/2002), Influenza B virus (B/Moscow/3/03), Influenza B virus (B/Nagoya/20/99), Influenza B virus (B/Nairobi/2032/2006), Influenza B virus (B/Nairobi/2033/2006), Influenza B virus (B/Nairobi/2034/2006), Influenza B virus (B/Nairobi/2035/2006), Influenza B virus (B/Nairobi/351/2005), Influenza B virus (B/Nairobi/670/2005), Influenza B virus (B/Nanchang/1/00), Influenza B virus (B/Nanchang/1/2000), Influenza B virus (B/Nanchang/12/98), Influenza B virus (B/Nanchang/15/95), Influenza B virus (B/Nanchang/15/97), Influenza B virus (B/Nanchang/195/94), Influenza B virus (B/Nanchang/2/97), Influenza B virus (B/Nanchang/20/96), Influenza B virus (B/Nanchang/26/93), Influenza B virus (B/Nanchang/3/95), Influenza B virus (B/Nanchang/4/97), Influenza B virus (B/Nanchang/480/94), Influenza B virus (B/Nanchang/5/97), Influenza B virus (B/Nanchang/560/94), Influenza B virus (B/Nanchang/560a/94), Influenza B virus (B/Nanchang/560b/94), Influenza B virus (B/Nanchang/6/96), Influenza B virus (B/Nanchang/6/98), Influenza B virus (B/Nanchang/630/94), Influenza B virus (B/Nanchang/7/98), Influenza B virus (B/Nanchang/8/95), Influenza B virus (B/Nashville/107/93), Influenza B virus (B/Nashville/3/96), Influenza B virus (B/Nashville/34/96), Influenza B virus (B/Nashville/45/91), Influenza B virus (B/Nashville/48/91), Influenza B virus (B/Nashville/6/89), Influenza B virus (B/Nebraska/1/01), Influenza B virus (B/Nebraska/1/2005), Influenza B virus (B/Nebraska/2/01), Influenza B virus (B/Nebraska/4/2001), Influenza B virus (B/Nebraska/5/2003), Influenza B virus (B/Nepal/1078/2005), Influenza B virus (B/Nepal/1079/2005), Influenza B virus (B/Nepal/1080/2005), Influenza B virus (B/Nepal/1087/2005), Influenza B virus (B/Nepal/1088/2005), Influenza B virus (B/Nepal/1089/2005), Influenza B virus (B/Nepal/1090/2005), Influenza B virus (B/Nepal/1092/2005), Influenza B virus (B/Nepal/1098/2005), Influenza B virus (B/Nepal/1101/2005), Influenza B virus (B/Nepal/1103/2005), Influenza B virus (B/Nepal/1104/2005), Influenza B virus (B/Nepal/1105/2005), Influenza B virus (B/Nepal/1106/2005), Influenza B virus (B/Nepal/1108/2005), Influenza B virus (B/Nepal/1114/2005), Influenza B virus (B/Nepal/1117/2005), Influenza B virus (B/Nepal/1118/2005), Influenza B virus (B/Nepal/1120/2005), Influenza B virus (B/Nepal/1122/2005), Influenza B virus (B/Nepal/1131/2005), Influenza B virus (B/Nepal/1132/2005), Influenza B virus (B/Nepal/1136/2005), Influenza B virus (B/Nepal/1137/2005), Influenza B virus (B/Nepal/1138/2005), Influenza B virus (B/Nepal/1139/2005), Influenza B virus (B/Nepal/1331/2005), Influenza B virus (B/Netherland/2781/90), Influenza B virus (B/Netherland/6357/90), Influenza B virus (B/Netherland/800/90), Influenza B virus (B/Netherland/801/90), Influenza B virus (B/Netherlands/1/97), Influenza B virus (B/Netherlands/13/94), Influenza B virus (B/Netherlands/2/95), Influenza B virus (B/Netherlands/31/95), Influenza B virus (B/Netherlands/32/94), Influenza B virus (B/Netherlands/384/95), Influenza B virus (B/Netherlands/429/98), Influenza B virus (B/Netherlands/580/89), Influenza B virus (B/Netherlands/6/96), Influenza B virus (B/Nevada/1/2001), Influenza B virus (B/Nevada/1/2002), Influenza B virus (B/Nevada/1/2005), Influenza B virus (B/Nevada/1/2006), Influenza B virus (B/Nevada/2/2003), Influenza B virus (B/Nevada/2/2006), Influenza B virus (B/Nevada/3/2006), Influenza B virus (B/Nevada/5/2005), Influenza B virus (B/New Jersey/1/2002), Influenza B virus (B/New Jersey/1/2004), Influenza B virus (B/New Jersey/1/2005), Influenza B virus (B/New Jersey/1/2006), Influenza B virus (B/New Jersey/3/2001), Influenza B virus (B/New Jersey/3/2005), Influenza B virus (B/New Jersey/4/2001), Influenza B virus (B/New Jersey/5/2005), Influenza B virus (B/New Jersey/6/2005), Influenza B virus (B/New Mexico/1/2001), Influenza B virus (B/New Mexico/1/2006), Influenza B virus (B/New Mexico/2/2005), Influenza B virus (B/New Mexico/9/2003), Influenza B virus (B/New York/1/2001), Influenza B virus (B/New York/1/2002), Influenza B virus (B/New York/1/2004), Influenza B virus (B/New York/1/2006), Influenza B virus (B/New York/10/2002), Influenza B virus (B/New York/11/2005), Influenza B virus (B/New York/12/2001), Influenza B virus (B/New York/12/2005), Influenza B virus (B/New York/12e/2005), Influenza B virus (B/New York/14e/2005), Influenza B virus (B/New York/17/2004), Influenza B virus (B/New York/18/2003), Influenza B virus (B/New York/19/2004), Influenza B virus (B/New York/2/2000), Influenza B virus (B/New York/2/2002), Influenza B virus (B/New York/2/2006), Influenza B virus (B/New York/20139/99), Influenza B virus (B/New York/24/1993), Influenza B virus (B/New York/2e/2005), Influenza B virus (B/New York/3/90), Influenza B virus (B/New York/39/1991), Influenza B virus (B/New York/40/2002), Influenza B virus (B/New York/47/2001), Influenza B virus (B/New York/6/2004), Influenza B virus (B/New York/7/2002), Influenza B virus (B/New York/8/2000), Influenza B virus (B/New York/9/2002), Influenza B virus (B/New York/9/2004), Influenza B virus (B/New York/C10/2004), Influenza B virus (B/NIB/48/90), Influenza B virus (B/Ningxia/45/83), Influenza B virus (B/North Carolina/1/2005), Influenza B virus (B/North Carolina/3/2005), Influenza B virus (B/North Carolina/4/2004), Influenza B virus (B/North Carolina/5/2004), Influenza B virus (B/Norway/1/84), Influenza B virus (B/Ohio/1/2005), Influenza B virus (B/Ohio/1/X-19/2005), Influenza B virus (B/Ohio/1e/2005), Influenza B virus (B/Ohio/1e4/2005), Influenza B virus (B/Ohio/2/2002), Influenza B virus (B/Ohio/2e/2005), Influenza B virus (B/Oita/15/1992), Influenza B virus (B/Oklahoma/1/2006), Influenza B virus (B/Oklahoma/2/2005), Influenza B virus (B/Oman/16291/2001), Influenza B virus (B/Oman/16296/2001), Influenza B virus (B/Oman/16299/2001), Influenza B virus (B/Oman/16305/2001), Influenza B virus (B/Oregon/1/2005), Influenza B virus (B/Oregon/1/2006), Influenza B virus (B/Oregon/5/80), Influenza B virus (B/Osaka/1036/97), Influenza B virus (B/Osaka/1058/97), Influenza B virus (B/Osaka/1059/97), Influenza B virus (B/Osaka/1146/1997), Influenza B virus (B/Osaka/1169/97), Influenza B virus (B/Osaka/1201/2000), Influenza B virus (B/Osaka/547/1997), Influenza B virus (B/Osaka/547/97), Influenza B virus (B/Osaka/710/1997), Influenza B virus (B/Osaka/711/97), Influenza B virus (B/Osaka/728/1997), Influenza B virus (B/Osaka/755/1997), Influenza B virus (B/Osaka/820/1997), Influenza B virus (B/Osaka/837/1997), Influenza B virus (B/Osaka/854/1997), Influenza B virus (B/Osaka/983/1997), Influenza B virus (B/Osaka/983/1997-M1), Influenza B virus (B/Osaka/983/1997-M2), Influenza B virus (B/Osaka/983/97-V1), Influenza B virus (B/Osaka/983/97-V2), Influenza B virus (B/Osaka/983/97-V3), Influenza B virus (B/Osaka/983/97-V4), Influenza B virus (B/Osaka/983/97-V5), Influenza B virus (B/Osaka/983/97-V6), Influenza B virus (B/Osaka/983/97-V7), Influenza B virus (B/Osaka/983/97-V8), Influenza B virus (B/Osaka/c19/93), Influenza B virus (B/Oslo/1072/2001), Influenza B virus (B/Oslo/1329/2002), Influenza B virus (B/Oslo/1510/2002), Influenza B virus (B/Oslo/1846/2002), Influenza B virus (B/Oslo/1847/2002), Influenza B virus (B/Oslo/1862/2001), Influenza B virus (B/Oslo/1864/2001), Influenza B virus (B/Oslo/1870/2002), Influenza B virus (B/Oslo/1871/2002), Influenza B virus (B/Oslo/2293/2001), Influenza B virus (B/Oslo/2295/2001), Influenza B virus (B/Oslo/2297/2001), Influenza B virus (B/Oslo/238/2001), Influenza B virus (B/Oslo/3761/2000), Influenza B virus (B/Oslo/47/2001), Influenza B virus (B/Oslo/668/2002), Influenza B virus (B/Oslo/71/04), Influenza B virus (B/Oslo/801/99), Influenza B virus (B/Oslo/805/99), Influenza B virus (B/Oslo/837/99), Influenza B virus (B/Panama/45/1990), Influenza B virus (B/Panama/45/90), Influenza B virus (B/Paraguay/636/2003), Influenza B virus (B/Paris/329/90), Influenza B virus (B/Paris/549/1999), Influenza B virus (B/Parma/1/03), Influenza B virus (B/Parma/1/04), Influenza B virus (B/Parma/13/02), Influenza B virus (B/Parma/16/02), Influenza B virus (B/Parma/2/03), Influenza B virus (B/Parma/2/04), Influenza B virus (B/Parma/23/02), Influenza B virus (B/Parma/24/02), Influenza B virus (B/Parma/25/02), Influenza B virus (B/Parma/28/02), Influenza B virus (B/Parma/3/04), Influenza B virus (B/Parma/4/04), Influenza B virus (B/Parma/5/02), Influenza B virus (B/Pennsylvania/1/2006), Influenza B virus (B/Pennsylvania/2/2001), Influenza B virus (B/Pennsylvania/2/2006), Influenza B virus (B/Pennsylvania/3/2003), Influenza B virus (B/Pennsylvania/3/2006), Influenza B virus (B/Pennsylvania/4/2004), Influenza B virus (B/Perth/211/2001), Influenza B virus (B/Perth/25/2002), Influenza B virus (B/Peru/1324/2004), Influenza B virus (B/Peru/1364/2004), Influenza B virus (B/Perugia/4/03), Influenza B virus (B/Philippines/5072/2001), Influenza B virus (B/Philippines/93079/2001), Influenza B virus (B/Pusan/250/99), Influenza B virus (B/Pusan/255/99), Influenza B virus (B/Pusan/270/99), Influenza B virus (B/Pusan/285/99), Influenza B virus (B/Quebec/1/01), Influenza B virus (B/Quebec/162/98), Influenza B virus (B/Quebec/173/98), Influenza B virus (B/Quebec/2/01), Influenza B virus (B/Quebec/3/01), Influenza B virus (B/Quebec/4/01), Influenza B virus (B/Quebec/452/98), Influenza B virus (B/Quebec/453/98), Influenza B virus (B/Quebec/465/98), Influenza B virus (B/Quebec/51/98), Influenza B virus (B/Quebec/511/98), Influenza B virus (B/Quebec/514/98), Influenza B virus (B/Quebec/517/98), Influenza B virus (B/Quebec/6/01), Influenza B virus (B/Quebec/7/01), Influenza B virus (B/Quebec/74199/99), Influenza B virus (B/Quebec/74204/99), Influenza B virus (B/Quebec/74206/99), Influenza B virus (B/Quebec/8/01), Influenza B virus (B/Quebec/9/01), Influenza B virus (B/Rabat/41/97), Influenza B virus (B/Rabat/45/97), Influenza B virus (B/Rabat/61/97), Influenza B virus (B/RiodeJaneiro/200/02), Influenza B virus (B/RiodeJaneiro/209/02), Influenza B virus (B/RiodeJaneiro/315/01), Influenza B virus (B/RiodeJaneiro/353/02), Influenza B virus (B/RiodeJaneiro/354/02), Influenza B virus (B/RioGdoSul/337/01), Influenza B virus (B/RioGdoSul/357/02), Influenza B virus (B/RioGdoSul/374/01), Influenza B virus (B/Roma/1/03), Influenza B virus (B/Roma/2/03), Influenza B virus (B/Roma/3/03), Influenza B virus (B/Roma/4/02), Influenza B virus (B/Roma/7/02), Influenza B virus (B/Romania/217/1999), Influenza B virus (B/Romania/318/1998), Influenza B virus (B/Russia/22/1995), Influenza B virus (B/Saga/S172/99), Influenza B virus (B/Seal/Netherlands/1/99), Influenza B virus (B/Seoul/1/89), Influenza B virus (B/Seoul/1163/2004), Influenza B virus (B/Seoul/12/88), Influenza B virus (B/seoul/12/95), Influenza B virus (B/Seoul/13/95), Influenza B virus (B/Seoul/16/97), Influenza B virus (B/Seoul/17/95), Influenza B virus (B/Seoul/19/97), Influenza B virus (B/Seoul/21/95), Influenza B virus (B/Seoul/232/2004), Influenza B virus (B/Seoul/28/97), Influenza B virus (B/Seoul/31/97), Influenza B virus (B/Seoul/37/91), Influenza B virus (B/Seoul/38/91), Influenza B virus (B/Seoul/40/91), Influenza B virus (B/Seoul/41/91), Influenza B virus (B/Seoul/6/88), Influenza B virus (B/Shandong/7/97), Influenza B virus (B/Shangdong/7/97), Influenza B virus (B/Shanghai/1/77), Influenza B virus (B/Shanghai/10/80), Influenza B virus (B/Shanghai/24/76), Influenza B virus (B/Shanghai/35/84), Influenza B virus (B/Shanghai/361/03), Influenza B virus (B/Shanghai/361/2002), Influenza B virus (B/Shenzhen/423/99), Influenza B virus (B/Shiga/51/98), Influenza B virus (B/Shiga/N18/98), Influenza B virus (B/Shiga/T30/98), Influenza B virus (B/Shiga/T37/98), Influenza B virus (B/Shizuoka/15/2001), Influenza B virus (B/Shizuoka/480/2000), Influenza B virus (B/Sichuan/281/96), Influenza B virus (B/Sichuan/317/2001), Influenza B virus (B/Sichuan/379/99), Influenza B virus (B/Sichuan/38/2000), Influenza B virus (B/Sichuan/8/92), Influenza B virus (B/Siena/1/02), Influenza B virus (B/Singapore/04/1991), Influenza B virus (B/Singapore/11/1994), Influenza B virus (B/Singapore/22/1998), Influenza B virus (B/Singapore/222/79), Influenza B virus (B/Singapore/31/1998), Influenza B virus (B/Singapore/35/1998), Influenza B virus (B/South Australia/5/1999), Influenza B virus (B/South Carolina/04/2003), Influenza B virus (B/South Carolina/25723/99), Influenza B virus (B/South Carolina/3/2003), Influenza B virus (B/South Carolina/4/2003), Influenza B virus (B/South Dakota/1/2000), Influenza B virus (B/South Dakota/3/2003), Influenza B virus (B/South Dakota/5/89), Influenza B virus (B/Spain/WV22/2002), Influenza B virus (B/Spain/WV26/2002), Influenza B virus (B/Spain/WV27/2002), Influenza B virus (B/Spain/WV29/2002), Influenza B virus (B/Spain/WV33/2002), Influenza B virus (B/Spain/WV34/2002), Influenza B virus (B/Spain/WV36/2002), Influenza B virus (B/Spain/WV41/2002), Influenza B virus (B/Spain/WV42/2002), Influenza B virus (B/Spain/WV43/2002), Influenza B virus (B/Spain/WV45/2002), Influenza B virus (B/Spain/WV50/2002), Influenza B virus (B/Spain/WV51/2002), Influenza B virus (B/Spain/WV56/2002), Influenza B virus (B/Spain/WV57/2002), Influenza B virus (B/Spain/WV65/2002), Influenza B virus (B/Spain/WV66/2002), Influenza B virus (B/Spain/WV67/2002), Influenza B virus (B/Spain/WV69/2002), Influenza B virus (B/Spain/WV70/2002), Influenza B virus (B/Spain/WV73/2002), Influenza B virus (B/Spain/WV78/2002), Influenza B virus (B/St. Petersburg/14/2006), Influenza B virus (B/StaCatarina/308/02), Influenza B virus (B/StaCatarina/315/02), Influenza B virus (B/StaCatarina/318/02), Influenza B virus (B/StaCatarina/345/02), Influenza B virus (B/Stockholm/10/90), Influenza B virus (B/Suzuka/18/2005), Influenza B virus (B/Suzuka/28/2005), Influenza B virus (B/Suzuka/32/2005), Influenza B virus (B/Suzuka/58/2005), Influenza B virus (B/Switzerland/4291/97), Influenza B virus (B/Switzerland/5219/90), Influenza B virus (B/Switzerland/5241/90), Influenza B virus (B/Switzerland/5441/90), Influenza B virus (B/Switzerland/5444/90), Influenza B virus (B/Switzerland/5812/90), Influenza B virus (B/Switzerland/6121/90), Influenza B virus (B/Taiwan/0002/03), Influenza B virus (B/Taiwan/0114/01), Influenza B virus (B/Taiwan/0202/01), Influenza B virus (B/Taiwan/0409/00), Influenza B virus (B/Taiwan/0409/02), Influenza B virus (B/Taiwan/0562/03), Influenza B virus (B/Taiwan/0569/03), Influenza B virus (B/Taiwan/0576/03), Influenza B virus (B/Taiwan/0600/02), Influenza B virus (B/Taiwan/0610/03), Influenza B virus (B/Taiwan/0615/03), Influenza B virus (B/Taiwan/0616/03), Influenza B virus (B/Taiwan/0654/02), Influenza B virus (B/Taiwan/0684/03), Influenza B virus (B/Taiwan/0699/03), Influenza B virus (B/Taiwan/0702/02), Influenza B virus (B/Taiwan/0722/02), Influenza B virus (B/Taiwan/0730/02), Influenza B virus (B/Taiwan/0735/03), Influenza B virus (B/Taiwan/0833/03), Influenza B virus (B/Taiwan/0874/02), Influenza B virus (B/Taiwan/0879/02), Influenza B virus (B/Taiwan/0880/02), Influenza B virus (B/Taiwan/0927/02), Influenza B virus (B/Taiwan/0932/02), Influenza B virus (B/Taiwan/0993/02), Influenza B virus (B/Taiwan/1013/02), Influenza B virus (B/Taiwan/1013/03), Influenza B virus (B/Taiwan/102/2005), Influenza B virus (B/Taiwan/103/2005), Influenza B virus (B/Taiwan/110/2005), Influenza B virus (B/Taiwan/1103/2001), Influenza B virus (B/Taiwan/114/2001), Influenza B virus (B/Taiwan/11515/2001), Influenza B virus (B/Taiwan/117/2005), Influenza B virus (B/Taiwan/1197/1994), Influenza B virus (B/Taiwan/121/2005), Influenza B virus (B/Taiwan/12192/2000), Influenza B virus (B/Taiwan/1243/99), Influenza B virus (B/Taiwan/1265/2000), Influenza B virus (B/Taiwan/1293/2000), Influenza B virus (B/Taiwan/13/2004), Influenza B virus (B/Taiwan/14/2004), Influenza B virus (B/Taiwan/1484/2001), Influenza B virus (B/Taiwan/1502/02), Influenza B virus (B/Taiwan/1503/02), Influenza B virus (B/Taiwan/1534/02), Influenza B virus (B/Taiwan/1536/02), Influenza B virus (B/Taiwan/1561/02), Influenza B virus (B/Taiwan/1574/03), Influenza B virus (B/Taiwan/1584/02), Influenza B virus (B/Taiwan/16/2004), Influenza B virus (B/Taiwan/1618/03), Influenza B virus (B/Taiwan/165/2005), Influenza B virus (B/Taiwan/166/2005), Influenza B virus (B/Taiwan/188/2005), Influenza B virus (B/Taiwan/1949/02), Influenza B virus (B/Taiwan/1950/02), Influenza B virus (B/Taiwan/202/2001), Influenza B virus (B/Taiwan/2026/99), Influenza B virus (B/Taiwan/2027/99), Influenza B virus (B/Taiwan/217/97), Influenza B virus (B/Taiwan/21706/97), Influenza B virus (B/Taiwan/2195/99), Influenza B virus (B/Taiwan/2551/03), Influenza B virus (B/Taiwan/2805/01), Influenza B virus (B/Taiwan/2805/2001), Influenza B virus (B/Taiwan/3143/97), Influenza B virus (B/Taiwan/31511/00), Influenza B virus (B/Taiwan/31511/2000), Influenza B virus (B/Taiwan/34/2004), Influenza B virus (B/Taiwan/3532/03), Influenza B virus (B/Taiwan/39/2004), Influenza B virus (B/Taiwan/41010/00), Influenza B virus (B/Taiwan/41010/2000), Influenza B virus (B/Taiwan/4119/02), Influenza B virus (B/Taiwan/4184/00), Influenza B virus (B/Taiwan/4184/2000), Influenza B virus (B/Taiwan/43/2005), Influenza B virus (B/Taiwan/4602/02), Influenza B virus (B/Taiwan/473/2005), Influenza B virus (B/Taiwan/52/2004), Influenza B virus (B/Taiwan/52/2005), Influenza B virus (B/Taiwan/54/2004), Influenza B virus (B/Taiwan/61/2004), Influenza B virus (B/Taiwan/635/2005), Influenza B virus (B/Taiwan/637/2005), Influenza B virus (B/Taiwan/68/2004), Influenza B virus (B/Taiwan/68/2005), Influenza B virus (B/Taiwan/69/2004), Influenza B virus (B/Taiwan/70/2005), Influenza B virus (B/Taiwan/74/2004), Influenza B virus (B/Taiwan/75/2004), Influenza B virus (B/Taiwan/77/2005), Influenza B virus (B/Taiwan/81/2005), Influenza B virus (B/Taiwan/872/2005), Influenza B virus (B/Taiwan/97271/2001), Influenza B virus (B/Taiwan/98/2005), Influenza B virus (B/Taiwan/H96/02), Influenza B virus (B/Taiwan/M214/05), Influenza B virus (B/Taiwan/M227/05), Influenza B virus (B/Taiwan/M24/04), Influenza B virus (B/Taiwan/M244/05), Influenza B virus (B/Taiwan/M251/05), Influenza B virus (B/Taiwan/M53/05), Influenza B virus (B/Taiwan/M71/01), Influenza B virus (B/Taiwan/N1013/99), Influenza B virus (B/Taiwan/N1115/02), Influenza B virus (B/Taiwan/N1207/99), Influenza B virus (B/Taiwan/N1316/01), Influenza B virus (B/Taiwan/N1549/01), Influenza B virus (B/Taiwan/N1582/02), Influenza B virus (B/Taiwan/N16/03), Influenza B virus (B/Taiwan/N1619/04), Influenza B virus (B/Taiwan/N1848/02), Influenza B virus (B/Taiwan/N1902/04), Influenza B virus (B/Taiwan/N200/05), Influenza B virus (B/Taiwan/N2050/02), Influenza B virus (B/Taiwan/N230/01), Influenza B virus (B/Taiwan/N232/00), Influenza B virus (B/Taiwan/N2333/02), Influenza B virus (B/Taiwan/N2335/01), Influenza B virus (B/Taiwan/N253/03), Influenza B virus (B/Taiwan/N2620/04), Influenza B virus (B/Taiwan/N2986/02), Influenza B virus (B/Taiwan/N3688/04), Influenza B virus (B/Taiwan/N371/05), Influenza B virus (B/Taiwan/N376/05), Influenza B virus (B/Taiwan/N384/03), Influenza B virus (B/Taiwan/N3849/02), Influenza B virus (B/Taiwan/N404/02), Influenza B virus (B/Taiwan/N473/00), Influenza B virus (B/Taiwan/N511/01), Influenza B virus (B/Taiwan/N559/05), Influenza B virus (B/Taiwan/N612/01), Influenza B virus (B/Taiwan/N701/01), Influenza B virus (B/Taiwan/N767/01), Influenza B virus (B/Taiwan/N798/05), Influenza B virus (B/Taiwan/N860/05), Influenza B virus (B/Taiwan/N872/04), Influenza B virus (B/Taiwan/N913/04), Influenza B virus (B/Taiwan/S117/05), Influenza B virus (B/Taiwan/S141/02), Influenza B virus (B/Taiwan/S76/02), Influenza B virus (B/Taiwan/S82/02), Influenza B virus (B/Taiwan/103/2005), Influenza B virus (B/Tehran/80/02), Influenza B virus (B/Temple/B10/1999), Influenza B virus (B/Temple/B1166/2001), Influenza B virus (B/Temple/B1181/2001), Influenza B virus (B/Temple/B1182/2001), Influenza B virus (B/Temple/B1188/2001), Influenza B virus (B/Temple/B1190/2001), Influenza B virus (B/Temple/B1193/2001), Influenza B virus (B/Temple/B17/2003), Influenza B virus (B/Temple/B18/2003), Influenza B virus (B/Temple/B19/2003), Influenza B virus (B/Temple/B20/2003), Influenza B virus (B/Temple/B21/2003), Influenza B virus (B/Temple/B24/2003), Influenza B virus (B/Temple/B3/1999), Influenza B virus (B/Temple/B30/2003), Influenza B virus (B/Temple/B7/1999), Influenza B virus (B/Temple/B8/1999), Influenza B virus (B/Temple/B9/1999), Influenza B virus (B/Texas/06/2000), Influenza B virus (B/Texas/1/2000), Influenza B virus (B/Texas/1/2004), Influenza B virus (B/Texas/1/2006), Influenza B virus (B/Texas/1/91), Influenza B virus (B/Texas/10/2005), Influenza B virus (B/Texas/11/2001), Influenza B virus (B/Texas/12/2001), Influenza B virus (B/Texas/14/1991), Influenza B virus (B/Texas/14/2001), Influenza B virus (B/Texas/16/2001), Influenza B virus (B/Texas/18/2001), Influenza B virus (B/Texas/2/2006), Influenza B virus (B/Texas/22/2001), Influenza B virus (B/Texas/23/2000), Influenza B virus (B/Texas/3/2001), Influenza B virus (B/Texas/3/2002), Influenza B virus (B/Texas/3/2006), Influenza B virus (B/Texas/37/1988), Influenza B virus (B/Texas/37/88), Influenza B virus (B/Texas/4/2006), Influenza B virus (B/Texas/4/90), Influenza B virus (B/Texas/5/2002), Influenza B virus (B/Texas/57/2002), Influenza B virus (B/Texas/6/2000), Influenza B virus (B/Tokushima/101/93), Influenza B virus (B/Tokyo/6/98), Influenza B virus (B/Trento/3/02), Influenza B virus (B/Trieste/1/02), Influenza B virus (B/Trieste/1/03), Influenza B virus (B/Trieste/15/02), Influenza B virus (B/Trieste/17/02), Influenza B virus (B/Trieste/19/02), Influenza B virus (B/Trieste/2/03), Influenza B virus (B/Trieste/25/02), Influenza B virus (B/Trieste/27/02), Influenza B virus (B/Trieste/28/02), Influenza B virus (B/Trieste/34/02), Influenza B virus (B/Trieste/37/02), Influenza B virus (B/Trieste/4/02), Influenza B virus (B/Trieste/8/02), Influenza B virus (B/Trieste14/02), Influenza B virus (B/Trieste18/02), Influenza B virus (B/Trieste23/02), Influenza B virus (B/Trieste24/02), Influenza B virus (B/Trieste7/02), Influenza B virus (B/Ulan Ude/4/02), Influenza B virus (B/Ulan-Ude/6/2003), Influenza B virus (B/UlanUde/4/02), Influenza B virus (B/United Kingdom/34304/99), Influenza B virus (B/United Kingdom/34520/99), Influenza B virus (B/Uruguay/19/02), Influenza B virus (B/Uruguay/19/05), Influenza B virus (B/Uruguay/2/02), Influenza B virus (B/Uruguay/28/05), Influenza B virus (B/Uruguay/33/05), Influenza B virus (B/Uruguay/4/02), Influenza B virus (B/Uruguay/5/02), Influenza B virus (B/Uruguay/65/05), Influenza B virus (B/Uruguay/7/02), Influenza B virus (B/Uruguay/74/04), Influenza B virus (B/Uruguay/75/04), Influenza B virus (B/Uruguay/NG/02), Influenza B virus (B/Ushuaia/15732/99), Influenza B virus (B/USSR/100/83), Influenza B virus (B/Utah/1/2005), Influenza B virus (B/Utah/20139/99), Influenza B virus (B/Utah/20975/99), Influenza B virus (B/Vermont/1/2006), Influenza B virus (B/Victoria/02/1987), Influenza B virus (B/Victoria/103/89), Influenza B virus (B/Victoria/19/89), Influenza B virus (B/Victoria/2/87), Influenza B virus (B/Victoria/504/2000), Influenza B virus (B/Vienna/l/99), Influenza B virus (B/Virginia/1/2005), Influenza B virus (B/Virginia/1/2006), Influenza B virus (B/Virginia/11/2003), Influenza B virus (B/Virginia/2/2006), Influenza B virus (B/Virginia/3/2003), Influenza B virus (B/Virginia/3/2006), Influenza B virus (B/Virginia/9/2005), Influenza B virus (B/Washington/1/2004), Influenza B virus (B/Washington/2/2000), Influenza B virus (B/Washington/2/2004), Influenza B virus (B/Washington/3/2000), Influenza B virus (B/Washington/3/2003), Influenza B virus (B/Washington/5/2005), Influenza B virus (B/Wellington/01/1994), Influenza B virus (B/Wisconsin/1/2004), Influenza B virus (B/Wisconsin/1/2006), Influenza B virus (B/Wisconsin/10/2006), Influenza B virus (B/Wisconsin/15e/2005), Influenza B virus (B/Wisconsin/17/2006), Influenza B virus (B/Wisconsin/2/2004), Influenza B virus (B/Wisconsin/2/2006), Influenza B virus (B/Wisconsin/22/2006), Influenza B virus (B/Wisconsin/26/2006), Influenza B virus (B/Wisconsin/29/2006), Influenza B virus (B/Wisconsin/3/2000), Influenza B virus (B/Wisconsin/3/2004), Influenza B virus (B/Wisconsin/3/2005), Influenza B virus (B/Wisconsin/3/2006), Influenza B virus (B/Wisconsin/31/2006), Influenza B virus (B/Wisconsin/4/2006), Influenza B virus (B/Wisconsin/5/2006), Influenza B virus (B/Wisconsin/6/2006), Influenza B virus (B/Wisconsin/7/2002), Influenza B virus (B/Wuhan/2/2001), Influenza B virus (B/Wuhan/356/2000), Influenza B virus (B/WV194/2002), Influenza B virus (B/Wyoming/15/2001), Influenza B virus (B/Wyoming/16/2001), Influenza B virus (B/Wyoming/2/2003), Influenza B virus (B/Xuanwu/1/82), Influenza B virus (B/Xuanwu/23/82), Influenza B virus (B/Yamagata/1/73), Influenza B virus (B/Yamagata/115/2003), Influenza B virus (B/Yamagata/1246/2003), Influenza B virus (B/Yamagata/1311/2003), Influenza B virus (B/Yamagata/16/1988), Influenza B virus (B/Yamagata/16/88), Influenza B virus (B/Yamagata/222/2002), Influenza B virus (B/Yamagata/K198/2001), Influenza B virus (B/Yamagata/K246/2001), Influenza B virus (B/Yamagata/K270/2001), Influenza B virus (B/Yamagata/K298/2001), Influenza B virus (B/Yamagata/K320/2001), Influenza B virus (B/Yamagata/K354/2001), Influenza B virus (B/Yamagata/K386/2001), Influenza B virus (B/Yamagata/K411/2001), Influenza B virus (B/Yamagata/K461/2001), Influenza B virus (B/Yamagata/K490/2001), Influenza B virus (B/Yamagata/K500/2001), Influenza B virus (B/Yamagata/K501/2001), Influenza B virus (B/Yamagata/K508/2001), Influenza B virus (B/Yamagata/K513/2001), Influenza B virus (B/Yamagata/K515/2001), Influenza B virus (B/Yamagata/K519/2001), Influenza B virus (B/Yamagata/K520/2001), Influenza B virus (B/Yamagata/K521/2001), Influenza B virus (B/Yamagata/K535/2001), Influenza B virus (B/Yamagata/K542/2001), Influenza B virus (B/Yamanashi/166/1998), Influenza B virus (B/Yamanashi/166/98), Influenza B virus (B/Yunnan/123/2001), Influenza B virus (strain B/Alaska/12/96), Influenza B virus (STRAIN B/ANN ARBOR/1/66 [COLD-ADAPTED]), Influenza B virus (STRAIN B/ANN ARBOR/1/66 [WILD-TYPE]), Influenza B virus (STRAIN B/BA/78), Influenza B virus (STRAIN B/BEIJING/1/87), Influenza B virus (STRAIN B/ENGLAND/222/82), Influenza B virus (strain B/finland/145/90), Influenza B virus (strain B/finland/146/90), Influenza B virus (strain B/finland/147/90), Influenza B virus (strain B/finland/148/90), Influenza B virus (strain B/finland/149/90), Influenza B virus (strain B/finland/150/90), Influenza B virus (strain B/finland/151/90), Influenza B virus (strain B/finland/24/85), Influenza B virus (strain B/finland/56/88), Influenza B virus (STRAIN B/FUKUOKA/80/81), Influenza B virus (STRAIN B/GA/86), Influenza B virus (STRAIN B/GL/54), Influenza B virus (STRAIN B/HONG KONG/8/73), Influenza B virus (STRAIN B/HT/84), Influenza B virus (STRAIN B/ID/86), Influenza B virus (STRAIN B/LENINGRAD/179/86), Influenza B virus (STRAIN B/MARYLAND/59), Influenza B virus (STRAIN B/MEMPHIS/6/86), Influenza B virus (STRAIN B/NAGASAKI/1/87), Influenza B virus (strain B/Osaka/491/97), Influenza B virus (STRAIN B/PA/79), Influenza B virus (STRAIN B/RU/69), Influenza B virus (STRAIN B/SINGAPORE/64), Influenza B virus (strain B/Tokyo/942/96), Influenza B virus (STRAIN B/VICTORIA/3/85), Influenza B virus (STRAIN B/VICTORIA/87), Influenza B virus(B/Rochester/02/2001), and other subtypes. In further embodiments, the influenza virus C belongs to but is not limited to subtype Influenza C virus (C/Aichi/1/81), Influenza C virus (C/Aichi/1/99), Influenza C virus (C/Ann Arbor/i/50), Influenza C virus (C/Aomori/74), Influenza C virus (C/California/78), Influenza C virus (C/England/83), Influenza C virus (C/Fukuoka/2/2004), Influenza C virus (C/Fukuoka/3/2004), Influenza C virus (C/Fukushima/1/2004), Influenza C virus (C/Greece/79), Influenza C virus (C/Hiroshima/246/2000), Influenza C virus (C/Hiroshima/247/2000), Influenza C virus (C/Hiroshima/248/2000), Influenza C virus (C/Hiroshima/249/2000), Influenza C virus (C/Hiroshima/250/2000), Influenza C virus (C/Hiroshima/251/2000), Influenza C virus (C/Hiroshima/252/2000), Influenza C virus (C/Hiroshima/252/99), Influenza C virus (C/Hiroshima/290/99), Influenza C virus (C/Hiroshima/4/2004), Influenza C virus (C/Hyogo/1/83), Influenza C virus (C/Johannesburg/1/66), Influenza C virus (C/Johannesburg/66), Influenza C virus (C/Kanagawa/1/76), Influenza C virus (C/Kanagawa/2/2004), Influenza C virus (C/Kansas/1/79), Influenza C virus (C/Kyoto/1/79), Influenza C virus (C/Kyoto/41/82), Influenza C virus (C/Mississippi/80), Influenza C virus (C/Miyagi/1/90), Influenza C virus (C/Miyagi/1/93), Influenza C virus (C/Miyagi/1/94), Influenza C virus (C/Miyagi/1/97), Influenza C virus (C/Miyagi/1/99), Influenza C virus (C/Miyagi/12/2004), Influenza C virus (C/Miyagi/2/2000), Influenza C virus (C/Miyagi/2/92), Influenza C virus (C/Miyagi/2/93), Influenza C virus (C/Miyagi/2/94), Influenza C virus (C/Miyagi/2/96), Influenza C virus (C/Miyagi/2/98), Influenza C virus (C/Miyagi/3/2000), Influenza C virus (C/Miyagi/3/91), Influenza C virus (C/Miyagi/3/92), Influenza C virus (C/Miyagi/3/93), Influenza C virus (C/Miyagi/3/94), Influenza C virus (C/Miyagi/3/97), Influenza C virus (C/Miyagi/3/99), Influenza C virus (C/Miyagi/4/2000), Influenza C virus (C/Miyagi/4/93), Influenza C virus (C/Miyagi/4/96), Influenza C virus (C/Miyagi/4/97), Influenza C virus (C/Miyagi/4/98), Influenza C virus (C/Miyagi/42/2004), Influenza C virus (C/Miyagi/5/2000), Influenza C virus (C/Miyagi/5/91), Influenza C virus (C/Miyagi/5/93), Influenza C virus (C/Miyagi/6/93), Influenza C virus (C/Miyagi/6/96), Influenza C virus (C/Miyagi/7/91), Influenza C virus (C/Miyagi/7/93), Influenza C virus (C/Miyagi/7/96), Influenza C virus (C/Miyagi/77), Influenza C virus (C/Miyagi/8/96), Influenza C virus (C/Miyagi/9/91), Influenza C virus (C/Miyagi/9/96), Influenza C virus (C/Nara/1/85), Influenza C virus (C/Nara/2/85), Influenza C virus (C/Nara/82), Influenza C virus (C/NewJersey/76), Influenza C virus (C/Niigata/1/2004), Influenza C virus (C/Osaka/2/2004), Influenza C virus (C/pig/Beijing/115/81), Influenza C virus (C/Saitama/1/2000), Influenza C virus (C/Saitama/1/2004), Influenza C virus (C/Saitama/2/2000), Influenza C virus (C/Saitama/3/2000), Influenza C virus (C/Sapporo/71), Influenza C virus (C/Shizuoka/79), Influenza C virus (C/Yamagata/1/86), Influenza C virus (C/Yamagata/1/88), Influenza C virus (C/Yamagata/10/89), Influenza C virus (C/Yamagata/13/98), Influenza C virus (C/Yamagata/15/2004), Influenza C virus (C/Yamagata/2/2000), Influenza C virus (C/Yamagata/2/98), Influenza C virus (C/Yamagata/2/99), Influenza C virus (C/Yamagata/20/2004), Influenza C virus (C/Yamagata/20/96), Influenza C virus (C/Yamagata/21/2004), Influenza C virus (C/Yamagata/26/81), Influenza C virus (C/Yamagata/27/2004), Influenza C virus (C/Yamagata/3/2000), Influenza C virus (C/Yamagata/3/2004), Influenza C virus (C/Yamagata/3/88), Influenza C virus (C/Yamagata/3/96), Influenza C virus (C/Yamagata/4/88), Influenza C virus (C/Yamagata/4/89), Influenza C virus (C/Yamagata/5/92), Influenza C virus (C/Yamagata/6/2000), Influenza C virus (C/Yamagata/6/98), Influenza C virus (C/Yamagata/64), Influenza C virus (C/Yamagata/7/88), Influenza C virus (C/Yamagata/8/2000), Influenza C virus (C/Yamagata/8/88), Influenza C virus (C/Yamagata/8/96), Influenza C virus (C/Yamagata/9/2000), Influenza C virus (C/Yamagata/9/88), Influenza C virus (C/Yamagata/9/96), Influenza C virus (STRAIN C/BERLIN/1/85), Influenza C virus (STRAIN C/ENGLAND/892/83), Influenza C virus (STRAIN C/GREAT LAKES/1167/54), Influenza C virus (STRAIN C/JJ/50), Influenza C virus (STRAIN C/PIG/BEIJING/10/81), Influenza C virus (STRAIN C/PIG/BEIJING/439/82), Influenza C virus (STRAIN C/TAYLOR/1233/47), Influenza C virus (STRAIN C/YAMAGATA/10/81), Isavirus or Infectious salmon anemia virus, Thogotovirus or Dhori virus, Batken virus, Dhori virus (STRAIN INDIAN/1313/61) or Thogoto virus, Thogoto virus (isolate SiAr 126) or unclassified Thogotovirus, Araguari virus, unclassified Orthomyxoviridae or Fowl plague virus or Swine influenza virus or unidentified influenza virus and other subtypes.

In various embodiments, the attenuated virus belongs to the delta virus family and all related genera.

In various embodiments, the attenuated virus belongs to the Adenoviridae virus family and all related genera, strains, types and isolates for example but not limited to human adenovirus A, B C.

In various embodiments, the attenuated virus belongs to the Herpesviridae virus family and all related genera, strains, types and isolates for example but not limited to herpes simplex virus.

In various embodiments, the attenuated virus belongs to the Reoviridae virus family and all related genera, strains, types and isolates.

In various embodiments, the attenuated virus belongs to the Papillomaviridae virus family and all related genera, strains, types and isolates.

In various embodiments, the attenuated virus belongs to the Poxviridae virus family and all related genera, strains, types and isolates.

In various embodiments, the attenuated virus belongs to the Retroviridae virus family and all related genera, strains, types and isolates. For example but not limited to Human Immunodeficiency Virus.

In various embodiments, the attenuated virus belongs to the Filoviridae virus family and all related genera, strains, types and isolates.

In various embodiments, the attenuated virus belongs to the Paramyxoviridae virus family and all related genera, strains, types and isolates.

In various embodiments, the attenuated virus belongs to the Orthomyxoviridae virus family and all related genera, strains, types and isolates.

In various embodiments, the attenuated virus belongs to the Picornaviridae virus family and all related genera, strains, types and isolates.

In various embodiments, the attenuated virus belongs to the Bunyaviridae virus family and all related genera, strains, types and isolates.

In various embodiments, the attenuated virus belongs to the Nidovirales virus family and all related genera, strains, types and isolates.

In various embodiments, the attenuated virus belongs to the Caliciviridae virus family and all related genera, strains, types and isolates.

In certain embodiments, the synonymous codon substitutions alter codon bias, codon pair bias, density of deoptimized codons and deoptimized codon pairs, RNA secondary structure, CpG dinucleotide content, C+G content, translation frameshift sites, translation pause sites, the presence or absence microRNA recognition sequences or any combination thereof, in the genome. The codon substitutions may be engineered in multiple locations distributed throughout the genome, or in the multiple locations restricted to a portion of the genome. In further embodiments, the portion of the genome is the capsid coding region.

In preferred embodiments of this invention, the virus retains the ability to induce a protective immune response in an animal host. In other preferred embodiments, the virulence of the virus does not revert to wild type.

### Poliovirus, Rhinovirus, and Influenza Virus

Poliovirus, a member of the Picornavirus family, is a small non-enveloped virus with a single stranded (+) sense RNA genome of 7.5 kb in length (Kitamura et al., 1981). Upon cell entry, the genomic RNA serves as an mRNA encoding a single polyprotein that after a cascade of autocatalytic cleavage events gives rise to full complement of functional poliovirus proteins. The same genomic RNA serves as a template for the synthesis of (−) sense RNA, an intermediary for the synthesis of new (+) strands that either serve as mRNA, replication template or genomic RNA destined for encapsidation into progeny virions (Mueller et al., 2005). As described herein, the well established PV system was used to address general questions of optimizing design strategies for the production of attenuated synthetic viruses. PV provides one of the most important and best understood molecular models for developing anti-viral strategies. In particular, a reverse genetics system exists whereby viral nucleic acid can be synthesized in vitro by completely synthetic methods and then converted into infectious virions (see below). Furthermore, a convenient mouse model is available (CD155tg mice, which express the human receptor for polio) for testing attenuation of synthetic PV designs as previously described (Cello et al., 2002).

Rhinoviruses are also members of the Picornavirus family, and are related to PV. Human Rhinoviruses (HRV) are the usual causative agent of the common cold, and as such they are responsible for more episodes of illness than any other infectious agent (Hendley, 1999). In addition to the common cold, HRV is also involved in ear and sinus infections, asthmatic attacks, and other diseases. Similar to PV, HRV comprises a single-stranded positive sense RNA virus, whose genome encodes a self-processing polyprotein. The RNA is translated through an internal initiation mechanism using an Internal Ribosome Entry Site (IRES) to produce structural proteins that form the capsid, as well as non-structural proteins such as the two viral proteases, 2A and 3C, and the RNA-dependent polymerase (Jang et al., 1989; Pelletier et al., 1988). Also like PV, HRV has a non-enveloped icosahedral capsid, formed by 60 copies of the four capsid proteins VP1-4 (Savolainen et al., 2003). The replication cycle of HRV is also identical to that of poliovirus. The close similarity to PV, combined with the significant, almost ubiquitous impact on human health, makes HRV an extremely attractive candidate for generating a novel attenuated virus useful for immunization.

Despite decades of research by pharmaceutical companies, no successful drug against HRV has been developed. This is partly due to the relatively low risk tolerance of federal regulators and the public for drugs that treat a mostly non-serious infection. That is, even minor side effects are unacceptable. Thus, in the absence of a drug, there is a clear desire for a safe and effective anti-rhinovirus vaccine. However, developing an anti-rhinovirus vaccine is extremely challenging, because there are over 100 serotypes of HRV, of which approximately 30 circulate widely and infect humans regularly. An effective vaccine must enable the immune system to recognize every single serotype in order to confer true immunity. The SAVE approach described herein offers a practical solution to the development of an effective rhinovirus vaccine. Based on the predictability of the SAVE design process, it would be inexpensive to design and synthesize 100 or more SAVE-attenuated rhinoviruses, which in combination would constitute a vaccine.

Influenza virus—Between 1990 and 1999, influenza viruses caused approximately 35,000 deaths each year in the U.S.A. (Thompson et al., 2003). Together with approximately 200,000 hospitalizations, the impact on the U.S. economy has been estimated to exceed $23 billion annually (Cram et al., 2001). Globally, between 300,000 to 500,000 people die each year due to influenza virus infections (Kamps et al., 2006). Although the virus causes disease amongst all age groups, the rates of serious complications are highest in children and persons over 65 years of age. Influenza has the potential to mutate or recombine into extremely deadly forms, as happened during the great influenza epidemic of 1918, in which about 30 million people died. This was possibly the single most deadly one-year epidemic in human history.

Influenza viruses are divided into three types A, B, and C. Antigenicity is determined by two glycoproteins at the surface of the enveloped virion: hemagglutinin (HA) and neuraminidase (NA). Both glycoproteins continuously change their antigenicity to escape humoral immunity. Altering the glycoproteins allows virus strains to continue infecting vaccinated individuals, which is the reason for yearly vaccination of high-risk groups. In addition, human influenza viruses can replace the HA or NA glycoproteins with those of birds and pigs, a reassortment of gene segments, known as genetic shift, leading to new viruses (H1N1 to H2N2 or H3N2, etc.) (Steinhauer and Skehel, 2002). These novel viruses, to which the global population is immunologically naive, are the cause of pandemics that kill millions of people (Kilbourne, 2006; Russell and Webster, 2005). The history of influenza virus, together with the current threat of the highly pathogenic avian influenza virus, H5N1 (Stephenson and Democratis, 2006), underscores the need for preventing influenza virus disease.

Currently, two influenza vaccines are in use: a live, attenuated vaccine (cold adapted; “FluMist”) and an inactivated virus. The application of the attenuated vaccine is restricted to healthy children, adolescents and adults (excluding pregnant females), ages 5-49. This age restriction leaves out precisely those who are at highest risks of influenza. Furthermore, the attenuated FluMist virus has the possibility of reversion, which is usual for a live virus. Production of the second, more commonly administered inactivated influenza virus vaccine is complex. Further, this vaccine appears to be less effective than hoped for in preventing death in the elderly (>65-year-old) population (Simonson et al., 2005). These facts underscore the need for novel strategies to generate influenza virus vaccines.

### Reverse Genetics of Picornaviruses

Reverse genetics generally refers to experimental approaches to discovering the function of a gene that proceeds in the opposite direction to the so-called forward genetic approaches of classical genetics. That is, whereas forward genetics approaches seek to determine the function of a gene by elucidating the genetic basis of a phenotypic trait, strategies based on reverse genetics begin with an isolated gene and seek to discover its function by investigating the possible phenotypes generated by expression of the wt or mutated gene. As used herein in the context of viral systems, “reverse genetics” systems refer to the availability of techniques that permit genetic manipulation of viral genomes made of RNA. Briefly, the viral genomes are isolated from virions or from infected cells, converted to DNA (“cDNA”) by the enzyme reverse transcriptase, possibly modified as desired, and reverted, usually via the RNA intermediate, back into infectious viral particles. This process in picornaviruses is extremely simple; in fact, the first reverse genetics system developed for any animal RNA virus was for PV (Racaniello and Baltimore, 1981). Viral reverse genetics systems are based on the historical finding that naked viral genomic RNA is infectious when transfected into a suitable mammalian cell (Alexander et al., 1958). The discovery of reverse transcriptase and the development of molecular cloning techniques in the 1970's enabled scientists to generate and manipulate cDNA copies of RNA viral genomes. Most commonly, the entire cDNA copy of the genome is cloned immediately downstream of a phage T7 RNA polymerase promoter that allows the in vitro synthesis of genome RNA, which is then transfected into cells for generation of virus (van der Wert, et al., 1986). Alternatively, the same DNA plasmid may be transfected into cells expressing the T7 RNA polymerase in the cytoplasm. This system can be used for various viral pathogens including both PV and HRV.

### Molecular Virology and Reverse Genetics of Influenza Virus

Influenza virus, like the picornaviruses, PV and HRV, is an RNA virus, but is otherwise unrelated to and quite different from PV. In contrast to the picornaviruses, influenza is a minus strand virus. Furthermore, influenza consists of eight separate gene segments ranging from 890 to 2341 nucleotides (Lamb and Krug, 2001). Partly because of the minus strand organization, and partly because of the eight separate gene segments, the reverse genetics system is more complex than for PV. Nevertheless, a reverse genetics system has been developed for influenza virus (Enami et al., 1990; Fodor et al., 1999; Garcia-Sastre and Palese, 1993; Hoffman et al., 2000; Luytjes et al., 1989; Neumann et al., 1999). Each of the eight gene segments is expressed from a separate plasmid. This reverse genetics system is extremely convenient for use in the SAVE strategy described herein, because the longest individual gene segment is less than 3 kb, and thus easy to synthesize and manipulate. Further, the different gene segments can be combined and recombined simply by mixing different plasmids. Thus, application of SAVE methods are possibly even more feasible for influenza virus than for PV.

A recent paradigm shift in viral reverse genetics occurred with the present inventors' first chemical synthesis of an infectious virus genome by assembly from synthetic DNA oligonucleotides (Cello et al., 2002). This achievement made it clear that most or all viruses for which a reverse genetics system is available can be synthesized solely from their genomic sequence information, and promises unprecedented flexibility in re-synthesizing and modifying these viruses to meet desired criteria.

### De Novo Synthesis of Viral Genomes

Computer-based algorithms are used to design and synthesize viral genomes de novo. These synthesized genomes, exemplified by the synthesis of attenuated PV described herein, encode exactly the same proteins as wild type(wt) viruses, but by using alternative synonymous codons, various parameters, including codon bias, codon pair bias, RNA secondary structure, and/or dinucleotide content, are altered. The presented data show that these coding-independent changes produce highly attenuated viruses, often due to poor translation of proteins. By targeting an elementary function of all viruses, namely protein translation, a very general method has been developed for predictably, safely, quickly and cheaply producing attenuated viruses, which are useful for making vaccines. This method, dubbed “SAVE” (Synthetic Attenuated Virus Engineering), is applicable to a wide variety of viruses other than PV for which there is a medical need for new vaccines. These viruses include, but are not limited to rhinovirus, influenza virus, SARS and other coronaviruses, HIV, HCV, infectious bronchitis virus, ebolavirus, Marburg virus, dengue fever virus, West Nile disease virus, EBV, yellow fever virus, enteroviruses other than poliovirus, such as echoviruses, coxsackie viruses, and entrovirus71; hepatitis A virus, aphthoviruses, such as foot-and-mouth-disease virus, myxoviruses, such as influenza viruses, paramyxoviruses, such as measles virus, mumps virus, respiratory syncytia virus, flaviviruses such as dengue virus, yellow fever virus, St. Louis encephalitis virus and tick-born virus, alphaviruses, such as Western- and Eastern encephalitis virus, hepatitis B virus, and bovine diarrhea virus, and ebolavirus.

Both codon and codon-pair deoptimization in the PV capsid coding region are shown herein to dramatically reduce PV fitness. The present invention is not limited to any particular molecular mechanism underlying virus attenuation via substitution of synonymous codons. Nevertheless, experiments are ongoing to better understand the underlying molecular mechanisms of codon and codon pair deoptimization in producing attenuated viruses. In particular, evidence is provided in this application that indicates that codon deoptimization and codon pair deoptimization can result in inefficient translation. High throughput methods for the quick generation and screening of large numbers of viral constructs are also being developed.

### Large-Scale DNA Assembly

In recent years, the plunging costs and increasing quality of oligonucleotide synthesis have made it practical to assemble large segments of DNA (at least up to about 10 kb) from synthetic oligonucleotides. Commercial vendors such as Blue Heron Biotechnology, Inc. (Bothwell, WA) (and also many others) currently synthesize, assemble, clone, sequence-verify, and deliver a large segment of synthetic DNA of known sequence for the relatively low price of about $1.50 per base. Thus, purchase of synthesized viral genomes from commercial suppliers is a convenient and cost-effective option, and prices continue to decrease rapidly. Furthermore, new methods of synthesizing and assembling very large DNA molecules at extremely low costs are emerging (Tian et al., 2004). The Church lab has pioneered a method that uses parallel synthesis of thousands of oligonucleotides (for instance, on photo-programmable microfluidics chips, or on microarrays available from Nimblegen Systems, Inc., Madison, WI, or Agilent Technologies, Inc., Santa Clara, CA), followed by error reduction and assembly by overlap PCR. These methods have the potential to reduce the cost of synthetic large DNAs to less than 1 cent per base. The improved efficiency and accuracy, and rapidly declining cost, of large-scale DNA synthesis provides an impetus for the development and broad application of the SAVE strategy.

### Alternative Encoding, Codon Bias, and Codon Pair Bias

### Alternative Encoding

A given peptide can be encoded by a large number of nucleic acid sequences. For example, even a typical short 10-mer oligopeptide can be encoded by approximately 4¹⁰ (about 10⁶) different nucleic acids, and the proteins of PV can be encoded by about 10⁴⁴² different nucleic acids. Natural selection has ultimately chosen one of these possible 10⁴⁴² nucleic acids as the PV genome. Whereas the primary amino acid sequence is the most important level of information encoded by a given mRNA, there are additional kinds of information within different kinds of RNA sequences. These include RNA structural elements of distinct function (e.g., for PV, the cis-acting replication element, or CRE (Goodfellow et al., 2000; McKnight, 2003), translational kinetic signals (pause sites, frame shift sites, etc.), polyadenylation signals, splice signals, enzymatic functions (ribozyme) and, quite likely, other as yet unidentified information and signals).

Even with the caveat that signals such as the CRE must be preserved, 10⁴⁴² possible encoding sequences provide tremendous flexibility to make drastic changes in the RNA sequence of polio while preserving the capacity to encode the same protein. Changes can be made in codon bias or codon pair bias, and nucleic acid signals and secondary structures in the RNA can be added or removed. Additional or novel proteins can even be simultaneously encoded in alternative frames (see, e.g., Wang et al., 2006).

### Codon Bias

Whereas most amino acids can be encoded by several different codons, not all codons are used equally frequently: some codons are “rare” codons, whereas others are “frequent” codons. As used herein, a “rare” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40% of the time, but use the synonymous CTA only 7% of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens (source:
http://www.kazusa.or.jp/codon/)

| Amino Acid | Codon | Number | /1000 | Fraction |
|---|---|---|---|---|
| Gly | GGG | 636457.00 | 16.45 | 0.25 |
| Gly | GGA | 637120.00 | 16.47 | 0.25 |
| Gly | GGT | 416131.00 | 10.76 | 0.16 |
| Gly | GGC | 862557.00 | 22.29 | 0.34 |
| Glu | GAG | 1532589.00 | 39.61 | 0.58 |
| Glu | GAA | 1116000.00 | 28.84 | 0.42 |
| Asp | GAT | 842504.00 | 21.78 | 0.46 |
| Asp | GAC | 973377.00 | 25.16 | 0.54 |
| Val | GTG | 1091853.00 | 28.22 | 0.46 |
| Val | GTA | 273515.00 | 7.07 | 0.12 |
| Val | GTT | 426252.00 | 11.02 | 0.18 |
| Val | GTC | 562086.00 | 14.53 | 0.24 |
| Ala | GCG | 286975.00 | 7.42 | 0.11 |
| Ala | GCA | 614754.00 | 15.89 | 0.23 |
| Ala | GCT | 715079.00 | 18.48 | 0.27 |
| Ala | GCC | 1079491.00 | 27.90 | 0.40 |
| Arg | AGG | 461676.00 | 11.93 | 0.21 |
| Arg | AGA | 466435.00 | 12.06 | 0.21 |
| Ser | AGT | 469641.00 | 12.14 | 0.15 |
| Ser | AGC | 753597.00 | 19.48 | 0.24 |
| Lys | AAG | 1236148.00 | 31.95 | 0.57 |
| Lys | AAA | 940312.00 | 24.30 | 0.43 |
| Asn | AAT | 653566.00 | 16.89 | 0.47 |
| Asn | AAC | 739007.00 | 19.10 | 0.53 |
| Met | ATG | 853648.00 | 22.06 | 1.00 |
| Ile | ATA | 288118.00 | 7.45 | 0.17 |
| Ile | ATT | 615699.00 | 15.91 | 0.36 |
| Ile | ATC | 808306.00 | 20.89 | 0.47 |
| Thr | ACG | 234532.00 | 6.06 | 0.11 |
| Thr | ACA | 580580.00 | 15.01 | 0.28 |
| Thr | ACT | 506277.00 | 13.09 | 0.25 |
| Thr | ACC | 732313.00 | 18.93 | 0.36 |
| Trp | TGG | 510256.00 | 13.19 | 1.00 |
| End | TGA | 59528.00 | 1.54 | 0.47 |
| Cys | TGT | 407020.00 | 10.52 | 0.45 |
| Cys | TGC | 487907.00 | 12.61 | 0.55 |
| End | TAG | 30104.00 | 0.78 | 0.24 |
| End | TAA | 38222.00 | 0.99 | 0.30 |
| Tyr | TAT | 470083.00 | 12.15 | 0.44 |
| Tyr | TAC | 592163.00 | 15.30 | 0.56 |
| Leu | TTG | 498920.00 | 12.89 | 0.13 |
| Leu | TTA | 294684.00 | 7.62 | 0.08 |
| Phe | TTT | 676381.00 | 17.48 | 0.46 |
| Phe | TTC | 789374.00 | 20.40 | 0.54 |
| Ser | TCG | 171428.00 | 4.43 | 0.05 |
| Ser | TCA | 471469.00 | 12.19 | 0.15 |
| Ser | TCT | 585967.00 | 15.14 | 0.19 |
| Ser | TCC | 684663.00 | 17.70 | 0.22 |
| Arg | CGG | 443753.00 | 11.47 | 0.20 |
| Arg | CGA | 239573.00 | 6.19 | 0.11 |
| Arg | CGT | 176691.00 | 4.57 | 0.08 |
| Arg | CGC | 405748.00 | 10.49 | 0.18 |
| Gln | CAG | 1323614.00 | 34.21 | 0.74 |
| Gln | CAA | 473648.00 | 12.24 | 0.26 |
| His | CAT | 419726.00 | 10.85 | 0.42 |
| His | CAC | 583620.00 | 15.08 | 0.58 |
| Leu | CTG | 1539118.00 | 39.78 | 0.40 |
| Leu | CTA | 276799.00 | 7.15 | 0.07 |
| Leu | CTT | 508151.00 | 13.13 | 0.13 |
| Leu | CTC | 759527.00 | 19.63 | 0.20 |
| Pro | CCG | 268884.00 | 6.95 | 0.11 |
| Pro | CCA | 653281.00 | 16.88 | 0.28 |
| Pro | CCT | 676401.00 | 17.48 | 0.29 |
| Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both. The PV capsid protein is very actively translated, and has a high codon bias.

### Codon Pair Bias

A distinct feature of coding sequences is their codon pair bias. This may be illustrated by considering the amino acid pair Ala-Glu, which can be encoded by 8 different codon pairs. If no factors other than the frequency of each individual codon (as shown in Table 2) are responsible for the frequency of the codon pair, the expected frequency of each of the 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 2). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes the frequencies of codon usage were re-calculated by dividing the number of occurrences of a codon by the number of all synonymous codons coding for the same amino acid. As expected the frequencies correlated closely with previously published ones such as the ones given in Table 2. Slight frequency variations are possibly due to an oversampling effect in the data provided by the codon usage database at Kazusa DNA Research Institute (http://www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 3 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.97 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of GC 14,795 human genes, are provided herewith as Supplemental Table 1.

TABLE 3
Codon Pair Scores Exemplified by the Amino
Acid Pair Ala-Glu

| amino | | | | |
|---|---|---|---|---|
| acid | codon | expected | observed | obs/exp |
| pair | pair | frequency | frequency | ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

Codon pair bias was discovered in prokaryotic cells (see Greve et al., 1989), but has since been seen in all other examined species, including humans. The effect has a very high statistical significance, and is certainly not just noise. However, its functional significance, if any, is a mystery. One proposal is that some pairs of tRNAs interact well when they are brought together on the ribosome, while other pairs interact poorly. Since different codons are usually read by different tRNAs, codon pairs might be biased to avoid putting together pairs of incompatible tRNAs (Greve et al., 1989). Another idea is that many (but not all) under-represented pairs have a central CG dinucleotide (e.g., GCCGAA, encoding AlaGlu), and the CG dinucleotide is systematically under-represented in mammals (Buchan et al., 2006; Curran et al., 1995; Fedorov et al., 2002). Thus, the effects of codon pair bias could be of two kinds—one an indirect effect of the under-representation of CG in the mammalian genome, and the other having to do with the efficiency, speed and/or accuracy of translation. It is emphasized that the present invention is not limited to any particular molecular mechanism underlying codon pair bias.

As discussed more fully below, codon pair bias takes into account the score for each codon pair in a coding sequence averaged over the entire length of the coding sequence. According to the invention, codon pair bias is determined by

$$CPB = {\sum }_{i = 1}^{k} \frac{CPSi}{k - 1}$$

Accordingly, similar codon pair bias for a coding sequence can be obtained, for example, by minimized codon pair scores over a subsequence or moderately diminished codon pair scores over the full length of the coding sequence.

Since all 61 sense codons and all sense codon pairs can certainly be used, it would not be expected that substituting a single rare codon for a frequent codon, or a rare codon pair for a frequent codon pair, would have much effect. Therefore, many previous investigations of codon and codon pair bias have been done via informatics, not experimentation. One investigation of codon pair bias that was based on experimental work was the study of Irwin et al. (1995), who found, counterintuitively, that certain over-represented codon pairs caused slower translation. However, this result could not be reproduced by a second group (Cheng and Goldman, 2001), and is also in conflict with results reported below. Thus, the present results (see below) may be the first experimental evidence for a functional role of codon pair bias.

Certain experiments disclosed herein relate to re-coding viral genome sequences, such as the entire capsid region of PV, involving around 1000 codons, to separately incorporate both poor codon bias and poor codon pair bias into the genome. The rationale underlying these experiments is that if each substitution creates a small effect, then all substitutions together should create a large effect. Indeed, it turns out that both deoptimized codon bias, and deoptimized codon pair bias, separately create non-viable viruses. As discussed in more detail in the Examples, preliminary data suggest that inefficient translation is the major mechanism for reducing the viability of a virus with poor codon bias or codon pair bias. Irrespective of the precise mechanism, the data indicate that the large-scale substitution of synonymous deoptimized codons into a viral genome results in severely attenuated viruses. This procedure for producing attenuated viruses has been dubbed SAVE (Synthetic Attenuated Virus Engineering).

According to the invention, viral attenuation can be accomplished by changes in codon pair bias as well as codon bias. However, it is expected that adjusting codon pair bias is particularly advantageous. For example, attenuating a virus through codon bias generally requires elimination of common codons, and so the complexity of the nucleotide sequence is reduced. In contrast, codon pair bias reduction or minimization can be accomplished while maintaining far greater sequence diversity, and consequently greater control over nucleic acid secondary structure, annealing temperature, and other physical and biochemical properties. The work disclosed herein includes attenuated codon pair bias-reduced or -minimized sequences in which codons are shuffled, but the codon usage profile is unchanged.

Viral attenuation can be confirmed in ways that are well known to one of ordinary skill in the art. Non-limiting examples include plaque assays, growth measurements, and reduced lethality in test animals. The instant application demonstrates that the attenuated viruses are capable of inducing protective immune responses in a host.

### Synthetic Attenuated Virus Engineering (SAVE)

SAVE employs specifically designed computer software and modern methods of nucleic acid synthesis and assembly to re-code and re-synthesize the genomes of viruses. This strategy provides an efficient method of producing vaccines against various medically important viruses for which efficacious vaccines are sought.

Two effective polio vaccines, an inactivated polio vaccine (IPV) developed by Jonas Salk and an oral polio vaccine (OPV) comprising live attenuated virus developed by Albert Sabin, respectively, have been available sine the 1950's. Indeed, a global effort to eradicate poliomyelitis, begun in 1988 and led by the World Health Organization (WHO), has succeeded in eradicating polio from most of the countries in the world. The number of annual diagnosed cases has been reduced from the hundreds of thousands to less that two thousand in 2005, occurring mainly in India and in Nigeria. However, a concern regarding the wide use of the OPV is that is can revert to a virulent form, and though believed to be a rare event, outbreaks of vaccine-derived polio have been reported (Georgescu et al., 1997; Kew et al., 2002; Shimizu et al., 2004). In fact, as long as the live poliovirus vaccine strains are used, each carrying less than 7 attenuating mutations, there is a possibility that this strain will revert to wt, and such reversion poses a serious threat to the complete eradication of polio. Thus, the WHO may well need a new polio vaccine to combat the potential of reversion in the closing stages of its efforts at polio eradication, and this provides one rationale for the studies disclosed herein on the application of SAVE to PV. However, PV was selected primarily because it is an excellent model system for developing SAVE.

During re-coding, essential nucleic acid signals in the viral genome are preserved, but the efficiency of protein translation is systematically reduced by deoptimizing codon bias, codon pair bias, and other parameters such as RNA secondary structure and CpG dinucleotide content, C+G content, translation frameshift sites, translation pause sites, or any combination thereof. This deoptimization may involve hundreds or thousands of changes, each with a small effect. Generally, deoptimization is performed to a point at which the virus can still be grown in some cell lines (including lines specifically engineered to be permissive for a particular virus), but where the virus is avirulent in a normal animal or human. Such avirulent viruses are excellent candidates for either a killed or live vaccine since they encode exactly the same proteins as the fully virulent virus and accordingly provoke exactly the same immune response as the fully virulent virus. In addition, the SAVE process offers the prospect for fine tuning the level of attenuation; that is, it provides the capacity to design synthetic viruses that are deoptimized to a roughly predictable extent. Design, synthesis, and production of viral particles is achievable in a timeframe of weeks once the genome sequence is known, which has important advantages for the production of vaccines in potential emergencies. Furthermore, the attenuated viruses are expected to have virtually no potential to revert to virulence because of the extremely large numbers of deleterious nucleotide changes involved. This method may be generally applicable to a wide range of viruses, requiring only knowledge of the viral genome sequence and a reverse genetics system for any particular virus.

### Viral Attenuation by Deoptimizing Codon Bias

If one uses the IC₅₀-ratio of control cells/test cells method as described above, then compounds with CSG values less than or equal to 1 would not generally be considered to be good clinical candidate compounds, whereas compounds with CSG values of greater than approximately 10 would be quite promising and worthy of further consideration.

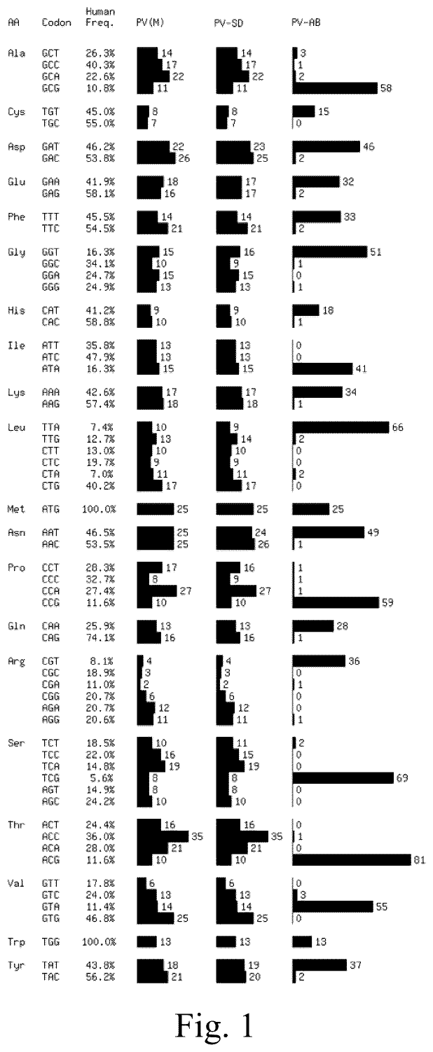

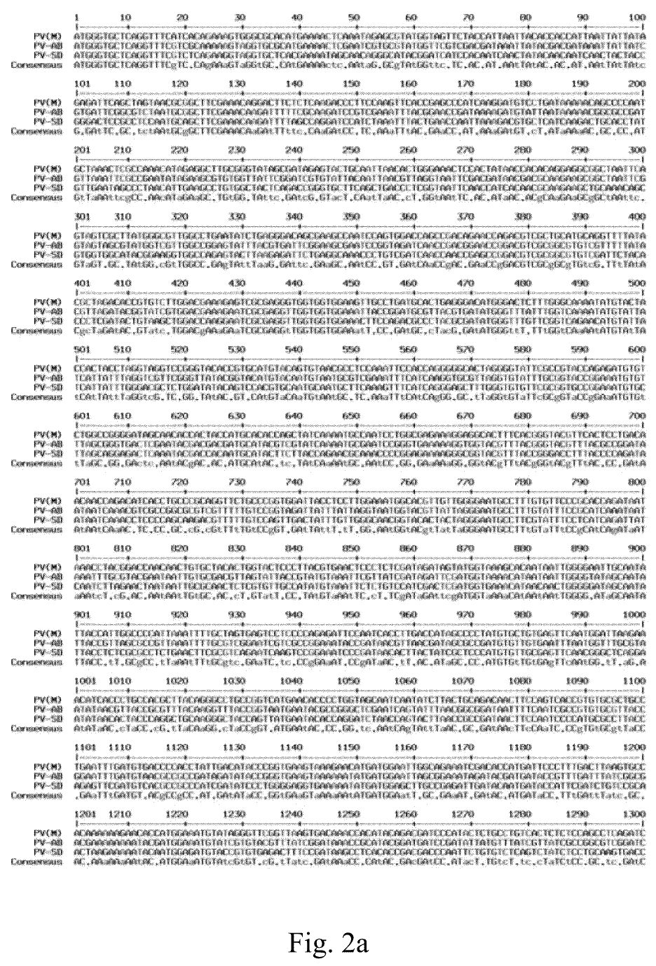

As a means of engineering attenuated viruses, the capsid coding region of poliovirus type 1 Mahoney [PV(M)] was re-engineered by making changes in synonymous codon usage. The capsid region comprises about a third of the virus and is very actively translated. One mutant virus (virus PV-AB), having a very low codon bias due to replacement of the largest possible number of frequently used codons with rare synonymous codons was created. As a control, another virus (PV-SD) was created having the largest possible number of synonymous codon changes while maintaining the original codon bias. See FIGS. 1 and 2. Thus, PV-SD is a virus having essentially the same codons as the wt, but in shuffled position while encoding exactly the same proteins. In PV-SD, no attempt was made to increase or reduce codon pair bias by the shuffling procedure. See Example 1. Despite 934 nucleotide changes in the capsid-coding region, PV-SD RNA produced virus with characteristics indistinguishable from wt. In contrast, no viable virus was recovered from PV-AB carrying 680 silent mutations. See Example 2.

A trivial explanation of the inviability of PV-AB is that just one of the nucleotide changes is somehow lethal, while the other 679 are harmless. For instance, a nucleotide change could be lethal for some catastrophic but unappreciated reason, such as preventing replication. This explanation is unlikely, however. Although PV does contain important regulatory elements in its RNA, such as the CRE, it is known that no such elements exist inside the capsid coding region. This is supported by the demonstration that the entire capsid coding region can be deleted without affecting normal replication of the residual genome within the cell, though of course viral particles cannot be formed (Kaplan and Racamiello, 1988).

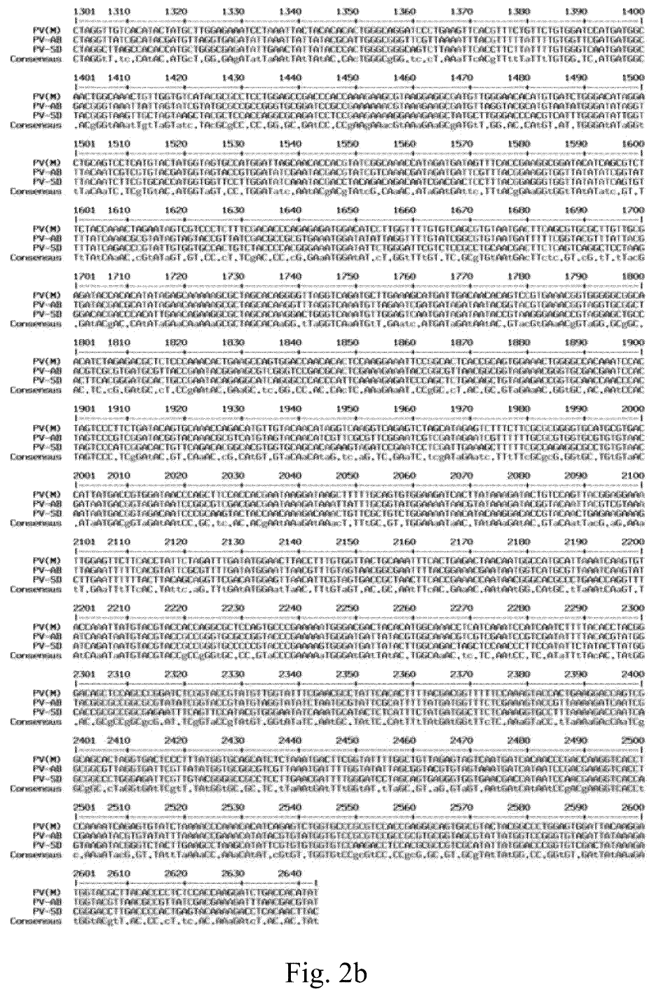

To address questions concerning the inviability of certain re-engineered viruses, sub-segments of the capsid region of virus PV-AB were subcloned into the wild type virus. See Example 1 and FIG. 3. Incorporating large subcloned segments (including non-overlapping segments) proved lethal, while small subcloned segments produced viable (with one exception) but sick viruses. “Sickness” is revealed by many assays: for example, segments of poor codon bias cause poor titers (FIG. 3B) and small plaques (FIGS. 3C-H). It is particularly instructive that in general, large, lethal segments can be divided into two sub-segments, both of which are alive but sick (FIG. 3). These results rule out the hypothesis that inviability is due to just one change; instead, at minimum, many changes must be contributing to the phenotype.

There is an exceptional segment from position 1513 to 2470. This segment is fairly small, but its inclusion in the PV genome causes inviability. It is not known at present whether or not this fragment can be subdivided into subfragments that merely cause sickness and do not inactivate the virus. It is conceivable that this segment does contain a highly deleterious change, possibly a translation frameshift site.

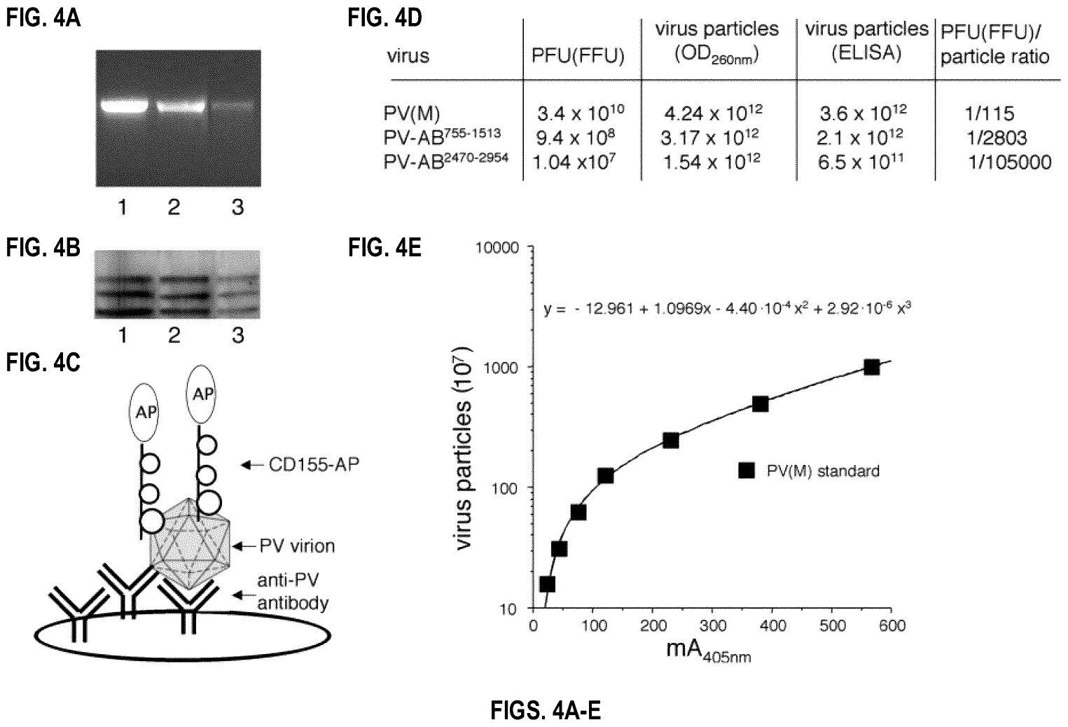

Since poor codon bias naturally suggests an effect on translation, translation of the proteins encoded by virus PV-AB was tested. See Example 5 and FIG. 5. Indeed, all the sick viruses translated capsid protein poorly (FIG. 5B). Translation was less efficient in the sicker viruses, consistent with poor translation being the cause of the sickness. Translation was improved essentially to wt levels in reactions that were supplemented with excess tRNAs and amino acids (FIG. 5A), consistent with the rate of recognition of rare codons being limiting.

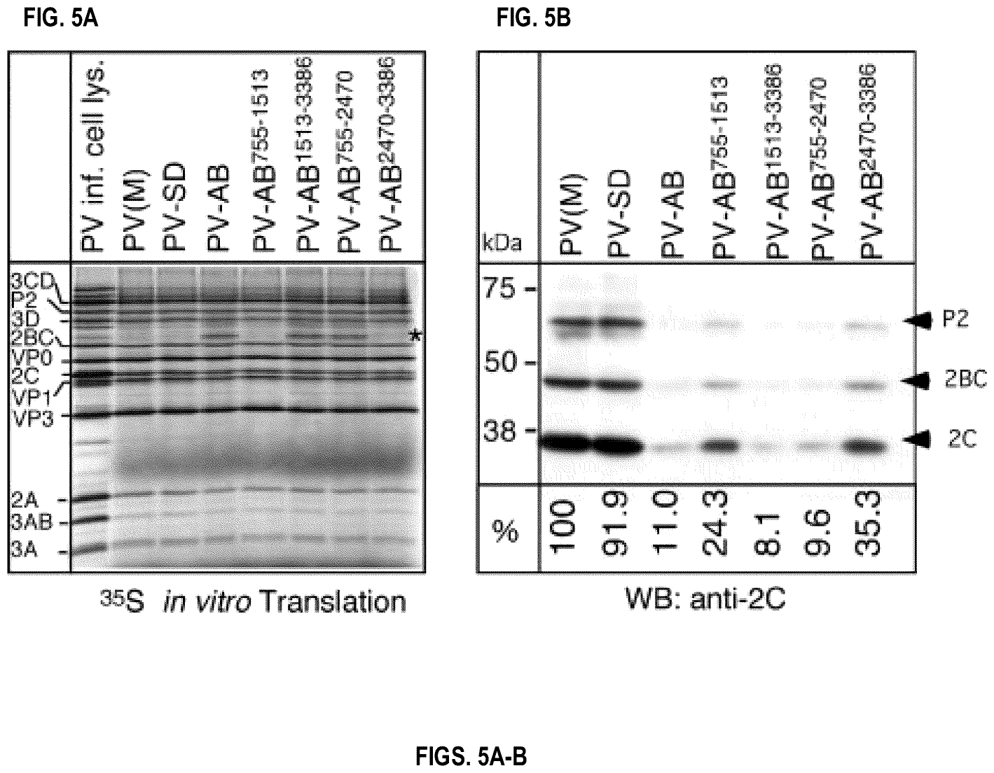

As a second test of whether deoptimized codon bias was causing inefficient translation, portions of wt and deoptimized capsid were fused to the N-terminus of firefly luciferase in a dicistronic reporter construct. See Example 5 and FIG. 6. In these fusion constructs, translation of luciferase depends on translation of the N-terminally fused capsid protein. Again, it was found that translation of the capsid proteins with deoptimized codons was poor, and was worse in the sicker viruses, suggesting a cause-and-effect relationship. Thus, the data suggest that the hundreds of rare codons in the PV-AB virus cause inviability largely because of poor translation. Further, the poor translation seen in vitro and the viral sickness seen in cultured cells are also reflected in infections of animals. Even for one of the least debilitated deoptimized viruses, PV-AB²⁴⁷⁰⁻²⁹⁵⁴, the number of viral particles needed to cause disease in mice was increased by about 100-fold. See Example 4, Table 4.

Burns et al. (2006) have recently described some similar experiments with the Sabin type 2 vaccine strain of PV and reached similar conclusions. Burns et al. synthesized a completely different codon-deoptimized virus (i.e., the nucleotide sequences of the PV-AB virus described herein and their “abcd” virus are very different), and yet got a similar degree of debilitation using similar assays. Burns et al. did not test their viral constructs in host organisms for attenuation. However, their result substantiates the view that SAVE is predictable, and that the results are not greatly dependent on the exact nucleotide sequence.

### Viral Attenuation by Deoptimizing Codon Pair Bias

According to the invention, codon pair bias can be altered independently of codon usage. For example, in a protein encoding sequence of interest, codon pair bias can be altered simply by directed rearrangement of its codons. In particular, the same codons that appear in the parent sequence, which can be of varying frequency in the host organism, are used in the altered sequence, but in different positions. In the simplest form, because the same codons are used as in the parent sequence, codon usage over the protein coding region being considered remains unchanged (as does the encoded amino acid sequence). Nevertheless, certain codons appear in new contexts, that is, preceded by and/or followed by codons that encode the same amino acid as in the parent sequence, but employing a different nucleotide triplet. Ideally, the rearrangement of codons results in codon pairs that are less frequent than in the parent sequence. In practice, rearranging codons often results in a less frequent codon pair at one location and a more frequent pair at a second location. By judicious rearrangement of codons, the codon pair usage bias over a given length of coding sequence can be reduced relative to the parent sequence. Alternatively, the codons could be rearranged so as to produce a sequence that makes use of codon pairs which are more frequent in the host than in the parent sequence.

Codon pair bias is evaluated by considering each codon pair in turn, scoring each pair according to the frequency that the codon pair is observed in protein coding sequences of the host, and then determining the codon pair bias for the sequence, as disclosed herein. It will be appreciated that one can create many different sequences that have the same codon pair bias. Also, codon pair bias can be altered to a greater or lesser extent, depending on the way in which codons are rearranged. The codon pair bias of a coding sequence can be altered by recoding the entire coding sequence, or by recoding one or more subsequences. As used herein, “codon pair bias” is evaluated over the length of a coding sequence, even though only a portion of the sequence may be mutated. Because codon pairs are scored in the context of codon usage of the host organism, a codon pair bias value can be assigned to wild type viral sequences and mutant viral sequences. According to the invention, a virus can be attenuated by recoding all or portions of the protein encoding sequences of the virus so a to reduce its codon pair bias.

According to the invention, codon pair bias is a quantitative property determined from codon pair usage of a host. Accordingly, absolute codon pair bias values may be determined for any given viral protein coding sequence. Alternatively, relative changes in codon pair bias values can be determined that relate a deoptimized viral protein coding sequence to a “parent” sequence from which it is derived. As viruses come in a variety of types (i.e., types I to VII by the Baltimore classification), and natural (i.e., virulent) isolates of different viruses yield different values of absolute codon pair bias, it is relative changes in codon pair bias that are usually more relevant to determining desired levels of attenuation. Accordingly, the invention provides attenuated viruses and methods of making such, wherein the attenuated viruses comprise viral genomes in which one or more protein encoding nucleotide sequences have codon pair bias reduced by mutation. In viruses that encode only a single protein (i.e., a polyprotein), all or part of the polyprotein can be mutated to a desired degree to reduce codon pair bias, and all or a portion of the mutated sequence can be provided in a recombinant viral construct. For a virus that separately encodes multiple proteins, one can reduce the codon pair bias of all of the protein encoding sequences simultaneously, or select only one or a few of the protein encoding sequences for modification. The reduction in codon pair bias is determined over the length of a protein encoding sequences, and is at least about 0.05, or at least about 0.1, or at least about 0.15, or at least about 0.2, or at least about 0.3, or at least about 0.4. Depending on the virus, the absolute codon pair bias, based on codon pair usage of the host, can be about −0.05 or less, or about −0.1 or less, or about −0.15 or less, or about −0.2 or less, or about −0.3 or less, or about −0.4 or less.

It will be apparent that codon pair bias can also be superimposed on other sequence variation. For example, a coding sequence can be altered both to encode a protein or polypeptide which contains one or more amino acid changes and also to have an altered codon pair bias. Also, in some cases, one may shuffle codons to maintain exactly the same codon usage profile in a codon-bias reduced protein encoding sequence as in a parent protein encoding sequence. This procedure highlights the power of codon pair bias changes, but need not be adhered to. Alternatively, codon selection can result in an overall change in codon usage is a coding sequence. In this regard, it is noted that in certain examples provided herein, (e.g., the design of PV-Min) even if the codon usage profile is not changed in the process of generating a codon pair bias minimized sequence, when a portion of that sequence is subcloned into an unmutated sequence (e.g., PV-MinXY or PV-MinZ), the codon usage profile over the subcloned portion, and in the hybrid produced, will not match the profile of the original unmutated protein coding sequence. However, these changes in codon usage profile have minimal effect of codon pair bias.

Similarly, it is noted that, by itself, changing a nucleotide sequence to encode a protein or polypeptide with one or many amino acid substitutions is also highly unlikely to produce a sequence with a significant change in codon pair bias. Consequently, codon pair bias alterations can be recognized even in nucleotide sequences that have been further modified to encode a mutated amino acid sequence. It is also noteworthy that mutations meant by themselves to increase codon bias are not likely to have more than a small effect on codon pair bias. For example, as disclosed herein, the codon pair bias for a poliovirus mutant recoded to maximize the use of nonpreferred codons (PV-AB) is decreased from wild type (PV-1(M)) by only about 0.05. Also noteworth is that such a protein encoding sequence have greatly diminished sequence diversity. To the contrary, substantial sequence diversity is maintained in codon pair bias modified sequences of the invention. Moreover, the significant reduction in codon pair bias obtainable without increased use of rare codons suggests that instead of maximizing the use of nonpreferred codons, as in PV-AB, it would be beneficial to rearrange nonpreferred codons with a sufficient number of preferred codons in order to more effectively reduce codon pair bias.

The extent and intensity of mutation can be varied depending on the length of the protein encoding nucleic acid, whether all or a portion can be mutated, and the desired reduction of codon pair bias. In an embodiment of the invention, a protein encoding sequence is modified over a length of at least about 100 nucleotide, or at least about 200 nucleotides, or at least about 300 nucleotides, or at least about 500 nucleotides, or at least about 1000 nucleotides.

As discussed above, the term “parent” virus or “parent” protein encoding sequence is used herein to refer to viral genomes and protein encoding sequences from which new sequences, which may be more or less attenuated, are derived. Accordingly, a parent virus can be a “wild type” or “naturally occurring” prototypes or isolate or variant or a mutant specifically created or selected on the basis of real or perceived desirable properties.

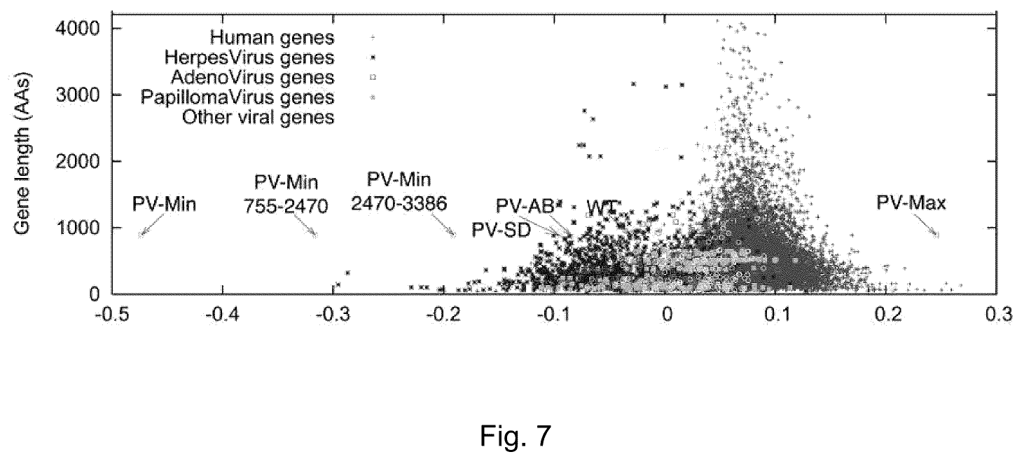

Using de novo DNA synthesis, the capsid coding region (the P1 region from nucleotide 755 to nucleotide 3385) of PV(M) was redesigned to introduce the largest possible number of rarely used codon pairs (virus PV-Min) (SEQ ID NO.4) or the largest possible number of frequently used codon pairs (virus PV-Max) (SEQ ID NO:5), while preserving the codon bias of the wild type virus. See Example 7. That is, the designed sequences use the same codons as the parent sequence, but they appear in a different order. The PV-Max virus exhibited one-step growth kinetics and killing of infected cells essentially identical to wild type virus. (That growth kinetics are not increased for a codon pair maximized virus relative to wild type appears to hold true for other viruses as well.) Conversely, cells transfected with PV-Min mutant RNA were not killed, and no viable virus could be recovered. Subcloning of fragments (PV-Min⁷⁵⁵⁻²⁴⁷⁰, PV-Min²⁴⁷⁰⁻³³⁸⁶) of the capsid region of PV-Min into the wt background produced very debilitated, but not dead, virus. See Example 7 and FIG. 8. This result substantiates the hypothesis that deleterious codon changes are preferably widely distributed and demonstrates the simplicity and effectiveness of varying the extent of the codon pair deoptimized sequence that is substituted into a wild type parent virus genome in order to vary the codon pair bias for the overall sequence and the attenuation of the viral product. As seen with PV-AB viruses, the phenotype of PV-Min viruses is a result of reduced specific infectivity of the viral particles rather than of lower production of progeny virus.

Virus with deoptimized codon pair bias are attenuated. As exemplified below, (see Example 8, and Table 5), CD155tg mice survived challenge by intracerebral injection of attenuated virus in amounts 1000-fold higher than would be lethal for wild type virus. These findings demonstrate the power of deoptimization of codon pair bias to minimize lethality of a virus. Further, the viability of the virus can be balanced with a reduction of infectivity by choosing the degree of codon pair bias deoptimization. Further, once a degree or ranges of degrees of codon pair bias deoptimization is determined that provides desired attenuation properties, additional sequences can be designed to attain that degree of codon pair bias. For example, SEQ ID NO:6 provides a poliovirus sequence with a codon pair bias of about −0.2, and mutations distributed over the region encompassing the mutated portions of PV-MinXY and PV-MinZ (i.e., PV⁷⁵⁵⁻³³⁸⁵)

### Algorithms for Sequence Design

The inventors have developed several novel algorithms for gene design that optimize the DNA sequence for particular desired properties while simultaneously coding for the given amino acid sequence. In particular, algorithms for maximizing or minimizing the desired RNA secondary structure in the sequence (Cohen and Skiena, 2003) as well as maximally adding and/or removing specified sets of patterns (Skiena, 2001), have been developed. The former issue arises in designing viable viruses, while the latter is useful to optimally insert restriction sites for technological reasons. The extent to which overlapping genes can be designed that simultaneously encode two or more genes in alternate reading frames has also been studied (Wang et al., 2006). This property of different functional polypeptides being encoded in different reading frames of a single nucleic acid is common in viruses and can be exploited for technological purposes such as weaving in antibiotic resistance genes.

The first generation of design tools for synthetic biology has been built, as described by Jayaraj et al. (2005) and Richardson et al. (2006). These focus primarily on optimizing designs for manufacturability (i.e., oligonucleotides without local secondary structures and end repeats) instead of optimizing sequences for biological activity. These first-generation tools may be viewed as analogous to the early VLSI CAD tools built around design rule-checking, instead of supporting higher-order design principles.

As exemplified herein, a computer-based algorithm can be used to manipulate the codon pair bias of any coding region. The algorithm has the ability to shuffle existing codons and to evaluate the resulting CPB, and then to reshuffle the sequence, optionally locking in particularly “valuable” codon pairs. The algorithm also employs a for of “simulated annealing” so as not to get stuck in local minima. Other parameters, such as the free energy of folding of RNA, may optional be under the control of the algorithm as well, in order to avoid creation of undesired secondary structures. The algorithm can be used to find a sequence with a minimum codon pair bias, and in the event that such a sequence does not provide a viable virus, the algorithm can be adjusted to find sequences with reduced, but not minimized biases. Of course, a viable viral sequence could also be produced using only a subsequence of the computer minimized sequence.

Whether or not performed with the aid of a computer, using, for example, a gradient descent, or simulated annealing, or other minimization routine. An example of the procedure that rearranges codons present in a starting sequence can be represented by the following steps:
1) Obtain wildtype viral genome sequence.
2) Select protein coding sequences to target for attenuated design.
3) Lock down known or conjectured DNA segments with non-coding functions.
4) Select desired codon distribution for remaining amino acids in redesigned proteins.
5) Perform random shuffle of unlocked codon positions and calculate codon-pair score.
6) Further reduce (or increase) codon-pair score optionally employing a simulated annealing procedure.
7) Inspect resulting design for excessive secondary structure and unwanted restriction site:
   if yes->go to step (5) or correct the design by replacing problematic regions with wildtype sequences and go to step (8).
8. Synthesize DNA sequence corresponding to virus design.
9. Create viral construct and assess expression:
if too attenuated, prepare subclone construct and goto 9;
if insufficiently attenuated, goto 2.

Source code (PERL script) of a computer based simulated annealing routine is provided.

Alternatively, one can devise a procedure which allows each pair of amino acids to be deoptimized by choosing a codon pair without a requirement that the codons be swapped out from elsewhere in the protein encoding sequence.

### Molecular Mechanisms of Viral Attenuation: Characterization of Attenuated PV Using High-Throughput Methods

As described above and in greater detail in the Examples, two synthetic, attenuated polioviruses encoding exactly the same proteins as the wildtype virus, but having altered codon bias or altered codon pair bias, were constructed. One virus uses deoptimized codons; the other virus uses deoptimized codon pairs. Each virus has many hundreds of nucleotide changes with respect to the wt virus.

The data presented herein suggest that these viruses are attenuated because of poor translation. This finding, if correct, has important consequences. First, the reduced fitness/virulence of each virus is due to small defects at hundreds of positions spread over the genome. Thus, there is essentially no chance of the virus reverting to wildtype, and so the virus is a good starting point for either a live or killed vaccine. Second, if the reduced fitness/virulence is due to additive effects of hundreds of small defects in translation, this method of reducing fitness with minimal risk of reversion should be applicable to many other viruses.

Though it is emphasized that the present invention is not limited to any particular mode of operation or underlying molecular mechanism, ongoing studies are aimed at distinguishing these alternative hypotheses. The ongoing investigations involve use of high throughput methods to scan through the genomes of various attenuated virus designs such as codon and codon pair deoptimized polioviurs and influenze virus, and to construct chimeras by placing overlapping 300-bp portions of each mutant virus into a wt context. See Example 11. The function of these chimeric viruses are then assayed. A finding that most chimeras are slightly, but not drastically, less fit than wild type, as suggested by the preliminary data disclosed herein, corroborates the “incremental loss of function” hypothesis, wherein many deleterious mutations are distributed throughout the regions covered by the chimeras. Conversely, a finding that most of the chimeras are similar or identical to wt, whereas one or only a few chimeras are attenuated like the parental mutant, suggests that there are relatively few positions in the sequence where mutation results in attenuation and that attenuation at those positions is significant.

As described in Example 12, experiments are performed to determine how codon and codon-pair deoptimization affect RNA stability and abundance, and to pinpoint the parameters that impair translation of the re-engineered viral genome. An understanding of the molecular basis of this impairment will further enhance the applicability of the SAVE approach to a broad range of viruses. Another conceivable mechanism underlying translation impairment is translational frameshifting, wherein the ribosome begins to translate a different reading frame, generating a spurious, typically truncated polypeptide up to the point where it encounters an in-frame stop codon. The PV genomes carrying the AB mutant segment from residue 1513 to 2470 are not only non-viable, but also produce a novel protein band during in vitro translation of approximately 42-44 kDa (see FIG. 5A). The ability of this AB¹⁵¹³⁻²⁴⁷⁰ fragment to inactivate PV, as well as its ability to induce production of the novel protein, may reflect the occurrence of a frameshift event and this possibility is also being investigated. A filter for avoiding the introduction of frameshifting sites is built into the SAVE design software.

More detailed investigations of translational defects are conducted using various techniques including, but not limited to, polysome profiling, toeprinting, and luciferase assays of fusion proteins, as described in Example 12.

### Molecular Biology of Poliovirus

While studies are ongoing to unravel the mechanisms underlying viral attenuation by SAVE, large-scale codon deoptimization of the PV capsid coding region revealed interesting insights into the biology of PV itself. What determines the PFU/particle ratio (specific infectivity) of a virus has been a longstanding question. In general, failure at any step during the infectious life cycle before the establishment of a productive infection will lead to an abortive infection and, therefore, to the demise of the infecting particle. In the case of PV, it has been shown that approximately 100 virions are required to result in one infectious event in cultured cells (Joklik and Darnell, 1961; Schwerdt and Fogh, 1957). That is, of 100 particles inoculated, only approximately one is likely to successfully complete all steps at the level of receptor binding (step 1), followed by internalization and uncoating (step 2), initiation of genome translation (step 3), polyprotein translation (step 4), RNA replication (step 5), and encapsidation of progeny (step 6).

In the infectious cycle of AB-type viruses described here, steps 1 and 2 should be identical to a PV(M) infection as their capsids are identical. Likewise, identical 5′ nontranslated regions should perform equally well in assembly of a translation complex (step 3). Viral polyprotein translation, on the other hand (step 4), is severely debilitated due to the introduction of a great number of suboptimal synonymous codons in the capsid region (FIGS. 5 and 6). It is thought that the repeated encounter of rare codons by the translational machinery causes stalling of the ribosome as, by the laws of mass action, rare aminoacyl-tRNA will take longer to diffuse into the A site on the ribosome. As peptide elongation to a large extent is driven by the concentration of available aminoacyl-tRNA, dependence of an mRNA on many rare tRNAs consequently lengthens the time of translation (Gustafsson et al., 2004). Alternatively, excessive stalling of the ribosome may cause premature dissociation of the translation complex from the RNA and result in a truncated protein destined for degradation. Both processes lead to a lower protein synthesis rate per mRNA molecule per unit of time. While the data presented herein suggest that the phenotypes of codon-deoptimized viruses are determined by the rate of genome translation, other mechanistic explanations may be possible. For example, it has been suggested that the conserved positions of rare synonymous codons throughout the viral capsid sequence in Hepatitis A virus are of functional importance for the proper folding of the nascent polypeptide by introducing necessary translation pauses (Sinchez et al., 2003). Accordingly, large-scale alteration of the codon composition may conceivably change some of these pause sites to result in an increase of misfolded capsid proteins.

Whether these considerations also apply to the PV capsid is not clear. If so, an altered phenotype would have been expected with the PV-SD design, in which the wt codons were preserved, but their positions throughout the capsid were completely changed. That is, none of the purported pause sites would be at the appropriate position with respect to the protein sequence. No change in phenotype, however, was observed and PV-SD translated and replicated at wild type levels (FIG. 3B).

Another possibility is that the large-scale codon alterations in the tested designs may create fortuitous dominant-negative RNA elements, such as stable secondary structures, or sequences that may undergo disruptive long-range interactions with other regions of the genome.

It is assumed that all steps prior to, and including, virus uncoating should be unchanged when wt and the mutant viruses, described herein are compared. This is supported by the observation that the eclipse period for all these isolates is similar (FIG. 3B). The dramatic reduction in PFU/particle ratio is, therefore, likely to be a result of the reduced translation capacity of the deoptimized genomes, i.e., the handicap of the mutant viruses is determined intracellularly.

It is generally assumed that the relatively low PFU/particle ratio of picornaviruses of 1/100 to 1/1,000 (Rueckert, 1985) is mainly determined by structural alterations at the receptor binding step, either prior to or at the level of cell entry. The formation of 135S particles that are hardly infectious may be the major culprit behind the inefficiency of poliovirus infectivity (Hogle, 2002). However, certain virus mutants seem to sidestep A particle conversion without resulting in a higher specific infectivity, an observation suggesting that other post-entry mechanisms may be responsible for the low PFU/particle ratio (Dove and Racaniello, 1997).

The present data provide clear evidence for such post-entry interactions between virus and cell, and suggest that these, and not pre-entry events, contribute to the distinct PFU/particle ratio of poliovirus. As all replication proteins in poliovirus are located downstream of P1 on the polyprotein, they critically depend upon successful completion of P1 translation. Lowering the rate of P1 translation therefore lowers translation of all replication proteins to the same extent. This, in turn, likely leads to a reduced capacity of the virus to make the necessary modifications to the host cell required for establishment of a productive infection, such as shutdown of host cell translation or prevention of host cell innate responses. While codon deoptimization, as described herein, is likely to effect translation at the peptide elongation step, reduced initiation of translation can also be a powerful attenuating determinant as well, as has been shown for mutations in the internal ribosomal entry site in the Sabin vaccine strains of poliovirus (Svitkin et al., 1993; 1985).

On the basis of these considerations, it is predicted that many mutant phenotypes attributable to defects in genome translation or early genome replication actually manifest themselves by lowering PFU/particle ratios. This would be the case as long as the defect results in an increased chance of abortive infection. Since in almost all studies the omnipresent plaque assay is the virus detection method of choice, a reduction in the apparent virus titer is often equated with a reduction in virus production per se. This may be an inherent pitfall that can be excused with the difficulties of characterizing virus properties at the single-cell level. Instead, most assays are done on a large population of cells. A lower readout of the chosen test (protein synthesis, RNA replication, virus production as measured in PFU) is taken at face value as an indicator of lower production on a per-cell basis, without considering that virus production in a cell may be normal while the number of cells producing virus is reduced.

The near-identical production of particles per cell by codon-deoptimized viruses indicates that the total of protein produced after extended period of times is not severely affected, whereas the rate of protein production has been drastically reduced. This is reflected in the delayed appearance of CPE, which may be a sign that the virus has to go through more RNA replication cycles to build up similar intracellular virus protein concentrations. It appears that codon-deoptimized viruses are severely handicapped in establishing a productive infection because the early translation rate of the incoming infecting genome is reduced. As a result of this lower translation rate, PV proteins essential for disabling the cell's antiviral responses (most likely proteinases 2A^{pro} and 3C^{pro}) are not synthesized at sufficient amounts to pass this crucial hurdle in the life cycle quickly enough. Consequently, there is a better chance for the cell to eliminate the infection before viral replication could unfold and take over the cell. Thus, the likelihood for productive infection events is reduced and the rate of abortive infection is increased. However, in the case where a codon-deoptimized virus does succeed in disabling the cell, this virus will produce nearly identical amounts of progeny to the wild type. The present data suggest that a fundamental difference may exist between early translation (from the incoming RNA genome) and late translation during the replicative phase, when the cell's own translation is largely shut down. Although this may be a general phenomenon, it might be especially important in the case of codon-deoptimized genomes. Host cell shutoff very likely results in an over-abundance of free aminoacyl-tRNAs, which may overcome the imposed effect of the suboptimal codon usage as the PV genomes no longer have to compete with cellular RNAs for translation resources. This, in fact, may be analogous to observations with the modified in vitro translation system described herein (FIG. 5B). Using a translation extract that was not nuclease-treated (and thus contained cellular mRNAs) and not supplemented with exogenous amino acids or tRNAs, clear differences were observed in the translation capacity of different capsid design mutants. Under these conditions, viral genomes have to compete with cellular mRNAs in an environment where supplies are limited. In contrast, in the traditional translation extract, in which endogenous mRNAs were removed and excess tRNAs and amino acids were added, all PV RNAs translated equally well regardless of codon bias (FIG. 5A). These two different in vitro conditions may be analogous to in vivo translation during the early and late phases in the PV-infected cell.

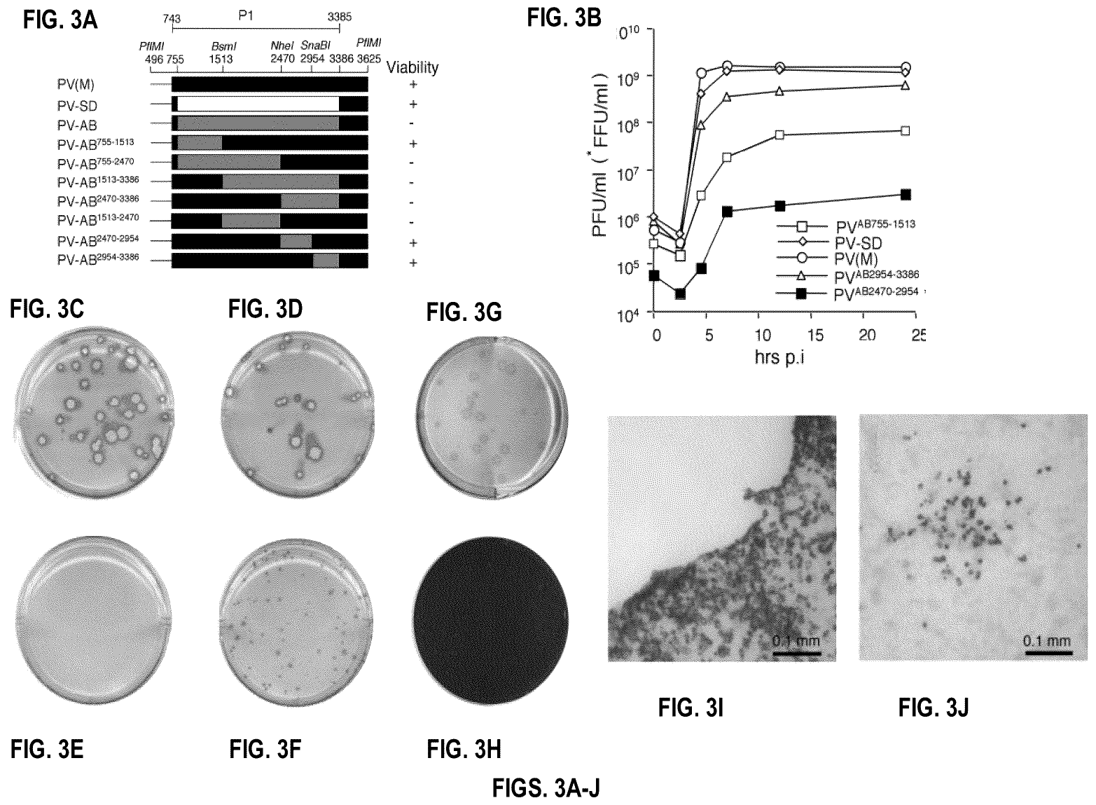

One key finding of the present study is the realization that, besides the steps during the physical interaction and uptake of virus, the PFU/particle ratio also largely reflects the virus' capacity to overcome host cell antiviral responses. This suggests that picornaviruses are actually quite inefficient in winning this struggle, and appear to have taken the path of evolving small genomes that can quickly replicate before the cell can effectively respond. As the data show, slowing down translation rates by only 30% in PV-AB²⁴⁷⁰⁻²⁹⁵⁴ (see FIG. 6) leads to a 1,000-fold higher rate of abortive infection as reflected in the lower specific infectivity (FIG. 4D). Picornaviruses apparently not only replicate at the threshold of error catastrophe (Crotty et al., 2001; Holland et al., 1990) but also at the threshold of elimination by the host cell's antiviral defenses. This effect may have profound consequences for the pathogenic phenotype of a picornavirus. The cellular antiviral processes responsible for the increased rate of aborted infections by codon-deoptimized viruses are not completely understood at present. PV has been shown to both induce and inhibit apoptosis (Belov et al., 2003; Girard et al., 1999; Tolskaya et al., 1995). Similarly PV interferes with the interferon pathway by cleaving NF-κB (Neznanov et al., 2005). It is plausible that a PV with a reduced rate of early genome translation still induces antiviral responses in the same way as a wt virus (induction of apoptosis and interferon by default) but then, due to low protein synthesis, has a reduced potential of inhibiting these processes. This scenario would increase the likelihood of the cell aborting a nascent infection and could explain the observed phenomena. At the individual cell level, PV infection is likely to be an all-or-nothing phenomenon. Viral protein and RNA syntheses likely need to be within a very close to maximal range in order to ensure productive infection.

### Attenuated Virus Vaccine Compositions

The present invention provides a vaccine composition for inducing a protective immune response in a subject comprising any of the attenuated viruses described herein and a pharmaceutically acceptable carrier.

It should be understood that an attenuated virus of the invention, where used to elicit a protective immune response in a subject or to prevent a subject from becoming afflicted with a virus-associated disease, is administered to the subject in the form of a composition additionally comprising a pharmaceutically acceptable carrier. Pharmaceutically acceptable carriers are well known to those skilled in the art and include, but are not limited to, one or more of 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments of the instant vaccine composition, the attenuated virus (i) does not substantially alter the synthesis and processing of viral proteins in an infected cell; (ii) produces similar amounts of virions per infected cell as wt virus; and/or (iii) exhibits substantially lower virion-specific infectivity than wt virus. In further embodiments, the attenuated virus induces a substantially similar immune response in a host animal as the corresponding wt virus.

This invention also provides a modified host cell line specially isolated or engineered to be permissive for an attenuated virus that is inviable in a wild type host cell. Since the attenuated virus cannot grow in normal (wild type) host cells, it is absolutely dependent on the specific helper cell line for growth. This provides a very high level of safety for the generation of virus for vaccine production. Various embodiments of the instant modified cell line permit the growth of an attenuated virus, wherein the genome of said cell line has been altered to increase the number of genes encoding rare tRNAs.

In preferred embodiments, the rare codons are CTA (coding for Leu), TCG (Ser), and CCG (Pro). In different embodiments, one, two, or all three of these rare codons are substituted for synonymous frequent codons in the viral genome. For example, all Leu codons in the virus may be changed to CTA; all Ser codons may be changed to TCG; all Pro codons may be changed to CCG; the Leu and Ser, or Leu and Pro, or Ser and Pro codons may be replaced by the identified rare codons; or all Leu, Ser, and Pro codons may be changed to CTA, TCG, and CCG, respectively, in a single virus. Further, a fraction of the relevant codons, i.e., less than 100%, may be changed to the rare codons. Thus, the proportion of codons substituted may be about 20%, 40%, 60%, 80% or 100% of the total number of codons.

In certain embodiments, these substitutions are made only in the capsid region of the virus, where a high rate of translation is most important. In other embodiments, the substitutions are made throughout the virus. In further embodiments, the cell line overexpresses tRNAs that bind to the rare codons.

This invention further provides a method of synthesizing any of the attenuated viruses described herein, the method comprising (a) identifying codons in multiple locations within at least one non-regulatory portion of the viral genome, which codons can be replaced by synonymous codons; (b) selecting a synonymous codon to be substituted for each of the identified codons; and (c) substituting a synonymous codon for each of the identified codons.

In certain embodiments of the instant methods, steps (a) and (b) are guided by a computer-based algorithm for Synthetic Attenuated Virus Engineering (SAVE) that permits design of a viral genome by varying specified pattern sets of deoptimized codon distribution and/or deoptimized codon-pair distribution within preferred limits. The invention also provides a method wherein, the pattern sets alternatively or additionally comprise, density of deoptimized codons and deoptimized codon pairs, RNA secondary structure, CpG dinucleotide content, C+G content, overlapping coding frames, restriction site distribution, frameshift sites, or any combination thereof.

In other embodiments, step (c) is achieved by de novo synthesis of DNA containing the synonymous codons and/or codon pairs and substitution of the corresponding region of the genome with the synthesized DNA. In further embodiments, the entire genome is substituted with the synthesized DNA. In still further embodiments, a portion of the genome is substituted with the synthesized DNA. In yet other embodiments, said portion of the genome is the capsid coding region.

In addition, the present invention provides a method for eliciting a protective immune response in a subject comprising administering to the subject a prophylactically or therapeutically effective dose of any of the vaccine compositions described herein. This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art, and is usually on the order of several weeks to months. The present invention is not limited, however, to any particular method, route or frequency of administration.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, and CD155tg transgenic mice expressing the human poliovirus receptor CD155. In a preferred embodiment, the subject is a human. Preferred embodiments of birds are domesticated poultry species, including, but not limited to, chickens, turkeys, ducks, and geese.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold. For example, if a subject has a 1% chance of becoming infected with a virus, a two-fold reduction in the likelihood of the subject becoming infected with the virus would result in the subject having a 0.5% chance of becoming infected with the virus. Most preferably, a “prophylactically effective dose” induces in the subject an immune response that completely prevents the subject from becoming infected by the virus or prevents the onset of the disorder in the subject entirely.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and an instructional material for the use thereof. In further embodiments, the attenuated virus may be one or more poliovirus, one or more rhinovirus, one or more influenza virus, etc. More than one virus may be preferred where it is desirable to immunize a host against a number of different isolates of a particular virus. The invention includes other embodiments of kits that are known to those skilled in the art. The instructions can provide any information that is useful for directing the administration of the attenuated viruses.

Of course, it is to be understood and expected that variations in the principles of invention herein disclosed can be made by one skilled in the art and it is intended that such modifications are to be included within the scope of the present invention. The following Examples further illustrate the invention, but should not be construed to limit the scope of the invention in any way. Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994). All references mentioned herein are incorporated in their entirety by reference into this application.

Full details for the various publications cited throughout this application are provided at the end of the specification immediately preceding the claims. The disclosures of these publications are hereby incorporated in their entireties by reference into this application. However, the citation of a reference herein should not be construed as an acknowledgement that such reference is prior art to the present invention.

### Example 1

### Re-Engineering of Capsid Region of Polioviruses by Altering Codon Bias

### Cells, Viruses, Plasmids, and Bacteria

HeLa R19 cell monolayers were maintained in Dulbecco's modified Eagle medium (DMEM) supplemented with 10% bovine calf serum (BCS) at 37° C. All PV infectious cDNA constructs are based on PV1(M) cDNA clone pT7PVM (Cao et al., 1993; van der Werf et al., 1986). Dicistronic reporter plasmids were constructed using pHRPF-Luc (Zhao and Wimmer, 2001). Escherichia coli DH5a was used for plasmid transformation and propagation. Viruses were amplified by infection of HeLa R19 cell monolayers with 5 PFU per cell. Infected cells were incubated in DMEM (2% BCS) at 37° C. until complete cytopathic effect (CPE) was apparent or for at least 4 days post-infection. After three rounds of freezing and thawing, the lysate was clarified of cell debris by low-speed centrifugation and the supernatant, containing the virus, was used for further passaging or analysis.

### Cloning of Synthetic Capsid Replacements and Dicistronic Reporter Replicons

Two PV genome cDNA fragments spanning the genome between nucleotides 495 and 3636, named SD and AB, were synthesized using GeneMaker® technology (Blue Heron Biotechnology). pPV-SD and pPV-AB were generated by releasing the replacement cassettes from the vendor's cloning vector by PflMI digestion and insertion into the pT7PVM vector in which the corresponding PflMI fragment had been removed. pPV-AB⁷⁵⁵⁻¹⁵¹³ and pPV-AB²⁴⁷⁰⁻³³⁸⁶ were obtained by inserting a BsmI fragment or an NheI-EcoRI fragment, respectively, from pPV-AB into equally digested pT7PVM vector. In pPV-AB¹⁵¹³⁻³³⁸⁶ and pPV-AB⁷⁵⁵⁻²⁴⁷⁰, the BsmI fragment or NheI-EcoRI fragment of pT7PVM, respectively, replaces the respective fragment of the pPV-AB vector. Replacement of the NheI-EcoRI fragment of pPV-AB¹⁵¹³⁻³³⁸⁶ with that of pT7PVM resulted in pPV-AB²⁴⁷⁰⁻³³⁸⁶. Finally, replacement of the SnaBI-EcoRI fragments of pPV-AB²⁴⁷⁰⁻³³⁸⁶ and pT7PVM with one another produced pPV-AB²⁹⁵⁴⁻³³⁸⁶ and pPV-AB²⁴⁷⁰⁻²⁹⁵⁴, respectively.

Cloning of dicistronic reporter constructs was accomplished by first introducing a silent mutation in pHRPF-Luc by site-directed mutagenesis using oligonucleotides Fluc-mutRI(+)/Fluc-mutRI(−) to mutate an EcoRI site in the firefly luciferase open reading frame and generate pdiLuc-mRI. The capsid regions of pT7PVM, pPV-AB¹⁵¹³⁻²⁴⁷⁰, and pPV-AB²⁴⁷⁰⁻²⁹⁵⁴ were PCR amplified using oligonucleotides RI-2A-P1wt(+)/P1wt-2A-RI(−). Capsid sequences of pPV-AB²⁴⁷⁰⁻³³⁸⁶ and pPV-AB²⁹⁵⁴⁻³³⁸⁶ or pPV-AB were amplified with RI-2A-P1wt(+)/P1AB-2A-RI(−) or RI-2A-P1AB(+)/P1AB-2A-RI(−), respectively. PCR products were digested with EcoRI and inserted into a now unique EcoRI site in pdiLuc-mRI to result in pdiLuc-PV, pdiLuc-AB¹⁵¹³⁻²⁴⁷⁰, pdiLuc-AB²⁴⁷⁰⁻²⁹⁵⁴ pdiLuc-AB²⁴⁷⁰⁻³³⁸⁶, pdiLuc-AB²⁹⁵⁴⁻³³⁸⁶, and pdiLuc-AB, respectively.

### Oligonucleotides

The following oligonucleotides were used:

(SEQ ID NO: 6)
Fluc-mutRI(+), 5′-GCACTGATAATGAACTCCTCTGGATCTACTGG-3′;
(SEQ ID NO: 7)
Fluc-mutRI(−), 5′-CCAGTAGATCCAGAGGAGTTCATTATCAGTGC-3′;
(SEQ ID NO: 8)
RI-2A-P1wt(+), 5′-CAAGAATTCCTGACCACATACGGTGCTCAGGTTTCATCACAGAAAGTGGG-3′;
(SEQ ID NO: 9)
RI-2A-P1AB(+), 5′-CAAGAATTCCTGACCACATACGGTGCGCAAGTATCGTCGCAAAAAGTAGG-3;
(SEQ ID NO: 10)
P1wt-2A-RI(−), 5′-TTCGAATTCTCCATATGTGGTCAGATCCTTGGTGG-AGAGG-3′;
and
(SEQ ID NO: 11)
P1AB-2A-RI(−), 5′-TTCGAATTCTCCATACGTCGTTAAATCTTTCGTCGATAACG-3′.

### In Vitro Transcription and RNA Transfection

Driven by the T7 promoter, 2 μg of EcoRI-linearized plasmid DNA were transcribed by T7 RNA polymerase (Stratagene) for 1 h at 37° C. One microgram of virus or dicistronic transcript RNA was used to transfect 10⁶ HeLa R19 cells on a 35-mm-diameter plate according to a modification of the DEAE-dextran method (van der Werf et al., 1986). Following a 30-min incubation at room temperature, the supernatant was removed and cells were incubated at 37° C. in 2 ml of DMEM containing 2% BCS until CPE appeared, or the cells were frozen 4 days post-transfection for further passaging. Virus titers were determined by standard plaque assay on HeLa R19 cells using a semisolid overlay of 0.6% tragacanth gum (Sigma-Aldrich) in minimal Eagle medium.

### Design and Synthesis of Codon-Deoptimized Polioviruses

Two different synonymous encodings of the poliovirus P1 capsid region were produced, each governed by different design criteria. The designs were limited to the capsid, as it has been conclusively shown that the entire capsid coding sequence can be deleted from the PV genome or replaced with exogenous sequences without affecting replication of the resulting sub-genomic replicon (Johansen and Morrow, 2000; Kaplan and Racaniello, 1988). It is therefore quite certain that no unidentified crucial regulatory RNA elements are located in the capsid region, which might be affected inadvertently by modulation of the RNA sequence.

The first design (PV-SD) sought to maximize the number of RNA base changes while preserving the exact codon usage distribution of the wild type P1 region (FIG. 1). To achieve this, synonymous codon positions were exchanged for each amino acid by finding a maximum weight bipartite match (Gabow, 1973) between the positions and the codons, where the weight of each position-codon pair is the number of base changes between the original codon and the synonymous candidate codon to replace it. To avoid any positional bias from the matching algorithm, the synonymous codon locations were randomly permuted before creating the input graph and the locations were subsequently restored. Rothberg's maximum bipartite matching program (Rothberg, 1985) was used to compute the matching. A total of 11 useful restriction enzyme sites, each 6 nucleotides, were locked in the viral genome sequence so as to not participate in the codon location exchange. The codon shuffling technique potentially creates additional restriction sites that should preferably remain unique in the resulting reconstituted full-length genome. For this reason, the sequence was further processed by substituting codons to eliminate the undesired sites. This resulted in an additional nine synonymous codon changes that slightly altered the codon frequency distribution. However, no codon had its frequency changed by more than 1 over the wild type sequence. In total, there were 934 out of 2,643 nucleotides changed in the PV-SD capsid design when compared to the wt P1 sequence while maintaining the identical protein sequence of the capsid coding domain (see FIGS. 1 and 2). As the codon usage was not changed, the GC content in the PVM-SD capsid coding sequence remained identical to that in the wt at 49%.

The second design, PV-AB, sought to drastically change the codon usage distribution over the wt P1 region. This design was influenced by recent work suggesting that codon bias may impact tissue-specific expression (Plotkin et al., 2004). The desired codon usage distribution was derived from the most unfavorable codons observed in a previously described set of brain-specific genes (Hsiao et al., 2001; Plotkin et al., 2004). A capsid coding region was synthesized maximizing the usage of the rarest synonymous codon for each particular amino acid as observed in this set of genes (FIG. 1). Since for all amino acids but one (Leu) the rarest codon in brain corresponds to the rarest codons among all human genes at large, in effect this design would be expected to discriminate against expression in other human tissues as well. Altogether, the PV-AB capsid differs from the wt capsid in 680 nucleotide positions (see FIG. 2). The GC content in the PVM-AB capsid region was reduced to 43% compared to 49% in the wt.

### Example 2

### Effects of Codon-Deoptimization on Growth and Infectivity of Polioviruses

### Determination of Virus Titer by Infected Focus Assay

Infections were done as for a standard plaque assay. After 48 or 72 h of incubation, the tragacanth gum overlay was removed and the wells were washed twice with phosphate-buffered saline (PBS) and fixed with cold methanol/acetone for 30 min. Wells were blocked in PBS containing 10% BCS followed by incubation with a 1:20 dilution of anti-3D mouse monoclonal antibody 125.2.3 (Paul et al., 1998) for 1 h at 37° C. After washing, cells were incubated with horseradish peroxidase-labeled goat anti-mouse antibody (Jackson ImmunoResearch, West Grove, PA) and infected cells were visualized using Vector VIP substrate kit (Vector Laboratories, Burlingame, CA). Stained foci, which are equivalent to plaques obtained with wt virus, were counted, and titers were calculated as in the plaque assay procedure.

### Codon-Deoptimized Polioviruses Display Severe Growth Phenotypes

Of the two initial capsid ORF replacement designs (FIG. 3A), only PV-SD produced viable virus. In contrast, no viable virus was recovered from four independent transfections with PV-AB RNA, even after three rounds of passaging (FIG. 3E). It appeared that the codon bias introduced into the PV-AB genome was too severe. Thus, smaller portions of the PV-AB capsid coding sequence were subcloned into the PV(M) background to reduce the detrimental effects of the nonpreferred codons. Of these subclones, PV-AB²⁹⁵⁴⁻³³⁸⁶ produced CPE 40 h after RNA transfection, while PV-AB⁷⁵⁵⁻¹⁵¹³ and PV-AB²⁴⁷⁰⁻²⁹⁵⁴ required one or two additional passages following transfection, respectively (compared to 24 h for the wild type virus). Interestingly, these chimeric viruses represent the three subclones with the smallest portions of the original AB sequence, an observation suggesting a direct correlation between the number of nonpreferred codons and the fitness of the virus.

One-step growth kinetics of all viable virus variants were determined by infecting HeLa monolayers at a multiplicity of infection (MOI) of 2 with viral cell lysates obtained after a maximum of two passages following RNA transfection (FIG. 3B). The MOI was chosen due to the low titer of PV-AB²⁴⁷⁰⁻²⁹⁵⁴ and to eliminate the need for further passaging required for concentrating and purifying the inoculum. Under the conditions used, all viruses had produced complete or near complete CPE by 24 h post-infection.

Despite 934 single-point mutations in its capsid region, PV-SD replicated at wt capacity (FIG. 3B) and produced similarly sized plaques as the wt (FIG. 3D). While PV-AB²⁹⁵⁴⁻³³⁸⁶ grew with near-wild type kinetics (FIG. 3B), PV-AB⁷⁵⁵⁻¹⁵¹³ produced minute plaques and approximately 22-fold less infectious virus (FIGS. 2. 3B and F, respectively). Although able to cause CPE in high-MOI infections, albeit much delayed (80 to 90% CPE after 20 to 24 h), PV-AB²⁴⁷⁰⁻²⁹⁵⁴ produced no plaques at all under the conditions of the standard plaque assay (FIG. 3H). This virus was therefore quantified using a focus-forming assay, in which foci of infected cells after 72 h of incubation under plaque assay conditions were counted after they were stained immunohistochemically with antibodies to the viral polymerase 3D (FIG. 3G). After 48 h of infection, PV-AB²⁴⁷⁰⁻²⁹⁵⁴-infected foci usually involved only tens to hundreds of cells (FIG. 3J) with a focus diameter of 0.2 to 0.5 mm, compared to 3-mm plaques for the wt (FIGS. 3C and D). However, after an additional 24 h, the diameter of the foci increased significantly (2 to 3 mm; FIG. 3G). When HeLa cells were infected with PV-AB⁷⁵⁵⁻¹⁵¹³ and PV-AB²⁴⁷⁰⁻²⁹⁵⁴ at an MOI of 1, the CPE appeared between 12 and 18 h and 3 and 4 days, respectively, compared to 8 h with the wt(data not shown).

In order to quantify the cumulative effect of a particular codon bias in a protein coding sequence, a relative codon deoptimization index (RCDI) was calculated, which is a comparative measure against the general codon distribution in the human genome. An RCDI of 1/codon indicates that a gene follows the normal human codon frequencies, while any deviation from the normal human codon bias results in an RCDI higher than 1. The RCDI was derived using the formula:

$$RCDI = [ \sum {C}_{i} {F}_{a} / {C}_{i} {F}_{h} ) \cdot {N}_{ci} ] / N ( i = 1 through 64 ) .$$

CᵢFₐ is the observed relative frequency in the test sequence of each codon i out of all synonymous codons for the same amino acid (0 to 1); CᵢFₕ is the normal relative frequency observed in the human genome of each codon i out of all synonymous codons for that amino acid (0.06 to 1); N_{ci} is the number of occurrences of that codon i in the sequence; and Nis the total number of codons (amino acids) in the sequence.

Thus, a high number of rare codons in a sequence results in a higher index. Using this formula, the RCDI values of the various capsid coding sequences were calculated to be 1.14 for PV(M) and PV-SD which is very close to a normal human distribution. The RCDI values for the AB constructs are 1.73 for PV-AB⁷⁵⁵⁻¹⁵¹³, 1.45 for PV-AB²⁴⁷⁰⁻²⁹⁵⁴, and 6.51 for the parental PV-AB. For comparison, the RCDI for probably the best known codon-optimized protein, “humanized” green fluorescent protein (GFP), was 1.31 compared to an RCDI of 1.68 for the original Aequorea victoria gfp gene (Zolotukhin et al., 1996). According to these calculations, a capsid coding sequence with an RCDI of <2 is associated with a viable virus phenotype, while an RCDI of >2 (PV-AB=6.51, PV-AB¹⁵¹³⁻³³⁸⁶=4.04, PV-AB⁷⁵⁵⁻²⁴⁷⁰=3.61) results in a lethal phenotype.

### Example 3

### Effects of Codon-Deoptimization on Specific Infectivity of Polioviruses

### Molecular Quantification of Viral Particles: Direct OD₂₆₀ Absorbance Method

Fifteen-centimeter dishes of HeLa cells (4×10⁷ cells) were infected with PV(M), PV-AB⁷⁵⁵⁻¹⁵¹³, or PV-AB²⁴⁷⁰⁻²⁹⁵⁴ at an MOI of 0.5 until complete CPE occurred (overnight versus 4 days). Cell-associated virus was released by three successive freeze/thaw cycles. Cell lysates were cleared by 10 min of centrifugation at 2,000×g followed by a second 10-min centrifugation at 14,000×g for 10 min. Supernatants were incubated for 1 h at room temperature in the presence of 10 μg/ml RNase A (Roche) to digest any extraviral or cellular RNA. After addition of 0.5% sodium dodecyl sulfate (SDS) and 2 mM EDTA, virus-containing supernatants were overlaid on a 6-ml sucrose cushion (30% sucrose in Hanks balanced salt solution [HBSS]; Invitrogen, Carlsbad, CA). Virus particles were sedimented by ultracentrifugation for 4 h at 28,000 rpm using an SW28 swinging bucket rotor. Supernatants were discarded and centrifuge tubes were rinsed twice with HBSS while leaving the sucrose cushion intact. After removal of the last wash and the sucrose cushion, virus pellets were resuspended in PBS containing 0.2% SDS and 5 mM EDTA. Virus infectious titers were determined by plaque assay/infected-focus assay (see above). Virus particle concentrations were determined with a NanoDrop spectrophotometer (NanoDrop Technologies, Inc., Wilmington, DE) at the optical density at 260 nm (OD₂₆₀) and calculated using the formula 1 OD₂₆₀ unit=9.4×10¹² particles/ml (Rueckert, 1985). In addition, virion RNA was extracted by three rounds of phenol extraction and one round of chloroform extraction. RNA was ethanol precipitated and resuspended in ultrapure water. RNA purity was confirmed by TAE-buffered agarose gel analysis, and the concentration was determined spectrophotometrically. The total number of genome equivalents of the corresponding virus preparation was calculated via the determined RNA concentration and the molecular weight of the RNA. Thus, the relative amount of virions per infectious units could be calculated, assuming that one RNase-protected viral genome equivalent corresponds to one virus particle.

### Molecular Quantification of Viral Particles: ELISA Method

Nunc Maxisorb 96-well plates were coated with 10 μg of rabbit anti-PV(M) antibody (Murdin and Wimmer, 1989) in 100 μl PBS for 2 h at 37° C. and an additional 14 h at 4° C., and then the plates were washed three times briefly with 350 μl of PBS and blocked with 350 μl of 10% bovine calf serum in PBS for 1 h at 37° C. Following three brief washes with PBS, wells were incubated with 100 μl of virus-containing cell lysates or controls in DMEM plus 2% BCS for 4 h at room temperature. Wells were washed with 350 μl of PBS three times for 5 min each. Wells were then incubated for 4 h at room temperature with 2 μg of CD155-alkaline phosphatase (AP) fusion protein (He et al., 2000) in 100 μl of DMEM-10% BCS. After the last of five washes with PBS, 100 μl of 10 mM Tris, pH 7.5, were added and plates were incubated for 1 h at 65° C. Colorimetric alkaline phosphatase determination was accomplished by addition of 100 μl of 9 mg/ml para-nitrophenylphosphate (in 2 M diethanolamine, 1 mM MgCl₂, pH 9.8). Alkaline phosphatase activity was determined, and virus particle concentrations were calculated in an enzyme-linked immunosorbent assay (ELISA) plate reader (Molecular Devices, Sunnyvale, CA) at a 405-nm wavelength on a standard curve prepared in parallel using two-fold serial dilutions of a known concentration of purified PV(M) virus stock.

### The PFU/Particle Ratio is Reduced in Codon-Deoptimized Viruses

The extremely poor growth phenotype of PV-AB²⁴⁷⁰⁻²⁹⁵⁴ in cell culture and its inability to form plaques suggested a defect in cell-to-cell spreading that may be consistent with a lower specific infectivity of the individual virus particles.

To test this hypothesis, PV(M), PV-AB⁷⁵⁵⁻¹⁵¹³, and PV-AB²⁴⁷⁰⁻²⁹⁵⁴ virus were purified and the amount of virus particles was determined spectrophotometrically. Purified virus preparations were quantified directly by measuring the OD₂₆₀, and particle concentrations were calculated according to the formula 1 OD₂₆₀ unit=9.4×10¹² particles/ml (FIG. 4D) (Rueckert, 1985). Additionally, genomic RNA was extracted from those virions (FIG. 4A) and quantified at OD₂₆₀ (data not shown). The number of virions (1 virion=1 genome) was then determined via the molecular size of 2.53×10⁶ g/mol for genomic RNA. Specifically, virus was prepared from 4×10⁷ HeLa cells that were infected with 0.5 MOI of virus until the appearance of complete CPE, as described above. Both methods of particle determinations produced similar results (FIG. 4D). Indeed, it was found that PV(M) and PV-AB⁷⁵⁵⁻¹⁵¹³ produced roughly equal amounts of virions, while PV-AB²⁴⁷⁰⁻²⁹⁵⁴ produced between ⅓ (by the direct UV method (FIG. 4D) to ⅛ of the number of virions compared to PV(M) (by genomic RNA method [data not shown]). In contrast, the wt virus sample corresponded to approximately 30 times and 3,000 times more infectious units than PV-AB⁷⁵⁵⁻¹⁵¹³ and PV-AB²⁴⁷⁰⁻²⁹⁵⁴, respectively (FIG. 4D). In addition, capsid proteins of purified virions were resolved by SDS-polyacrylamide gel electrophoresis (PAGE) and visualized by silver staining (FIG. 4B). These data also support the conclusion that on a per-cell basis, PV-AB²⁴⁷⁰⁻²⁹⁵⁴ and PV-AB⁷⁵⁵⁻¹⁵¹³ produce similar or only slightly reduced amounts of progeny per cell (FIG. 4B, lane 3), while their PFU/particle ratio is reduced. The PFU/particle ratio for a virus can vary significantly depending on the methods to determine either plaques (cell type for plaque assay and the particular plaque assay technique) or particle count (spectrophotometry or electron microscopy). A PFU/particle ratio of 1/115 for PV1(M) was determined using the method described herein, which compares well to previous determinations of 1/272 (Joklik and Darnell, 1961) (done on HeLa cells) and 1/87 (Schwerdt and Fogh, 1957) (in primary monkey kidney cells).

### Development of a Virion-Specific ELISA

To confirm the reduced PFU/particle ratio observed with codon-deoptimized polioviruses, a novel virion-specific ELISA was developed (FIGS. 4C and E) as a way to determine the physical amount of intact viral particles in a sample rather than the infectious titer, which is a biological variable. The assay is based on a previous observation that the ectodomain of the PV receptor CD155 fused to heat-stable placental alkaline phosphatase (CD155-AP) binds very tightly and specifically to the intact 160S particle (He et al., 2000). Considering that PV 130S particles (A particles) lose their ability to bind CD155 efficiently (Hogle, 2002), it is expected that no other capsid intermediate or capsid subunits would interact with CD155-AP, thus ensuring specificity for intact particles. In support of this notion, lysates from cells that were infected with a vaccinia virus strain expressing the P1 capsid precursor (Ansardi et al., 1993) resulted in no quantifiable signal (data not shown).

The ELISA method allows for the quantification of virus particles in a crude sample such as the cell lysate after infection, which should minimize possible alteration of the PFU/particle ratio by other mechanisms during sample handling and purification (thermal/chemical inactivation, oxidation, degradation, etc.). Under the current conditions, the sensitivity of this assay is approximately 10⁷ viral particles, as there is no signal amplification step involved. This, in turn, resulted in an exceptionally low background. With this ELISA, PV particle concentrations could be determined in samples by back calculation on a standard curve prepared with purified PV(M) of known concentration (FIG. 4E). The particle determinations by ELISA agreed well with results obtained by the direct UV method (FIG. 4D).

### Implications of Results

The present study has demonstrated the utility of large-scale codon deoptimization of PV capsid coding sequences by de novo gene synthesis for the generation of attenuated viruses. The initial goal was to explore the potential of this technology as a tool for generating live attenuated virus vaccines. Codon-deoptimized viruses were found to have very low specific infectivity (FIG. 4). The low specific infectivity (that is the chance of a single virus particle to successfully initiate an infectious cycle in a cell) results in a more slowly spreading virus infection within the host. This in turn allows the host organism more time to mount an immune response and clear the infection, which is a most desirable feature in an attenuated virus vaccine. On the other hand, codon-deoptimized viruses generated similar amounts of progeny per cell as compared the wild type virus, while being 2 to 3 orders of magnitude less infectious (FIG. 4). This allows the production of virus particles antigenically indistinguishable from the wt as effectively and cost-efficiently as the production of the wt virus itself. However due to the low specific infectivity the actual handling and processing of such a virus preparation is much safer. Since, there are increasing concerns about the production of virulent virus in sufficient quantities under high containment conditions and the associated risk of virus escape from the production facility either by accident or by malicious intent.viruses as described herein may prove very useful as safer alternatives in the production of inactivated virus vaccines. Since they are 100% identical to the wt virus at the protein level, an identical immune response in hosts who received inactivated virus is guaranteed.

### Example 4

### Effects of Codon-Deoptimization on Neuropathogenicity of Polioviruses

### Mouse Neuropathogenicity Tests

Groups of four to five CD155tg mice (strain Tg21) (Koike et al., 1991) between 6 and 8 weeks of age were injected intracerebrally with virus dilutions between 10² and 10⁶ PFU/focus-forming units (FFU) in 30 μl PBS. Fifty percent lethal dose (LD₅₀) values were calculated by the method of Reed and Muench (1938). Virus titers in spinal cord tissues at the time of death or paralysis were determined by plaque or infected-focus assay.

### Codon-Deoptimized Polioviruses are Neuroattenuated on a Particle Basis in CD155tg Mice

To test the pathogenic potential of viruses constructed in this study, CD155 transgenic mice (Koike et al., 1991) were injected intracerebrally with PV(M), PV-SD, PV-AB⁷⁵⁵⁻¹⁵¹³ and PV-AB²⁴⁷⁰⁻²⁹⁵⁴ at doses between 10² and 10⁵ PFU/FFU. Initial results were perplexing, as quite counterintuitively PV-AB⁷⁵⁵⁻¹⁵¹³ and especially PV-AB²⁴⁷⁰⁻²⁹⁵⁴ were initially found to be as neuropathogenic as, or even slightly more neuropathogenic, than the wt virus. See Table 4.

TABLE 4
Neuropathogenicity in CD155tg mice.

| | LD₅₀ | | Spinal cord titer | |
|---|---|---|---|---|
| Construct | PFU or FFU^{a} | No. of virions^{b} | PFU or FFU/g^{c} | No. of virions/g^{d} |
| PV(M) wt | 3.2 × 10² PFU | 3.7 × 10⁴ | 1.0 × 10⁹ PFU | 1.15 × 10¹¹ |
| PV-AB⁷⁵⁵⁻¹⁵¹⁵ | 2.6 × 10² PFU | 7.3 × 10⁵ | 3.5 × 10⁷ PFU | 9.8 × 10¹⁰ |
| PV-AB²⁴⁷⁰⁻²⁹⁵⁴ | 4.6 × 10² PFU | 4.8 × 10⁶ | 3.4 × 10⁶ FFU | 3.57 × 10¹¹ |

^{a}LD₅₀ expressed as the number of infectious units, as determined by plaque or infectious focus assay, that results in 50% lethality after intracerebral inoculation.
^{b}LD₅₀ expressed as the number of virus particles, as determined by OD₂₆₀ measurement, that results in 50% lethality after intracerebral inoculation.
^{c}Virus recovered from the spinal cord of infected mice at the time of death or paralysis; expressed in PFU or FFU/g of tissue, as determined by plaque or infectious focus assay.
^{d}Virus recovered from the spinal cord of infected mice at the time of death or paralysis, expressed in particles/g of tissue, derived by multiplying values in the third column by the particle/PFU ratio characteristic for each virus (FIG. 4D).

In addition, times of onset of paralysis following infection with PV-AB⁷⁵⁵⁻¹⁵¹³ and PV-AB²⁴⁷⁰⁻²⁹⁵⁴ were comparable to that of wt virus (data not shown). Similarly confounding was the observation that at the time of death or paralysis, the viral loads, as determined by plaque assay, in the spinal cords of mice infected with PV-AB⁷⁵⁵⁻¹⁵¹³ and PV-AB²⁴⁷⁰⁻²⁹⁵⁴ were 30- and 300-fold lower, respectively, than those in the mice infected with the wt virus (Table 4). Thus, it seemed unlikely that PV-AB²⁴⁷⁰⁻²⁹⁵⁴, apparently replicating at only 0.3% of the wt virus, would have the same neuropathogenic potential as the wt. However, after having established the altered PFU/particle relationship in PV-AB⁷⁵⁵⁻¹⁵¹³ and PV-AB²⁴⁷⁰⁻²⁹⁵⁴ (see Example 3), the amount of inoculum could now be correlated with the actual number of particles inoculated. After performing this correction, it was established that on a particle basis, PV-AB⁷⁵⁵⁻¹⁵¹³ and PV-AB²⁴⁷⁰⁻²⁹⁵⁴ are 20-fold and 100-fold neuroattenuated, respectively, compared to the wt. See Table 4. Furthermore, on a particle basis the viral loads in the spinal cords of paralyzed mice were very similar with all three viruses (Table 4).

It was also concluded that it was not possible to redesign the PV capsid gene with synonymous codons that would specifically discriminated against expression in the central nervous system. This may be because tissue-specific differences in codon bias described by others (Plotkin et al., 2004) are too small to bring about a tissue-restrictive virus phenotype. In a larger set of brain-specific genes than the one used by Plotkin et al., no appreciable tissue-specific codon bias was detected (data not shown). However, this conclusion should not detract from the fact that polioviruses produced by the method of this invention are indeed neuroattenauted in mice by a factor of up to 100 fold. That is, 100 fold more of the codon or codon-pair deoptimized viral particles are needed to result in the same damage in the central nervous system as the wt virus.

### Example 5

### Effects of Codon Deoptimization on Genomic Translation of Polioviruses

### In Vitro and In Vivo Translation

Two different HeLa cell S10 cytoplasmic extracts were used in this study. A standard extract was prepared by the method of Molla et al. (1991). [³⁵S]methionine-labeled translation products were analyzed by gel autoradiography. The second extract was prepared as described previously (Kaplan and Racaniello, 1988), except that it was not dialyzed and endogenous cellular mRNAs were not removed with micrococcal nuclease. Reactions with the modified extract were not supplemented with exogenous amino acids or tRNAs. Translation products were analyzed by western blotting with anti-2C monoclonal antibody 91.23 (Pfister and Wimmer, 1999). Relative intensities of 2BC bands were determined by a pixel count of the scanned gel image using the NIH-Image 1.62 software. In all cases, translation reactions were programmed with 200 ng of the various in vitro-transcribed viral genomic RNAs.

For analysis of in vivo translation, HeLa cells were transfected with in vitro-transcribed dicistronic replicon RNA as described above. In order to assess translation isolated from RNA replication, transfections were carried out in the presence of 2 mM guanidine hydrochloride. Cells were lysed after 7 h in passive lysis buffer (Promega, Madison, WI) followed by a dual firefly (F-Luc) and Renilla (R-Luc) luciferase assay (Promega). Translation efficiency of the second cistron (P1-Fluc-P2-P3 polyprotein) was normalized through division by the Renilla luciferase activity of the first cistron expressed under control of the Hepatitis C Virus (HCV) internal ribosome entry site (IRES).

### Codon-Deoptimized Viruses are Deficient at the Level of Genome Translation

Since the synthetic viruses and the wt PV(M) are indistinguishable in their protein makeup and no known RNA-based regulatory elements were altered in the modified RNA genomes, these designs enabled study of the effect of reduced genome translation/replication on attenuation without affecting cell and tissue tropism or immunological properties of the virus. The PV-AB genome was designed under the hypothesis that introduction of many suboptimal codons into the capsid coding sequence should lead to a reduction of genome translation. Since the P1 region is at the N-terminus of the polyprotein, synthesis of all downstream nonstructural proteins is determined by the rate of translation through the P1 region. To test whether in fact translation is affected, in vitro translations were performed (FIG. 5).

Unexpectedly, the initial translations in a standard HeLa-cell based cytoplasmic S10 extract (Molla et al., 1991) showed no difference in translation capacities for any of the genomes tested (FIG. 5A). However, as this translation system is optimized for maximal translation, it includes the exogenous addition of excess amino acids and tRNAs, which could conceivably compensate for the genetically engineered codon bias. Therefore, in vitro translations were repeated with a modified HeLa cell extract, which was not dialyzed and in which cellular mRNAs were not removed by micrococcal nuclease treatment (FIG. 5B). Translations in this extract were performed without the addition of exogenous tRNAs or amino acids. Thus, an environment was created that more closely resembles that in the infected cell, where translation of the PV genomes relies only on cellular supplies while competing for resources with cellular mRNAs. Due to the high background translation from cellular mRNA and the low [35S]Met incorporation rate in nondialyzed extract, a set of virus-specific translation products were detected by western blotting with anti-2C antibodies (Pfister and Wimmer, 1999). These modified conditions resulted in dramatic reduction of translation efficiencies of the modified genomes which correlated with the extent of the deoptimized sequence. Whereas translation of PV-SD was comparable to that of the wt, translation of three noninfectious genomes, PV-AB, PV-AB¹⁵¹³⁻³³⁸⁶, and PV-AB⁷⁵⁵⁻²⁴⁷⁰, was reduced by approximately 90% (FIG. 5B).

Burns et al. (2006) recently reported experiments related to those described herein. These authors altered codon usage to a much more limited extent than in the present study, and none of their mutant viruses expressed a lethal phenotype. Interestingly, Burns et al. determined that translation did not play a major role in the altered phenotypes of their mutant viruses, a conclusion at variance with the data presented herein. It is likely that the in vitro translation assay used by Burns et al. (2006), which employed a nuclease-treated rabbit reticulocyte lysate supplemented with uninfected HeLa cell extract and excess amino acids, explains their failure to detect any significant reduction in translation. Cf FIG. 5A.

Considering the ultimately artificial nature of the in vitro translation system, the effect of various capsid designs on translation in cells was also investigated. For this purpose, dicistronic poliovirus reporter replicons were constructed (FIG. 6A) based on a previously reported dicistronic replicon (Zhao and Wimmer, 2001). Various P1 cassettes were inserted immediately upstream and in-frame with the firefly luciferase (F-Luc) gene. Thus, the poliovirus IRES drives expression of a single viral polyprotein similar to the one in the viral genome, with the exception of the firefly luciferase protein between the capsid and the 2A^{pro} proteinase. Expression of the Renilla luciferase (R-Luc) gene under the control of the HCV IRES provides an internal control. All experiments were carried out in the presence of 2 mM guanidine hydrochloride, which completely blocks genome replication (Wimmer et al., 1993). Using this type of construct allowed an accurate determination of the relative expression of the second cistron by calculating the F-Luc/R-Luc ratio. As F-Luc expression depends on successful transit of the ribosome through the upstream P1 region, it provides a measure of the effect of the inserted P1 sequence on the rate of polyprotein translation. Using this method, it was indeed found that the modified capsid coding regions, which were associated with a lethal phenotype in the virus background (e.g., PV-AB, PV-AB¹⁵¹³⁻²⁴⁷⁰, and PV-AB²⁴⁷⁰⁻³³⁸⁶) reduced the rate of translation by approximately 80 to 90% (FIG. 6B). Capsids from two viable virus constructs, PV-AB²⁴⁷⁰⁻²⁹⁵⁴ and PV-AB²⁹⁵⁴⁻³³⁸⁶, allowed translation at 68% and 83% of wt levels, respectively. In vivo translation rates of the first cistron remained constant in all constructs over a time period between 3 and 12 h, suggesting that RNA stability is not affected by the codon alterations (data not shown). In conclusion, the results of these experiments suggest that poliovirus is extremely dependent on very efficient translation as a relatively small drop in translation efficiency through the P1 region of 30%, as seen in PV-AB²⁴⁷⁰⁻²⁹⁵⁴, resulted in a severe virus replication phenotype.

### Example 6

### Genetic Stability of Codon-Deoptimized Polioviruses

Due to the distributed effect of many mutations over large genome segments that contribute to the phenotype, codon deoptimized viruses should have genetically stable phenotypes. To study the genetic stability of codon deoptimized viruses, and to test the premise that these viruses are genetically stable, viruses are passaged in suitable host cells. A benefit of the present “death by 1000 cuts” theory of vaccine design is the reduced risk of reversion to wild type. Typical vaccine strains differ by only few point mutations from the wt viruses, and only a small subset of these may actually contribute to attenuation. Viral evolution quickly works to revert such a small number of active mutations. Indeed, such reversion poses a serious threat for the World Health Organization (WHO) project to eradicate poliovirus from the globe. So long as a live vaccine strain is used, there is a very real chance that this strain will revert to wt. Such reversion has already been observed as the source of new polio outbreaks (Georgescu et al., 1997; Kew et al., 2002; Shimizu et al., 2004).

With hundreds to thousands of point mutations in the present synthetic designs, there is little risk of reversion to wt strains. However, natural selection is powerful, and upon passaging, the synthetic viruses inevitably evolve. Studies are ongoing to determine the end-point of this evolution, but a likely outcome is that they get trapped in a local optimum, not far from the original design.

To validate this theory, representative re-engineered viruses are passaged in a host cell up to 50 times. The genomes of evolved viruses are sequenced after 10, 20 and 50 passages. More specifically, at least one example chimera from each type of deoptimized virus is chosen. The starting chimera is very debilitated, but not dead. For example, for PV the chimeras could be PV-AB²⁴⁷⁰⁻²⁹⁵⁴ and PV-Min⁷⁵⁵⁻²⁴⁷⁰. From each starting virus ten plaques are chosen. Each of the ten plaque-derived virus populations are bulk passaged a total of 50 times. After the 10^{th}, 20^{th} and 50^{th} passages, ten plaque-purified viruses are again chosen and their genomes are sequenced together with the genomes of the ten parent viruses. After passaging, the fitness of the 40 (30+10 per parent virus) chosen viruses is compared to that of their parents by examining plaque size, and determining plaque forming units/ml as one-step growth kinetics. Select passage isolates are tested for their pathogenicity in appropriate host organisms. For example, the pathogenicity of polioviruses is tested in CD155tg mice.

Upon sequencing of the genomes, a finding that all 10 viral lines have certain mutations in common would suggest that these changes are particularly important for viral fitness. These changes may be compared to the sites identified by toeprinting as the major pause sites (see Example 9); the combination of both kinds of assay may identify mutant codons that are most detrimental to viral fitness. Conversely, a finding that the different lines have all different mutations would support the view that many of the mutant codon changes are very similar in their effect on fitness. Thus far, after 10 passages in HeLa cells, PV-AB⁷⁵⁵⁻¹⁵¹³ and PV-AB²⁴⁷⁰⁻²⁹⁵⁴ have not undergone any perceivable gain of fitness. Viral infectious titers remained as low (10⁷ PFU/ml and 10⁶ FFU/ml) as at the beginning of the passage experiment, and plaque phenotype did not change (data not shown). Sequence analysis of these passaged viruses is now in progress, to determine if and what kind of genetic changes occur during passaging.

Burns et al. (2006) reported that their altered codon compositions were largely conserved during 25 serial passages in HeLa cells. They found that whereas the fitness for replication in HeLa cells of both the unmodified Sabin 2 virus and the codon replacement viruses increased with higher passage numbers, the relative fitness of the modified viruses remained lower than that of the unmodified virus. Thus, all indications are that viruses redesigned by SAVE are genetically very stable. Preliminary data for codon and codon-pair deoptimized viruses of the invention suggest that less severe codon changes distributed over a larger number of codons improves the genetic stability of the individual virus phenotypes and thus improves their potential for use in vaccines.

### Example 7

### Re-Engineering of Capsid Region of Polioviruses by Deoptimizing Codon Pairs

### Calculation of Codon Pair Bias.

Every individual codon pair of the possible 3721 non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurrences over the number that would have been expected in this set of genes (in this example the human genome). Determining the actual number of occurrences of a particular codon pair (or in other words the likelihood of a particular amino acid pair being encoded by a particular codon pair) is simply a matter of counting the actual number of occurrences of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

To perform these calculations within the human context, the most recent Consensus CDS (CCDS) database of consistently annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair.

$$S ( {P}_{ij} ) = ln ( \frac{{N}_{O} ( {P}_{ij} )}{{N}_{E} ( {P}_{ij} )} ) = ln ( \frac{{N}_{O} ( {P}_{ij} )}{F ( {C}_{i} ) F ( {C}_{j} ) {N}_{O} ( {X}_{ij} )} )$$

In the calculation, Pᵢⱼ is a codon pair occurring with a frequency of N_{O}(Pᵢⱼ) in its synonymous group. Cᵢ and Cⱼ are the two codons comprising Pᵢⱼ, occurring with frequencies F(Cᵢ) and F(Cⱼ) in their synonymous groups respectively. More explicitly, F(Cᵢ) is the frequency that corresponding amino acid Xᵢ is coded by codon Cᵢ throughout all coding regions and F(Cᵢ)=N_{O}(Cᵢ)/N_{O}(Xᵢ), where N_{O}(Cᵢ) and N_{O}(Xᵢ) are the observed number of occurrences of codon Cᵢ and amino acid Xᵢ respectively. F(Cⱼ) is calculated accordingly. Further, N_{O}(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency Nₒ(Pᵢⱼ) over the expected number of occurrences of Nₑ(Pᵢⱼ).

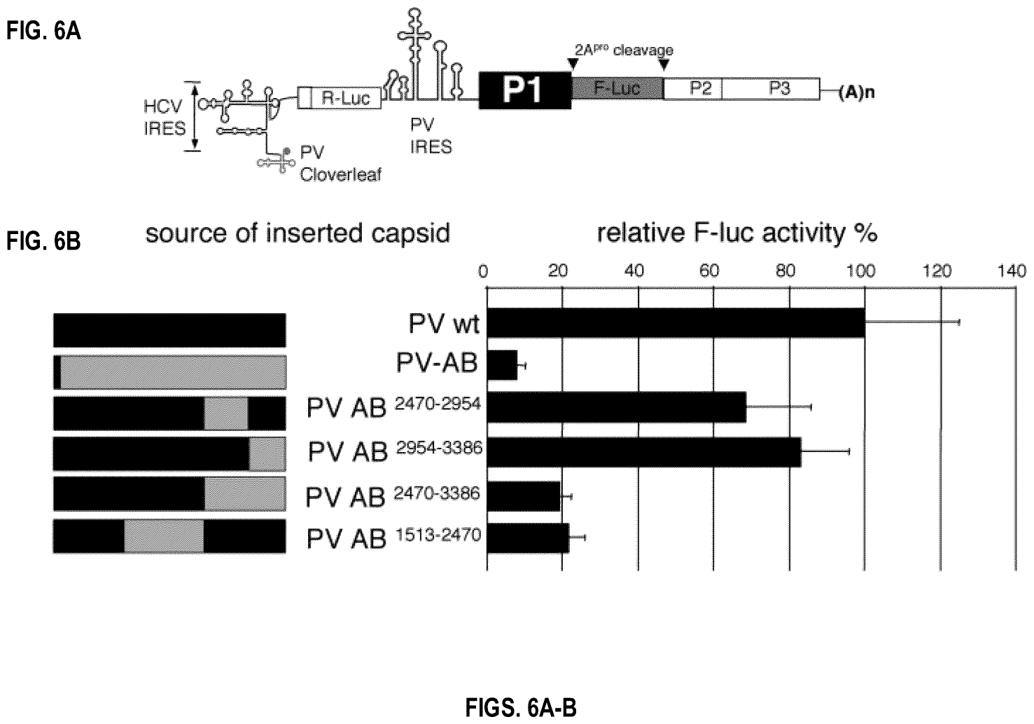

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions (FIG. 7).

The “combined” codon pair bias of an individual coding sequence was calculated by averaging all codon pair scores according to the following formula:

$$S ( {P}_{ij} ) = {\sum }_{l = 1}^{k} \frac{S ( {P}_{ij} ) l}{k - 1} .$$

The codon pair bias of an entire coding region is thus calculated by adding all of the individual codon pair scores comprising the region and deviding this sum by the length of the coding sequence.

### Changing of Codon Pair Bias.

The capsid-coding region of PV(M) was re-engineered to change codon pair bias. The largest possible number of rarely used codon pairs (creating virus PV-Min) or the largest possible number of widely used codon pairs (creating virus PV-Max) was introduced, while preserving the codon bias and all other features of the wt virus genome. The following explains our method in detail.

Two sequences were designed to vary the poliovirus P1 region codon pair score in the positive (PV-Max; SEQ ID NO:4) and negative (PV-Min; SEQ ID NO:5) directions. By leaving the amino acid sequence unaltered and the codon bias minimally modified, a simulated annealing algorithm was used for shuffling codons, with the optimization goal of a minimum or maximum codon pair score for the P1 capsid region. The resulting sequences were processed for elimination of splice sites and reduction of localized secondary structures. These sequences were then synthesized by a commercial vendor, Blue Heron Biotechnology, and sequence-verified. The new capsid genes were used to replace the equivalent wt sequence in an infectious cDNA clone of wt PV via two PflMI restriction sites. Virus was derived as described in Example 1.

For the PV-Max virus, death of infected cells was seen after 24 h, a result similar to that obtained with wt virus. Maximal viral titer and one-step growth kinetics of PV-Max were also identical to the wt. In contrast, no cell death resulted in cells transfected with PV-Min mutant RNA and no viable virus could be recovered. The transfections were repeated multiple times with the same result. Lysates of PV-Min transfected cells were subjected to four successive blind passages, and still no virus was obtained.

The capsid region of PV-Min was divided into two smaller sub-fragments (PV-Min⁷⁵⁵⁻²⁴⁷⁰ and PV-Min²⁴⁷⁰⁻³³⁸⁶) as had been done for PV-AB (poor codon bias), and the sub-fragments were cloned into the wt background. As with the PV-AB subclones, subclones of PV-Min were very sick, but not dead (FIG. 8). As observed with PV-AB viruses, the phenotype of PV-Min viruses is a result of reduced specific infectivity of the viral particles rather than to lower production of progeny virus. Ongoing studies involve testing the codon pair-attenuated chimeras in CD155tg mice to determine their pathogenicity. Also, additional chimeric viruses comprising subclones of PV-Min cDNAs are being made, and their ability to replicate is being determined (see example 8 and 9 below). Also, the effect of distributing intermediate amounts of codon pair bias over a longer sequence are being confirmed. For example, a poliovirus derivative is designed to have a codon pair bias of about −0.2 (PV-0.2; SEQ ID NO:6), and the mutations from wild type are distributed over the full length of the P1 capsid region. This is in contrast to PV-MinZ (PV-Min²⁴⁷⁰⁻³³⁸⁶) which has a similar codon pair bias, but with codon changes distributed over a shorter sequence.

It is worth pointing out that PV-Min and PV-0.2 are sequences in which there is little change in codon usage relative to wild type. For the most part, the sequences employ the same codons that appear in the wild type PV(M) virus. PV-MinZ is somewhat different in that it contains a portion of PV-Min subcloned into PV(M). As with PV-Min and PV-0.2, the encoded protein sequence is unchanged, but codon usage as determined in either the subcloned region, or over the entire P1 capsid region, is not identical to PV-Min (or PV-0.2), because only a portion of the codon rearranged sequence (which has identical codons over its full length, but not within smaller segments) has been substituted into the PV(M) wild type sequence. Of course, a mutated capsid sequence could be designed to have a codon pair bias over the entire P1 gene while shuffling codons only in the region from nucleotides 2470-3386.

### Example 8

### Viruses Constructed by a Change of Codon-Pair Bias are Attenuated in CD155tg Mice

### Mice Intracerebral Injections, Survival

To test the attenuation of PV-Min⁷⁵⁵⁻²⁴⁷⁰ and PV-Min²⁴⁷⁰⁻³³⁸⁵ in an animal model, these viruses were purified and injected intra-cerebrally into CD 155 (PVR/poliovirus receptor) transgenic mice (See Table 5). Indeed these viruses showed a significantly attenuated phenotype due to the customization of codon pair bias using our algorithm. PVM-wt was not injected at higher dose because all mice challenged at 10e5 virions died because of PVM-wt. This attenuated phenotype is due to the customization of codon pair bias using our algorithm. This reaffirms that the customization of codon-pair bias is applicable for a means to create live vaccines.

TABLE 5
Mice Intracerebral Injections, Survival.

| | 10e4 | 10e5 | 10e6 | 10e7 |
|---|---|---|---|---|
| Virus | Virions | Virions | Virions | Virions |
| PV-Min⁷⁵⁵⁻²⁴⁷⁰ | 4/4 | 3/4 | 3/5 | 3/4 |
| PV-Min²⁴⁷⁰⁻³³⁸⁵ | 4/4 | 4/4 | 5/5 | 3/4 |
| PVM-wt | 3/4 | 0/4 | — | — |

These findings are significant in two respects. First, they are the first clear experimental evidence that codon pair bias is functionally important, i.e., that a deleterious phenotype can be generated by disturbing codon pair bias. Second, they provide an additional dimension of synonymous codon changes that can be used to attenuate a virus. The in vivo pathogenicity of these codon-pair attenuated chimeras have been tested in CD155tg and have shown an attenuated phenotype (See Table 5). Additional chimeric viruses comprising subclones of PV-Min capsid cDNAs have been assayed for replication in infected cells and have also shown an attenuated phenotype.

### Example 9

Construction of Synthetic Poliovirus with Altered Codon-Pair Bias: Implications for Vaccine Development

### Calculation of Codon Pair Bias, Implementation of Algorithm to Produce Codon Pair Deoptimized Sequences.

We developed an algorithm to quantify codon pair bias. Every possible individual codon pair was given a “codon pair score”, or “CPS”. We define the CPS as the natural log of the ratio of the observed over the expected number of occurrences of each codon pair over all human coding regions.

$$CPS = ln ( \frac{F ( AB ) o}{\frac{F ( A ) \times F ( B )}{F ( X ) \times F ( Y )} \times F ( XY )} )$$

Although the calculation of the observed occurrences of a particular codon pair is straightforward (the actual count within the gene set), the expected number of occurrences of a codon pair requires additional calculation. We calculate This expected number is calculated to be independent both of amino acid frequency and of codon bias, similar to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome

Using these calculated CPSs, any coding region can then be rated as using over- or under-represented codon pairs by taking the average of the codon pair scores, thus giving a Codon Pair Bias (CPB) for the entire gene.

$$CPB = {\sum }_{i = 1}^{k} \frac{CPSi}{k - 1}$$

The CPB has been calculated for all annotated human genes using the equations shown and plotted (FIG. 7). Each point in the graph corresponds to the CPB of a single human gene. The peak of the distribution has a positive codon pair bias of 0.07, which is the mean score for all annotated human genes. Also there are very few genes with a negative codon pair bias. Equations established to define and calculate CPB were then used to manipulate this bias.

### Development and Implementation of Computer-Based Algorithm to Produce Codon Pair Deoptimized Sequences.

Using these formulas we next developed a computer based algorithm to manipulate the CPB of any coding region while maintaining the original amino acid sequence. The algorithm has the critical ability to maintain the codon usage of a gene (i.e. preserve the frequency of use of each existing codon) but “shuffle” the existing codons so that the CPB can be increased or decreased. The algorithm uses simulated annealing, a mathematical process suitable for full-length optimization (Park, S. et al., 2004). Other parameters are also under the control of this algorithm; for instance, the free energy of the folding of the RNA. This free energy is maintained within a narrow range, to prevent large changes in secondary structure as a consequence of codon re-arrangement. The optimization process specifically excludes the creation of any regions with large secondary structures, such as hairpins or stem loops, which could otherwise arise in the customized RNA. Using this computer software the user simply needs to input the cDNA sequence of a given gene and the CPB of the gene can be customized as the experimenter sees fit.

### De Novo Synthesis of P1 Encoded by Either Over-Represented or Under-Represented Codon-Pairs.

To obtain novel, synthetic poliovirus with its P1 encoded by either over-represented or under-represented codon pairs, we entered the DNA sequence corresponding to the P1 structural region of poliovirus type I Mahoney (PV(M)-wt) into our program yielding—PV-Max-P1 using over-represented codon pairs (566 mutations) and PV-Min-P1 using under-represented codon pairs (631 mutations). The CPB scores of these customized, novel synthetic P-1 regions are PV-Max=+0.25 and PV-Min=−0.48, whereas the CPB of PV(M)-wt is −0.02 (FIG. 7).

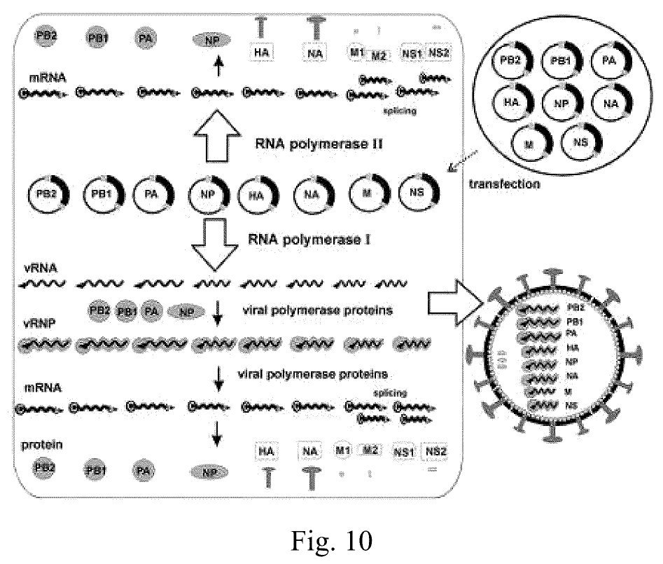

Additional customization included inclusion of restriction sites that were designed into both synthetic sequences at given intervals, to allow for sub-cloning of the P1 region. These synthetic P1 fragments were synthesized de novo by Blue Herron Corp. and incorporated into a full-length cDNA construct of poliovirus (FIG. 11) (Karlin et al., 1994). A small fragment (3 codons, 9 nucleotides) of PV(M)-wt sequence was left after the AUG start codon in both constructs to allow translation to initiate equally for all synthetic viruses; thus providing more accurate measurement of the effect of CPB on the elongation phase of translation.

### DNA Synthesis, Plasmids, Sub Cloning of Synthetic Capsids and Bacteria.

Large codon-pair altered PV cDNA fragments, corresponding to nucleotides 495 to 3636 of the PV genome, were synthesized by Blue Heron Corp. using their proprietary GeneMaker® system (http://www.blueheronbio.com/). All subsequent poliovirus cDNA clones/sub clones were constructed from PV1(M) cDNA clone pT7PVM using unique restriction sites (van der Wert, et al., 1986). The full-length PV-Min, PV-Max cassette was released from Blue Heron's carrier vector via PflMI digestion and insertion into the pT7PVM vector with its PflMI fragment removed. The PV-MinXY and PV-MinZ constructs were obtained by digestion with NheI and BglII simultaneously, then swapping this fragment with a pT7PVM vector digested similarly. PV-MinXY and PV-MinZ were constructed via BsmI digestion and exchanging the fragment/vector with the similarly digested pT7PVM. PV-MinY was constructed by digesting the PV-MinXY construct with BsmI and swapping this fragment with the BsmI fragment for a digested pT7PVM. Plasmid transformation and amplification were all achieved via Escherichia coli DH5α.

### Creation of Chimeric Viruses Containing CPB-Altered Capsid Regions: Under-Represented Codon Pair Bias Throughout the P1 Results in a Null Phenotype.

Using the T7 RNA polymerase promoter upstream of the poliovirus genomic sequence, positive-sense RNA was transcribed. 1.5 μg of a given plasmid cDNA clone from above was linearized via an EcoRI digestion and than was transcribed into RNA via T7 RNA polymerase (Stratagene) driven by its promoter upstream of the cDNA for 2 hours at 37° C. (van der Werf et al., 1986). This RNA was transfected into 1×10⁶ HeLa R19 cells using a modified DEAE-Dextran method (van der Werf et al., 1986). These cells were than incubate at room-temperature (RT) for 30-minutes. The transfection supernatant was removed and Dulbecco's modified Eagle medium (DMEM) containing 2% bovine calf serum (BCS) was added and the cells were incubated at 37° C. and observed (up to 4 days) for the onset of cytopathic effect (CPE).

The PV-Max RNA transfection produced 90% cytopathic effect (CPE) in 24 hours, which is comparable to the transfection of PV(M)-wt RNA. The PV-Max virus generated plaques identical in size to the wild type. In contrast, the PV-Min RNA produced no visible cytopathic effect after 96 hours, and no viable virus could be isolated even after four blind passages of the supernatant from transfected cells.

The subsequent use of the supernatant from cells subjected to PV-Max RNA transfection also produced 95% CPE in 12 hours, thus indicating that the transfected genomic material successfully produced PV-Max poliovirus virions. In contrast, the PV-Min viral RNA yielded no visible CPE after 96 hours and four blind passages of the supernatant, possibly containing extremely low levels of virus, also did not produce CPE. Therefore the full-length PV-Min synthetic sequence, utilizing under-represented codon pairs, in the P1 region cannot generate viable virus and so it would need to be sub-cloned.

HeLa R19 cells were maintained as a monolayer in DMEM containing 10% BCS. Virus amplification was achieved on (1.0×10⁸ cells) HeLa R19 mononlayers using 1 M.O.I. Infected cells were incubated at 37° C. in DMEM with 2% BCS for three days or until CPE was observed. After three freeze/thaw cycles cell debris was removed form the lysates via low speed centrifugation and the supernatant containing virus was used for further experiments.

One-Step growth curves were achieved by infecting a monolayer of HeLa R19 cells with 5 M.O.I of a given virus, the inoculums was removed, cells washed 2× with PBS and then incubating at 37° C. for 0, 2, 4, 7, 10, 24, and 48 hours. These time points were then analyzed via plaque assay. All Plaque assay were performed on monolayers of HeLa R19 cells. These cells were infected with serial dilution of a given growth curve time point or purified virus. These cells were then overlaid with a 0.6% tragenthum gum in Modified Eagle Medium containing 2% BCS and then incubated at 37° C. for either 2 days for PV(M)-wt and PV-Max, or 3 days for PV-Min (X, Y, XY, or Z) viruses. These were then developed via crystal violet staining and the PFU/ml titer was calculated by counting visible plaques.

### Small Regions of Under-Represented Codon Pair Bias Rescues Viability, but Attenuate the Virus.

Using the restriction sites designed within the PV-Min sequence we subcloned portions of the PV-Min P1 region into an otherwise wild-type virus, producing chimeric viruses where only sub-regions of P1 had poor codon pair bias (FIG. 11) (van der Werf et al., 1986). From each of these sub-clones, RNA was produced via in vitro transcription and then transfected into HeLa R19 cells, yielding viruses with varying degrees of attenuation (Viability scores, FIG. 11). P1 fragments X and Y are each slightly attenuated; however when added together they yield a virus (PV-Min⁷⁵⁵⁻²⁴⁷⁰, PV-MinXY) that is substantially attenuated (FIGS. 3, 4). Virus PVMin²⁴⁷⁰⁻³³⁸⁵ (PV-MinZ) is about as attenuated as PV-MinXY. Construct PV-Min¹⁵¹³⁻³³⁸⁵ (YZ) did not yield plaques, and so apparently is too attenuated to yield viable virus. These virus constructs, which cosplayed varying degrees of attenuation were further investigated to determine their actual growth kinetics.

### One-Step Growth Kinetics and the Mechanism of Attenuation: Specific Infectivity is Reduced.

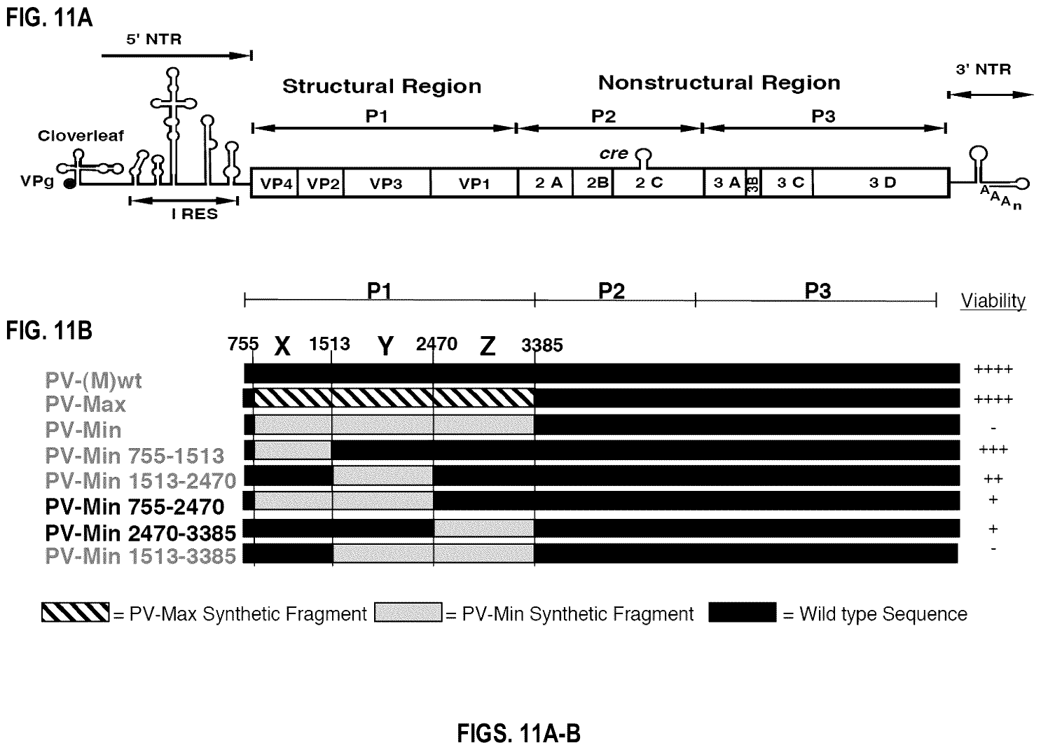

For each viable construct, one step-growth kinetics were examined. These kinetics are generally similar to that of wild-type in that they proceed in the same basic manner (i.e. an eclipse phase followed by rapid, logarithmic growth). However, for all PV-Min constructs, the final titer in terms of Plaque Forming Units (PFU) was typically lower than that of wild-type viruses by one to three orders of magnitude (FIG. 12A).

When virus is measured in viral particles per ml (FIG. 12B) instead of PFU, a slightly different result is obtained and suggests these viruses produce nearly equivalent numbers of particles per cell per cycle of infection as the wild-type virus. In terms of viral particles per ml, the most attenuated viruses are only 78% (PV-MinXY) or 82% (PV-MinZ) attenuated which on a log scale is less than one order of magnitude. Thus these viruses appear to be attenuated by about two orders of magnitude in their specific infectivity (the number of virions required to generate a plaque).

To confirm that specific infectivity was reduced, we re-measured the ratio of viral particles per PFU using highly purified virus particles. Selected viruses were amplified on 10⁸ HeLa R19 cells. Viral lysates were treated with RNAse A to destroy exposed viral genomes and any cellular RNAs, that would obscure OD values. Also the viral lysates were then incubated for 1 hour with 0.2% SDS and 2 mM EDTA to denature cellular and non-virion viral proteins. A properly folded and formed poliovirus capsid survives this harsh SDS treatment, were as alph particles do not (Mueller et al., 2005). Virions from these treated lysates were then purified via ultracentrifugation over a sucrose gradient. The virus particle concentration was measured by optical density at 260 nm using the formula 9.4×10¹² particles/ml=1 OD₂₆₀ unit (Rueckert, 1985). A similar number of particles was produced for each of the four viruses (Table 6). A plaque assay was then performed using these purified virions. Again, PV-MinXY and PV-MinZ required many more viral particles than wild-type to generate a plaque (Table 6).

For wild-type virus, the specific infectivity was calculated to be 1 PFU per 137 particles (Table 6), consistent with the literature (Mueller et al., 2006; Schwerdt and Fogh, 1957; Joklik and Darnell, 1961). The specific infectivities of viruses PV-MinXY and PV-MinZ are in the vicinity of 1 PFU per 10,000 particles (Table 6).

Additionally the heat stability of the synthetic viruses was compared to that of PV(M)-wt to reaffirm the SDS treatment data, that these particles with portions of novel RNA were equally as stable. Indeed these synthetic viruses had the same temperature profile as PV(M)-wt when incubated at 50° C. and quantified as a time course (data not shown).

### Under-Represented Codon Pairs Reduce Translation Efficiency, Whereas Over-Represented Pairs Enhance Translation.

One hypothesis for the existence of codon pair bias is that the utilization of under-represented pairs causes poor or slow translation rates. Our synthetic viruses are, to our knowledge, the first molecules containing a high concentration of under-represented codon pairs, and as such are the first molecules suitable for a test of the translation hypothesis.

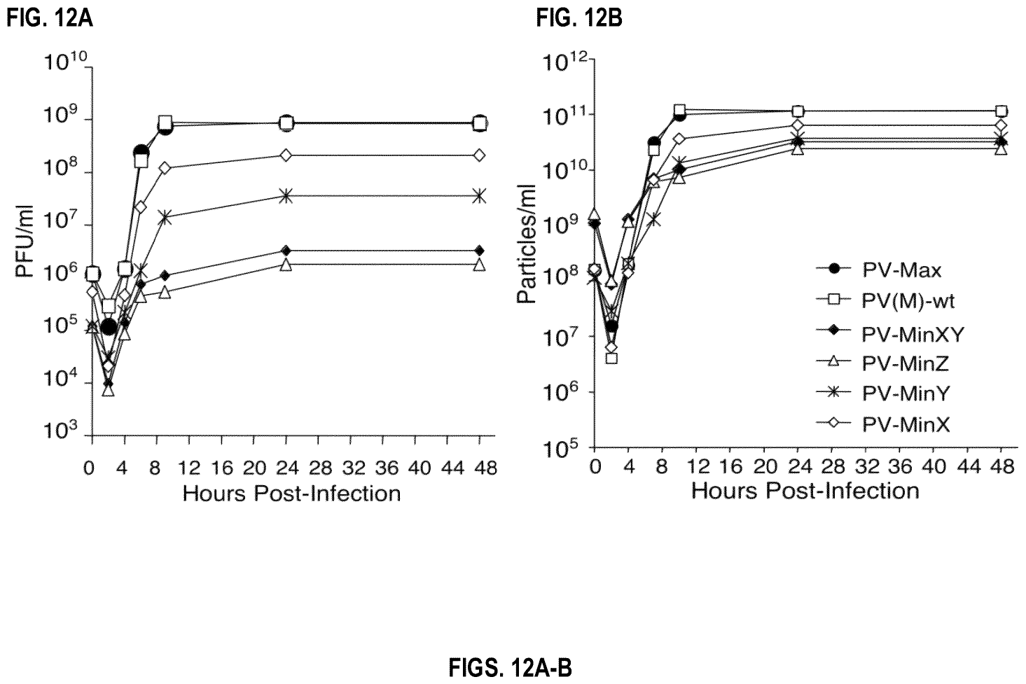

To measure the effect of codon pair bias on translation, we used a dicistronic reporter (Mueller et al., 2006) (FIG. 13). The first cistron expresses Renilla luciferase (R-Luc) under the control of the hepatitis C virus internal ribosome entry site (IRES) and is used as a normalization control. The second cistron expresses firefly luciferase (F-Luc) under the control of the poliovirus IRES. However, in this second cistron, the F-Luc is preceded by the P1 region of poliovirus, and this P1 region could be encoded by any of the synthetic sequence variants described here. Because F-Luc is translated as a fusion protein with the proteins of the P1 region, the translatability of the P1 region directly affects the amount of F-Luc protein produced. Thus the ratio of F-Luc luminescence to R-Luc luminescence is a measure of the translatability of the various P1 encodings.

The P1 regions of wild-type, PV-Max, PV-Min, PV-MinXY and PV-MinZ were inserted into the region labeled “P1” (FIG. 13A). PV-MinXY, PV-MinZ, and PV-Min produce much less F-Luc per unit of R-Luc than does the wild-type P1 region, strongly suggesting that the under-represented codon pairs are causing poor or slow translation rates (FIG. 13). In contrast, PV-Max P1 (which uses over-represented codon pairs) produced more F-Luc per unit of R-Luc, suggesting translation is actually better for PV-Max P1 compared to PV(M)-wt P1.

### Dicistronic Reporter Construction, and In Vivo Translation.

The dicistronic reporter constructs were all constructed based upon pdiLuc-PV (Mueller et al., 2006). PV-Max and PV-Min capsid regions were amplified via PCR using the oligonucleotides P1max-2A-RI (+)/P1max-2A-RI (−) or P1 min-2A-RI (+)/P1 min-2A-RI (−) respectively. The PCR fragment was gel purified and then inserted into an intermediate vector pCR-®-XL-TOPO® (Invitrogen). This intermediate vector was than amplified in One Shot® TOP10 chemically competent cells. After preparation of the plasmid via Quiagen miniprep the intermediate vectors containing PV-Min was digested with EcoRI and these fragments were ligated into the pdiLuc-PV vector that was equally digested with EcoRI (Mueller et al., 2006). These plasmids were also amplified in One Shot® TOP10 chemically competent cells (Invitrogen). To construct pdiLuc-PV-MinXY and pdiLuc-PV-MinZ, pdiLuc-PV and pdiLuc-PV-Min were equally digested with NheI and the resulting restriction fragments were exchanged between the respective vectors. These were than transformed into One Shot® TOP10 chemically competent cells and then amplified. From all four of these clones RNA was transcribed via the T7 polymerase method (van der Werf et al., 1986).

To analyze the in vivo translation efficiency of the synthetic capsids the RNA of the dicistronic reporter constructs were transfected into 2×10⁵ HeLa R19 cells on 12-well dishes via Lipofectamine 2000 (Invitrogen). In order to quantify the translation of only input RNA the transfection was accomplished in the presence of 2 mM guanidine hydrochloride (GuHCL). Six hours after transfection cells were lysed via passive lysis buffer (Promega) and then these lysates were analyzed by a dual firefly (F-Luc) Renilla (R-Luc) luciferase assay (Promega).

### Genetic Stability of PV-MinXY and PV-MinZ.

Because PV-MinXY and PV-MinZ each contain hundreds of mutations (407 and 224, respectively), with each mutation causing a miniscule decrease in overall codon pair bias, we believe it should be very difficult for these viruses to revert to wild-type virulence. As a direct test of this idea, viruses PV-MinXY and PV-MinZ were serially-passaged 15 times, respectively, at an MOI of 0.5. The titer was monitored for phenotypic reversion, and the sequence of the passaged virus was monitored for reversions or mutation. After 15 passages there was no phenotypic change in the viruses (i.e. same titer, induction of CPE) and there were no fixed mutations in the synthetic region.

### Heat Stability and Passaging.

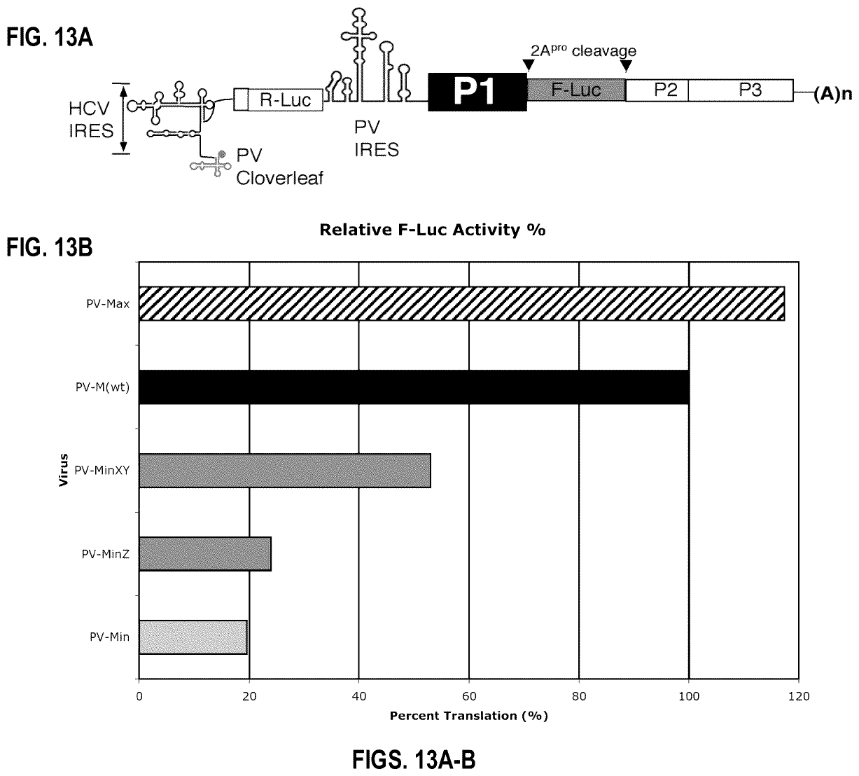

The stability of the synthetic viruses, PV-MinXY and PV-Min Z, was tested and compared to PV(M)-wt. This was achieved by heating 1×10⁸ particles suspended in PBS to 50° C. for 60 minutes and then measuring the decrease in intact viral particles via plaque assay at 5, 15, 30 and 60 minutes (FIG. 14). In order to test the genetic stability of the synthetic portions of the P1 region of the viruses PV-MinXY and PV-MinZ these viruses were serial passaged. This was achieved by infecting a monolayer of 1×10⁶ HeLa R19 cells with 0.5 MOI of viruses, PV-MinXY and PV-MinZ, and then waiting for the induction of CPE. Once CPE initiated, which remained constant throughout passages, the lysates were used to infect new monolayers of HeLa R19 cells. The titer and sequence was monitored at passages 5, 9, and 15 (data not shown).

### Virus Purification and Determination of Viral Particles Via OD₂₆₀ Absorbance.

A monolayer of HeLa R19 cells on a 15 cm dish (1×10⁸ cells) were infected with PV(M)-wt, PV-Max, PV-MinXY or PV-Min Z until CPE was observed. After three freeze/thaw cycles the cell lysates were subjected to two initial centrifugations at 3,000×g for 15 minutes and then 10,000×g for 15 minutes. Then 10 μg/ml of RNAse A (Roche) was added to supernatant and incubated at RT for 1 hour; Subsequently 0.5% sodium dodecyl sulfate (SDS) and 2 mM EDTA was added to the supernatant, gently mixed and incubated at RT for 30 minutes. These supernatants containing virus particles were placed above a 6 ml sucrose cushion [30% sucrose in Hank's Buffered Salt Solution (HBSS)]. Sedimentation of virus particles was achieved by ultracentrifugation through the sucrose gradient for 3.5 hours at 28,000 rpm using an SW28 swing-bucket rotor.

After centrifugation, the sucrose cushion was left intact and the supernatant was removed and the tube was washed two times with HBBS. After washing, the sucrose was removed and the virus “pearl” was re-suspended in PBS containing 0.1% SDS. Viral titers were determined via plaque assay (above). Virus particles concentration was determined via the average of three measurements of the optical density at 260 nm of the solution via the NanoDrop spectrophotometer (NanoDrop Technologies) using the formula 9.4×10¹² particles/ml=1 OD₂₆₀ unit (Mueller et al., 2006; Rueckert, 1985).

### Neuroattenuation of PV-MinXY and PV-MinZ in CD155tg Mice.

The primary site of infection of wild-type poliovirus is the oropharynx and gut, but this infection is relatively asymptomatic. However, when the infection spreads to motor neurons in the CNS in 1% of PV(M)-wt infections, the virus destroys these neurons, causing death or acute flaccid paralysis know as poliomyelitis (Landsteiner and Popper, 1909; Mueller et al., 2005). Since motor neurons and the CNS are the critical targets of poliovirus, we wished to know whether the synthetic viruses were attenuated in these tissues. Therefore these viruses were administered to CD155tg mice (transgenic mice expressing the poliovirus receptor) via intracerebral injection (Koike et al., 1991). The PLD₅₀ value was calculated for the respective viruses and the PV-MinXY and PV-MinZ viruses were attenuated either 1,000 fold based on particles or 10 fold based on PFU (Table 6) (Reed and Muench, 1938). Since these viruses did display neuroattenuation they could be used as a possible vaccine.

TABLE 6
Reduced Specific Infectivity and Neuroattenuation in CD155tg mice.

| | | Purified | Purified | Specific | PLD₅₀ | PLD₅₀ |
|---|---|---|---|---|---|---|
| Virus | A₂₆₀ | Particles / ml^{a} | PFU / ml | Infectivity^{b} | (Particles)^{c} | (PFU)^{d} |
| PV-M(wt) | 0.956 | 8.97 × 10¹² | 6.0 × 10¹⁰ | 1/137 | 10^{4.0} | 10^{1.9} |
| PV-Max | 0.842 | 7.92 × 10¹² | 6.0 × 10¹⁰ | 1/132 | 10^{4.1} | 10^{1.9} |
| PV-MinXY | 0.944 | 8.87 × 10¹² | 9.6 × 10⁸ | 1/9,200 | 10^{7.1} | 10^{3.2} |
| PV-MinZ | 0.731 | 6.87 × 10¹² | 5.1 × 10⁸ | 1/13,500 | 10^{7.3} | 10^{3.2} |

^{a}The A₂₆₀ was used to determine particles/ml via the formula 9.4 × 10¹² particles/ml = 1 OD₂₆₀ unit
^{b}Calculated by dividing the PFU/ml of purified virus by the Particles/ml
^{c, d}calculated after administration of virus via intracerebral injection to CD155tg mice at varying doses

### Vaccination of CD155tg Mice Provides Immunity and Protection Against Lethal Challenge.

Groupings of 4-6, 6-8 week old CD155tg mice (Tg21 strain) were injected intracerebrally with purified virus dilutions from 10² particles to 10⁹ particles in 30 ul PBS to determine neuropathogenicity (Koike, et al., 1991).

The lethal dose (LD₅₀) was calculated by the Reed and Muench method (Reed and Muench, 1938). Viral titers in the spinal chord and brain were quantified by plaque assay (data not shown).

PV-MinZ and PV-MinXY encode exactly the same proteins as wild-type virus, but are attenuated in several respects, both a reduced specific infectivity and neuroattenuation.

To test PV-Min Z, PV-MinXY as a vaccine, three sub-lethal dose (10⁸ particles) of this virus was administered in 100 ul of PBS to 8, 6-8 week old CD155tg mice via intraperitoneal injection once a week for three weeks. One mouse from the vaccine cohort did not complete vaccine regimen due to illness. Also a set of control mice received three mock vaccinations with 100 ul PBS. Approximately one week after the final vaccination, 30 ul of blood was extracted from the tail vein. This blood was subjected to low speed centrifugation and serum harvested. Serum conversion against PV(M)-wt was analyzed via micro-neutralization assay with 100 plaque forming units (PFU) of challenge virus, performed according to the recommendations of WHO (Toyoda et al., 2007; Wahby, A. F., 2000). Two weeks after the final vaccination the vaccinated and control mice were challenged with a lethal dose of PV(M)-wt by intramuscular injection with a 10⁶ PFU in 100 ul of PBS (Toyoda et al., 2007). All experiments utilizing CD155tg mice were undertaken in compliance with Stony Brook University's IACUC regulations as well as federal guidelines. All 14 vaccinated mice survived and showed no signs of paralysis or parasia; in contrast, all mock-vaccinated mice died (Table 7). These data suggest that indeed the CPB virus using de-optimized codon pairs is able to immunize against the wild-type virus, providing both a robust humeral response, and also allowing complete survival following challenge.

TABLE 7
Protection Against Lethal Challenge

| Virus ^{a} | Mice Protected (out of 7) ^{b} |
|---|---|
| PV-MinZ | 7 |
| PV-MinXY | 7 |
| Mock vaccinated | 0 |

^{a} CD155tg mice received three vaccination doses (10⁸ particles) of respective virus
^{b} challenged with 10⁶ PFU of PV(M)-wt via intramuscular injection.

### Example 10

### Application of SAVE to Influenza Virus

Influenza virus has 8 separate genomic segments. GenBank deposits disclosing the segment sequences for Influenza A virus (A/Puerto Rico/8/34/Mount Sinai(H1N1)) include AF389115 (segment 1, Polymerase PB2), AF389116 (segment 2, Polymerase PB1), AF389117 (segment 3, Polymerase PA), AF389118 (segment 4, hemagglutinin HA), AF389119 (segment 5, nucleoprotein NP), AF389120 (segment 6, neuraminidase NA), AF389121 (segment 7, matrix proteins M1 and M2), and AF389122 (segment 8, nonstructural protein NS1).

In initial studies, the genomic segment of strain A/PR/8/34 (also referred to herein as A/PR8) encoding the nucleoprotein NP, a major structural protein and the second most abundant protein of the virion (1,000 copies per particle) that binds as monomer to full-length viral RNAs to form coiled ribonucleoprotein, was chosen for deoptimization. (See Table 8, below, for parent and deoptimized sequences). Moreover, NP is involved in the crucial switch from mRNA to template and virion RNA synthesis (Palese and Shaw, 2007). Two synonymous encodings were synthesized, the first replacing frequently used codons with rare synonymous codons (NP^{CD}) (i.e., de-optimized codon bias) and, the second, de-optimizing codon pairs (NP^{CPmin}). The terminal 120 nucleotides at either end of the segment were not altered so as not to interfere with replication and encapsidation. NP^{CD} contains 338 silent mutations and NP^{CPmin} (SEQ ID NO:23) contains 314 silent mutations. The mutant NP segments were introduced into ambisense vectors as described (below), and together with the other seven wt influenza plasmids co-transfected into 293T/MDCK co-cultured cells. As a control, cells were transfected with all 8 wt A/PR8 plasmids. Cells transfected with the NP^{CD} segment and the NP^{CPmin} segment produced viable influenza virus similarly to cells transfected with wild-type NP. These new de-optimized viruses, referred to as A/PR8-NP^{CD} or A/PR8-NP^{CPmin}, respectively, appear to be attenuated: The titer (in terms of PFU) is 3- to 10-fold lower than the wild-type virus, and the mutant viruses both make small plaques.

Although the de-optimized influenza viruses are not as severely attenuated as a poliovirus containing a similar number of de-optimized codons, there is a difference in the translational strategies of the two viruses. Poliovirus has a single long mRNA, translated into a single polyprotein. Slow translation through the beginning of this long mRNA (as in our capsid de-optimized viruses) will reduce translation of the entire message, and thus affect all proteins. In contrast, influenza has eight separate segments, and de-optimization of one will have little if any effect on translation of the others. Moreover, expression of the NP protein is particularly favored early in influenza virus infection (Palese and Shaw, 2007).

### Characterization of Influenza Virus Carrying a Codon Pair Deoptimized NP Segment

The growth characteristics of A/PR8-NP^{CPmin} were analyzed by infecting confluent monolayers of Madin Darby Canine Kidney cells (MDCK cells) in 100 mm dishes with 0.001 multiplicities of infection (MOI). Virus inoculums were allowed to adsorb at room temperature for 30 minutes on a rocking platform, then supplemented with 10 ml of Dulbecco Modified Eagle Medium (DMEM) containing 0.2% Bovine Serum Albumin (BSA) and 2 ug/ml TPCK treated Trypsin and incubated at 37 C. After 0, 3, 6, 9, 12, 24, and 48 hours, 100 μl of virus containing medium was removed and virus titers determined by plaque assay.

Viral titers and plaque phenotypes were determined by plaque assay on confluent monolayers of MDCK cells in 35 mm six well plates. 10-fold serial dilutions of virus were prepared in Dulbecco Modified Eagle Medium (DMEM) containing 0.2% Bovine Serum Albumin (BSA) and 2 μg/ml TPCK treated Trypsin. Virus dilutions were plated out on MDCK cells and allowed to adsorb at room temperature for 30 minutes on a rocking platform, followed by a one hour incubation at 37 C in a cell culture incubator. The inoculum was then removed and 3 ml of Minimal Eagle Medium containing 0.6% tragacanth gum (Sigma-Aldrich) 0.2% BSA and 2 ug/ml TPCK treated Trypsin. After 72 hours of incubation at 37 C, plaques were visualized by staining the wells with crystal violet.

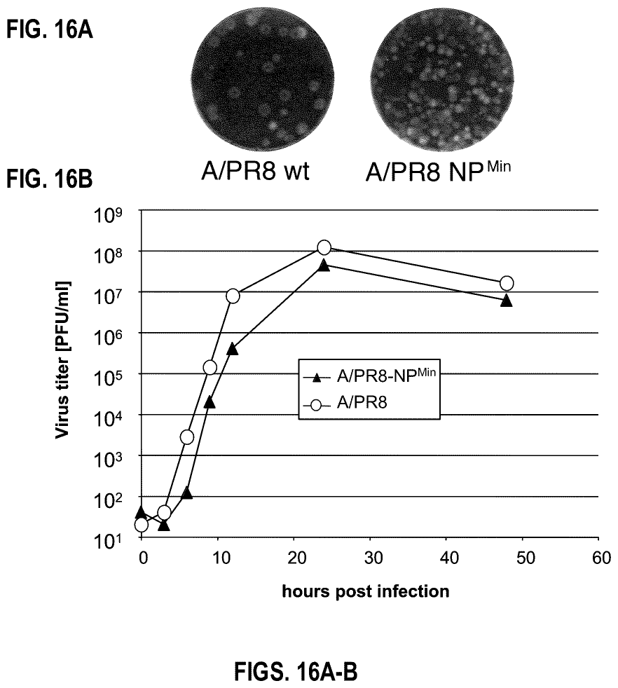

A/PR8-NP^{Min} produced viable virus that produced smaller plaques on MDCK cells compared to the A/PR8 wt (FIG. 16A). Furthermore, upon low MOI infection A/PR8-NP^{Min} manifests a delayed growth kinetics, between 3-12 hrs post infection, where A/PR8-NP^{Min} titers lags 1.5 logs behind A/PR8 (FIG. 16B). Final titers are were 3-5 fold lower than that of A/PR8 (average of three different experiments).

Characterization of Influenza Viruses A/PR8-PB1^{Min-RR}, A/PR8-HA^{Min} and A/PR8-HA^{Min}/NP^{Min} Carrying Codon Pair Deoptimized PB1, HA, or HA and NP Segments.

Codon pair de-optimized genomic segments of strain A/PR/8/34 encoding the hemagglutinin protein HA and the polymerase subunit PB1 were produced. HA is a viral structural protein protruding from the viral surface mediating receptor attachment and virus entry. PB1 is a crucial component of the viral RNA replication machinery. Specifically a synonymous encoding of PB1 (SEQ ID NO:15) was synthesized by de-optimizing codon pairs between codons 190-488 (nucleotides 531-1488 of the PB1 segment) while retaining the wildtype codon usage (PB1^{Min-RR}). Segment PB1^{Min-RR} contains 236 silent mutations compared the wt PB1 segment.

A second synonymous encoding of HA (SEQ ID NO:21) was synthesized by de-optimizing codon pairs between codons 50-541 (nucleotides 180-1655 of the HA segment) while retaining the wildtype codon usage (HA^{Min}). HA^{Min} contains 355 silent mutations compared the to wt PB1 segment.

The mutant PB1^{Min-RR} and HA^{Min} segments were introduced into an ambisense vector as described above and together with the other seven wt influenza plasmids co-transfected into 293T/MDCK co-cultured cells. In addition the HA^{Min} segment together with the NP^{Min} segment and the remaining six wt plasmids were co-transfected. As a control, cells were transfected with all 8 wt A/PR8 plasmids. Cells transfected with either PB1^{Min-RR} or HA^{Min} segments produced viable virus as did the combination of the codon pair deoptimized segments HA^{Min} and NP^{Min}. The new de-optimized viruses are referred to as A/PR8-PB1^{Min-RR}, A/PR8-HA^{Min}, and A/PR8-HA^{Min}/NP^{Min}, respectively.

Growth characteristics and plaque phenotypes were assessed as described above.

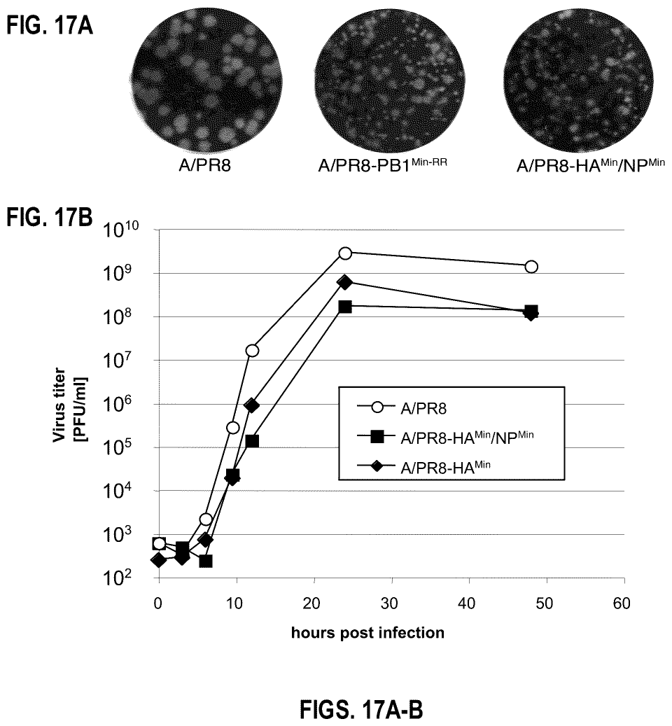

A/PR8-PB1^{Min-RR}, A/PR8-HA^{Min}, and A/PR8-HA^{Min}/NP^{Min} all produced viable virus. A/PR8-PB1^{Min-RR} and A/PR8-HA^{Min}/NP^{Min} produced smaller plaques on MDCK cells compared to the A/PR8 wt (FIG. 17A). Furthermore, upon low MOI infection on MDCK cells A/PR8-HA^{Min} and A/PR8-HA^{Min}/NP^{Min} display much reduced growth kinetics, especially from 3-12 hrs post infection, where A/PR8-HA^{Min}/NP^{Min} titers lag 1 to 2 orders of magnitude behind A/PR8 (FIG. 17B). Final titers for both A/PR8-HA^{Min} and A/PR8-HA^{Min}/NP^{Min} were 10 fold lower than that of A/PR8. As A/PR8-HA^{Min}/NP^{Min} is more severely growth retarded than A/PR8-HA^{Min}, it can be concluded that the effect of deoptimizing two segments is additive.

### Attenuation of A/PR8-NP^{Min} in a BALB/c Mouse Model

Groups of 6-8 anesthetized BALB/c mice 6 weeks of age were given 12.5 μl of A/PR8 or A/PR8-NP^{Min} virus solution to each nostril containing 10-fold serial dilutions between 10² and 10⁶ PFU of virus. Mortality and morbidity (weight loss, reduced activity, death) was monitored. The lethal dose 50, LD₅₀, was calculated by the method of Reed and Muench (Reed, L. J., and M. Muench. 1938. Am. J. Hyg. 27:493-497).

Eight mice were vaccinated once by intranasal inoculation with 10² PFU of A/PR8-NP^{Min} virus. A control group of 6 mice was not vaccinated with any virus (mock). 28 days following this initial vaccination the mice were challenged with a lethal dose of the wt virus A/PR8 corresponding to 100 times the LD50.

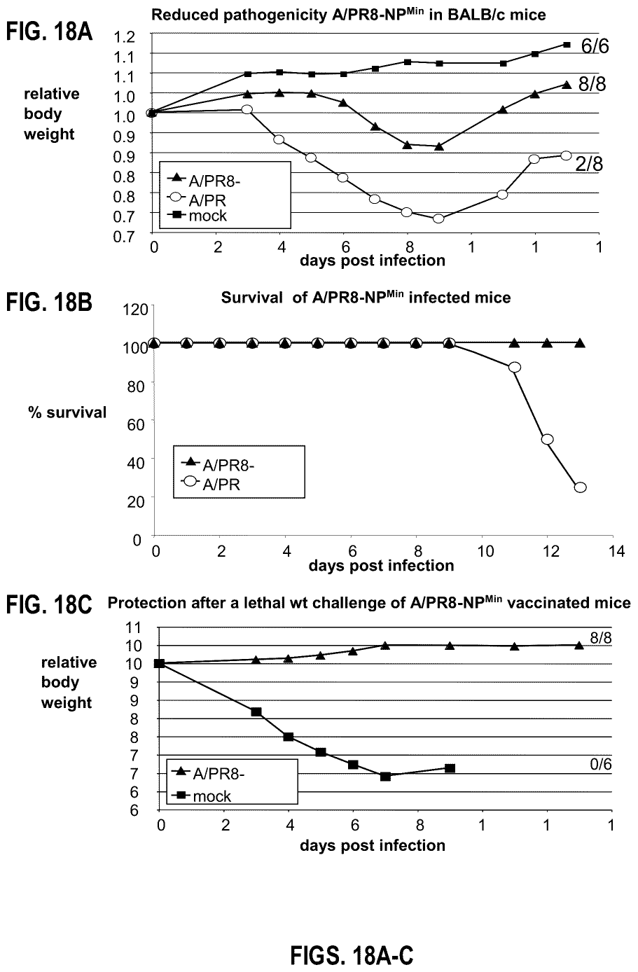

The LD50 for A/PR8 was 4.6×10¹ PFU while the LD50 for A/PR8-NP^{Min} was 1×10³ PFU. At a dose of 10² all A/PR8-NP^{Min} infected mice survived. It can be concluded that A/PR8-NP^{Min} is attenuated in mice by more than 10 fold compared to the wt A/PR8 virus. This concentration was thus chosen for vaccination experiments. Vaccination of mice with 10² A/PR8-NP^{Min} resulted in a mild and brief illness, as indicated by a relative weight loss of less than 10% (FIG. 18A). All 8 out of 8 vaccinated mice survived. Mice infected with A/PR8 at the same dose experienced rapid weight loss with severe disease. 6 of 8 mice infected with A/PR8 died between 10 and 13 days post infection (FIG. 18B). Two mice survived and recovered from the wildtype infection.

Upon challenge with 100 times LD50 of wt virus, all A/PR8-NP^{Min} vaccinated were protected, and survived the challenge without disease symptoms or weight loss (FIG. 18C). Mock vaccinated mice on the other hand showed severe symptoms, and succumbed to the infection between 9 and 11 days after challenge. It can be concluded that A/PR8-NP^{Min} induced protective immunity in mice and, thus, has potential as a live attenuated influenza vaccine. Other viruses such as A/PR8-PB1^{Min-RR} and A/PR8-HA^{Min}/NP^{Min}, yet to be tested in mice, may lead to improve further the beneficial properties of codon-pair deoptimized influenza viruses as vaccines.

### Example 11

### Development of Higher-Throughput Methods for Making and Characterizing Viral Chimeras

### Constructing Chimeric Viruses by Overlapping PCR

The “scan” through each attenuated mutant virus is performed by placing approximately 300-bp fragments from each mutant virus into a wt context using overlap PCR. Any given 300-bp segment overlaps the preceding segment by ˜200 bp, i.e., the scanning window is ˜300 bp long, but moves forward by ˜100 bp for each new chimeric virus. Thus, to scan through one mutant virus (where only the ˜3000 bp of the capsid region has been altered) requires about 30 chimeric viruses. The scan is performed in 96-well dish format which has more than sufficient capacity to analyze two viruses simultaneously.

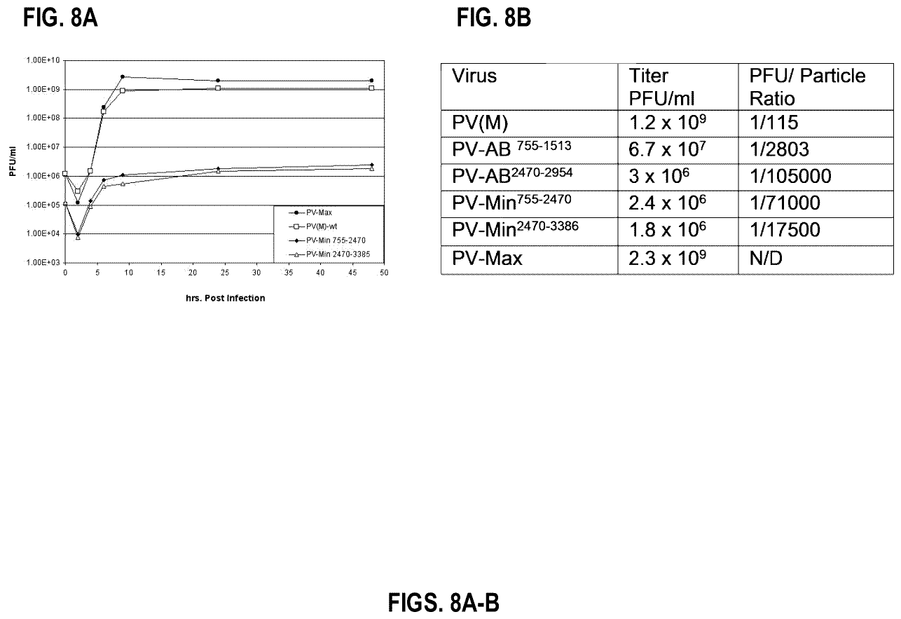

The starting material is picogram amounts of two plasmids, one containing the sequence of the wt virus, and the other the sequence of the mutant virus. The plasmids include all the necessary elements for the PV reverse genetics system (van der Werf et al., 1986), including the T7 RNA polymerase promoter, the hammerhead ribozyme (Herold and Aldino, 2000), and the DNA-encoded poly(A) tail. Three pairs of PCR primers are used, the A, M (for Mutant), and B pairs. See FIG. 9. The M pair amplifies the desired 300 bp segment of the mutant virus; it does not amplify wt, because the M primer pairs are designed based on sequences that have been significantly altered in the mutant. The A and B pairs amplify the desired flanks of the wt viral genome. Importantly, about 20-25 bp of overlap sequence is built into the 5′ ends of each M primer as well as A2 and B1, respectively; these 20-25 bps overlap (100% complementarity) with the 3′ end of the A segment and the 5′ end of the B segment, respectively.

To carry out the overlapping PCR, one 96-well dish contains wt plasmid DNA, and the 30 different A and B pairs in 30 different wells. A separate but matching 96-well plate contains mutant plasmid DNA and the 30 different M primer pairs. PCR is carried out with a highly processive, low error rate, heat-stable polymerase. After the first round of PCR, each reaction is treated with DpnI, which destroys the template plasmid by cutting at methylated GmATC sites. An aliquot from each wt and matching mutant reaction is then mixed in PCR reaction buffer in a third 96-well dish. This time, primers flanking the entire construct are used (i.e., the A1 and B2 primers). Since each segment (A, M, and B) is designed to overlap each adjacent segment by at least 20 bp, and since the reaction is being driven by primers that can only amplify a full-length product, the segments anneal and mutually extend, yielding full-length product after two or three cycles. This is a “3-tube” (three 96-well dish) design that may be compacted to a “1-tube” (one 96-well dish) design.

### Characterization of Chimeric Viruses

Upon incubation with T7 RNA polymerase, the full length linear chimeric DNA genomes produced above with all needed upstream and downstream regulatory elements yields active viral RNA, which produces viral particles upon incubation in HeLa S10 cell extract (Molla et al., 1991) or upon transfection into HeLa cells. Alternatively, it is possible to transfect the DNA constructs directly into HeLa cells expressing the T7 RNA polymerase in the cytoplasm.

The functionality of each chimeric virus is then assayed using a variety of relatively high-throughput assays, including visual inspection of the cells to assess virus-induced CPE in 96-well format; estimation of virus production using an ELISA; quantitative measurement of growth kinetics of equal amounts of viral particles inoculated into cells in a series of 96-well plates; and measurement of specific infectivity(infectious units/particle [IU/P] ratio).

The functionality of each chimeric virus can then be assayed. Numerous relatively high-throughput assays are available. A first assay may be to visually inspect the cells using a microscope to look for virus-induced CPE (cell death) in 96-well format. This can also be run an automated 96-well assay using a vital dye, but visual inspection of a 96-well plate for CPE requires less than an hour of hands-on time, which is fast enough for most purposes.

Second, 3 to 4 days after transfection, virus production may be assayed using the ELISA method described in Example 3. Alternatively, the particle titer is determined using sandwich ELISA with capsid-specific antibodies. These assays allow the identification of non-viable constructs (no viral particles), poorly replicating constructs (few particles), and efficiently replicating constructs (many particles), and quantification of these effects.

Third, for a more quantitative determination, equal amounts of viral particles as determined above are inoculated into a series of fresh 96-well plates for measuring growth kinetics. At various times (0, 2, 4, 6, 8, 12, 24, 48, 72 h after infection), one 96-well plate is removed and subjected to cycles of freeze-thawing to liberate cell-associated virus. The number of viral particles produced from each construct at each time is determined by ELISA as above.

Fourth, the IU/P ratio can be measured (see Example 3).

### Higher Resolution Scans

If the lethality of the viruses is due to many small defects spread through the capsid region, as the preliminary data indicate, then many or most of the chimeras are sick and only a few are non-viable. If this is the case, higher-resolution scans are probably not necessary. Conversely, if one or more of the 300 bp segments do cause lethality (as is possible for the codon-deoptimized virus in the segment between 1513 and 2470 which, as described below, may carry a translation frameshift signal that contribute to the strong phenotype of this segment), the genome scan is repeated at higher resolution, for instance a 30 bp window moving 10 bp between constructs over the 300-bp segment, followed by phenotypic analysis. A 30-bp scan does not involve PCR of the mutant virus; instead, the altered 30-bp segment is designed directly into PCR primers for the wt virus. This allows the changes responsible for lethality to be pinpointed.

### Example 12

Ongoing Investigations into the Molecular Mechanisms Underlying SAVE

### Choice of Chimeras

Two to four example chimeras from each of the two parental inviable viruses (i.e., 4 to 8 total viruses) are used in the following experiments. Viable chimeras having relatively small segments of mutant DNA, but having strong phenotypes are selected. For instance, viruses PV-AB⁷⁵⁵⁻¹⁵¹³, PVAB²⁴⁷⁰⁻²⁹⁵⁴ and PV-AB²⁹⁵⁴⁻³³⁸⁶ from the deoptimized codon virus (see Example 1), and PV-Min⁷⁵⁵⁻²⁴⁷⁰ and PV-Min²⁴⁷⁰⁻³³⁸⁶ (see Example 7), are suitable. Even better starting chimeras, with smaller inserts that will make analysis easier, may also be obtained from the experiments described above (Example 8).

### RNA Abundance/Stability

Conceivably the altered genome sequence destabilizes the viral RNA. Such destabilization could be a direct effect of the novel sequence, or an indirect effect of a pause in translation, or other defect in translation (see, e.g., Doma and Parker, 2006). The abundance of the mutant viral RNA is therefore examined. Equal amounts of RNA from chimeric mutant virus, and wt virus are mixed and transfected into HeLa cells. Samples are taken after 2, 4, 8, and 12 h, and analyzed by Northern blotting or quantitative PCR for the two different viral RNAs, which are easily distinguishable since there are hundreds of nucleotide differences. A control with wt viral RNA compared to PV-SD (the codon-shuffled virus with a wt phenotype) is also done. A reduced ratio of mutant to wt virus RNA indicates that the chimera has a destabilized RNA.

### In Vitro Translation

Translation was shown to be reduced for the codon-deoptimized virus and some of its derivatives. See Example 5. In vitro translation experiments are repeated with the codon pair-deoptimized virus (PV-Min) and its chosen chimeras. There is currently no good theory, much less any evidence, as to why deoptimized codon pairs should lead to viral inviability, and hence, investigating the effect on translation may help illuminate the underlying mechanism.

In vitro translations were performed in two kinds of extracts in Example 5. One was a “souped up” extract (Molla et al., 1991), in which even the codon-deoptimized viruses gave apparently good translation. The other was an extract more closely approximating normal in vivo conditions, in which the deoptimized-codon viruses were inefficiently translated. There were four differences between the extracts: the more “native” extract was not dialyzed; endogenous cellular mRNAs were not destroyed with micrococcal nuclease; the extract was not supplemented with exogenous amino acids; and the extract was not supplemented with exogenous tRNA. In the present study, these four parameters are altered one at a time (or in pairs, as necessary) to see which have the most significant effect on translation. For instance, a finding that it is the addition of amino acids and tRNA that allows translation of the codon-deoptimized virus strongly supports the hypothesis that translation is inefficient simply because rare aminoacyl-tRNAs are limiting. Such a finding is important from the point of view of extending the SAVE approach to other kinds of viruses.

### Translational Frameshifting

Another possible defect is that codon changes could promote translational frameshifting; that is, at some codon pairs, the ribosome could shift into a different reading frame, and then arrive at an in-frame stop codon after translating a spurious peptide sequence. This type frameshifting is an important regulatory event in some viruses. The present data reveal that all PV genomes carrying the AB mutant segment from residue 1513 to 2470 are non-viable. Furthermore, all genomes carrying this mutant region produce a novel protein band during in vitro translation of approximately 42-44 kDa (see FIG. 5A, marked by asterisk). This novel protein could be the result of a frameshift.

Examination of the sequence in the 1513-2470 interval reveals three potential candidate sites that conform to the slippery heptameric consensus sequence for −1 frameshifting in eukaryotes (X-XXY-YYZ) (Farabaugh, 1996). These sites are A-AAA-AAT at positions 1885 and 1948, and T-TTA-TTT at position 2119. They are followed by stop codons in the −1 frame at 1929, 1986 or 2149, respectively. The former two seem the more likely candidates to produce a band of the observed size.

To determine whether frameshifting is occurring, each of the three candidate regions is separately mutated so that it becomes unfavorable for frameshifting. Further, each of the candidate stop codons is separately mutated to a sense codon. These six new point mutants are tested by in vitro translation. Loss of the novel 42-44 kDa protein upon mutation of the frameshifting site to an unfavorable sequence, and an increase in molecular weight of that protein band upon elimination of the stop codon, indicate that frameshifting is occurring. If frameshifting is the cause of the aberrant translation product, the viability of the new mutant that lacks the frameshift site is tested in the context of the 1513-2470 mutant segment. Clearly such a finding would be of significance for future genome designs, and if necessary, a frameshift filter may be incorporated in the software algorithm to avoid potential frameshift sites.

More detailed investigations of translational defects are conducted using various techniques including, but not limited to, polysome profiling, toeprinting, and luciferase assays of fusion proteins.

### Polysome Profiling

Polysome profiling is a traditional method of examining translation. It is not high-throughput, but it is very well developed and understood. For polysome profiling, cell extracts are made in a way that arrests translation (with cycloheximide) and yet preserves the set of ribosomes that are in the act of translating their respective mRNAs (the “polysomes”). These polysomes are fractionated on a sucrose gradient, whereby messages associated with a larger number of ribosomes sediment towards the bottom. After fractionation of the gradient and analysis of RNA content using UV absorption, a polysome profile is seen where succeeding peaks of absorption correspond to mRNAs with N+1 ribosomes; typically 10 to 15 distinct peaks (representing the 40S ribosomal subunit, the 60S subunit, and 1, 2, 3, . . . 12, 13 ribosomes on a single mRNA) can be discerned before the peaks smudge together. The various fractions from the sucrose gradient are then run on a gel, blotted to a membrane, and analyzed by Northern analysis for particular mRNAs. This then shows whether that particular mRNA is primarily engaged with, say, 10 to 15 ribosomes (well translated), or 1 to 4 ribosomes (poorly translated).

In this study, for example, the wt virus, the PV-AB (codon deoptimized) virus, and its derivatives PV-AB⁷⁵⁵⁻¹⁵¹³, and PV-AB²⁹⁵⁴⁻³³⁸⁶, which have primarily N-terminal or C-terminal deoptimized segments, respectively, are compared. The comparison between the N-terminal and C-terminal mutant segments is particularly revealing. If codon deoptimization causes translation to be slow, or paused, then the N-terminal mutant RNA is associated with relatively few ribosomes (because the ribosomes move very slowly through the N-terminal region, preventing other ribosomes from loading, then zip through the rest of the message after traversing the deoptimized region). In contrast, the C-terminal mutant RNA are associated with a relatively large number of ribosomes, because many ribosomes are able to load, but because they are hindered near the C-terminus, they cannot get off the transcript, and the number of associated ribosomes is high.

Polysome analysis indicates how many ribosomes are actively associated with different kinds of mutant RNAs, and can, for instance, distinguish models where translation is slow from models where the ribosome actually falls off the RNA prematurely. Other kinds of models can also be tested.

### Toeprinting

Toeprinting is a technique for identifying positions on an mRNA where ribosomes are slow or paused. As in polysome profiling, actively translating mRNAs are obtained, with their ribosomes frozen with cycloheximide but still associated; the mRNAs are often obtained from an in vitro translation reaction. A DNA oligonucleotide primer complementary to some relatively 3′ portion of the mRNA is used, and then extended by reverse transcriptase. The reverse transcriptase extends until it collides with a ribosome. Thus, a population of translating mRNA molecules generates a population of DNA fragments extending from the site of the primer to the nearest ribosome. If there is a site or region where ribosomes tend to pause (say, 200 bases from the primer), then this site or region will give a disproportionate number of DNA fragments (in this case, fragments 200 bases long). This then shows up as a “toeprint” (a band, or dark area) on a high resolution gel. This is a standard method for mapping ribosome pause sites (to within a few nucleotides) on mRNAs.

Chimeras with segments of deoptimized codons or codon pairs, wherein in different chimeras the segments are shifted slightly 5′ or 3′, are analyzed. If the deoptimized segments cause ribosomes to slow or pause, the toeprint shifts 5′ or 3′ to match the position of the deoptimized segment. Controls include wt viral RNA and several (harmlessly) shuffled viral RNAs. Controls also include pure mutant viral RNA (i.e., not engaged in translation) to rule out ribosome-independent effects of the novel sequence on reverse transcription.

The toeprint assay has at least two advantages. First, it can provide direct evidence for a paused ribosome. Second, it has resolution of a few nucleotides, so it can identify exactly which deoptimized codons or deoptimized codon pairs are causing the pause. That is, it may be that only a few of the deoptimized codons or codon pairs are responsible for most of the effect, and toe-printing can reveal that.

### Dual Luciferase Reporter Assays of Fusion Proteins

The above experiments may suggest that certain codons or codon pairs are particularly detrimental for translation. As a high-throughput way to analyze effects of particular codons and codon pairs on translation, small test peptides are designed and fused to the N-terminus of sea pansy luciferase. Luciferase activity is then measured as an assay of the translatability of the peptide. That is, if the N-terminal peptide is translated poorly, little luciferase will be produced.

A series of eight 25-mer peptides are designed based on the experiments above. Each of the eight 25-mers is encoded 12 different ways, using various permutations of rare codons and/or rare codon pairs of interest. Using assembly PCR, these 96 constructs (8 25-mers×12 encodings) are fused to the N-terminus of firefly luciferase (F-luc) in a dicistronic, dual luciferase vector described above (see Example 5 and FIG. 6). A dual luciferase system uses both the firefly luciferase (F-Luc) and the sea pansy (Renilla) luciferase (R-Luc); these emit light under different biochemical conditions, and so can be separately assayed from a single tube or well. A dicistronic reporter is expressed as a single mRNA, but the control luciferase (R-Luc) is translated from one internal ribosome entry site (IRES), while the experimental luciferase (F-luc) (which has the test peptides fused to its N-terminus) is independently translated from its own IRES. Thus, the ratio of F-Luc activity to R-Luc activity is an indication of the translatability of the test peptide. See FIG. 6.

The resulting 96 dicistronic reporter constructs are transfected directly from the PCR reactions into 96 wells of HEK293 or HeLa cells. The firefly luciferase of the upstream cistron serves as an internal transfection control. Codon- or codon-pair-dependent expression of the sea pansy luciferase in the second cistron can be accurately determined as the ratio between R-Luc and F-Luc. This assay is high-throughput in nature, and hundreds or even thousands of test sequences can be assayed, as necessary.

### Example 13

### Design and Synthesis of Attenuated Viruses Using Novel Alternative-Codon Strategy

The SAVE approach to re-engineering viruses for vaccine production depends on large-scale synonymous codon substitution to reduce translation of viral proteins. This can be achieved by appropriately modulating the codon and codon pair bias, as well as other parameters such as RNA secondary structure and CpG content. Of the four de novo PV designs, two (the shuffled codon virus, PV-SD, and the favored codon pair virus, PV-Max) resulted in little phenotypic change over the wt virus. The other two de novo designs (PV-AB and PV-Min) succeeded in killing the virus employing only synonymous substitutions through two different mechanisms (drastic changes in codon bias and codon pair bias, respectively). The live-but-attenuated strains were constructed by subcloning regions from the inactivated virus strains into the wt.

A better understanding of the underlying mechanisms of viral attenuation employing large scale synonymous substitutions facilitates predictions of the phenotype and expression level of a synthetic virus. Ongoing studies address questions relating to the effect of the total number of alterations or the density of alterations on translation efficiency; the effect of the position of dense regions on translation; the interaction of codon and codon pair bias; and the effect of engineering large numbers of short-range RNA secondary structures into the genome. It is likely that there is a continuum between the wt and inactivated strains, and that any desired attenuation level can be engineered into a weakened strain. However, there may be hard limits on the attenuation level that can be achieved for any infection to be at self-sustaining and hence detectable. The 15⁴⁴² encodings of PV proteins constitutes a huge sequence space to explore, and various approaches are being utilized to explore this sequence space more systematically. These approaches include, first, developing a software platform to help design novel attenuated viruses, and second, using this software to design, and then synthesize and characterize, numerous new viruses that explore more of the sequence space, and answer specific questions about how alternative encodings cause attenuation. Additionally, an important issue to consider is whether dangerous viruses might accidentally be created by apparently harmless shuffling of synonymous codons.

### Development of Software for Computer-Based Design of Viral Genomes and Data Analysis

Designing synthetic viruses requires substantial software support for (1) optimizing codon and codon-pair usage and monitoring RNA secondary structure while preserving, embedding, or removing sequence specific signals, and (2) partitioning the sequence into oligonucleotides that ensure accurate sequence-assembly. The prototype synthetic genome design software tools are being expanded into a full environment for synthetic genome design. In this expanded software, the gene editor is conceptually built around constraints instead of sequences. The gene designer works on the level of specifying characteristics of the desired gene (e.g., amino acid sequence, codon/codon-pair distribution, distribution of restriction sites, and RNA secondary structure constraints), and the gene editor algorithmically designs a DNA sequence realizing these constraints. There are many constraints, often interacting with each other, including, but not limited to, amino acid sequence, codon bias, codon pair bias, CG dinucleotide content, RNA secondary structure, cis-acting nucleic acid signals such as the CRE, splice sites, polyadenylation sites, and restriction enzyme recognition sites. The gene designer recognizes the existence of these constraints, and designs genes with the desired features while automatically satisfying all constraints to a pre-specified level.

The synthesis algorithms previously developed for embedding/removing patterns, secondary structures, overlapping coding frames, and adhering to codon/codon-pair distributions are implemented as part of the editor, but more important are algorithms for realizing heterogeneous combinations of such preferences. Because such combinations lead to computationally intractable (NP-complete) problems, heuristic optimization necessarily plays an important role in the editor. Simulated annealing techniques are employed to realize such designs; this is particularly appropriate as simulated annealing achieved its first practical use in the early VLSI design tools.

The full-featured gene design programming environment is platform independent, running in Linux, Windows and MacOS. The system is designed to work with genomes on a bacterial or fungal (yeast) scale, and is validated through the synthesis and evaluation of the novel attenuated viral designs described below.

Virus Designs with Extreme Codon Bias in One or a Few Amino Acids

For a live vaccine, a virus should be as debilitated as possible, short of being inactivated, in which case there is no way to grow and manufacture the virus. One way of obtaining an optimally debilitated is to engineer the substitution of rare codons for just one or a few amino acids, and to create a corresponding cell line that overexpresses the rare tRNAs that bind to those rare codons. The virus is then able to grow efficiently in the special, permissive cell line, but is inviable in normal host cell lines. Virus is grown and manufactured using the permissive cell line, which is not only very convenient, but also safer than methods used currently used for producing live attenuated vaccines.

With the sequencing of the human genome, information regarding copy number of the various tRNA genes that read rare codons is available. Based on the literature (e.g., Lavner and Kotlar, 2005), the best rare codons for present purposes are CTA (Leu), a very rare codon which has just two copies of the cognate tRNA gene; TCG (Ser), a rare codon with four copies of the cognate tRNA gene; and CCG (Pro), a rare codon with four copies of the cognate tRNA gene (Lavner and Kotlar, 2005). The median number of copies for a tRNA gene of a particular type is 9, while the range is 2 to 33 copies (Lavner and Kotlar, 2005). Thus, the CTA codon is not just a rare codon, but is also the one codon with the fewest cognate tRNA genes. These codons are not read by any other tRNA; for instance, they are not read via wobble base pairing.

Changing all the codons throughout the virus genome coding for Leu (180 codons), Ser (153), and Pro (119) to the rare synonymous codons CTA, TCG, or CCG, respectively, is expected to create severely debilitated or even non-viable viruses. Helper cells that overexpress the corresponding rare tRNAs can then be created. The corresponding virus is absolutely dependent on growing only in this artificial culture system, providing the ultimate in safety for the generation of virus for vaccine production.

Four high-priority viruses are designed and synthesized: all Leu codons switched to CTA; all Ser codons switched to TCG; all Pro codons switched to CCG; and all Leu, Ser, and Pro codons switched to CTA, TCG, and CCG, respectively, in a single virus. In one embodiment, these substitutions are made only in the capsid region of the virus, where a high rate of translation is most important. In another embodiment, the substitutions are made throughout the virus.

### CG Dinucleotide Bias Viruses

With few exceptions, virus genomes under-represent the dinucleotide CpG, but not GpC (Karlin et al., 1994). This phenomenon is independent of the overall G+C content of the genome. CpG is usually methylated in the human genome, so that single-stranded DNA containing non-methylated CpG dinucleotides, as often present in bacteria and DNA viruses, are recognized as a pathogen signature by the Toll-like receptor 9. This leads to activation of the innate immune system. Although a similar system has not been shown to operate for RNA viruses, inspection of the PV genome suggests that PV has selected against synonymous codons containing CpG to an even greater extent than the significant under-representation of CpG dinucleotides in humans. This is particularly striking for arginine codons. Of the six synonymous Arg codons, the four CG containing codons (CGA, CGC CGG, CCU) together account for only 24 of all 96 Arg codons while the remaining two (AGA, AGG) account for 72. This in contrast to the average human codon usage, which would predict 65 CG containing codons and 31 AGA/AGO codons. In fact, two of the codons under-represented in PV are frequently used in human cells (CGC, CGG). There are two other hints that CG may be a disadvantageous dinucleotide in PV. First, in the codon pair-deoptimized virus, many of the introduced rare codon pairs contain CG as the central dinucleotide of the codon pair hexamer. Second, when Burns et al. (2006) passaged their codon bias-deoptimized virus and sequenced the genomes, it was observed that these viruses evolved to remove some CG dinucleotides.

Thus, in one high-priority redesigned virus, most or all Arg codons are changed to CGC or CGG (two frequent human codons). This does not negatively affect translation and allows an assessment of the effect of the CpG dinucleotide bias on virus growth. The increased C+G content of the resulting virus requires careful monitoring of secondary structure; that is, changes in Arg codons are not allowed to create pronounced secondary structures.

### Modulating Codon-Bias and Codon-Pair Bias Simultaneously.

Codon bias and codon-pair bias could conceivably interact with each other at the translational level. Understand this interaction may enable predictably regulation of the translatability of any given protein, possibly over an extreme range.

If we represent wild type polio codon bias and codon pair bias as 0, and the worst possible codon bias and codon pair bias as −1, then four high-priority viruses are the (−0.3, −0.3), (−0.3, −0.6), (−0.6, −0.3), and (−0.6, −0.6) viruses. These viruses reveal how moderately poor or very poor codon bias interacts with moderately poor or very poor codon pair virus. These viruses are compared to the wild type and also to the extreme PV-AB (−1, 0) and PV-Min (0, −1) designs.

### Modulating RNA Secondary Structure

The above synthetic designs guard against excessive secondary structures. Two additional designs systematically avoid secondary structures. These viruses are engineered to have wt codon and codon-pair bias with (1) provably minimal secondary structure, and (2) many small secondary structures sufficient to slow translation.

### Additional Viral Designs

Additional viral designs include full-genome codon bias and codon-pair bias designs; non-CG codon pair bias designs; reduced density rare codon designs; and viruses with about 150 rare codons, either spread through the capsid region, or grouped at the N-terminal end of the capsid, or grouped at the C-terminal end of the capsid.

### Example 14

### Testing the Potential for Accidentally Creating Viruses of Increased Virulence

It is theoretically possible that redesigning of viral genomes with the aim of attenuating these viruses could accidentally make a virus more virulent than the wt virus. Because protein sequences are not altered in the SAVE procedure, this outcome is unlikely. Nevertheless, it is desirable to experimentally demonstrate that the SAVE approach is benign.

Out of the possible 10⁴⁴² sequences that could possibly encode PV proteins, some reasonably fit version of PV likely arose at some point in the past, and evolved to a local optimum (as opposed to a global optimum). The creation of a new version of PV with the same protein coding capacity but a very different set of codons places this new virus in a different location on the global fitness landscape, which could conceivably be close to a different local optimum than wt PV. Conceivably, this new local optimum could be better than the wild type local optimum. Thus, it is just barely possible that shuffling synonymous codons might create a fitter virus.

To investigate this possibility, 13 PV genomes are redesigned and synthesized: one virus with the best possible codon bias; one virus with the best possible codon pair bias (i.e., PV-Max); one virus with the best possible codon and codon pair bias; and 10 additional viruses with wt codon and codon pair bias, but shuffled synonymous codons. Other parameters, such as secondary structure, C+G content, and CG dinucleotide content are held as closely as possible to wt levels.

These 13 viruses may each be in a very different location of the global fitness landscape from each other and from the wild type. But none of them is at a local optimum because they have not been subject to selection. The 13 viruses and the wt are passaged, and samples viruses are taken at the 1^{st}, 10^{th}, 20^{th}, and 50^{th} passages. Their fitness is compared to each other and to wt by assessing plaque size, plaque-forming units/ml in one-step growth curves, and numbers of particles formed per cell. See Example 1. Five examples of each of the 13 viruses are sequenced after the 10^{th}, 20^{th}, and 50^{th} passage. Select passage isolates are tested for pathogenicity in CD155tg mice, and LD₅₀'s are determined. These assays reveal whether any of the viruses are fitter than wt, and provide a quantitative measure of the risk of accidental production of especially virulent viruses. The 10 viruses with wt levels of codon and codon pair bias also provide information on the variability of the fitness landscape at the encoding level.

In view of the possibility that a fitter virus could emerge, and that a fitter virus may be a more dangerous virus, these experiments are conducted in a BSL3 laboratory. After the 10^{th} passage, phenotypes and sequences are evaluated and the susceptibility of emerging viruses to neutralization by PV-specific antibodies is verified. The experiment is stopped and reconsidered if any evidence of evolution towards a significantly fitter virus, or of systematic evolution towards new protein sequences that evade antibody neutralization, is obtained. Phenotypes and sequences are similarly evaluated after passage 20 before proceeding to passage 50. Because the synthetic viruses are created to encode exactly the same proteins as wt virus, the scope for increased virulence seems very limited, even if evolution towards (slightly) increased fitness is observed.

### Example 15

Extension of SAVE Approach to Virus Systems Other than Poliovirus

Notwithstanding the potential need for a new polio vaccine to combat the potential of reversion in the closing stages of the global effort at polio eradication, PV has been selected in the present studies primarily as a model system for developing SAVE. SAVE has, however, been developed with the expectation that this approach can be extended to other viruses where vaccines are needed. This extension of the SAVE strategy is herein exemplified by application to Rhinovirus, the causative agent of the common cold, and to influenza virus.

### Adaptation of SAVE to Human Rhinovirus—a Virus Closely Related to Poliovirus

Two model rhinoviruses, HRV2 and HRV14, were selected to test the SAVE approach for several reasons: (1) HRV2 and HRV14 represent two members of the two different genetic subgroups, A and B (Ledford et al., 2004); (2) these two model viruses use different receptors, LDL-receptor and ICAM-1, respectively (Greve et al., 1989; Hofer et al., 1994); both viruses as well as their infectious cDNA clones have been used extensively, and most applicable materials and methods have been established (Altmeyer et al., 1991; Gerber et al., 2001); and (4) much of the available molecular knowledge of rhinoviruses stems from studies of these two serotypes.

The most promising SAVE strategies developed through the PV experiments are applied to the genomes of HRV2 and HRV14. For example, codons, codon pairs, secondary structures, or combinations thereof, are deoptimized. Two to three genomes with varying degrees of attenuation are synthesized for each genotype. Care is taken not to alter the CRE, a critical RNA secondary structure of about 60 nucleotides (Gerber et al., 2001; Goodfellow et al., 2000; McKnight, 2003). This element is vital to the replication of picornaviruses and thus the structure itself must be maintained when redesigning genomes. The location of the CRE within the genome varies for different picornaviruses, but is known for most families (Gerber et al., 2001; Goodfellow et al., 2000; McKnight, 2003), and can be deduced by homology modeling for others where experimental evidence is lacking. In the case of HRV2 the CRE is located in the RNA sequence corresponding to the nonstructural protein 2A^{pro}; and the CRE of HRV14 is located in the VP1 capsid protein region (Gerber et al., 2001; McKnight, 2003).

The reverse genetics system to derive rhinoviruses from DNA genome equivalents is essentially the same as described above for PV, with the exception that transfections are done in HeLa-H1 cells at 34° C. in Hepes-buffered culture medium containing 3 mM Mg++ to stabilize the viral capsid. The resulting synthetic viruses are assayed in tissue culture to determine the PFU/IU ratio. See Example 3. Plaque size and kinetics in one-step growth curves are also assayed as described. See Example 2. Because the SAVE process can be applied relatively cheaply to all 100 or so relevant rhinoviruses, it is feasible to produce a safe and effective vaccine for the common cold using this approach.

### Adaptation of SAVE to Influenza a Virus—a Virus Unrelated to Poliovirus

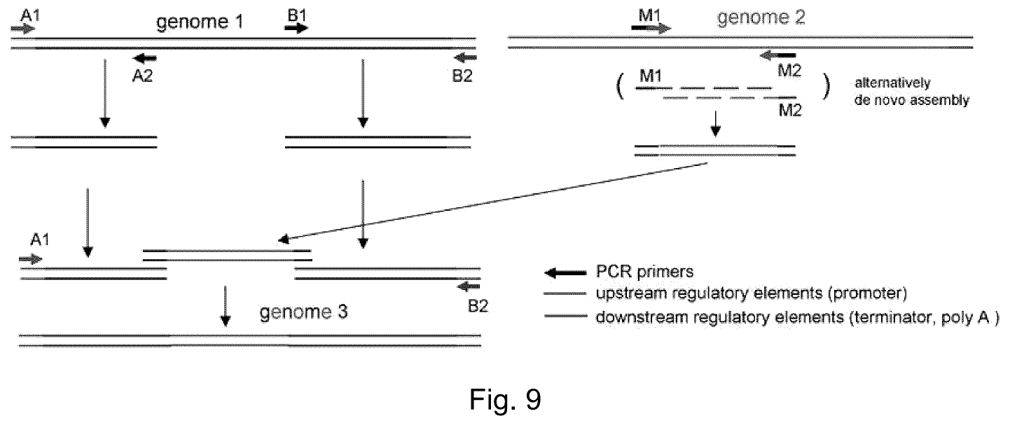

The most promising SAVE design criteria identified from PV experimentation are used to synthesize codon-deoptimized versions of influenza virus. The influenza virus is a “segmented” virus consisting of eight separate segments of RNA; each of these can be codon-modified. The well established ambisense plasmid reverse genetics system is used for generating variants of influenza virus strain A/PR/8/34. This eight-plasmid system is a variation of what has been described previously (Hoffmann et al., 2000), and has been kindly provided by Drs. P. Palese and A. Garcia-Sastre. Briefly, the eight genome segments of influenza each contained in a separate plasmid are flanked by a Pol I promoter at the 3′ end and Pol I terminator at the 5′ end on the antisense strand. This cassette in turn is flanked by a cytomegalovirus promoter (a Pol II promoter) at the 5′ end and a polyadenylation signal at the 3′ end on the forward strand (Hoffmann et al., 2000). Upon co-transfection into co-cultured 293T and MDCK cells, each ambisense expression cassette produces two kinds of RNA molecules. The Pol II transcription units on the forward strand produce all influenza mRNAs necessary for protein synthesis of viral proteins. The Pol I transcription unit on the reverse strand produces (−) sense genome RNA segments necessary for assembly of ribonucleoprotein complexes and encapsidation. Thus, infectious influenza A/PR/8/34 particles are formed (FIG. 10). This particular strain of the H1 N1 serotype is relatively benign to humans. It has been adapted for growth in tissue culture cells and is particularly useful for studying pathogenesis, as it is pathogenic in BALB/c mice.

When synthesizing segments that are alternatively spliced (NS and M), care is taken not to destroy splice sites and the alternative reading frames. In all cases the terminal 120 nt at either end of each segment are excluded, as these sequences are known to contain signals for RNA replication and virus assembly. At least two versions of each fragment are synthesized (moderate and maximal deoptimization). Viruses in which only one segment is modified are generated, the effect is assessed, and more modified segments are introduced as needed. This is easy in this system, since each segment is on a separate plasmid.

Virus infectivity is titered by plaque assay on MDCK cells in the presence of 1 ug/ml (TPCK)-trypsin. Alternatively, depending on the number of different virus constructs, a 96-well ELISA is used to determine the titer of various viruses as cell infectious units on MDCK cells essentially as described above for PV. See Example 3. The only difference is that now a HA-specific antibody is used to stain infected cells. In addition, the relative concentration of virions are determined via hemagglutination (HA) assay using chicken red blood cells (RBC) (Charles River Laboratories) using standard protocols (Kendal et al., 1982). Briefly, virus suspensions are 2-fold serially diluted in PBS in a V-bottom 96 well plates. PBS alone is used as an assay control. A standardized amount of RBCs is added to each well, and the plates are briefly agitated and incubated at room temperature for 30 minutes. HA titers are read as the reciprocal dilution of virus in the last well with complete hemagglutination. While HA-titer is a direct corollary of the amount of particles present, PFU-titer is a functional measure of infectivity. By determining both measures, a relative PFU/HA-unit ratio is calculated similar to the PFU/particle ratio described in the PV experiments. See Example 3. This addresses the question whether codon- and codon pair-deoptimized influenza viruses also display a lower PFU/particle as observed for PV.

### Virulence Test

The lethal dose 50 (LD₅₀) of the parental NPR/8/34 virus is first determined for mice and synthetic influenza viruses are chosen for infection of BALB/c mice by intranasal infection. Methods for determining LD₅₀ values are well known to persons of ordinary skill in the art (see Reed and Muench, 1938, and Example 4). The ideal candidate viruses display a low infectivity (low PFU titer) with a high virion concentration (high HA-titer). Anesthetized mice are administered 25 μl of virus solution in PBS to each nostril containing 10-fold serial dilutions between 10² to 10⁷ PFU of virus. Mortality and morbidity (weight loss, reduced activity) are monitored twice daily for up to three weeks. LD₅₀ is calculated by the method of Reed and Muench (1938). For the A/PR/8/34 wt virus the expected LD₅₀ is around 10³ PFU (Talon et al., 2000), but may vary depending on the particular laboratory conditions under which the virus is titered.

### Adaptation of SAVE to Dengue, HIV, Rotavirus, and SARS

Several viruses were selected to further test the SAVE approach. Table 8 identifies the coding regions of each of Dengue, HIV, Rotavirus (two segments), and SARS, and provides nucleotide sequences for parent viruses and exemplary viral genome sequences having deoptimized codon pair bias. As described above, codon pair bias is determined for a coding sequence, even though only a portion (subsequence) may contain the deoptimizing mutations.

TABLE 8
Nucleotide sequence and codon pair bias of parent
and codon pair bias-reduced coding regions

| | Parent sequence | | | Codon pair bias-reduced sequence | | |
|---|---|---|---|---|---|---|
| | SEQ ID | | | SEQ ID | deoptimized | |
| Virus | NO: | CDS | CPB | NO: | segment* | CPB* |
| Flu PB1 | 13 | 25-2298 | 0.0415 | 14 | 531-2143 | −0.2582 |
| Flu PB1-RR | ″ | ″ | ″ | 15 | 531-1488 | −0.1266 |
| Flu PB2 | 16 | 28-2307 | 0.0054 | 17 | 33-2301 | −0.3718 |
| Flu PA | 18 | 25-2175 | 0.0247 | 19 | 30-2171 | −0.3814 |
| Flu HA | 20 | 33-1730 | 0.0184 | 21 | 180-1655 | −0.3627 |
| Flu NP | 22 | 46-1542 | 0.0069 | 23 | 126-1425 | −0.3737 |
| Flu NA | 24 | 21-1385 | 0.0037 | 25 | 123-1292 | −0.3686 |
| Flu M | 26 | | 0.0024 | | | |
| Flu NS | 27 | 27-719 | −0.0036 | 28 | 128-479 | −0.1864 |
| Rhinovirus | 29 | 619-7113 | 0.051 | 30 | | −0.367 |
| 89 | | | | | | |
| Rhinovirus | 31 | 629-7168 | 0.046 | 32 | | −0.418 |
| 14 | | | | | | |
| Dengue | 33 | 95-10273 | 0.0314 | 34 | | −0.4835 |
| HIV | 35 | 336-1634 | 0.0656 | 36 | | −0.3544 |
| | | 1841-4585 | | | | |
| | | 4644-5102 | | | | |
| | | 5858-7924 | | | | |
| | | 8343-8963 | | | | |
| Rotavirus | 37 | 12-3284 | 0.0430 | 38 | | −0.2064 |
| Seg. 1 | | | | | | |
| Rotavirus | 39 | 37-2691 | 0.0375 | 40 | | −0.2208 |
| Seg. 2 | | | | | | |
| SARS | 41 | 265-13398 | 0.0286 | 42 | | −0.4393 |
| | | 13416-21485 | | | | |
| | | 21492-25259 | | | | |
| | | 26398-27063 | | | | |

*CPB can be reduced by deoptimizing an internal segment smaller than the complete coding sequence. Nevertheless, CPB is calculated for the complete CDS.

### Example 16

### Assessment of Poliovirus and Influenza Virus Vaccine Candidates in Mice

The ability of deoptimized viruses to vaccinate mice against polio or influenza is tested.

### Poliovirus Immunizations, Antibody Titers, and Wt Challenge Experiments

The working hypothesis is that a good vaccine candidate combines a low infectivity titer with a high virion titer. This ensures that a high amount of virus particles (i.e., antigen) can be injected while at the same time having a low risk profile. Thus, groups of five CD155tg mice will be injected intraperitoneally with 10³, 10⁴, 10⁵, and 10⁶ PFU of PV(Mahoney) (i.e., wild-type), PV1 Sabin vaccine strain, PVAB²⁴⁷⁰⁻²⁹⁵⁴, PV-Min⁷⁵⁵⁻²⁴⁷⁰, or other promising attenuated polioviruses developed during this study. For the wild-type, 1 PFU is about 100 viral particles, while for the attenuated viruses, 1 PFU is roughly 5,000 to 100,000 particles. Thus, injection with equal number of PFUs means that 50 to 1000-fold more particles of attenuated virus are being injected. For wt virus injected intraperitoneally, the LD₅₀ is about 10⁶ PFU, or about 10⁸ particles. Accordingly, some killing is expected with the highest doses but not with the lower doses.

Booster shots of the same dose are given one week after and four weeks after the initial inoculation. One week following the second booster, PV-neutralizing antibody titers are determined by plaque reduction assay. For this purpose, 100 PFU of wt PV(M) virus are incubated with 2-fold serial dilutions of sera from immunized mice. The residual number of PFU is determined by plaque assays. The neutralizing antibody titer is expressed as the reciprocal of the lowest serum dilution at which no plaques are observed.

Four weeks after the last booster, immunized mice and non-immunized controls are challenged with a lethal dose of PV(M) wt virus (10⁶ PFU intraperitoneally; this equals 100 times LD₅₀, and survival is monitored.

### Influenza Immunizations, Antibody Titers, and Wt Challenge Experiments

For vaccination experiments, groups of 5 BALB/c mice are injected with wt and attenuated influenza viruses intraperitoneally at a dose of 0.001, 0.01, 0.1, and 1.0 LD₅₀. Booster vaccinations are given at the same intervals described above for PV. Influenza antibody titers one week after the second booster are determined by an inhibition of hemagglutination (HI) assay following standard protocols (Kendal et al., 1982). Briefly, sera from immunized and control mice treated with receptor destroying enzyme (RDE; Sigma, St Louis, MO) are 2-fold serially diluted and mixed with 5 HA-units of A/PR/8/34 virus in V-bottom 96 wells. RBCs are then added and plates are processed as above for the standard HA-assay. Antibody titers are expressed as the reciprocal dilution that results in complete inhibition of hemagglutination.

Three weeks after the last booster vaccination, mice are challenged infra-nasally with 100 or 1000 LD₅₀ of A/PR/8/34 parental virus (approximately 10⁵ and 10⁶ PFU), and survival is monitored.

### Animal Handling

Transgenic mice expressing the human poliovirus receptor CD155 (CD155tg) were obtained from Dr. Nomoto, The Tokyo University. The CD155tg mouse colony is maintained by the State University of New York (SUNY) animal facility. BALB/c mice are obtained from Taconic (Germantown, NY). Anesthetized mice are inoculated using 25-gauge hypodermic needles with 30 μl of viral suspension by intravenous, intraperitoneal or intracerebral route or 50 ul by the intranasal route. Mice of both sexes between 6-24 weeks of age are used. Mice are the most economical model system for poliovirus and influenza virus research. In addition, in the case of PV, the CD155tg mouse line is the only animal model except for non-human primates. Mice also provide the safest animal model since no virus spread occurs between animals for both poliovirus and influenza virus.

All mice are housed in SUNY's state of the art animal facility under the auspices of the Department of Laboratory Animal Research (DLAR) and its veterinary staff. All animals are checked twice weekly by the veterinary staff. Virus-infected animals are checked twice daily by the investigators and daily by the veterinary staff. All infection experiments are carried out in specially designated maximum isolation rooms within the animal facility. After conclusion of an experiment, surviving mice are euthanized and cadavers are sterilized by autoclaving. No mouse leaves the virus room alive.

In the present study, mice are not subjected to any surgical procedure besides intravenous, intracerebral, intraperitoneal, intramuscular or intranasal inoculation, the injection of anesthetics, and the collection of blood samples. For vaccination experiments, blood samples are taken prior and after vaccination for detection of virus-specific antibodies. To this end, 50-100 μl are collected from mice the day before injection and one week following the second booster vaccination. A maximum of two blood samples on individual animals are collected at least four weeks apart. Animals are anesthetized and a sharp scalpel is used to cut off 2 mm of tail. Blood is collected with a capillary tube. Subsequent sampling is obtained by removing scab on the tail. If the tail is healed, a new 2-mm snip of tail is repeated.

All animal experiments are carried out following protocols approved by the SUNY Institutional Animal Care and Use Committee (IACUC). Euthanasia is performed by trained personnel in a CO₂ gas chamber according to the recommendation of the American Veterinary Medical Association. Infection experiments are conducted under the latest the ABSL 2/polio recommendations issued by the Centers for Disease Control and Prevention (CDC).

### Example 17

### Codon Pair Bias Algorithm—Codon Pair Bias and Score Matrix

In most organisms, there exists a distinct codon bias, which describes the preferences of amino acids being encoded by particular codons more often than others. It is widely believed that codon bias is connected to protein translation rates. In addition, each species has specific preferences as to whether a given pair of codons appear as neighbors in gene sequences, something that is called codon-pair bias.

To quantify codon pair bias, we define a codon pair distance as the log ratio of the observed over the expected number of occurrences (frequency) of codon pairs in the genes of an organism. Although the calculation of the observed frequency of codon pairs in a set of genes is straightforward, the expected frequency of a codon pair is calculated as in Gutman and Hatfield, Proc. Natl. Acad. Sci. USA, 86:3699-3703, 1989, and is independent of amino acid and codon bias. To achieve that, the expected frequency is calculated based on the relative proportion of the number of times an amino acid is encoded by a specific codon. In short:

$$codon pair score = log ( \frac{F ( AB )}{\frac{F ( A ) \times F ( B )}{F ( X ) \times F ( Y )} \times F ( XY )} ) ,$$

where the codon pair AB encodes for amino acid pair XY and F denotes frequency (number of occurrences).

In this scheme we can define a 64×64 codon-pair distance matrix with all the pairwise costs as defined above. Any m-residue protein can be rated as using over-or under-represented codon pairs by the average of the codon pair scores that comprise its encoding.

### Optimization of a Gene Encoding Based on Codon Pair Bias

To examine the effects of codon pair bias on the translation of specific proteins, we decided to change the codon pairs while keeping the same codon distribution. So we define the following problem: Given an amino acid sequence and a set of codon frequencies (codon distribution), change the DNA encoding of the sequence such that the codon pair score is optimized (usually minimized or maximized).

Our problem, as defined above, can be associated with the Traveling Salesman Problem (TSP). The traveling salesman problem is the most notorious NP-complete problem. This is a function of its general usefulness, and because it is easy to explain to the public at large. Imagine a traveling salesman who has to visit each of a given set of cities by car. What is the shortest route that will enable him to do so and return home, thus minimizing his total driving?

### TSP Heuristics

Almost any flavor of TSP is going to be NP-complete, so the right way to proceed is with heuristics. These are often quite successful, typically coming within a few percent of the optimal solution, which is close enough for most applications and in particular for our optimized encoding.

Minimum spanning trees—A simple and popular heuristic, especially when the sites represent points in the plane, is based on the minimum spanning tree of the points. By doing a depth-first search of this tree, we walk over each edge of the tree exactly twice, once going down when we discover the new vertex and once going up when we backtrack. We can then define a tour of the vertices according to the order in which they were discovered and use the shortest path between each neighboring pair of vertices in this order to connect them. This path must be a single edge if the graph is complete and obeys the triangle inequality, as with points in the plane. The resulting tour is always at most twice the length of the minimum TSP tour. In practice, it is usually better, typically 15% to 20% over optimal. Further, the time of the algorithm is bounded by that of computing the minimum spanning tree, only O(n lg n) in the case of points in the plane.

Incremental insertion methods—A different class of heuristics inserts new points into a partial tour one at a time (starting from a single vertex) until the tour is complete. The version of this heuristic that seems to work best is furthest point insertion: of all remaining points, insert the point v into partial tour T such that

$${max}_{v ∈ V} {min}_{i = 1}^{\vert T \vert} ( d ( {v}_{,} {v}_{i} ) + d ( v , {v}_{i + 1} ) ) .$$

The minimum ensures that we insert the vertex in the position that adds the smallest amount of distance to the tour, while the maximum ensures that we pick the worst such vertex first. This seems to work well because it first “roughs out” a partial tour before filling in details. Typically, such tours are only 5% to 10% longer than optimal.

k-optimal tours—Substantially more powerful are the Kernighan-Lin, or k-opt class of heuristics. Starting from an arbitrary tour, the method applies local refinements to the tour in the hopes of improving it. In particular, subsets of k≥2 edges are deleted from the tour and the k remaining subchains rewired in a different way to see if the resulting tour is an improvement. A tour is k-optimal when no subset of k edges can be deleted and rewired so as to reduce the cost of the tour. Extensive experiments suggest that 3 optimal tours are usually within a few percent of the cost of optimal tours. For k>3, the computation time increases considerably faster than solution quality. Two-opting a tour is a fast and effective way to improve any other heuristic. Simulated annealing provides an alternate mechanism to employ edge flips to improve heuristic tours.

### Algorithm for Solving the Optimum Encoding Problem

Our problem as defined is associated with the problem of finding a traveling salesman path (not tour) under a 64-country metric. In this formulation, each of the 64 possible codons is analogous to a country, and the codon multiplicity modeled as the number of cities in the country. The codon-pair bias measure is reflected as the country distance matrix.

The real biological problem of the design of genes encoding specific proteins using a given set of codon multiplicities so as to optimize the gene/DNA sequence under a codon-pair bias measure is slightly different. What is missing in our model in the country TSP model is the need to encode specific protein sequences. The DNA triplet code partitions the 64 codons into 21 equivalence classes (coding for each of the 20 possible amino acids and a stop symbol). Any given protein/amino acid sequence can be specified by picking an arbitrary representative of the associated codon equivalence class to encode it.

Because of the special restrictions and the nature of our problem, as well as its adaptability to application of additional criteria in the optimization, we selected the Simulated annealing heuristic to optimize sequences. The technique is summarized below.

### Simulated Annealing Heuristic

Simulated annealing is a heuristic search procedure that allows occasional transitions leading to more expensive (and hence inferior) solutions. This may not sound like a win, but it serves to help keep our search from getting stuck in local optima.

The inspiration for simulated annealing comes from the physical process of cooling molten materials down to the solid state. In thermodynamic theory, the energy state of a system is described by the energy state of each of the particles constituting it. The energy state of each particle jumps about randomly, with such transitions governed by the temperature of the system. In particular, the probability P(eᵢ, eⱼ, T) of transition from energy eᵢ to eⱼ at temperature T is given by:

$$P ( {e}_{i} , {e}_{j} , T ) = {e}^{( {e}_{i} - {e}_{j} ) / ( {k}_{B} T )}$$

where kB is a constant, called Boltzmann's constant. What does this formula mean? Consider the value of the exponent under different conditions. The probability of moving from a high-energy state to a lower-energy state is very high. However, there is also a nonzero probability of accepting a transition into a high-energy state, with small energy jumps much more likely than big ones. The higher the temperature, the more likely such energy jumps will occur.

What relevance does this have for combinatorial optimization? A physical system, as it cools, seeks to go to a minimum-energy state. For any discrete set of particles, minimizing the total energy is a combinatorial optimization problem. Through random transitions generated according to the above probability distribution, we can simulate the physics to solve arbitrary combinatorial optimization problems.

As with local search, the problem representation includes both a representation of the solution space and an appropriate and easily computable cost function C(s) measuring the quality of a given solution. The new component is the cooling schedule, whose parameters govern how likely we are to accept a bad transition as a function of time.

At the beginning of the search, we are eager to use randomness to explore the search space widely, so the probability of accepting a negative transition should be high. As the search progresses, we seek to limit transitions to local improvements and optimizations. The cooling schedule can be regulated by the following parameters:

Initial system temperature—Typically t₁=1.

Temperature decrement function—Typically tₖ=α·tk−1, where 0.8≤α≤0.99. This implies an exponential decay in the temperature, as opposed to a linear decay.

Number of iterations between temperature change—Typically, 100 to 1,000 iterations might be permitted before lowering the temperature.

Acceptance criteria—A typical criterion is to accept any transition from sᵢ to sᵢ+1 when C(sᵢ+1)<C(sᵢ) and to accept a negative transition whenever

$${e}^{- \frac{( C ( {s}_{i} ) - C ( {s}_{i + 1} ) )}{\text{?}}} \geq r ,$$

$$\text{?} \text{indicates text missing or illegible when filed}$$

where r is a random number 0≤r<1. The constant c normalizes this cost function, so that almost all transitions are accepted at the starting temperature.

Stop criteria—Typically, when the value of the current solution has not changed or improved within the last iteration or so, the search is terminated and the current solution reported.

In expert hands, the best problem-specific heuristics for TSP can slightly outperform simulated annealing, but the simulated annealing solution works easily and admirably.

### REFERENCES

Alexander, H. E., G. Koch, I. M. Mountain, K. Sprunt, and O. Van Damme. 1958. Infectivity of ribonucleic acid of poliovirus on HeLa cell mono-layers Virology. 5:172-3.
Altmeyer, R., A. D. Murdin, J. J. Harber, and E. Wimmer. 1991. Construction and characterization of poliovirus/rhinovirus antigenic hybrid. Virology. 184:636-44.
Ansardi, D. C., D. C. Porter, and C. D. Morrow. 1993. Complementation of a poliovirus defective genome by a recombinant vaccinia virus which provides poliovirus P1 capsid precursor in trans. J. Virol. 67:3684-3690.
Belov, G. A., L. I. Romanova, E. A. Tolskaya, M. S. Kolesnikova, Y. A. Lazebnik, and V. I. Agol. 2003. The major apoptotic pathway activated and suppressed by poliovirus. J. Virol. 77:45-56.
Buchan, J. R., L. S. Aucott, and I. Stansfield. 2006. tRNA properties help shape codon pair preferences in open reading frames. Nucl. Acids Res. 34:1015-27.
Burns, C. C., J. Shaw, R. Campagnoli, J. Jorba, A. Vincent, J. Quay, and O. Kew. 2006. Modulation of poliovirus replicative fitness in HeLa cells by deoptimization of synonymous codon usage in the capsii region. J. Virol. 80:3259-72.
Cao, X., R. J. Kuhn, and E. Wimmer. 1993. Replication of poliovirus RNA containing two VPg coding sequences leads to a specific deletion event. J. Virol. 67:5572-5578.
Carlini, D. B., and W. Stephan. 2003. In vivo introduction of unpreferred synonymous codons into the Drosophila Adh gene results in reduced levels of ADH protein. Genetics 163:239-243.
Cello, J., A. V. Paul, and E. Wimmer. 2002. Chemical synthesis of poliovirus cDNA: generation of infectious virus in the absence of natural template Science. 297:1016-1018.
Cheng, L., and E. Goldman. 2001. Absence of effect of varying Thr-Leu codon pairs on protein synthesis in a T7 system. Biochemistry. 40:6102-6.
Cohen, B., and S. Skiena. 2003. Natural selection and algorithmic design of mRNA. J. Comput Biol. 10:419-432.
Coligan, J., A. Kruisbeek, D. Margulies, E. Shevach, and W. Strober, eds. (1994) Current Protocols in Immunology, Wiley & Sons, Inc., New York.
Corpet, F. 1988. Multiple sequence alignment with hierarchical clustering. Nucl. Acids Res. 16:10881-90.
Cram, P., S. G. Blitz, A. Monte, and A. M. Fendrick. 2001. Influenza. Cost of illness and consideration in the economic evaluation of new and emerging therapies. Pharmacoeconomics. 19:223-30.
Crotty, S., C. E. Cameron, and R. Andino. 2001. RNA virus error catastrophe: direct molecular test by using ribavirin. Proc. Natl. Acad. Sci. U.S.A. 98:6895-6900.
Curran, J. F., E. S. Poole, W. P. Tate, and B. L. Gross. 1995. Selection of aminoacyl-tRNAs at sense codons: the size of the tRNA variable loop determines whether the immediate 3′ nucleotide to the coder has a context effect. Nucl. Acids Res. 23:4104-8.
Doma, M. K., and R. Parker. 2006. Endonucleolytic cleavage of eukaryotic mRNAs with stalls in translation elongation. Nature. 440:561-4.
Dove, A. W., and V. R. Racaniello. 1997. Cold-adapted poliovirus mutants bypass a postentry replication block. J. Virol. 71:4728-4735.
Enami, M., W. Luytjes, M. Krystal, and P. Palese. 1990. Introduction of site-specific mutations into the genome of influenza virus. Proc. Natl. Acad. Sci. U.S.A. 87:3802-5.
Farabaugh, P. J. 1996. Programmed translational frameshifting Microbiol Rev. 60:103-34.
Fedorov, A., S. Saxonov, and W. Gilbert. 2002. Regularities of context-dependent codon bias in eukaryotic genes. Nucl. Acids Res. 30:1192-7.
Fodor, E., L. Devenish, O. G. Engelhardt, P. Palese, G. G. Brownlee, and A. Garcia-Sastre. 1999. Rescue of influenza A virus from recombinant DNA. J Virol. 73:9679-82.
Gabow, H. 1973. Ph.D. thesis. Stanford University, Stanford, Calif.
Garcia-Sastre, A., and P. Palese. 1993. Genetic manipulation of negative-strand RNA virus genomes. Annu. Rev. Microbiol. 47:765-90.
Georgescu, M. M., J. Balanant, A. Macadam, D. Otelea, M. Combiescu, A. A. Combiescu, R. Crainic, and F. Delpeyroux. 1997. Evolution of the Sabin type 1 poliovirus in humans: characterization of strains isolated from patients with vaccine-associated paralytic poliomyelitis. J. Virol. 71:7758-68.
Gerber, K., E. Wimmer, and A. V. Paul. 2001. Biochemical and genetic studies of the initiation of human rhinovirus 2 RNA replication: identification of a cis-replicating element in the coding sequence of 2A(pro). J. Virol. 75:10979-10990.
Girard, S., T. Couderc, J. Destombes, D. Thiesson, F. Delpeyroux, and B. Blondel. 1999. Poliovirus induces apoptosis in the mouse central nervous system. J. Virol. 73:6066-6072.
Goodfellow, I., Y. Chaudhry, A. Richardson, J. Meredith, J. W. Almond, W. Barclay, and D. J. Evans. 2000. Identification of a cis-acting replication element within the poliovirus coding region. J. Virol. 74:4590-600.
Greve, J. M., G. Davis, A. M. Meyer, C. P. Forte, S. C. Yost, C. W. Marlor, M. E. Kamarck, and A. McClelland. 1989. The major human rhinovirus receptor is ICAM-1. Cell. 56:839-47.
Gustafsson, C., S. Govindarajan, and J. Minshull. 2004. Codon bias and heterologous protein expression. Trends Biotechnol. 22:346-353.
Gutman, G. A., and G. W. Hatfield. 1989. Nonrandom utilization of codon pairs in Escherichia coli. Proc. Natl. Acad. Sci. U.S.A 86:3699-703.
He, Y., V. D. Bowman, S. Mueller, C. M. Bator, J. Bella, X. Peng, T. S. Baker, E. Wimmer, R. J. Kuhn, and M. G. Rossmann. 2000. Interaction of the poliovirus receptor with poliovirus. Proc. Natl. Acad. Sci. USA 97:79-84.
Hendley, J. O. 1999. Clinical virology of rhinoviruses Adv Virus Res. 54:453-66.
Herold, J., and R. Andino. 2000. Poliovirus requires a precise 5′ end for efficient positive-strand RNA synthesis. J. Virol. 74:6394-400.
Hoekema, A., R. A. Kastelein, M. Vasser, and H. A. de Boer. 1987. Codon replacement in the PGK1 gene of Saccharomyces cerevisiae: experimental approach to study the role of biased codon usage in gene expression. Mol. Cell. Biol. 7:2914-2924.
Hofer, F., M. Gruenberger, H. Kowalski, H. Machat, M. Huettinger, E. Kuechler, and D. Blaas. 1994 Members of the low density lipoprotein receptor family mediate cell entry of a minor-group common cold virus. Proc. Natl. Acad. Sci. U.S.A. 91:1839-42.
Hoffmann, E., G. Neumann, Y. Kawaoka, G. Hobom, and R. G. Webster. 2000. A DNA transfection system for generation of influenza: A virus from eight plasmids. Proc. Natl. Acad. Sci. U.S.A. 97:6108-13.
Hogle, J. M. 2002. Poliovirus cell entry: common structural themes in viral cell entry pathways. Annu. Rev. Microbiol. 56:677-702.
Holland, J. J., E. Domingo, J. C. de la Torre, and D. A. Steinhauer. 1990. Mutation frequencies at defined single codon sites in vesicular stomatitis virus and poliovirus can be increased only slightly by chemical mutagenesis. J. Virol. 64:3960-3962.
Hsiao, L. L., F. Dangond, T. Yoshida, R. Hong, R. V. Jensenet al. 2001. A compendium of gene expression in normal human tissues. Physiol. Genomics 7:97-104.
Irwin, B., J. D. Heck, and G. W. Hatfield. 1995. Codon pair utilization biases influence translational elongation step times. J. Biol Chem. 270:22801-6.
Jang, S. K., M. V. Davies, R. J. Kaufman, and E. Wimmer. 1989. Initiation of protein synthesis by internal entry of ribosomes into the 5′ nontranslated region of encephalomyocarditis virus RNA in vitro. J. Virol. 63:1651-1660.
Jayaraj, S., R. Reid, and D. V. Santi. 2005. GeMS: an advanced software package for designing synthetic genes. Nucl. Acids Res. 33:3011-3016.
Johansen, L. K., and C. D. Morrow. 2000. The RNA encompassing the internal ribosome entry site in the poliovirus 5′ nontranslated region enhances the encapsidation of genomic RNA. Virology 273:391-399.
Joklik, W., and J. Darnell. 1961. The adsorption and early fate of purified poliovirus in HeLa cells. Virology 13:439-447.
Kamps, B. S., C. Hoffmann, and W. Preiser (eds.) 2006. Influenza Report, 2006. Flying Publisher.
Kaplan, G., and V. R. Racaniello. 1988. Construction and characterization of poliovirus subgenomic replicons. J. Virol. 62:1687-96.
Karlin, S., W. Doerfler, and L. R. Cardon. 1994. Why is CpG suppressed in the genomes of virtually al small eukaryotic viruses but not in those of large eukaryotic viruses?J Virol. 68:2889-97.
Kendal, A. P., J. J. Skehel, and M. S. Pereira (eds.) 1982 Concepts and procedures for laboratory-based influenza surveillance. World Health Organization Collaborating Centers for Reference and Research on Influenza, Geneva.
Kew, O., V. Morris-Glasgow, M. Landaverde, C. Burns, J. Shaw, Z. Garib, J. Andre, E. Blackman, C. J. Freeman, J. Jorba, R. Sutter, G. Tambini, L. Venczel, C. Pedreira, F. Laender, H. Shimizu, T. Yoneyama, T. Miyamura, H. van Der Avoort, M. S. Oberste, D. Kilpatrick, S. Cochi, M. Pallansch, and C. de Quadros. 2002. Outbreak of poliomyelitis in Hispaniola associated with circulating type 1 vaccine-derived poliovirus. Science. 296:356-9.
Kilbourne, E. D. 2006. Influenza pandemics of the 20^{th} century. Emerg. Infect. Dis. 12:9-14.
Kitamura, N., B. L. Semler, P. G. Rothberg, G. R. Larsen, C. J. Adler, A. J. Dorner, E. A. Emini, R. Hanecak, J. Lee, S. van der Well, C. W. Anderson, and E. Wimmer. 1981. Primary structure, gene organization and polypeptide expression of poliovirus RNA. Nature. 291:547-553.
Koike, S., C. Taya, T. Kurata, S. Abe, I. Ise, H. Yonekawa, and A. Nomoto. 1991. Transgenic mice susceptible to poliovirus. Proc. Natl. Acad. Sci. U.S.A. 88:951-955.
Landsteiner, K. and E. Popper. 1909. Ubertragung der Poliomyelitis acuta auf Affen. Z. ImmunnitatsForsch Orig. 2:377-90.
Lavner, Y., and D. Kotlar. 2005. Codon bias as a factor in regulating expression via translation rate in the human genome. Gene. 345:127-38.
Ledford, R. M., N. R. Patel, T. M. Demenczuk, A. Watanyar, T. Herbertz, M. S. Collett, and D. C. Pevear. 2004. VP1 sequencing of all human rhinovirus serotypes: insights into genus phylogeny and susceptibility to antiviral capsid-binding compounds. J. Virol. 78:3663-74.
Luytjes, W., M. Krystal, M. Enami, J. D. Pavin, and P. Palese. 1989. Amplification, expression, and packaging of foreign gene by influenza virus. Cell. 59:1107-13.
McKnight, K. L. 2003. The human rhinovirus internal cis-acting replication element (cre) exhibits disparate properties among serotypes. Arch. Virol. 148:2397-418.
Molla, A., A. V. Paul, and E. Wimmer. 1991. Cell-free, de novo synthesis of poliovirus. Science 254:1647-1651.
Mueller, S., D. Papamichail, J. R. Coleman, S. Skiena, and E. Wimmer. 2006. Reduction of the Rate of Poliovirus Protein Synthesis through Large-Scale Codon Deoptimization Causes Attenuation of Viral Virulence by lowering specific infectivity. J. Virol. 80:9687-9696.
Mueller, S., E. Wimmer, and J. Cello. 2005. Poliovirus and poliomyelitis: a tale of guts, brains, and an accidental event. Virus Res. 111:175-193.
Murdin, A. D., and E. Wimmer. 1989. Construction of a poliovirus type 1/type 2 antigenic hybrid by manipulation of neutralization antigenic site II. J. Virol. 63:5251-5257.
Neumann, G., T. Watanabe, H. Ito, S. Watanabe, H. Goto, P. Gao, M. Hughes, D. R. Perez, R. Donis E. Hoffmann, G. Hobom, and Y. Kawaoka. 1999. Generation of influenza A viruses entirely from clone cDNAs. Proc. Natl. Acad. Sci. U.S.A. 96:9345-50.
Neznanov, N., K. M. Chumakov, L. Neznanova, A. Almasan, A. K. Banerjee, and A. V. Gudkov. 2005. Proteolytic cleavage of the p65-RelA subunit of NF-kappaB during poliovirus infection. J. Biol. Chem. 280:24153-24158.
Palese, P., and M. L. Shaw. 2007. Orthomyxoviridae: the viruses and their replication, p. 1647-1689. In D. M. Knipe and P. M. Howley (ed.), Fields virology. Lippincott Williams & Wilkins, Philadelphia, PA.
Park, S., X. Yang, and J. G. Saven. 2004. Advances in computational protein design. Curr Opin Struct Biol 14:487-94.
Paul, A. V., J. A. Mugavero, A. Molla, and E. Wimmer. 1998. Internal ribosomal entry site scanning of the poliovirus polyprotein: implications for proteolytic processing. Virology 250:241-253.
Pelletier, J., and N. Sonenberg. 1988. Internal initiation of translation of eukaryotic mRNA directed by; sequence derived from poliovirus RNA. Nature. 334:320-325.
Pfister, T., and E. Wimmer. 1999. Characterization of the nucleoside triphosphatase activity of poliovirus protein 2C reveals a mechanism by which guanidine inhibits poliovirus replication. J. Biol. Chem. 274:6992-7001.
Plotkin, J. B., H. Robins, and A. J. Levine. 2004. Tissue-specific codon usage and the expression of human genes. Proc. Natl. Acad. Sci. U.S.A. 101:12588-12591.
Racaniello, V. R., and D. Baltimore. 1981. Cloned poliovirus complementary DNA is infectious in mammalian cells. Science. 214:916-9.
Reed, L. J., and M. Muench. 1938. A simple method for estimating fifty percent endpoints. Am. J. Hyg. 27:493-497.
Richardson, S. M., S. J. Wheelan, R. M. Yarrington, and J. D. Boeke. 2006. GeneDesign: rapid, automated design of multikilobase synthetic genes. Genome Res. 16:550-556.
Robinson, M., R. Lilley, S. Little, J. S. Emtage, G. Yarronton, P. Stephens, A. Millican, M. Eaton, and G. Humphreys. 1984. Codon usage can affect efficiency of translation of genes in Escherichia coli. Nucl. Acids Res. 12:6663-6671.
Rothberg, E. 1985. wmatch: a C program to solve maximum-weight matching. [Online.]
Rueckert, R. R. 1985. Picornaviruses and their replication, p. 705-738. In B. N. Fields, D. M. Knipe, R. M. Chanock, J. L. Melnick, B. Roizman, and R. E. Shope (ed.), Fields virology, vol. 1. Raven Press, New York, N.Y.
Russell, C. J., and R. G. Webster. 2005. The genesis of a pandemic influenza virus. Cell. 123:368-371.
Sambrook, J., E. F. Fritsch, and T. Maniatis. (1989) Molecular Cloning: A Laboratory Manual, 2^{nd} ed., Cold Spring Harbor Laboratory Press, Cold Spring Harbor, NY.
Sänchez, G., A. Bosch, and R. M. Pinto. 2003. Genome variability and capsid structural constraints of hepatitis A virus. J. Virol. 77:452-459.
Savolainen, C., S. Blomqvist, and T. Novi. 2003. Human rhinoviruses. Paediatr. Respir. Rev. 4:91-98.
Schwerdt, C., and J. Fogh. 1957. The ratio of physical particles per infectious unit observed for poliomyelitis viruses. Virology 4:41-52.
Shimizu, H., B. Thorley, F. J. Paladin, K. A. Brussen, and V. Stambos et al. 2004. Circulation of type 1 vaccine-derived poliovirus in the Philippines in 2001. J. Virol. 78:13512-13521.
Simonsen, L., T. A. Reichert, C. Viboud, W. C. Blackwelder, R. J. Taylor, and M. A. Miller. 2005. Impact of influenza vaccination on seasonal mortality in the US elderly population. Arch. Intern. Med. 165:265-272.
Skiena, S. S. 2001. Designing better phages Bioinformatics. 17 Suppl 1:5253-61.
Steinhauer, D. A., and J. J. Skehel. 2002. Genetics of influenza viruses. Annu. Rev. Genet. 36:305-332.
Stephenson, I., and J. Democratis. 2006. Influenza: current threat from avian influenza. Br. Med. Bull. 75-76:63-80.
Svitkin, Y. V., G. A. Alpatova, G. A. Lipskaya, S. V. Maslova, V. I. Agol, O. Kew, K. Meerovitch, and N. Sonenberg. 1993. Towards development of an in vitro translation test for poliovirus neurovirulence. Dev. Biol. Stand. 78:27-32.
Svitkin, Y. V., S. V. Maslova, and V. I. Agol. 1985. The genomes of attenuated and virulent poliovirus strains differ in their in vitro translation efficiencies. Virology 147:243-252.
Talon, J., M. Salvatore, R. E. O'Neill, Y. Nakaya, H. Zheng, T. Muster, A. Garcia-Sastre, and P. Palese. 2000. Influenza A and B viruses expressing altered NS1 proteins: A vaccine approach. Proc. Natl. Acad. Sci. U.S.A. 97:4309-4314.
Thompson, W. W., D. K. Shay, E. Weintraub, L. Brammer, N. Cox, L. J. Anderson, and K. Fukuda. 2003. Mortality associated with influenza and respiratory syncytial virus in the United States. JAMA. 289:179-186.
Tian, J., H. Gong, N. Shang, X. Zhou, E. Gulari, X. Gao, and G. Church. 2004. Accurate multiplex gene synthesis from programmable DNA microchips. Nature. 432:1050-1054.
Tolskaya, E. A., L. I. Romanova, M. S. Kolesnikova, T. A. Ivannikova, E. A. Smirnova, N. T. Raikhlin, and V. I. Agol. 1995. Apoptosis-inducing and apoptosis-preventing functions of poliovirus. J. Virol. 69:1181-1189.
Toyoda, H., J. Yin, S. Mueller, E. Wimmer, and J. Cello. 2007. Oncolytic treatment and cure of neuroblastoma by a novel attenuated poliovirus in a novel poliovirus-susceptible animal model. Cancer Res. 67:2857-64.
van der Wert, S., J. Bradley, E. Wimmer, F. W. Studier, and J. J. Dunn. 1986. Synthesis of infectious poliovirus RNA by purified T7 RNA polymerase. Proc. Natl. Acad. Sci. U.S.A. 78:2330-2334.
Wahby, A. F. 2000. Combined cell culture enzyme-linked immunosorbent assay for quantification of poliovirus neutralization—relevant antibodies. Clin. Diagn. Lab. Immunol. 7:915-9.
Wang, B., D. Papamichail, S. Mueller, and S. Skiena. 2006. Two Proteins for the Price of One: The Design of Maximally Compressed Coding Sequences Natural Computing. Eleventh International Meeting on DNA Based Computers (DNA11), 2005. Lecture Notes in Computer Science (LNCS), 3892:387-398.
Zhao, W. D., and E. Wimmer. 2001. Genetic analysis of a poliovirus/hepatitis C virus chimera: new structure for domain II of the internal ribosomal entry site of hepatitis C virus. J. Virol. 75:3719-3730.
Zhou, J., W. J. Liu, S. W. Peng, X. Y. Sun, and I. Frazer. 1999. Papillomavirus capsid protein expression level depends on the match between codon usage and tRNA availability. J. Virol. 73:4972-4982.
Zolotukhin, S., M. Potter, W. W. Hauswirth, J. Guy, and N. Muzyczka. 1996. A “humanized” green fluorescent protein cDNA adapted for high-level expression in mammalian cells. J. Virol. 70:4646-4654.

1.-81. (canceled)

82. A modified virus comprising a viral genome comprising a virus protein-encoding sequence, wherein the protein-encoding sequence has a codon pair bias (CPB) less than −0.1; and the codon pair bias is calculated relative to an viral host.

83. The modified virus of claim 82, wherein the protein-encoding sequence has a CPB less than −0.15.

84. The modified virus of claim 82, wherein the protein-encoding sequence has a CPB less than −0.2.

85. The modified virus of claim 82, wherein the protein-encoding sequence has a CPB less than −0.3.

86. The modified virus of claim 82, wherein the protein-encoding sequence has a CPB less than −0.5.

87. The modified virus of claim 82, wherein the viral host is a mammal or a bird.

88. The modified virus of claim 87, wherein the viral host is a human.

89. The modified virus of claim 82, wherein the viral host is a domesticated poultry, a domesticated life stock, or an exotic zoo animal.

90. The modified virus of claim 89, wherein the domesticated life stock is a cow, a pig, a sheep, or a horse.

91. The modified virus of claim 82, wherein the virus is a DNA, RNA, double-stranded, or single-stranded virus.

92. The modified virus of claim 82, wherein the virus is a picornavirus, orthomyxovirus, flavivirus, retrovirus, reovirus, coronavirus, filovirus, herpesvirus, poxvirus, adenovirus, or papillomavirus.

93. The modified virus of claim 92, wherein the picornavirus is a poliovirus or rhinovirus.

94. The modified virus of claim 93, wherein the poliovirus is derived from poliovirus type 1 (Mahoney), poliovirus type 2 (Lansing), poliovirus type 3 (Leon), monovalent oral poliovirus vaccine (OPV) virus, or trivalent OPV virus.

95. The modified virus of claim 92, wherein the orthomyxovirus is an influenza virus.

96. The modified virus of claim 92, wherein the flavivirus is a Dengue Fever virus, hepatitis C virus, West Nile Disease virus, or Yellow Fever virus.

97. The modified virus of claim 92, wherein the retrovirus is a Human Immunodeficiency Virus.

98. The modified virus of claim 92, wherein the reovirus is a rotavirus.

99. The modified virus of claim 92, wherein the coronavirus is a Severe Acute Respiratory Syndrome (SARS) virus or an infectious bronchitis virus.

100. The modified virus of claim 92, wherein the filovirus is an Ebolavirus or Marburg virus.

101. The modified virus of claim 92, wherein the herpesvirus is an Epstein-Barr virus.

102. A vaccine composition for inducing a protective immune response in a subject in need thereof, the vaccine composition comprising the modified virus of claim 82 and a pharmaceutically acceptable carrier.

103. A method of eliciting a protective immune response in a subject in need thereof, the method comprising administering to the subject the vaccine composition of claim 102.

104. The method of claim 103, wherein the subject has been exposed to a pathogenic virus.

105. A method for preventing a subject in need thereof from becoming afflicted with a virus-associated disease, the method comprising administering to the subject the vaccine composition of claim 102.

106. The method of claim 105, wherein the subject has been exposed to a pathogenic virus.

107. A method for delaying the onset of, or slowing the rate of progression of, a virus-associated disease in a virus-infected subject in need thereof, the method comprising administering to the subject the vaccine composition of claim 102.

108. The method of claim 107, the method further comprising administering to the subject at least one adjuvant.
